(19) United States
(12) Patent Application Publication
(10) Pub. No.: US 2025/0277022 A1
(43) Pub. Date: Sep. 4, 2025

(54) ANTIBODIES SPECIFICALLY RECOGNIZING THYMIC STROMAL LYMPHOPOIETIN AND USES THEREOF

(71) Applicant: STAIDSON (BEIJING) BIOPHARMACEUTICALS CO., LTD., Beijing (CN)

(72) Inventors: Debin Yu, Beijing (CN); Chao Zhang, Beijing (CN)

(21) Appl. No.: 18/275,953

(22) PCT Filed: Jan. 27, 2022
(86) PCT No.: PCT/CN2022/074230

§ 371 Date: Aug. 4, 2023

(30) Foreign Application Priority Data
Feb. 4, 2021 (WO) PCT/CN2021/075182

(51) Int. Cl. C07K 16/24; A61K 39/00; A61P 37/08

(52) U.S. Cl. CPC C07K 16/244; A61P 37/08; A61K 2039/505; C07K 2317/14; C07K 2317/24; C07K 2317/565; C07K 2317/622; C07K 2317/76; C07K 2317/92

(57) ABSTRACT

The present application provides antibodies including antigen-binding fragment thereof that specifically recognizing thymic stromal lymphopoietin (TSLP). Also provided are methods of making and using these antibodies.

### SUBMISSION OF SEQUENCE LISTING ON ASCII TEXT FILE

The content of the following submission on ASCII text file is incorporated herein by reference in its entirety: a computer readable form (CRF) of the Sequence Listing (file name: 202102022277_SEQLIST.txt, date recorded: Feb. 2, 2021, size: 72 KB).

### FIELD OF THE APPLICATION

The present application pertains to antibodies that specifically recognize thymic stromal lymphopoietin (TSLP), pharmaceutical compositions and kits comprising said TSLP antibodies, and methods of manufacture and uses thereof, including methods of treating diseases associated TSLP signaling, such as inflammatory diseases.

### BACKGROUND OF THE APPLICATION

Full length thymic stromal lymphopoietin (TSLP) was originally identified as a factor in supernatants from mouse thymic stromal cells which could induce the proliferation of pre-B cells (Friend, et al., Exp Hematol. 22(3):321, 1994). The murine protein was later identified (Sims. et al., J Exp Med. 2000 Sep. 4; 192(5):671-80), closely followed by identification of human TSLP in 2001 by two separate groups (Quentmeier, et al., Leukemia. 2001 August; 15(8):1286-92, Reche, et al., J Immunol. 2001 Jul. 1; 167(1):336-43).

Thymic stromal lymphopoietin (TSLP) is a cytokine that signals through a heterodimeric receptor consisting of the IL-7Rα subunit and TSLP-R, a unique component with homology to the common γ-receptor-like chain (Pandey et al., Nat. Immunol. 2000, 1(1):59-64). TSLP produced by epithelial cells at barrier surfaces activates TSLP receptor (TSLPR)-expressing DCs to induce functional Th2 cells (Roan F et al., J Clin Invest 2019; 129:1441-51). In addition, TSLP is known to be associated with other diseases, including autoimmune disorders and cancer (Varricchi G et al., Front Immunol 2018; 9:1595). TSLP receptor (TSLPR) complex consists of TSLPR and IL-7 receptor alpha (IL-7Rα)(Park L S, et al., J Exp Med 2000; 192:659-670). A combination of TSLPR and IL-7R chain results in high affinity binding and activation of signal transducer and activator of transcription 3 (STAT3) and STAT5 (Pandey A, et al., Nat Immunol 2000; 1:59-64). Such a broad pathophysiological profile has motivated the therapeutic targeting of TSLP- and TSLPR-mediated signaling in type 2 cytokine mediated allergic diseases. AMG157 is a fully human antibody against TSLP, under development by Amgen for the treatment of asthma and atopic dermatitis (used as a control in the Examples), is described in U.S. Pat. No. 7,982,016.

The disclosures of all publications, patents, patent applications and published patent applications referred to herein are hereby incorporated herein by reference in their entirety.

### BRIEF SUMMARY OF THE APPLICATION

In one aspect, the present application provides an isolated anti-TSLP antibody that specifically binds to human and/or cynomolgus monkey TSLP. In some embodiments, the isolated anti-TSLP antibody comprises: a heavy chain variable domain (V_{H}) comprising a heavy chain complementarity determining region (HC-CDR) 1 comprising SGYGWS (SEQ ID NO: 1); an HC-CDR2 comprising YX₁SYYGSX₂SYNPSLKS (SEQ ID NO: 103), wherein X₁ is I or F, and X₂ is I or T; and an HC-CDR3 comprising TNLLYFX₁X₂ (SEQ ID NO: 104), wherein X₁ is D or E, and X₂ is S or Y; and a light chain variable domain (V_{L}) comprising a light chain complementarity determining region (LC-CDR) 1 comprising RASQX₁X₂SX₃X₄LA (SEQ ID NO: 105), wherein X₁ is G or S, X₂ is V or I, X₃ is N or S, and X₄ is N or Y; a LC-CDR2 comprising DX₁SX₂X₃X₄X₅(SEQ ID NO: 106), wherein X₁ is A or T, X₂ is S or N, X₃ is R or L, X₄ is A or Q, and X₅ is T or S; and a LC-CDR3 comprising QQYSX₁WPX₂YX₃ (SEQ ID NO: 107), wherein X₁ is D or N, X₂ is Q or E, and X₃ is T or S.

In some embodiments, there is provided an isolated anti-TSLP antibody comprising: a V_{H} comprising an HC-CDR1 comprising the amino acid sequence of SEQ ID NO: 1, or a variant thereof comprising up to about 3 amino acid substitutions; an HC-CDR2 comprising the amino acid sequence of any one of SEQ ID NOs: 7-9, or a variant thereof comprising up to about 3 amino acid substitutions; and an HC-CDR3 comprising the amino acid sequence of any one of SEQ ID NOs: 16-18, or a variant thereof comprising up to about 3 amino acid substitutions; and a V_{L} comprising a LC-CDR1 comprising the amino acid sequence of any one of SEQ ID NOs: 25-28, or a variant thereof comprising up to about 3 amino acid substitutions; a LC-CDR2 comprising the amino acid sequence of any one of SEQ ID NOs: 39-41, or a variant thereof comprising up to about 3 amino acid substitutions; and a LC-CDR3 comprising the amino acid sequence of any one of SEQ ID NOs: 50-53, or a variant thereof comprising up to about 3 amino acid substitutions.

In some embodiments, there is provided an isolated anti-TSLP antibody comprising a V_{H} comprising an HC-CDR1, an HC-CDR2, and an HC-CDR3 of a V_{H} comprising the amino acid sequence of any one of SEQ ID NOs: 65-72; and a V_{L} comprising a LC-CDR1, a LC-CDR2, and a LC-CDR3 of a V_{L} comprising the amino acid sequence of any one of SEQ ID NOs: 84-90.

In some embodiments, there is provided an isolated anti-TSLP antibody comprising: (i) a V_{H} comprising an HC-CDR1, an HC-CDR2, and an HC-CDR3 of a V_{H} comprising the amino acid sequence of SEQ ID NO: 65; and a V_{L} comprising a LC-CDR1, a LC-CDR2, and a LC-CDR3 of a V_{L} comprising the amino acid sequence of SEQ ID NO: 84; (ii) a V_{H} comprising an HC-CDR1, an HC-CDR2, and an HC-CDR3 of a V_{H} comprising the amino acid sequence of SEQ ID NO: 65; and a V_{L} comprising a LC-CDR1, a LC-CDR2, and a LC-CDR3 of a V_{L} comprising the amino acid sequence of SEQ ID NO: 85; (iii) a V_{H} comprising an HC-CDR1, an HC-CDR2, and an HC-CDR3 of a V_{H} comprising the amino acid sequence of SEQ ID NO: 66; and a V_{L} comprising a LC-CDR1, a LC-CDR2, and a LC-CDR3 of a V_{L} comprising the amino acid sequence of SEQ ID NO: 84; (iv) a V_{H} comprising an HC-CDR1, an HC-CDR2, and an HC-CDR3 of a V_{H} comprising the amino acid sequence of SEQ ID NO: 66; and a V_{L} comprising a LC-CDR1, a LC-CDR2, and a LC-CDR3 of a V_{L} comprising the amino acid sequence of SEQ ID NO: 85; (v) a V_{H} comprising an HC-CDR1, an HC-CDR2, and an HC-CDR3 of a V_{H} comprising the amino acid sequence of SEQ ID NO: 67; and a V_{L} comprising a LC-CDR1, a LC-CDR2, and a LC-CDR3 of a V_{L} comprising the amino acid sequence of SEQ ID NO: 84; (vi) a V_{H} comprising an HC-CDR1, an HC-CDR2, and an HC-CDR3 of a V_{H} comprising the amino acid sequence of SEQ ID NO: 67; and a V_{L} comprising a LC-CDR1, a LC-CDR2, and a LC-CDR3 of a V_{L} comprising the amino acid sequence of SEQ ID NO: 85; (vii) a V_{H} comprising an HC-CDR1, an HC-CDR2, and an HC-CDR3 of a V_{H} comprising the amino acid sequence of SEQ ID NO: 68; and a V_{L} comprising a LC-CDR1, a LC-CDR2, and a LC-CDR3 of a V_{L} comprising the amino acid sequence of SEQ ID NO: 84; (viii) a V_{H} comprising an HC-CDR1, an HC-CDR2, and an HC-CDR3 of a V_{H} comprising the amino acid sequence of SEQ ID NO: 68; and a V_{L} comprising a LC-CDR1, a LC-CDR2, and a LC-CDR3 of a V_{L} comprising the amino acid sequence of SEQ ID NO: 85; (ix) a V_{H} comprising an HC-CDR1, an HC-CDR2, and an HC-CDR3 of a V_{H} comprising the amino acid sequence of SEQ ID NO: 69; and a V_{L} comprising a LC-CDR1, a LC-CDR2, and a LC-CDR3 of a V_{L} comprising the amino acid sequence of SEQ ID NO: 86; (x) a V_{H} comprising an HC-CDR1, an HC-CDR2, and an HC-CDR3 of a V_{H} comprising the amino acid sequence of SEQ ID NO: 70; and a V_{L} comprising a LC-CDR1, a LC-CDR2, and a LC-CDR3 of a V_{L} comprising the amino acid sequence of SEQ ID NO: 87; (xi) a V_{H} comprising an HC-CDR1, an HC-CDR2, and an HC-CDR3 of a V_{H} comprising the amino acid sequence of SEQ ID NO: 69; and a V_{L} comprising a LC-CDR1, a LC-CDR2, and a LC-CDR3 of a V_{L} comprising the amino acid sequence of SEQ ID NO: 88; (xii) a V_{H} comprising an HC-CDR1, an HC-CDR2, and an HC-CDR3 of a V_{H} comprising the amino acid sequence of SEQ ID NO: 71; and a V_{L} comprising a LC-CDR1, a LC-CDR2, and a LC-CDR3 of a V_{L} comprising the amino acid sequence of SEQ ID NO: 89; or (xiii) a V_{H} comprising an HC-CDR1, an HC-CDR2, and an HC-CDR3 of a V_{H} comprising the amino acid sequence of SEQ ID NO: 72; and a V_{L} comprising a LC-CDR1, a LC-CDR2, and a LC-CDR3 of a V_{L} comprising the amino acid sequence of SEQ ID NO: 90.

In some embodiments, there is provided an isolated anti-TSLP antibody comprising: (i) a V_{H} comprising an HC-CDR1 comprising the amino acid sequence of SEQ ID NO: 1, an HC-CDR2 comprising the amino acid sequence of SEQ ID NO: 7, and an HC-CDR3 comprising the amino acid sequence of SEQ ID NO: 16, or a variant thereof comprising up to about 5 amino acid substitutions in the HC-CDRs; and a V_{L} comprising a LC-CDR1 comprising the amino acid sequence of SEQ ID NO: 25, a LC-CDR2 comprising the amino acid sequence of SEQ ID NO: 39, and a LC-CDR3 comprising the amino acid sequence of SEQ ID NO: 50, or a variant thereof comprising up to about 5 amino acid substitutions in the LC-CDRs; (ii) a V_{H} comprising an HC-CDR1 comprising the amino acid sequence of SEQ ID NO: 1, an HC-CDR2 comprising the amino acid sequence of SEQ ID NO: 8, and an HC-CDR3 comprising the amino acid sequence of SEQ ID NO: 16, or a variant thereof comprising up to about 5 amino acid substitutions in the HC-CDRs; and a V_{L} comprising a LC-CDR1 comprising the amino acid sequence of SEQ ID NO: 25, a LC-CDR2 comprising the amino acid sequence of SEQ ID NO: 39, and a LC-CDR3 comprising the amino acid sequence of SEQ ID NO: 51, or a variant thereof comprising up to about 5 amino acid substitutions in the LC-CDRs; (iii) a V_{H} comprising an HC-CDR1 comprising the amino acid sequence of SEQ ID NO: 1, an HC-CDR2 comprising the amino acid sequence of SEQ ID NO: 8, and an HC-CDR3 comprising the amino acid sequence of SEQ ID NO: 17, or a variant thereof comprising up to about 5 amino acid substitutions in the HC-CDRs; and a V_{L} comprising a LC-CDR1 comprising the amino acid sequence of SEQ ID NO: 26, a LC-CDR2 comprising the amino acid sequence of SEQ ID NO: 40, and a LC-CDR3 comprising the amino acid sequence of SEQ ID NO: 52, or a variant thereof comprising up to about 5 amino acid substitutions in the LC-CDRs; (iv) a V_{H} comprising an HC-CDR1 comprising the amino acid sequence of SEQ ID NO: 1, an HC-CDR2 comprising the amino acid sequence of SEQ ID NO: 8, and an HC-CDR3 comprising the amino acid sequence of SEQ ID NO: 16, or a variant thereof comprising up to about 5 amino acid substitutions in the HC-CDRs; and a V_{L} comprising a LC-CDR1 comprising the amino acid sequence of SEQ ID NO: 27, a LC-CDR2 comprising the amino acid sequence of SEQ ID NO: 41, and a LC-CDR3 comprising the amino acid sequence of SEQ ID NO: 52, or a variant thereof comprising up to about 5 amino acid substitutions in the LC-CDRs; (v) a V_{H} comprising an HC-CDR1 comprising the amino acid sequence of SEQ ID NO: 1, an HC-CDR2 comprising the amino acid sequence of SEQ ID NO: 9, and an HC-CDR3 comprising the amino acid sequence of SEQ ID NO: 16, or a variant thereof comprising up to about 5 amino acid substitutions in the HC-CDRs; and a V_{L} comprising a LC-CDR1 comprising the amino acid sequence of SEQ ID NO: 28, a LC-CDR2 comprising the amino acid sequence of SEQ ID NO: 39, and a LC-CDR3 comprising the amino acid sequence of SEQ ID NO: 52, or a variant thereof comprising up to about 5 amino acid substitutions in the LC-CDRs; or (vi) a V_{H} comprising an HC-CDR1 comprising the amino acid sequence of SEQ ID NO: 1, an HC-CDR2 comprising the amino acid sequence of SEQ ID NO: 8, and an HC-CDR3 comprising the amino acid sequence of SEQ ID NO: 18, or a variant thereof comprising up to about 5 amino acid substitutions in the HC-CDRs; and a V_{L} comprising a LC-CDR1 comprising the amino acid sequence of SEQ ID NO: 27, a LC-CDR2 comprising the amino acid sequence of SEQ ID NO: 39, and a LC-CDR3 comprising the amino acid sequence of SEQ ID NO: 53, or a variant thereof comprising up to about 5 amino acid substitutions in the LC-CDRs.

In some embodiments, according to any one of the isolated anti-TSLP antibodies described above, the isolated anti-TSLP antibody comprises: a V_{H} comprising the amino acid sequence of any one of SEQ ID NOs: 65-72, or a variant thereof having at least about 90% (for example at least about any of 91%, 92%, 93%, 94%, 95%, 96%, 97%, 98%, or 99%) sequence identity to the amino acid sequence of any one of SEQ ID NOs: 65-72; and a V_{L} comprising the amino acid sequence of any one of SEQ ID NOs: 84-90, or a variant thereof having at least about 90% (for example at least about any of 91%, 92%, 93%, 94%, 95%, 96%, 97%, 98%, or 99%) sequence identity to the amino acid sequence of any one of SEQ ID NOs:84-90. In some embodiments, the isolated anti-TSLP antibody comprises: (i) a V_{H} comprising the amino acid sequence of SEQ ID NO: 65; and a V_{L} comprising the amino acid sequence of SEQ ID NO: 84; (ii) a V_{H} comprising the amino acid sequence of SEQ ID NO: 65; and a V_{L} comprising the amino acid sequence of SEQ ID NO: 85; (iii) a V_{H} comprising the amino acid sequence of SEQ ID NO: 66; and a V_{L} comprising the amino acid sequence of SEQ ID NO: 84; (iv) a V_{H} comprising the amino acid sequence of SEQ ID NO: 66; and a V_{L}comprising the amino acid sequence of SEQ ID NO: 85; (v) a V_{H} comprising the amino acid sequence of SEQ ID NO: 67; and a V_{L} comprising the amino acid sequence of SEQ ID NO: 84; (vi) a V_{H} comprising the amino acid sequence of SEQ ID NO: 67; and a V_{L} comprising the amino acid sequence of SEQ ID NO: 85; (vii) a V_{H} comprising the amino acid sequence of SEQ ID NO: 68; and a V_{L} comprising the amino acid sequence of SEQ ID NO: 84; (viii) a V_{H} comprising the amino acid sequence of SEQ ID NO: 68; and a V_{L} comprising the amino acid sequence of SEQ ID NO: 85; (ix) a V_{H} comprising the amino acid sequence of SEQ ID NO: 69; and a V_{L} comprising the amino acid sequence of SEQ ID NO: 86; (x) a V_{H} comprising the amino acid sequence of SEQ ID NO: 70; and a V_{L} comprising the amino acid sequence of SEQ ID NO: 87; (xi) a V_{H} comprising the amino acid sequence of SEQ ID NO: 69; and a V_{L} comprising the amino acid sequence of SEQ ID NO: 88; (xii) a V_{H} comprising the amino acid sequence of SEQ ID NO: 71; and a V_{L} comprising the amino acid sequence of SEQ ID NO: 89; or (xiii) a V_{H} comprising the amino acid sequence of SEQ ID NO: 72; and a V_{L} comprising the amino acid sequence of SEQ ID NO: 90.

In one aspect, the present application provides an isolated anti-TSLP antibody that specifically binds on human and/or cynomolgus monkey TSLP, the isolated anti-TSLP antibody comprises: a heavy chain variable domain (V_{H}) comprising a heavy chain complementarity determining region (HC-CDR) 1 comprising SYGIX₁ (SEQ ID NO: 108), wherein X₁ is N or S; an HC-CDR2 comprising VIX₁PLX₂X₃VX₄X₅YAEKFQG (SEQ ID NO: 109), wherein X₁ is V or I, X₂ is V or L, X₃ is G or D, X₄ is T or P, and X₅ is I or N; and an HC-CDR3 comprising GX₁EYFYWYFDL (SEQ ID NO: 110), wherein X₁ is Q or A; and a light chain variable domain (V_{L}) comprising a light chain complementarity determining region (LC-CDR) 1 comprising X₁GX₂X₃SDIGGYX₄RVS (SEQ ID NO: 111), wherein X₁ is S or T, X₂ is S or T, X₃ is S, T, I or N, and X₄ is N or D; a LC-CDR2 comprising X₁X₂X₃KRX₄S (SEQ ID NO: 112), wherein X₁ is D, G or E, X₂ is V, F or I, X₃ is S or N, and X₄ is P or S; and a LC-CDR3 comprising X₁SYAX₂X₃X₄X₅FX₆X₇(SEQ ID NO: 113), wherein X₁ is S or T, X₂ is G or S, X₃ is T or G, X₄ is D or H, X₅ is T or I, X₆ is I, G, V or A, and X₇ is L, I or F.

In some embodiments, there is provided an isolated anti-TSLP antibody comprising: a V_{H} comprising an HC-CDR1 comprising the amino acid sequence of any one of SEQ ID NOs: 2-3, or a variant thereof comprising up to about 3 amino acid substitutions; an HC-CDR2 comprising the amino acid sequence of any one of SEQ ID NOs: 10-12, or a variant thereof comprising up to about 3 amino acid substitutions; and an HC-CDR3 comprising the amino acid sequence of any one of SEQ ID NOs: 19-20, or a variant thereof comprising up to about 3 amino acid substitutions; and a V_{L} comprising a LC-CDR1 comprising the amino acid sequence of any one of SEQ ID NOs: 29-34, or a variant thereof comprising up to about 3 amino acid substitutions; a LC-CDR2 comprising the amino acid sequence of any one of SEQ ID NOs: 42-47, or a variant thereof comprising up to about 3 amino acid substitutions; and a LC-CDR3 comprising the amino acid sequence of any one of SEQ ID NOs: 54-60, or a variant thereof comprising up to about 3 amino acid substitutions.

In some embodiments, there is provided an isolated anti-TSLP antibody comprising a V_{H} comprising an HC-CDR1, an HC-CDR2, and an HC-CDR3 of a V_{H} comprising the amino acid sequence of any one of SEQ ID NOs: 73-78; and a V_{L} comprising a LC-CDR1, a LC-CDR2, and a LC-CDR3 of a V_{L} comprising the amino acid sequence of any one of SEQ ID NOs: 91-97.

In some embodiments, there is provided an isolated anti-TSLP antibody comprising: (i) a V_{H} comprising an HC-CDR1, an HC-CDR2, and an HC-CDR3 of a V_{H} comprising the amino acid sequence of SEQ ID NO: 73; and a V_{L} comprising a LC-CDR1, a LC-CDR2, and a LC-CDR3 of a V_{L} comprising the amino acid sequence of SEQ ID NO: 91; (ii) a V_{H} comprising an HC-CDR1, an HC-CDR2, and an HC-CDR3 of a V_{H} comprising the amino acid sequence of SEQ ID NO: 74; and a V_{L} comprising a LC-CDR1, a LC-CDR2, and a LC-CDR3 of a V_{L} comprising the amino acid sequence of SEQ ID NO: 91; (iii) a V_{H} comprising an HC-CDR1, an HC-CDR2, and an HC-CDR3 of a V_{H} comprising the amino acid sequence of SEQ ID NO: 75; and a V_{L} comprising a LC-CDR1, a LC-CDR2, and a LC-CDR3 of a V_{L} comprising the amino acid sequence of SEQ ID NO: 91; (iv) a V_{H} comprising an HC-CDR1, an HC-CDR2, and an HC-CDR3 of a V_{H} comprising the amino acid sequence of SEQ ID NO: 73; and a V_{L} comprising a LC-CDR1, a LC-CDR2, and a LC-CDR3 of a V_{L} comprising the amino acid sequence of SEQ ID NO: 92; (v) a V_{H} comprising an HC-CDR1, an HC-CDR2, and an HC-CDR3 of a V_{H} comprising the amino acid sequence of SEQ ID NO: 73; and a V_{L} comprising a LC-CDR1, a LC-CDR2, and a LC-CDR3 of a V_{L} comprising the amino acid sequence of SEQ ID NO: 93; (vi) a V_{H} comprising an HC-CDR1, an HC-CDR2, and an HC-CDR3 of a V_{H} comprising the amino acid sequence of SEQ ID NO: 73; and a V_{L} comprising a LC-CDR1, a LC-CDR2, and a LC-CDR3 of a V_{L} comprising the amino acid sequence of SEQ ID NO: 94; (vii) a V_{H} comprising an HC-CDR1, an HC-CDR2, and an HC-CDR3 of a V_{H} comprising the amino acid sequence of SEQ ID NO: 76; and a V_{L} comprising a LC-CDR1, a LC-CDR2, and a LC-CDR3 of a V_{L} comprising the amino acid sequence of SEQ ID NO: 95; (viii) a V_{H} comprising an HC-CDR1, an HC-CDR2, and an HC-CDR3 of a V_{H} comprising the amino acid sequence of SEQ ID NO: 77; and a V_{L} comprising a LC-CDR1, a LC-CDR2, and a LC-CDR3 of a V_{L} comprising the amino acid sequence of SEQ ID NO: 96; or (ix) a V_{H} comprising an HC-CDR1, an HC-CDR2, and an HC-CDR3 of a V_{H} comprising the amino acid sequence of SEQ ID NO: 78; and a V_{L} comprising a LC-CDR1, a LC-CDR2, and a LC-CDR3 of a V_{L} comprising the amino acid sequence of SEQ ID NO: 97.

In some embodiments, there is provided an isolated anti-TSLP antibody comprising: (i) a V_{H} comprising an HC-CDR1 comprising the amino acid sequence of SEQ ID NO: 2, an HC-CDR2 comprising the amino acid sequence of SEQ ID NO: 10, and an HC-CDR3 comprising the amino acid sequence of SEQ ID NO: 19, or a variant thereof comprising up to about 5 amino acid substitutions in the HC-CDRs; and a V_{L} comprising a LC-CDR1 comprising the amino acid sequence of SEQ ID NO: 29, a LC-CDR2 comprising the amino acid sequence of SEQ ID NO: 42, and a LC-CDR3 comprising the amino acid sequence of SEQ ID NO: 54, or a variant thereof comprising up to about 5 amino acid substitutions in the LC-CDRs; (ii) a V_{H} comprising an HC-CDR1 comprising the amino acid sequence of SEQ ID NO: 2, an HC-CDR2 comprising the amino acid sequence of SEQ ID NO: 10, and an HC-CDR3 comprising the amino acid sequence of SEQ ID NO: 19, or a variant thereof comprising up to about 5 amino acid substitutions in the HC-CDRs; and a V_{L} comprising a LC-CDR1 comprising the amino acid sequence of SEQ ID NO: 30, a LC-CDR2 comprising the amino acid sequence of SEQ ID NO: 43, and a LC-CDR3 comprising the amino acid sequence of SEQ ID NO: 55, or a variant thereof comprising up to about 5 amino acid substitutions in the LC-CDRs; (iii) a V_{H} comprising an HC-CDR1 comprising the amino acid sequence of SEQ ID NO: 2, an HC-CDR2 comprising the amino acid sequence of SEQ ID NO: 10, and an HC-CDR3 comprising the amino acid sequence of SEQ ID NO: 19, or a variant thereof comprising up to about 5 amino acid substitutions in the HC-CDRs; and a V_{L} comprising a LC-CDR1 comprising the amino acid sequence of SEQ ID NO: 31, a LC-CDR2 comprising the amino acid sequence of SEQ ID NO: 44, and a LC-CDR3 comprising the amino acid sequence of SEQ ID NO: 56, or a variant thereof comprising up to about 5 amino acid substitutions in the LC-CDRs; (iv) a V_{H} comprising an HC-CDR1 comprising the amino acid sequence of SEQ ID NO: 2, an HC-CDR2 comprising the amino acid sequence of SEQ ID NO: 10, and an HC-CDR3 comprising the amino acid sequence of SEQ ID NO: 19, or a variant thereof comprising up to about 5 amino acid substitutions in the HC-CDRs; and a V_{L} comprising a LC-CDR1 comprising the amino acid sequence of SEQ ID NO: 32, a LC-CDR2 comprising the amino acid sequence of SEQ ID NO: 45, and a LC-CDR3 comprising the amino acid sequence of SEQ ID NO: 57, or a variant thereof comprising up to about 5 amino acid substitutions in the LC-CDRs; (v) a V_{H} comprising an HC-CDR1 comprising the amino acid sequence of SEQ ID NO: 3, an HC-CDR2 comprising the amino acid sequence of SEQ ID NO: 11, and an HC-CDR3 comprising the amino acid sequence of SEQ ID NO: 19, or a variant thereof comprising up to about 5 amino acid substitutions in the HC-CDRs; and a V_{L} comprising a LC-CDR1 comprising the amino acid sequence of SEQ ID NO: 33, a LC-CDR2 comprising the amino acid sequence of SEQ ID NO: 45, and a LC-CDR3 comprising the amino acid sequence of SEQ ID NO: 58, or a variant thereof comprising up to about 5 amino acid substitutions in the LC-CDRs; or (vi) a V_{H} comprising an HC-CDR1 comprising the amino acid sequence of SEQ ID NO: 3, an HC-CDR2 comprising the amino acid sequence of SEQ ID NO: 10, and an HC-CDR3 comprising the amino acid sequence of SEQ ID NO: 19, or a variant thereof comprising up to about 5 amino acid substitutions in the HC-CDRs; and a V_{L} comprising a LC-CDR1 comprising the amino acid sequence of SEQ ID NO: 31, a LC-CDR2 comprising the amino acid sequence of SEQ ID NO: 46, and a LC-CDR3 comprising the amino acid sequence of SEQ ID NO: 59, or a variant thereof comprising up to about 5 amino acid substitutions in the LC-CDRs; or (vii) a V_{H} comprising an HC-CDR1 comprising the amino acid sequence of SEQ ID NO: 3, an HC-CDR2 comprising the amino acid sequence of SEQ ID NO: 12, and an HC-CDR3 comprising the amino acid sequence of SEQ ID NO: 20, or a variant thereof comprising up to about 5 amino acid substitutions in the HC-CDRs; and a V_{L} comprising a LC-CDR1 comprising the amino acid sequence of SEQ ID NO: 34, a LC-CDR2 comprising the amino acid sequence of SEQ ID NO: 47, and a LC-CDR3 comprising the amino acid sequence of SEQ ID NO: 60, or a variant thereof comprising up to about 5 amino acid substitutions in the LC-CDRs.

In some embodiments, according to any one of the isolated anti-TSLP antibodies described above, the isolated anti-TSLP antibody comprises: a V_{H} comprising the amino acid sequence of any one of SEQ ID NOs: 73-78, or a variant thereof having at least about 90% (for example at least about any of 91%, 92%, 93%, 94%, 95%, 96%, 97%, 98%, or 99%) sequence identity to the amino acid sequence of any one of SEQ ID NOs: 73-78; and a V_{L} comprising the amino acid sequence of any one of SEQ ID NOs: 91-97, or a variant thereof having at least about 90% (for example at least about any of 91%, 92%, 93%, 94%, 95%, 96%, 97%, 98%, or 99%) sequence identity to the amino acid sequence of any one of SEQ ID NOs: 91-97. In some embodiments, the isolated anti-TSLP antibody comprises: (i) a V_{H} comprising the amino acid sequence of SEQ ID NO: 73; and a V_{L} comprising the amino acid sequence of SEQ ID NO: 91; (ii) a V_{H} comprising the amino acid sequence of SEQ ID NO: 74; and a V_{L} comprising the amino acid sequence of SEQ ID NO: 91; (iii) a V_{H} comprising the amino acid sequence of SEQ ID NO: 75; and a V_{L} comprising the amino acid sequence of SEQ ID NO: 91; (iv) a V_{H} comprising the amino acid sequence of SEQ ID NO: 73; and a V_{L} comprising the amino acid sequence of SEQ ID NO: 92; (v) a V_{H} comprising the amino acid sequence of SEQ ID NO: 73; and a V_{L} comprising the amino acid sequence of SEQ ID NO: 93; (vi) a V_{H} comprising the amino acid sequence of SEQ ID NO: 73; and a V_{L} comprising the amino acid sequence of SEQ ID NO: 94; (vii) a V_{H} comprising the amino acid sequence of SEQ ID NO: 76; and a V_{L} comprising the amino acid sequence of SEQ ID NO: 95; (viii) a V_{H} comprising the amino acid sequence of SEQ ID NO: 77; and a V_{L} comprising the amino acid sequence of SEQ ID NO: 96; or (ix) a V_{H} comprising the amino acid sequence of SEQ ID NO: 78; and a V_{L} comprising the amino acid sequence of SEQ ID NO: 97.

In one aspect, the present application provides an isolated anti-TSLP antibody that specifically binds on human and/or cynomolgus monkey TSLP, the isolated anti-TSLP antibody comprises: a heavy chain variable domain (V_{H}) comprising a heavy chain complementarity determining region (HC-CDR) 1 comprising NYX₁MT (SEQ ID NO: 114), wherein X₁ is D or G; an HC-CDR2 comprising SITFASSYIYYADSVKG (SEQ ID NO: 13); and an HC-CDR3 comprising GGGAYX₁GGSLDV (SEQ ID NO: 115), wherein X₁ is H or Y; and a light chain variable domain (V_{L}) comprising a light chain complementarity determining region (LC-CDR) 1 comprising RSSQSLLHX₁X₂X₃YTYLH (SEQ ID NO: 116), wherein X₁ is I or S, X₂ is N or Y, and X₃ is G or E; a LC-CDR2 comprising LVSX₁RAS (SEQ ID NO: 117), wherein X₁ is H, or Y; and a LC-CDR3 comprising EQTLQTPX₁X₂ (SEQ ID NO: 118), wherein X₁ is Y or F, X₂ is S or T.

In some embodiments, there is provided an isolated anti-TSLP antibody comprising: a V_{H} comprising an HC-CDR1 comprising the amino acid sequence of any one of SEQ ID NOs: 4-5, or a variant thereof comprising up to about 3 amino acid substitutions; an HC-CDR2 comprising the amino acid sequence of SEQ ID NO: 13, or a variant thereof comprising up to about 3 amino acid substitutions; and an HC-CDR3 comprising the amino acid sequence of any one of SEQ ID NOs: 21-22, or a variant thereof comprising up to about 3 amino acid substitutions; and a V_{L} comprising a LC-CDR1 comprising the amino acid sequence of any one of SEQ ID NOs: 35-37, or a variant thereof comprising up to about 3 amino acid substitutions; a LC-CDR2 comprising the amino acid sequence of any one of SEQ ID NOs: 48-49, or a variant thereof comprising up to about 3 amino acid substitutions; and a LC-CDR3 comprising the amino acid sequence of any one of SEQ ID NOs: 61-63, or a variant thereof comprising up to about 3 amino acid substitutions.

In some embodiments, there is provided an isolated anti-TSLP antibody comprising a V_{H} comprising an HC-CDR1, an HC-CDR2, and an HC-CDR3 of a V_{H} comprising the amino acid sequence of any one of SEQ ID NOs: 79-81; and a V_{L} comprising a LC-CDR1, a LC-CDR2, and a LC-CDR3 of a V_{L} comprising the amino acid sequence of any one of SEQ ID NOs: 98-100.

In some embodiments, there is provided an isolated anti-TSLP antibody comprising: (i) a V_{H} comprising an HC-CDR1, an HC-CDR2, and an HC-CDR3 of a V_{H} comprising the amino acid sequence of SEQ ID NO: 79; and a V_{L} comprising a LC-CDR1, a LC-CDR2, and a LC-CDR3 of a V_{L} comprising the amino acid sequence of SEQ ID NO: 98; (ii) a V_{H} comprising an HC-CDR1, an HC-CDR2, and an HC-CDR3 of a V_{H} comprising the amino acid sequence of SEQ ID NO: 80; and a V_{L} comprising a LC-CDR1, a LC-CDR2, and a LC-CDR3 of a V_{L} comprising the amino acid sequence of SEQ ID NO: 99; or (iii) a V_{H} comprising an HC-CDR1, an HC-CDR2, and an HC-CDR3 of a V_{H} comprising the amino acid sequence of SEQ ID NO: 81; and a V_{L} comprising a LC-CDR1, a LC-CDR2, and a LC-CDR3 of a V_{L} comprising the amino acid sequence of SEQ ID NO: 100.

In some embodiments, there is provided an isolated anti-TSLP antibody comprising: (i) a V_{H} comprising an HC-CDR1 comprising the amino acid sequence of SEQ ID NO: 4, an HC-CDR2 comprising the amino acid sequence of SEQ ID NO: 13, and an HC-CDR3 comprising the amino acid sequence of SEQ ID NO: 21, or a variant thereof comprising up to about 5 amino acid substitutions in the HC-CDRs; and a V_{L} comprising a LC-CDR1 comprising the amino acid sequence of SEQ ID NO: 35, a LC-CDR2 comprising the amino acid sequence of SEQ ID NO: 48, and a LC-CDR3 comprising the amino acid sequence of SEQ ID NO: 61, or a variant thereof comprising up to about 5 amino acid substitutions in the LC-CDRs; (ii) a V_{H} comprising an HC-CDR1 comprising the amino acid sequence of SEQ ID NO: 4, an HC-CDR2 comprising the amino acid sequence of SEQ ID NO: 13, and an HC-CDR3 comprising the amino acid sequence of SEQ ID NO: 22, or a variant thereof comprising up to about 5 amino acid substitutions in the HC-CDRs; and a V_{L} comprising a LC-CDR1 comprising the amino acid sequence of SEQ ID NO: 36, a LC-CDR2 comprising the amino acid sequence of SEQ ID NO: 48, and a LC-CDR3 comprising the amino acid sequence of SEQ ID NO: 62, or a variant thereof comprising up to about 5 amino acid substitutions in the LC-CDRs; or (iii) a V_{H} comprising an HC-CDR1 comprising the amino acid sequence of SEQ ID NO: 5, an HC-CDR2 comprising the amino acid sequence of SEQ ID NO: 13, and an HC-CDR3 comprising the amino acid sequence of SEQ ID NO: 22, or a variant thereof comprising up to about 5 amino acid substitutions in the HC-CDRs; and a V_{L} comprising a LC-CDR1 comprising the amino acid sequence of SEQ ID NO: 37, a LC-CDR2 comprising the amino acid sequence of SEQ ID NO: 49, and a LC-CDR3 comprising the amino acid sequence of SEQ ID NO: 63, or a variant thereof comprising up to about 5 amino acid substitutions in the LC-CDRs.

In some embodiments, according to any one of the isolated anti-TSLP antibodies described above, the isolated anti-TSLP antibody comprises: a V_{H} comprising the amino acid sequence of any one of SEQ ID NOs: 79-81, or a variant thereof having at least about 90% (for example at least about any of 91%, 92%, 93%, 94%, 95%, 96%, 97%, 98%, or 99%) sequence identity to the amino acid sequence of any one of SEQ ID NOs: 79-81; and a V_{L} comprising the amino acid sequence of any one of SEQ ID NOs: 98-100, or a variant thereof having at least about 90% (for example at least about any of 91%, 92%, 93%, 94%, 95%, 96%, 97%, 98%, or 99%) sequence identity to the amino acid sequence of any one of SEQ ID NOs: 98-100. In some embodiments, the isolated anti-TSLP antibody comprises: (i) a V_{H} comprising the amino acid sequence of SEQ ID NO: 79; and a V_{L} comprising the amino acid sequence of SEQ ID NO: 98; (ii) a V_{H} comprising the amino acid sequence of SEQ ID NO: 80; and a V_{L} comprising the amino acid sequence of SEQ ID NO: 99; or (iii) a V_{H} comprising the amino acid sequence of SEQ ID NO: 81; and a V_{L} comprising the amino acid sequence of SEQ ID NO: 100.

In one aspect, the present application provides an isolated anti-TSLP antibody that specifically binds on human and/or cynomolgus monkey TSLP, the isolated anti-TSLP antibody comprises: a heavy chain variable domain (V_{H}) comprising a heavy chain complementarity determining region (HC-CDR) 1 comprising SYAIS (SEQ ID NO: 6); an HC-CDR2 comprising MX₁X₂PLLGVTX₃YAEKFQG (SEQ ID NO: 119), wherein X₁ is L or I, X₂ is V or I, and X₃ is N or D; and an HC-CDR3 comprising GGX₁NYLYWYFDL (SEQ ID NO: 120), wherein X₁ is S or T; and a light chain variable domain (V_{L}) comprising a light chain complementarity determining region (LC-CDR) 1 comprising TGTSSDIGGYNRX₁S (SEQ ID NO: 121), wherein X₁ is V or I; a LC-CDR2 comprising X₁VSKRPS (SEQ ID NO: 122), wherein X₁ is D or E; and a LC-CDR3 comprising SX₁YAGTDTFVL (SEQ ID NO: 123), wherein X₁ is S or A.

In some embodiments, there is provided an isolated anti-TSLP antibody comprising: a V_{H} comprising an HC-CDR1 comprising the amino acid sequence of SEQ ID NO: 6, or a variant thereof comprising up to about 3 amino acid substitutions; an HC-CDR2 comprising the amino acid sequence of any one of SEQ ID NOs: 14-15, or a variant thereof comprising up to about 3 amino acid substitutions; and an HC-CDR3 comprising the amino acid sequence of any one of SEQ ID NOs: 23-24, or a variant thereof comprising up to about 3 amino acid substitutions; and a V_{L} comprising a LC-CDR1 comprising the amino acid sequence of any one of SEQ ID NOs: 31 and 38, or a variant thereof comprising up to about 3 amino acid substitutions; a LC-CDR2 comprising the amino acid sequence of any one of SEQ ID NOs: 42 and 45, or a variant thereof comprising up to about 3 amino acid substitutions; and a LC-CDR3 comprising the amino acid sequence of any one of SEQ ID NOs: 60 and 64, or a variant thereof comprising up to about 3 amino acid substitutions.

In some embodiments, there is provided an isolated anti-TSLP antibody comprising a V_{H} comprising an HC-CDR1, an HC-CDR2, and an HC-CDR3 of a V_{H} comprising the amino acid sequence of any one of SEQ ID NOs: 82-83; and a V_{L} comprising a LC-CDR1, a LC-CDR2, and a LC-CDR3 of a V_{L} comprising the amino acid sequence of any one of SEQ ID NOs: 101-102.

In some embodiments, there is provided an isolated anti-TSLP antibody comprising: (i) a V_{H} comprising an HC-CDR1, an HC-CDR2, and an HC-CDR3 of a V_{H} comprising the amino acid sequence of SEQ ID NO: 82; and a V_{L} comprising a LC-CDR1, a LC-CDR2, and a LC-CDR3 of a V_{L} comprising the amino acid sequence of SEQ ID NO: 101; or (ii) a V_{H} comprising an HC-CDR1, an HC-CDR2, and an HC-CDR3 of a V_{H} comprising the amino acid sequence of SEQ ID NO: 83; and a V_{L} comprising a LC-CDR1, a LC-CDR2, and a LC-CDR3 of a V_{L} comprising the amino acid sequence of SEQ ID NO: 102.

In some embodiments, there is provided an isolated anti-TSLP antibody comprising: (i) a V_{H} comprising an HC-CDR1 comprising the amino acid sequence of SEQ ID NO: 6, an HC-CDR2 comprising the amino acid sequence of SEQ ID NO: 14, and an HC-CDR3 comprising the amino acid sequence of SEQ ID NO: 23, or a variant thereof comprising up to about 5 amino acid substitutions in the HC-CDRs; and a V_{L} comprising a LC-CDR1 comprising the amino acid sequence of SEQ ID NO: 38, a LC-CDR2 comprising the amino acid sequence of SEQ ID NO: 42, and a LC-CDR3 comprising the amino acid sequence of SEQ ID NO: 60, or a variant thereof comprising up to about 5 amino acid substitutions in the LC-CDRs; or (ii) a V_{H} comprising an HC-CDR1 comprising the amino acid sequence of SEQ ID NO: 6, an HC-CDR2 comprising the amino acid sequence of SEQ ID NO: 15, and an HC-CDR3 comprising the amino acid sequence of SEQ ID NO: 24, or a variant thereof comprising up to about 5 amino acid substitutions in the HC-CDRs; and a V_{L} comprising a LC-CDR1 comprising the amino acid sequence of SEQ ID NO: 31, a LC-CDR2 comprising the amino acid sequence of SEQ ID NO: 45, and a LC-CDR3 comprising the amino acid sequence of SEQ ID NO: 64, or a variant thereof comprising up to about 5 amino acid substitutions in the LC-CDRs.

In some embodiments, according to any one of the isolated anti-TSLP antibodies described above, the isolated anti-TSLP antibody comprises: a V_{H} comprising the amino acid sequence of any one of SEQ ID NOs: 82-83, or a variant thereof having at least about 90% (for example at least about any of 91%, 92%, 93%, 94%, 95%, 96%, 97%, 98%, or 99%) sequence identity to the amino acid sequence of any one of SEQ ID NOs: 82-83; and a V_{L} comprising the amino acid sequence of any one of SEQ ID NOs: 101-102, or a variant thereof having at least about 90% (for example at least about any of 91%, 92%, 93%, 94%, 95%, 96%, 97%, 98%, or 99%) sequence identity to the amino acid sequence of any one of SEQ ID NOs: 101-102. In some embodiments, the isolated anti-TSLP antibody comprises: (i) a V_{H} comprising the amino acid sequence of SEQ ID NO: 82; and a V_{L} comprising the amino acid sequence of SEQ ID NO: 101; or (ii) a V_{H} comprising the amino acid sequence of SEQ ID NO: 83; and a V_{L} comprising the amino acid sequence of SEQ ID NO: 102.

In some embodiments, there is provided an isolated anti-TSLP antibody that specifically binds to the human TSLP with a Kd from about 0.1 pM to about 1 nM.

In some embodiments, there is provided an isolated anti-TSLP antibody that specifically binds to TSLP competitively with any one of the isolated anti-TSLP antibodies as described above. In some embodiments, there is provided an isolated anti-TSLP antibody that specifically binds to the same epitope as any one of isolated anti-TSLP antibodies as described above.

In some embodiments according to any of the isolated anti-TSLP antibodies described above, the isolated anti-TSLP antibody comprises an Fc fragment. In some embodiments, the isolated anti-TSLP antibody is a full-length IgG antibody. In some embodiments, the isolated anti-TSLP antibody is a full-length IgG1, IgG2, IgG3 or IgG4 antibody. In some embodiments, the anti-TSLP antibody is chimeric, human, or humanized antibody. In some embodiments, the anti-TSLP antibody is an antigen binding fragment selected from the group consisting of a Fab, a Fab′, a F(ab)′₂, a Fab′-SH, a single-chain Fv (scFv), an Fv fragment, a dAb, a Fd, a nanobody, a diabody, and a linear antibody.

In some embodiments, there is provided isolated nucleic acid molecule(s) that encodes any one of the anti-TSLP antibodies described above. In some embodiments, there is provided a vector comprising any one of the nucleic acid molecules described above. In some embodiments, there is provided a host cell comprising any one of the anti-TSLP antibodies described above, any one of the nucleic acid molecules described above, or any one of the vectors described above. In some embodiments, there is provided a method of producing an anti-TSLP antibody, comprising: a) culturing any one of the host cells described above under conditions effective to express the anti-TSLP antibody; and b) obtaining the expressed anti-TSLP antibody from the host cell. In some embodiments, there is provided a pharmaceutical composition comprising the isolated anti-TSLP antibodies, the nucleic acids, the vector, or the isolated host cell described herein, and a pharmaceutically acceptable carrier.

In some embodiments, there is provided a method of treating a disease or condition in an individual in need thereof, comprising administering to the individual an effective amount of an anti-TSLP antibody according to any one of the anti-TSLP antibodies or the pharmaceutical composition described herein. In some embodiments, there is provided the anti-TSLP antibodies or a pharmaceutical composition described herein for use in the treatment of a disease or condition. In some embodiments, there is provided the use of any one of the anti-TSLP antibodies described herein for the preparation of pharmaceutical compositions for treating a disease or condition in an individual in need. In some embodiments, there is provided the use of the anti-TSLP antibodies or a pharmaceutical composition described herein, for the manufacture of a medicament for the treatment of a disease or condition. In some embodiments, the disease or condition is associated with TSLP, comprising inflammatory or autoimmune disease or condition. In some embodiments, the disease or condition is selected from the group consisting of asthma, chronic obstructive pulmonary disease, allergic rhinitis, allergic rhinosinusitis, allergic conjunctivitis, eosinophilic esophagitis, and atopic dermatitis.

Also provided are pharmaceutical compositions, kits and articles of manufacture comprising any one of the anti-TSLP antibodies described above.

### BRIEF DESCRIPTION OF THE DRAWINGS

FIGS. 1A-1F show the binding affinity of exemplary anti-TSLP antibodies to human TSLP as analyzed by ELISA.

FIGS. 2A-2F show the binding affinity of exemplary anti-TSLP antibodies to cynomolgus monkey TSLP as analyzed by ELISA.

FIG. 3A shows the affinity Kd of the detected anti-TSLP antibodies TSLP-0107, or TSLP-0202 to human TSLP. FIG. 3B shows the affinity Kd of the detected anti-TSLP antibodies TSLP-0107, or TSLP-0202 to cynomolgus monkey TSLP.

FIG. 4 shows the results of the exemplary anti-TSLP antibodies cross-reactivity to TSLP homologous protein IL-7 as analyzed by ELISA.

FIG. 5A shows that the rabbit anti-TSLP polyclonal antibody can bind to human short isoform TSLP. FIG. 5B shows that the rabbit anti-TSLP polyclonal antibody can bind to human full length isoform TSLP. FIG. 5C shows that the anti-TSLP antibody TSLP-01 cannot bind to human short isoform TSLP. FIG. 5D shows that the anti-TSLP antibody TSLP-01 can bind to human full length isoform TSLP.

FIG. 6 shows the result of the inhibition of TARC release in human PBMCs assay for the exemplary anti-TSLP antibodies.

FIGS. 7A-7D show results of pharmacokinetics analysis of the exemplary anti-TSLP antibodies upon subcutaneously injection 30 mg/kg or 10 mg/kg of the respective antibodies in rat as measured by ELISA. FIG. 7A shows the pharmacokinetics results of the anti-TSLP antibody TSLP-0107 or AMG157 upon subcutaneously injection 30 mg/kg in rats. FIG. 7B shows the pharmacokinetics results of the anti-TSLP antibody TSLP-0107 or AMG157 upon subcutaneously injection 10 mg/kg in rats. FIG. 7C shows the pharmacokinetics results of the anti-TSLP antibody TSLP-0202 or AMG157 upon subcutaneously injection 30 mg/kg in rats.

FIG. 7D shows the pharmacokinetics results of the anti-TSLP antibody TSLP-0202 or AMG157 upon subcutaneously injection 10 mg/kg in rats.

### DETAILED DESCRIPTION OF THE APPLICATION

The present application in one aspect provides an isolated anti-TSLP antibody that specifically binds to human and/or cynomolgus monkey TSLP. By using a combination of selections on naïve scFv phage libraries, affinity maturation and appropriately designed biochemical and biological assays, highly potent antibody molecules that bind to human and/or cynomolgus monkey TSLP and inhibit the action of human and/or cynomolgus monkey TSLP at its receptor were identified in the present application. The results presented herein indicate that the antibodies bind human and/or cynomolgus monkey TSLP with high affinity, compared with the known anti-TSLP antibodies AMG157 (Amgen), and surprisingly are even more potent than AMG157 as demonstrated in a variety of biological assays.

The anti-TSLP antibodies provided by the present application include, for example, full-length anti-TSLP antibodies, anti-TSLP scFvs, anti-TSLP Fc fusion proteins, multi-specific (such as bispecific) anti-TSLP antibodies, anti-TSLP immunoconjugates, and the like.

Also provided are nucleic acids encoding the anti-TSLP antibodies, compositions comprising the anti-TSLP antibodies, and methods of making and using the anti-TSLP antibodies.

### Definitions

As used herein, “treatment” or “treating” is an approach for obtaining beneficial or desired results, including clinical results. For purposes of this application, beneficial or desired clinical results include, but are not limited to, one or more of the following: alleviating one or more symptoms resulting from the disease, diminishing the extent of the disease, stabilizing the disease (e.g., preventing or delaying the worsening of the disease), preventing or delaying the spread (e.g., metastasis) of the disease, preventing or delaying the recurrence of the disease, delaying or slowing the progression of the disease, ameliorating the disease state, providing a remission (partial or total) of the disease, decreasing the dose of one or more other medications required to treat the disease, delaying the progression of the disease, increasing or improving the quality of life, increasing weight gain, and/or prolonging survival. Also encompassed by “treatment” is a reduction of pathological consequence of the disease (such as, for example, tumor volume for cancer). The methods of the application contemplate any one or more of these aspects of treatment.

The term “antibody” includes full-length antibodies and antigen-binding fragments thereof. A full-length antibody comprises two heavy chains and two light chains. The variable regions of the light and heavy chains are responsible for antigen binding. The variable regions in both chains generally contain three highly variable loops called the complementarity determining regions (CDRs) (light chain (LC) CDRs including LC-CDR1, LC-CDR2, and LC-CDR3, heavy chain (HC) CDRs including HC-CDR1, HC-CDR2, and HC-CDR3). CDR boundaries for the antibodies and antigen-binding fragments disclosed herein may be defined or identified by the conventions of Kabat, Chothia, or Al-Lazikani (Al-Lazikani 1997; Chothia 1985; Chothia 1987; Chothia 1989; Kabat 1987; Kabat 1991). The three CDRs of the heavy or light chains are interposed between flanking stretches known as framework regions (FRs), which are more highly conserved than the CDRs and form a scaffold to support the hypervariable loops. The constant regions of the heavy and light chains are not involved in antigen binding, but exhibit various effector functions. Antibodies are assigned to classes based on the amino acid sequence of the constant region of their heavy chain. The five major classes or isotypes of antibodies are IgA, IgD, IgE, IgG, and IgM, which are characterized by the presence of α, δ, ε, γ, and heavy chains, respectively. Several of the major antibody classes are divided into subclasses such as IgG1 (γ1 heavy chain), IgG2 (γ2 heavy chain), IgG3 (γ3 heavy chain), IgG4 (γ4 heavy chain), IgA1 (α1 heavy chain), or IgA2 (α2 heavy chain).

The term “antigen-binding fragment” as used herein includes an antibody fragment including, for example, a diabody, a Fab, a Fab′, a F(ab′)₂, an Fv fragment, a disulfide stabilized Fv fragment (dsFv), a (dsFv)2, a bispecific dsFv (dsFv-dsFv′), a disulfide stabilized diabody (ds diabody), a single-chain Fv (scFv), an scFv dimer (bivalent diabody), a multispecific antibody formed from a portion of an antibody comprising one or more CDRs, a single domain antibody, a nanobody, a domain antibody, a bivalent domain antibody, or any other antibody fragments that bind to an antigen but do not comprise a complete antibody structure. An antigen-binding fragment also includes a fusion protein comprising the antibody fragment described above. An antigen-binding fragment is capable of binding to the same antigen to which the parent antibody or a parent antibody fragment (e.g., a parent scFv) binds. In some embodiments, an antigen-binding fragment may comprise one or more CDRs from a particular human antibody grafted to a framework region from one or more different human antibodies.

The term “epitope” as used herein refers to the specific group of atoms or amino acids on an antigen to which an antibody or antibody moiety binds. Two antibodies or antibody moieties may bind the same epitope within an antigen if they exhibit competitive binding for the antigen.

As used herein, a first antibody “competes” for binding to a target TSLP with a second antibody when the first antibody inhibits target TSLP binding of the second antibody by at least about 50% (such as at least about any of 55%, 60%, 65%, 70%, 75%, 80%, 85%, 90%, 95%, 98% or 99%) in the presence of an equimolar concentration of the first antibody, or vice versa. A high throughput process for “binning” antibodies based upon their cross-competition is described in PCT Publication No. WO 03/48731.

As used herein, the term “specifically binds,” “specifically recognizing,” or “is specific for” refers to measurable and reproducible interactions, such as binding between a target and an antibody that is determinative of the presence of the target in the presence of a heterogeneous population of molecules, including biological molecules. For example, an antibody that specifically recognizes a target (which can be an epitope) is an antibody that binds to this target with greater affinity, avidity, more readily, and/or with greater duration than its bindings to other targets. In some embodiments, an antibody that specifically recognizes an antigen reacts with one or more antigenic determinants of the antigen with a binding affinity that is at least about 10 times its binding affinity for other targets.

An “isolated” anti-TSLP antibody as used herein refers to an anti-TSLP antibody that (1) is not associated with proteins found in nature, (2) is free of other proteins from the same source, (3) is expressed by a cell from a different species, or, (4) does not occur in nature.

The term “isolated nucleic acid” as used herein is intended to mean a nucleic acid of genomic, cDNA, or synthetic origin or some combination thereof, which by virtue of its origin the “isolated nucleic acid” (1) is not associated with all or a portion of a polynucleotide in which the “isolated nucleic acid” is found in nature, (2) is operably linked to a polynucleotide which it is not linked to in nature, or (3) does not occur in nature as part of a larger sequence.

As used herein, the term “CDR” or “complementarity determining region” is intended to mean the non-contiguous antigen combining sites found within the variable region of both heavy and light chain polypeptides. These particular regions have been described by Kabat et al., J. Biol. Chem. 252:6609-6616 (1977); Kabat et al., U.S. Dept. of Health and Human Services, “Sequences of proteins of immunological interest” (1991); Chothia et al., J. Mol. Biol. 196:901-917 (1987); Al-Lazikani B. et al., J. Mol. Biol., 273: 927-948 (1997); MacCallum et al., J. Mol. Biol. 262:732-745 (1996); Abhinandan and Martin, Mol. Immunol., 45: 3832-3839 (2008); Lefranc M. P. et al., Dev. Comp. Immunol., 27: 55-77 (2003); and Honegger and Pluckthun, J. Mol. Biol., 309:657-670 (2001), where the definitions include overlapping or subsets of amino acid residues when compared against each other. Nevertheless, application of either definition to refer to a CDR of an antibody or grafted antibodies or variants thereof is intended to be within the scope of the term as defined and used herein. The amino acid residues which encompass the CDRs as defined by each of the above cited references are set forth below in Table 1 as a comparison. CDR prediction algorithms and interfaces are known in the art, including, for example, Abhinandan and Martin, Mol. Immunol., 45: 3832-3839 (2008); Ehrenmann F. et al., Nucleic Acids Res., 38: D301-D307 (2010); and Adolf-Bryfogle J. et al., Nucleic Acids Res., 43: D432-D438 (2015). The contents of the references cited in this paragraph are incorporated herein by reference in their entireties for use in the present application and for possible inclusion in one or more claims herein.

TABLE 1
CDR DEFINITIONS

| | Kabat¹ | Chothia² | MacCallum³ | IMGT⁴ | AHo⁵ |
|---|---|---|---|---|---|
| V_{H} CDR1 | 31-35 | 26-32 | 30-35 | 27-38 | 25-40 |
| V_{H} CDR2 | 50-65 | 53-55 | 47-58 | 56-65 | 58-77 |
| V_{H} CDR3 | 95-102 | 96-101 | 93-101 | 105-117 | 109-137 |
| V_{L} CDR1 | 24-34 | 26-32 | 30-36 | 27-38 | 25-40 |
| V_{L} CDR2 | 50-56 | 50-52 | 46-55 | 56-65 | 58-77 |
| V_{L} CDR3 | 89-97 | 91-96 | 89-96 | 105-117 | 109-137 |

¹Residue numbering follows the nomenclature of Kabat et al., supra
²Residue numbering follows the nomenclature of Chothia et al., supra
³Residue numbering follows the nomenclature of MacCallum et al., supra
⁴Residue numbering follows the nomenclature of Lefranc et al., supra
⁵Residue numbering follows the nomenclature of Honegger and Plückthun, supra

The term “chimeric antibodies” refer to antibodies in which a portion of the heavy and/or light chain is identical with or homologous to corresponding sequences in antibodies derived from a particular species or belonging to a particular antibody class or subclass, while the remainder of the chain(s) is identical with or homologous to corresponding sequences in antibodies derived from another species or belonging to another antibody class or subclass, as well as fragments of such antibodies, so long as they exhibit a biological activity of this application (see U.S. Pat. No. 4,816,567; and Morrison et al., Proc. Natl. Acad. Sci. USA, 81:6851-6855 (1984)).

“Fv” is the minimum antibody fragment which contains a complete antigen-recognition and -binding site. This fragment consists of a dimer of one heavy- and one light-chain variable region domain in tight, non-covalent association. From the folding of these two domains emanate six hypervariable loops (3 loops each from the heavy and light chain) that contribute the amino acid residues for antigen binding and confer antigen binding specificity to the antibody. However, even a single variable domain (or half of an Fv comprising only three CDRs specific for an antigen) has the ability to recognize and bind antigen, although at a lower affinity than the entire binding site.

“Single-chain Fv,” also abbreviated as “sFv” or “scFv,” are antibody fragments that comprise the V_{H} and V_{L} antibody domains connected into a single polypeptide chain. In some embodiments, the scFv polypeptide further comprises a polypeptide linker between the V_{H} and V_{L} domains which enables the scFv to form the desired structure for antigen binding. For a review of scFv, see Pluckthun in The Pharmacology of Monoclonal Antibodies, vol. 113, Rosenburg and Moore eds., Springer-Verlag, New York, pp. 269-315 (1994).

The term “diabodies” refers to small antibody fragments prepared by constructing scFv fragments (see preceding paragraph) typically with short linkers (such as about 5 to about 10 residues) between the V_{H} and V_{L} domains such that inter-chain but not intra-chain pairing of the V domains is achieved, resulting in a bivalent fragment, i.e., fragment having two antigen-binding sites. Bispecific diabodies are heterodimers of two “crossover” scFv fragments in which the V_{H} and V_{L} domains of the two antibodies are present on different polypeptide chains. Diabodies are described more fully in, for example, EP 404,097; WO 93/11161; and Hollinger et al., Proc. Natl. Acad. Sci. USA, 90:6444-6448 (1993).

“Humanized” forms of non-human (e.g., rodent) antibodies are chimeric antibodies that contain minimal sequence derived from the non-human antibody. For the most part, humanized antibodies are human immunoglobulins (recipient antibody) in which residues from a hypervariable region (HVR) of the recipient are replaced by residues from a hypervariable region of a non-human species (donor antibody) such as mouse, rat, rabbit or non-human primate having the desired antibody specificity, affinity, and capability. In some instances, framework region (FR) residues of the human immunoglobulin are replaced by corresponding non-human residues. Furthermore, humanized antibodies can comprise residues that are not found in the recipient antibody or in the donor antibody. These modifications are made to further refine antibody performance. In general, the humanized antibody will comprise substantially all of at least one, and typically two, variable domains, in which all or substantially all of the hypervariable loops correspond to those of a non-human immunoglobulin and all or substantially all of the FRs are those of a human immunoglobulin sequence. The humanized antibody optionally also will comprise at least a portion of an immunoglobulin constant region (Fc), typically that of a human immunoglobulin. For further details, see Jones et al., Nature 321:522-525 (1986); Riechmann et al., Nature 332:323-329 (1988); and Presta, Curr. Op. Struct. Biol. 2:593-596 (1992).

“Percent (%) amino acid sequence identity” or “homology” with respect to the polypeptide and antibody sequences identified herein is defined as the percentage of amino acid residues in a candidate sequence that are identical with the amino acid residues in the polypeptide being compared, after aligning the sequences considering any conservative substitutions as part of the sequence identity. Alignment for purposes of determining percent amino acid sequence identity can be achieved in various ways that are within the skilled in the art, for instance, using publicly available computer software such as BLAST, BLAST-2, ALIGN, Megalign (DNASTAR), or MUSCLE software. Those skilled in the art can determine appropriate parameters for measuring alignment, including any algorithms needed to achieve maximal alignment over the full-length of the sequences being compared. For purposes herein, however, % amino acid sequence identity values are generated using the sequence comparison computer program MUSCLE (Edgar, R. C., Nucleic Acids Research 32(5):1792-1797, 2004; Edgar, R. C., BMC Bioinformatics 5(1):113, 2004).

The terms “Fc receptor” or “FcR” are used to describe a receptor that binds to the Fc region of an antibody. In some embodiments, an FcR of this application is one that binds to an IgG antibody (a 7 receptor) and includes receptors of the FcγRI, FcγRII, and FcγRIII subclasses, including allelic variants and alternatively spliced forms of these receptors. FcγRII receptors include FcγRIIA (an “activating receptor”) and FcγRIIB (an “inhibiting receptor”), which have similar amino acid sequences that differ primarily in the cytoplasmic domains thereof. Activating receptor FcγRIIA contains an immunoreceptor tyrosine-based activation motif (ITAM) in its cytoplasmic domain. Inhibiting receptor FcγRIIB contains an immunoreceptor tyrosine-based inhibition motif (ITIM) in its cytoplasmic domain (see review M. in Daeron, Annu. Rev. Immunol. 15:203-234 (1997)). The term includes allotypes, such as FcγRIIIA allotypes: FcγRIIIA-Phe158, FcγRIIIA-Val158, FcγRIIA-R131 and/or FcγRIIA-H131. FcRs are reviewed in Ravetch and Kinet, Annu. Rev. Immunol 9:457-92 (1991); Capel et al., Immunomethods 4:25-34 (1994); and de Haas et al., J. Lab. Clin. Med. 126:330-41 (1995). Other FcRs, including those to be identified in the future, are encompassed by the term “FcR” herein. The term also includes the neonatal receptor, FcRn, which is responsible for the transfer of maternal IgGs to the fetus (Guyer et al., J. Immunol. 117:587 (1976) and Kim et al., J. Immunol. 24:249 (1994)).

The term “FcRn” refers to the neonatal Fc receptor (FcRn). FcRn is structurally similar to major histocompatibility complex (MHC) and consists of an α-chain noncovalently bound to β2-microglobulin. The multiple functions of the neonatal Fc receptor FcRn are reviewed in Ghetie and Ward (2000) Annu. Rev. Immunol. 18, 739-766. FcRn plays a role in the passive delivery of immunoglobulin IgGs from mother to young and the regulation of serum IgG levels. FcRn can act as a salvage receptor, binding and transporting pinocytosed IgGs in intact form both within and across cells, and rescuing them from a default degradative pathway.

The “CH1 domain” of a human IgG Fc region usually extends from about amino acid 118 to about amino acid 215 (EU numbering system).

“Hinge region” is generally defined as stretching from Glu216 to Pro230 of human IgG1 (Burton, Molec. Immunol. 22:161-206 (1985)). Hinge regions of other IgG isotypes may be aligned with the IgG1 sequence by placing the first and last cysteine residues forming inter-heavy chain S—S bonds in the same positions.

The “CH2 domain” of a human IgG Fc region usually extends from about amino acid 231 to about amino acid 340. The CH2 domain is unique in that it is not closely paired with another domain. Rather, two N-linked branched carbohydrate chains are interposed between the two CH2 domains of an intact native IgG molecule. It has been speculated that the carbohydrate may provide a substitute for the domain-domain pairing and help stabilize the CH2 domain. Burton, Molec Immunol. 22:161-206 (1985).

The “CH3 domain” comprises the stretch of residues of C-terminal to a CH2 domain in an Fc region (i.e. from about amino acid residue 341 to the C-terminal end of an antibody sequence, typically at amino acid residue 446 or 447 of an IgG).

A “functional Fc fragment” possesses an “effector function” of a native sequence Fc region. Exemplary “effector functions” include C1q binding; complement dependent cytotoxicity (CDC); Fc receptor binding; antibody-dependent cell-mediated cytotoxicity (ADCC); phagocytosis; down regulation of cell surface receptors (e.g. B cell receptor; BCR), etc. Such effector functions generally require the Fc region to be combined with a binding domain (e.g. an antibody variable domain) and can be assessed using various assays known in the art.

An antibody with a variant IgG Fc with “altered” FcR binding affinity or ADCC activity is one which has either enhanced or diminished FcR binding activity (e.g., FcγR or FcRn) and/or ADCC activity compared to a parent polypeptide or to a polypeptide comprising a native sequence Fc region. The variant Fc which “exhibits increased binding” to an FcR binds at least one FcR with higher affinity (e.g., lower apparent Kd or IC₅₀ value) than the parent polypeptide or a native sequence IgG Fc. According to some embodiments, the improvement in binding compared to a parent polypeptide is about 3-fold, such as about any of 5, 10, 25, 50, 60, 100, 150, 200, or up to 500-fold, or about 25% to 1000% improvement in binding. The polypeptide variant which “exhibits decreased binding” to an FcR, binds at least one FcR with lower affinity (e.g., higher apparent Kd or higher IC₅₀ value) than a parent polypeptide. The decrease in binding compared to a parent polypeptide may be about 40% or more decrease in binding.

“Antibody-dependent cell-mediated cytotoxicity” or “ADCC” refers to a form of cytotoxicity in which secreted Ig bound to Fc receptors (FcRs) present on certain cytotoxic cells (e.g., Natural Killer (NK) cells, neutrophils, and macrophages) enable these cytotoxic effector cells to bind specifically to an antigen-bearing target cell and subsequently kill the target cell with cytotoxins. The antibodies “arm” the cytotoxic cells and are required for such killing. The primary cells for mediating ADCC, NK cells, express FcγRIII only, whereas monocytes express FcγRI, FcγRII and FcγRIII. FcR expression on hematopoietic cells is summarized in Table 3 on page 464 of Ravetch and Kinet, Annu. Rev. Immunol 9:457-92 (1991). To assess ADCC activity of a molecule of interest, an in vitro ADCC assay, such as that described in U.S. Pat. No. 5,500,362 or 5,821,337 may be performed. Useful effector cells for such assays include peripheral blood mononuclear cells (PBMC) and Natural Killer (NK) cells. Alternatively, or additionally, ADCC activity of the molecule of interest may be assessed in vivo, e.g., in an animal model such as that disclosed in Clynes et al. PNAS (USA) 95:652-656 (1998).

The polypeptide comprising a variant Fc region which “exhibits increased ADCC” or mediates ADCC in the presence of human effector cells more effectively than a polypeptide having wild type IgG Fc or a parent polypeptide is one which in vitro or in vivo is substantially more effective at mediating ADCC, when the amounts of polypeptide with variant Fc region and the polypeptide with wild type Fc region (or the parent polypeptide) in the assay are essentially the same. Generally, such variants will be identified using any in vitro ADCC assay known in the art, such as assays or methods for determining ADCC activity, e.g., in an animal model etc. In some embodiments, the variant is from about 5 fold to about 100 fold, e.g. from about 25 to about 50 fold, more effective at mediating ADCC than the wild type Fc (or parent polypeptide).

“Complement dependent cytotoxicity” or “CDC” refers to the lysis of a target cell in the presence of complement. Activation of the classical complement pathway is initiated by the binding of the first component of the complement system (C1q) to antibodies (of the appropriate subclass) which are bound to their cognate antigen. To assess complement activation, a CDC assay, e.g. as described in Gazzano-Santoro et al., J. Immunol. Methods 202:163 (1996), may be performed. Polypeptide variants with altered Fc region amino acid sequences and increased or decreased C1q binding capability are described in U.S. Pat. No. 6,194,551B1 and WO99/51642. The contents of those patent publications are specifically incorporated herein by reference. See also, Idusogie et al. J. Immunol. 164: 4178-4184 (2000).

Unless otherwise specified, a “nucleotide sequence encoding an amino acid sequence” includes all nucleotide sequences that are degenerate versions of each other and that encode the same amino acid sequence. The phrase nucleotide sequence that encodes a protein or a RNA may also include introns to the extent that the nucleotide sequence encoding the protein may in some version contain an intron(s).

The term “operably linked” refers to functional linkage between a regulatory sequence and a heterologous nucleic acid sequence resulting in expression of the latter. For example, a first nucleic acid sequence is operably linked with a second nucleic acid sequence when the first nucleic acid sequence is placed in a functional relationship with the second nucleic acid sequence. For instance, a promoter is operably linked to a coding sequence if the promoter affects the transcription or expression of the coding sequence. Generally, operably linked DNA sequences are contiguous and, where necessary to join two protein coding regions, in the same reading frame.

“Homologous” refers to the sequence similarity or sequence identity between two polypeptides or between two nucleic acid molecules. When a position in both of the two compared sequences is occupied by the same base or amino acid monomer subunit, e.g., if a position in each of two DNA molecules is occupied by adenine, then the molecules are homologous at that position. The percent of homology between two sequences is a function of the number of matching or homologous positions shared by the two sequences divided by the number of positions compared times 100. For example, if 6 of 10 of the positions in two sequences are matched or homologous then the two sequences are 60% homologous. By way of example, the DNA sequences ATTGCC and TATGGC share 50% homology. Generally, a comparison is made when two sequences are aligned to give maximum homology.

An “effective amount” of an anti-TSLP antibody or composition as disclosed herein, is an amount sufficient to carry out a specifically stated purpose. An “effective amount” can be determined empirically and by known methods relating to the stated purpose.

The term “therapeutically effective amount” refers to an amount of an anti-TSLP antibody or composition as disclosed herein, effective to “treat” a disease or disorder in an individual. In the case of cancer, the therapeutically effective amount of the anti-TSLP antibody or composition as disclosed herein can reduce the number of cancer cells; reduce the tumor size or weight; inhibit (i.e., slow to some extent and preferably stop) cancer cell infiltration into peripheral organs; inhibit (i.e., slow to some extent and preferably stop) tumor metastasis; inhibit, to some extent, tumor growth; and/or relieve to some extent one or more of the symptoms associated with the cancer. To the extent the anti-TSLP antibody or composition as disclosed herein can prevent growth and/or kill existing cancer cells, it can be cytostatic and/or cytotoxic. In some embodiments, the therapeutically effective amount is a growth inhibitory amount. In some embodiments, the therapeutically effective amount is an amount that extends the survival of a patient. In some embodiments, the therapeutically effective amount is an amount that improves progression free survival of a patient.

As used herein, by “pharmaceutically acceptable” or “pharmacologically compatible” is meant a material that is not biological or otherwise undesirable, e.g., the material may be incorporated into a pharmaceutical composition administered to a patient without causing any significant undesirable biological effects or interacting in a deleterious manner with any of the other components of the composition in which it is contained. Pharmaceutically acceptable carriers or excipients have preferably met the required standards of toxicological and manufacturing testing and/or are included on the Inactive Ingredient Guide prepared by the U.S. Food and Drug Administration.

It is understood that embodiments of the application described herein include “consisting of” and/or “consisting essentially of” embodiments.

Reference to “about” a value or parameter herein includes (and describes) variations that are directed to that value or parameter per se. For example, description referring to “about X” includes description of “X”.

As used herein, reference to “not” a value or parameter generally means and describes “other than” a value or parameter. For example, the method is not used to treat cancer of type X means the method is used to treat cancer of types other than X.

As used herein and in the appended claims, the singular forms “a,” “an,” and “the” include plural referents unless the context clearly dictates otherwise.

### Anti-TSLP Antibodies

In one aspect, the present application provides anti-TSLP antibodies that specifically bind to human and/or cynomolgus monkey TSLP. Anti-TSLP antibodies include, but are not limited to, humanized antibodies, chimeric antibodies, mouse antibodies, human antibodies, and antibodies comprising the heavy chain and/or light chain CDRs discussed herein. In one aspect, the present application provides isolated antibodies that bind to TSLP. Contemplated anti-TSLP antibodies include, for example, full-length anti-TSLP antibodies (e.g., full-length IgG1 or IgG4), anti-TSLP scFvs, anti-TSLP Fc fusion proteins, multi-specific (such as bispecific) anti-TSLP antibodies, anti-TSLP immunoconjugates, and the like. In some embodiments, the anti-TSLP antibody is a full-length antibody (e.g., full-length IgG1 or IgG4) or antigen-binding fragment thereof, which specifically binds to TSLP. In some embodiments, the anti-TSLP antibody is a Fab, a Fab′, a F(ab)′₂, a Fab′-SH, a single-chain Fv (scFv), an Fv fragment, a dAb, a Fd, a nanobody, a diabody, or a linear antibody. In some embodiments, reference to an antibody that specifically binds to TSLP means that the antibody binds to TSLP with an affinity that is at least about 10 times (including for example at least about any one of 10, 10², 10³, 10⁴, 10⁵, 10⁶, or 10⁷ times) more tightly than its binding affinity for a non-target. In some embodiments, the non-target is an antigen that is not TSLP. Binding affinity can be determined by methods known in the art, such as ELISA, fluorescence activated cell sorting (FACS) analysis, or radioimmunoprecipitation assay (RIA). Kd can be determined by methods known in the art, such as surface plasmon resonance (SPR) assay or biolayer interferometry (BLI).

Although anti-TSLP antibodies containing human sequences (e.g., human heavy and light chain variable domain sequences comprising human CDR sequences) are extensively discussed herein, non-human anti-TSLP antibodies are also contemplated. In some embodiments, non-human anti-TSLP antibodies comprise human CDR sequences from an anti-TSLP antibody as described herein and non-human framework sequences. Non-human framework sequences include, in some embodiments, any sequence that can be used for generating synthetic heavy and/or light chain variable domains using one or more human CDR sequences as described herein, including, e.g., mammals, e.g., mouse, rat, rabbit, pig, bovine (e.g., cow, bull, buffalo), deer, sheep, goat, chicken, cat, dog, ferret, primate (e.g., marmoset, rhesus monkey), etc. In some embodiments, a non-human anti-TSLP antibody includes an anti-TSLP antibody generated by grafting one or more human CDR sequences as described herein onto a non-human framework sequence (e.g., a mouse or chicken framework sequence).

The complete amino acid sequence of an exemplary native human TSLP comprises or consists of the amino acid sequence of SEQ ID NO: 128. The amino acid sequence of an exemplary modified human TSLP comprises or consists of the amino acid sequence of SEQ ID NO: 129.

In some embodiments, the anti-TSLP antibody described herein specifically recognizes an epitope within human TSLP. In some embodiments, the anti-TSLP antibody cross-reacts with TSLP from species other than human. In some embodiments, the anti-TSLP antibody is completely specific for human TSLP and does not exhibit cross-reactivity with TSLP from other non-human species.

In some embodiments, the anti-TSLP antibody cross-reacts with at least one allelic variant of the TSLP protein (or fragments thereof). In some embodiments, the allelic variant has up to about 30 (such as about any of 1, 2, 3, 4, 5, 6, 7, 8, 9, 10, 15, 20, 25, or 30) amino acid substitutions (such as a conservative substitution) when compared to the naturally occurring TSLP (or fragments thereof). In some embodiments, the anti-TSLP antibody does not cross-react with any allelic variant of the TSLP protein (or fragments thereof).

In some embodiments, the anti-TSLP antibody cross-reacts with at least one interspecies variant of the TSLP protein. In some embodiments, for example, the TSLP protein (or fragments thereof) is human TSLP and the interspecies variant of the TSLP protein (or fragments thereof) is a cynomolgus monkey variant thereof. In some embodiments, the anti-TSLP antibody does not cross-react with any interspecies variant of the TSLP protein.

In some embodiments, according to any of the anti-TSLP antibodies described herein, the anti-TSLP antibody comprises an antibody heavy chain constant region and an antibody light chain constant region. In some embodiments, the anti-TSLP antibody comprises an IgG1 heavy chain constant region. In some embodiments, the anti-TSLP antibody comprises an IgG2 heavy chain constant region. In some embodiments, the anti-TSLP antibody comprises an IgG3 heavy chain constant region. In some embodiments, the anti-TSLP antibody comprises an IgG4 heavy chain constant region. In some embodiments, the heavy chain constant region comprises (including consisting of or consisting essentially of) the amino acid sequence of SEQ ID NO: 124. In some embodiments, the heavy chain constant region comprises (including consisting of or consisting essentially of) the amino acid sequence of SEQ ID NO: 125. In some embodiments, the anti-TSLP comprises a lambda light chain constant region. In some embodiments, the anti-TSLP antibody comprises a kappa light chain constant region. In some embodiments, the light chain constant region comprises (including consisting of or consisting essentially of) the amino acid sequence of SEQ ID NO: 126. In some embodiments, the anti-TSLP antibody comprises a lambda light chain constant region. In some embodiments, the light chain constant region comprises (including consisting of or consisting essentially of) the amino acid sequence of SEQ ID NO: 127. In some embodiments, the anti-TSLP antibody comprises an antibody heavy chain variable domain and an antibody light chain variable domain.

In some embodiments, the anti-TSLP antibody comprises a V_{H} comprising an HC-CDR1 comprising SGYGWS (SEQ ID NO: 1); an HC-CDR2 comprising YX₁SYYGSX₂SYNPSLKS (SEQ ID NO: 103), wherein X₁ is I or F, and X₂ is I or T; and an HC-CDR3 comprising TNLLYFX₁X₂ (SEQ ID NO: 104), wherein X₁ is D or E, and X₂ is S or Y; and a light chain variable domain (V_{L}) comprising a light chain complementarity determining region (LC-CDR) 1 comprising RASQX₁X₂SX₃X₄LA (SEQ ID NO: 105), wherein X₁ is G or S, X₂ is V or I, X₃ is N or S, and X₄ is N or Y; a LC-CDR2 comprising DX1SX₂X₃X₄X₅(SEQ ID NO: 106), wherein X₁ is A or T, X₂ is S or N, X₃ is R or L, X₄ is A or Q, and X₅ is T or S; and a LC-CDR3 comprising QQYSX₁WPX₂YX₃ (SEQ ID NO: 107), wherein X₁ is D or N, X₂ is Q or E, and X₃ is T or S.

In some embodiments, the anti-TSLP antibody comprises a V_{H} comprising: an HC-CDR1 comprising the amino acid sequence of SEQ ID NO: 1, or a variant thereof comprising up to about 3 (such as about any of 1, 2, or 3) amino acid substitutions, an HC-CDR2 comprising the amino acid sequence of any one of SEQ ID NOs: 7-9, or a variant thereof comprising up to about 3 (such as about any of 1, 2, or 3) amino acid substitutions, and an HC-CDR3 comprising the amino acid sequence of any one of SEQ ID NOs: 16-18, or a variant thereof comprising up to about 3 (such as about any of 1, 2, or 3) amino acid substitutions.

In some embodiments, the anti-TSLP antibody comprises a V_{H} comprising: an HC-CDR1 comprising the amino acid sequence of SEQ ID NO: 1, an HC-CDR2 comprising the amino acid sequence of any one of SEQ ID NOs: 7-9, and an HC-CDR3 comprising the amino acid sequence of any one of SEQ ID NOs: 16-18.

In some embodiments, the anti-TSLP antibody comprises a V_{L} comprising: an LC-CDR1 comprising the amino acid sequence of any one of SEQ ID NOs: 25-28, or a variant thereof comprising up to about 3 (such as about any of 1, 2, or 3) amino acid substitutions, an LC-CDR2 comprising the amino acid sequence of any one of SEQ ID NOs: 39-41, or a variant thereof comprising up to about 3 (such as about any of 1, 2, or 3) amino acid substitutions, and an LC-CDR3 comprising the amino acid sequence of any one of SEQ ID NOs: 50-53, or a variant thereof comprising up to about 3 (such as about any of 1, 2, or 3) amino acid substitutions.

In some embodiments, the anti-TSLP antibody comprises a V_{L} comprising: an LC-CDR1 comprising the amino acid sequence of any one of SEQ ID NOs: 25-28, an LC-CDR2 comprising the amino acid sequence of any one of SEQ ID NOs: 39-41, and an LC-CDR3 comprising the amino acid sequence of any one of SEQ ID NOs: 50-53.

In some embodiments, the anti-TSLP antibody comprises a V_{H} comprising: an HC-CDR1 comprising the amino acid sequence of SEQ ID NO: 1, or a variant thereof comprising up to about 3 (such as about any of 1, 2, or 3) amino acid substitutions, an HC-CDR2 comprising the amino acid sequence of any one of SEQ ID NOs: 7-9, or a variant thereof comprising up to about 3 (such as about any of 1, 2, or 3) amino acid substitutions, and an HC-CDR3 comprising the amino acid sequence of any one of SEQ ID NOs: 16-18, or a variant thereof comprising up to about 3 (such as about any of 1, 2, or 3) amino acid substitutions; and a V_{L} comprising: an LC-CDR1 comprising the amino acid sequence of any one of SEQ ID NOs: 25-28, or a variant thereof comprising up to about 3 (such as about any of 1, 2, or 3) amino acid substitutions, an LC-CDR2 comprising the amino acid sequence of any one of SEQ ID NOs: 39-41, or a variant thereof comprising up to about 3 (such as about any of 1, 2, or 3) amino acid substitutions, and an LC-CDR3 comprising the amino acid sequence of any one of SEQ ID NOs: 50-53, or a variant thereof comprising up to about 3 (such as about any of 1, 2, or 3) amino acid substitutions.

In some embodiments, the anti-TSLP antibody comprises a V_{H} comprising: an HC-CDR1 comprising the amino acid sequence of SEQ ID NO: 1, an HC-CDR2 comprising the amino acid sequence of any one of SEQ ID NOs: 7-9, and an HC-CDR3 comprising the amino acid sequence of any one of SEQ ID NOs: 16-18; and a V_{L} comprising: an LC-CDR1 comprising the amino acid sequence of any one of SEQ ID NOs: 25-28, an LC-CDR2 comprising the amino acid sequence of any one of SEQ ID NOs: 39-41, and an LC-CDR3 comprising the amino acid sequence of any one of SEQ ID NOs: 50-53.

In some embodiments, the anti-TSLP antibody comprises a V_{H} comprising: an HC-CDR1 comprising the amino acid sequence of SEQ ID NO: 1, an HC-CDR2 comprising the amino acid sequence of SEQ ID NO: 7, and an HC-CDR3 comprising the amino acid sequence of SEQ ID NO: 16, or a variant thereof comprising up to about 5 amino acid substitutions in the HC-CDRs; and a V_{L} comprising a LC-CDR1 comprising the amino acid sequence of SEQ ID NO: 25, a LC-CDR2 comprising the amino acid sequence of SEQ ID NO: 39, and a LC-CDR3 comprising the amino acid sequence of SEQ ID NO: 50, or a variant thereof comprising up to about 5 amino acid substitutions in the LC-CDRs.

In some embodiments, the anti-TSLP antibody comprises a V_{H} comprising: an HC-CDR1 comprising the amino acid sequence of SEQ ID NO: 1, an HC-CDR2 comprising the amino acid sequence of SEQ ID NO: 7, and an HC-CDR3 comprising the amino acid sequence of SEQ ID NO: 16; and a V_{L} comprising an LC-CDR1 comprising the amino acid sequence of SEQ ID NO: 25, an LC-CDR2 comprising the amino acid sequence of SEQ ID NO: 39, and an LC-CDR3 comprising the amino acid sequence of SEQ ID NO: 50.

In some embodiments, the anti-TSLP antibody comprises a V_{H} comprising: an HC-CDR1 comprising the amino acid sequence of SEQ ID NO: 1, an HC-CDR2 comprising the amino acid sequence of SEQ ID NO: 8, and an HC-CDR3 comprising the amino acid sequence of SEQ ID NO: 16, or a variant thereof comprising up to about 5 amino acid substitutions in the HC-CDRs; and a V_{L} comprising a LC-CDR1 comprising the amino acid sequence of SEQ ID NO: 25, a LC-CDR2 comprising the amino acid sequence of SEQ ID NO: 39, and a LC-CDR3 comprising the amino acid sequence of SEQ ID NO: 51, or a variant thereof comprising up to about 5 amino acid substitutions in the LC-CDRs.

In some embodiments, the anti-TSLP antibody comprises a V_{H} comprising: an HC-CDR1 comprising the amino acid sequence of SEQ ID NO: 1, an HC-CDR2 comprising the amino acid sequence of SEQ ID NO: 8, and an HC-CDR3 comprising the amino acid sequence of SEQ ID NO: 16; and a V_{L} comprising an LC-CDR1 comprising the amino acid sequence of SEQ ID NO: 25, an LC-CDR2 comprising the amino acid sequence of SEQ ID NO: 39, and an LC-CDR3 comprising the amino acid sequence of SEQ ID NO: 51.

In some embodiments, the anti-TSLP antibody comprises a V_{H} comprising: an HC-CDR1 comprising the amino acid sequence of SEQ ID NO: 1, an HC-CDR2 comprising the amino acid sequence of SEQ ID NO: 8, and an HC-CDR3 comprising the amino acid sequence of SEQ ID NO: 17, or a variant thereof comprising up to about 5 amino acid substitutions in the HC-CDRs; and a V_{L} comprising a LC-CDR1 comprising the amino acid sequence of SEQ ID NO: 26, a LC-CDR2 comprising the amino acid sequence of SEQ ID NO: 40, and a LC-CDR3 comprising the amino acid sequence of SEQ ID NO: 52, or a variant thereof comprising up to about 5 amino acid substitutions in the LC-CDRs.

In some embodiments, the anti-TSLP antibody comprises a V_{H} comprising an HC-CDR1 comprising the amino acid sequence of SEQ ID NO: 1, an HC-CDR2 comprising the amino acid sequence of SEQ ID NO: 8, and an HC-CDR3 comprising the amino acid sequence of SEQ ID NO: 17; and a V_{L} comprising an LC-CDR1 comprising the amino acid sequence of SEQ ID NO: 26, an LC-CDR2 comprising the amino acid sequence of SEQ ID NO: 40, and an LC-CDR3 comprising the amino acid sequence of SEQ ID NO: 52.

In some embodiments, the anti-TSLP antibody comprises a V_{H} comprising: an HC-CDR1 comprising the amino acid sequence of SEQ ID NO: 1, an HC-CDR2 comprising the amino acid sequence of SEQ ID NO: 8, and an HC-CDR3 comprising the amino acid sequence of SEQ ID NO: 16, or a variant thereof comprising up to about 5 amino acid substitutions in the HC-CDRs; and a V_{L} comprising a LC-CDR1 comprising the amino acid sequence of SEQ ID NO: 27, a LC-CDR2 comprising the amino acid sequence of SEQ ID NO: 41, and a LC-CDR3 comprising the amino acid sequence of SEQ ID NO: 52, or a variant thereof comprising up to about 5 amino acid substitutions in the LC-CDRs.

In some embodiments, the anti-TSLP antibody comprises a V_{H} comprising: an HC-CDR1 comprising the amino acid sequence of SEQ ID NO: 1, an HC-CDR2 comprising the amino acid sequence of SEQ ID NO: 8, and an HC-CDR3 comprising the amino acid sequence of SEQ ID NO: 16; and a V_{L} comprising an LC-CDR1 comprising the amino acid sequence of SEQ ID NO: 27, an LC-CDR2 comprising the amino acid sequence of SEQ ID NO: 41, and an LC-CDR3 comprising the amino acid sequence of SEQ ID NO: 52.

In some embodiments, the anti-TSLP antibody comprises a V_{H} comprising: an HC-CDR1 comprising the amino acid sequence of SEQ ID NO: 1, an HC-CDR2 comprising the amino acid sequence of SEQ ID NO: 9, and an HC-CDR3 comprising the amino acid sequence of SEQ ID NO: 16, or a variant thereof comprising up to about 5 amino acid substitutions in the HC-CDRs; and a V_{L} comprising a LC-CDR1 comprising the amino acid sequence of SEQ ID NO: 28, a LC-CDR2 comprising the amino acid sequence of SEQ ID NO: 39, and a LC-CDR3 comprising the amino acid sequence of SEQ ID NO: 52, or a variant thereof comprising up to about 5 amino acid substitutions in the LC-CDRs.

In some embodiments, the anti-TSLP antibody comprises a V_{H} comprising: an HC-CDR1 comprising the amino acid sequence of SEQ ID NO: 1, an HC-CDR2 comprising the amino acid sequence of SEQ ID NO: 9, and an HC-CDR3 comprising the amino acid sequence of SEQ ID NO: 16; and a V_{L} comprising an LC-CDR1 comprising the amino acid sequence of SEQ ID NO: 28, an LC-CDR2 comprising the amino acid sequence of SEQ ID NO: 39, and an LC-CDR3 comprising the amino acid sequence of SEQ ID NO: 52.

In some embodiments, the anti-TSLP antibody comprises a V_{H} comprising: an HC-CDR1 comprising the amino acid sequence of SEQ ID NO: 1, an HC-CDR2 comprising the amino acid sequence of SEQ ID NO: 8, and an HC-CDR3 comprising the amino acid sequence of SEQ ID NO: 18, or a variant thereof comprising up to about 5 amino acid substitutions in the HC-CDRs; and a V_{L} comprising a LC-CDR1 comprising the amino acid sequence of SEQ ID NO: 27, a LC-CDR2 comprising the amino acid sequence of SEQ ID NO: 39, and a LC-CDR3 comprising the amino acid sequence of SEQ ID NO: 53, or a variant thereof comprising up to about 5 amino acid substitutions in the LC-CDRs.

In some embodiments, the anti-TSLP antibody comprises a V_{H} comprising: an HC-CDR1 comprising the amino acid sequence of SEQ ID NO: 1, an HC-CDR2 comprising the amino acid sequence of SEQ ID NO: 8, and an HC-CDR3 comprising the amino acid sequence of SEQ ID NO: 18; and a V_{L} comprising an LC-CDR1 comprising the amino acid sequence of SEQ ID NO: 27, an LC-CDR2 comprising the amino acid sequence of SEQ ID NO: 39, and an LC-CDR3 comprising the amino acid sequence of SEQ ID NO: 53.

In some embodiments, the anti-TSLP antibody comprises a V_{H} comprising the amino acid sequences of SEQ ID NOs: 1, 7-9, 16-18 or a variant thereof comprising up to about 5 amino acid substitutions; and a V_{L} comprising the amino acid sequences of SEQ ID NOs: 25-28, 39-41, 50-53 or a variant thereof comprising up to about 5 amino acid substitutions. In some embodiments, the anti-TSLP antibody comprises a V_{H} comprising the amino acid sequences of SEQ ID NOs: 1, 7-9, 16-18; and a V_{L} comprising the amino acid sequences of SEQ ID NOs: 25-28, 39-41, 50-53.

In some embodiments, the anti-TSLP antibody comprises a V_{H} comprising the amino acid sequences of SEQ ID NOs: 1, 7 and 16, or a variant thereof comprising up to about 5 amino acid substitutions; and a V_{L} comprising the amino acid sequences of SEQ ID NOs: 25, 39 and 50, or a variant thereof comprising up to about 5 amino acid substitutions. In some embodiments, the anti-TSLP antibody comprises a V_{H} comprising the amino acid sequences of SEQ ID NOs: 1, 7 and 16; and a V_{L} comprising the amino acid sequences of SEQ ID NOs: 25, 39 and 50.

In some embodiments, the anti-TSLP antibody comprises a V_{H} comprising the amino acid sequences of SEQ ID NOs: 1, 8 and 16, or a variant thereof comprising up to about 5 amino acid substitutions; and a V_{L} comprising the amino acid sequences of SEQ ID NOs: 25, 39 and 51, or a variant thereof comprising up to about 5 amino acid substitutions. In some embodiments, the anti-TSLP antibody comprises a V_{H} comprising the amino acid sequences of SEQ ID NOs: 1, 8 and 16; and a V_{L} comprising the amino acid sequences of SEQ ID NOs: 25, 39 and 51.

In some embodiments, the anti-TSLP antibody comprises a V_{H} comprising the amino acid sequences of SEQ ID NOs: 1, 8 and 17, or a variant thereof comprising up to about 5 amino acid substitutions; and a V_{L} comprising the amino acid sequences of SEQ ID NOs: 26, 40 and 52, or a variant thereof comprising up to about 5 amino acid substitutions. In some embodiments, the anti-TSLP antibody comprises a V_{H} comprising the amino acid sequences of SEQ ID NOs: 1, 8 and 17; and a V_{L} comprising the amino acid sequences of SEQ ID NOs: 26, 40 and 52.

In some embodiments, the anti-TSLP antibody comprises a V_{H} comprising the amino acid sequences of SEQ ID NOs: 1, 8 and 16, or a variant thereof comprising up to about 5 amino acid substitutions; and a V_{L} comprising the amino acid sequences of SEQ ID NOs: 27, 41 and 52, or a variant thereof comprising up to about 5 amino acid substitutions. In some embodiments, the anti-TSLP antibody comprises a V_{H} comprising the amino acid sequences of SEQ ID NOs: 1, 8 and 16; and a V_{L} comprising the amino acid sequences of SEQ ID NOs: 27, 41 and 52.

In some embodiments, the anti-TSLP antibody comprises a V_{H} comprising the amino acid sequences of SEQ ID NOs: 1, 9 and 16, or a variant thereof comprising up to about 5 amino acid substitutions; and a V_{L} comprising the amino acid sequences of SEQ ID NOs: 28, 39 and 52, or a variant thereof comprising up to about 5 amino acid substitutions. In some embodiments, the anti-TSLP antibody comprises a V_{H} comprising the amino acid sequences of SEQ ID NOs: 1, 9 and 16; and a V_{L} comprising the amino acid sequences of SEQ ID NOs: 28, 39 and 52.

In some embodiments, the anti-TSLP antibody comprises a V_{H} comprising the amino acid sequences of SEQ ID NOs: 1, 8 and 18, or a variant thereof comprising up to about 5 amino acid substitutions; and a V_{L} comprising the amino acid sequences of SEQ ID NOs: 27, 39 and 53, or a variant thereof comprising up to about 5 amino acid substitutions. In some embodiments, the anti-TSLP antibody comprises a V_{H} comprising the amino acid sequences of SEQ ID NOs: 1, 8 and 18; and a V_{L} comprising the amino acid sequences of SEQ ID NOs: 27, 39 and 53.

In some embodiments, the anti-TSLP antibody comprises a V_{H} comprising an HC-CDR1, an HC-CDR2 and an HC-CDR3 of the V_{H} comprising the amino acid sequence of any one of SEQ ID NOs: 65-72; and a V_{L} comprising a LC-CDR1, a LC-CDR2, and a LC-CDR3 of the V_{L} comprising the amino acid sequence of any one of SEQ ID NOs: 84-90.

In some embodiments, the anti-TSLP antibody comprises a V_{H} comprising one, two or three HC-CDRs of SEQ ID NO: 65. In some embodiments, the anti-TSLP antibody comprises a V_{H} comprising one, two or three HC-CDRs of SEQ ID NO: 66. In some embodiments, the anti-TSLP antibody comprises a V_{H} comprising one, two or three HC-CDRs of SEQ ID NO: 67. In some embodiments, the anti-TSLP antibody comprises a V_{H} comprising one, two or three HC-CDRs of SEQ ID NO: 68. In some embodiments, the anti-TSLP antibody comprises a V_{H} comprising one, two or three HC-CDRs of SEQ ID NO: 69.

In some embodiments, the anti-TSLP antibody comprises a V_{H} comprising one, two or three HC-CDRs of SEQ ID NO: 70. In some embodiments, the anti-TSLP antibody comprises a V_{H} comprising one, two or three HC-CDRs of SEQ ID NO: 71. In some embodiments, the anti-TSLP antibody comprises a V_{H} comprising one, two or three HC-CDRs of SEQ ID NO: 72.

In some embodiments, the anti-TSLP antibody comprises a V_{L} comprising one, two or three LC-CDRs of SEQ ID NO: 84. In some embodiments, the anti-TSLP antibody comprises a V_{L} comprising one, two or three LC-CDRs of SEQ ID NO: 85. In some embodiments, the anti-TSLP antibody comprises a V_{L} comprising one, two or three LC-CDRs of SEQ ID NO: 86. In some embodiments, the anti-TSLP antibody comprises a V_{L} comprising one, two or three LC-CDRs of SEQ ID NO: 87. In some embodiments, the anti-TSLP antibody comprises a V_{L} comprising one, two or three LC-CDRs of SEQ ID NO: 88. In some embodiments, the anti-TSLP antibody comprises a V_{L} comprising one, two or three LC-CDRs of SEQ ID NO: 89. In some embodiments, the anti-TSLP antibody comprises a V_{L} comprising one, two or three LC-CDRs of SEQ ID NO: 90.

In some embodiments, the anti-TSLP antibody comprises a V_{H} comprising HC-CDR1, HC-CDR2 and HC-CDR3 of the V_{H} of SEQ ID NO: 65, and a V_{L} comprising LC-CDR1, LC-CDR2 and LC-CDR3 of the V_{L} of SEQ ID NO: 84. In some embodiments, the anti-TSLP antibody comprises a V_{H} comprising HC-CDR1, HC-CDR2 and HC-CDR3 of the V_{H} of SEQ ID NO: 65, and a V_{L} comprising LC-CDR1, LC-CDR2 and LC-CDR3 of the V_{L} of SEQ ID NO: 85. In some embodiments, the anti-TSLP antibody comprises a V_{H} comprising HC-CDR1, HC-CDR2 and HC-CDR3 of the V_{H} of SEQ ID NO: 66, and a V_{L} comprising LC-CDR1, LC-CDR2 and LC-CDR3 of the V_{L} of SEQ ID NO: 84. In some embodiments, the anti-TSLP antibody comprises a V_{H} comprising HC-CDR1, HC-CDR2 and HC-CDR3 of the V_{H} of SEQ ID NO: 66, and a V_{L} comprising LC-CDR1, LC-CDR2 and LC-CDR3 of the V_{L} of SEQ ID NO: 85. In some embodiments, the anti-TSLP antibody comprises a V_{H} comprising HC-CDR1, HC-CDR2 and HC-CDR3 of the V_{H} of SEQ ID NO: 67, and a V_{L} comprising LC-CDR1, LC-CDR2 and LC-CDR3 of the V_{L} of SEQ ID NO: 84. In some embodiments, the anti-TSLP antibody comprises a V_{H} comprising HC-CDR1, HC-CDR2 and HC-CDR3 of the V_{H} of SEQ ID NO: 67, and a V_{L} comprising LC-CDR1, LC-CDR2 and LC-CDR3 of the V_{L} of SEQ ID NO: 85. In some embodiments, the anti-TSLP antibody comprises a V_{H} comprising HC-CDR1, HC-CDR2 and HC-CDR3 of the V_{H} of SEQ ID NO: 68, and a V_{L} comprising LC-CDR1, LC-CDR2 and LC-CDR3 of the V_{L} of SEQ ID NO: 84. In some embodiments, the anti-TSLP antibody comprises a V_{H} comprising HC-CDR1, HC-CDR2 and HC-CDR3 of the V_{H} of SEQ ID NO: 68, and a V_{L} comprising LC-CDR1, LC-CDR2 and LC-CDR3 of the V_{L} of SEQ ID NO: 85. In some embodiments, the anti-TSLP antibody comprises a V_{H} comprising HC-CDR1, HC-CDR2 and HC-CDR3 of the V_{H} of SEQ ID NO: 69, and a V_{L} comprising LC-CDR1, LC-CDR2 and LC-CDR3 of the V_{L} of SEQ ID NO: 86. In some embodiments, the anti-TSLP antibody comprises a V_{H} comprising HC-CDR1, HC-CDR2 and HC-CDR3 of the V_{H} of SEQ ID NO: 70, and a V_{L} comprising LC-CDR1, LC-CDR2 and LC-CDR3 of the V_{L} of SEQ ID NO: 87. In some embodiments, the anti-TSLP antibody comprises a V_{H} comprising HC-CDR1, HC-CDR2 and HC-CDR3 of the V_{H} of SEQ ID NO: 69, and a V_{L} comprising LC-CDR1, LC-CDR2 and LC-CDR3 of the V_{L} of SEQ ID NO: 88. In some embodiments, the anti-TSLP antibody comprises a V_{H} comprising HC-CDR1, HC-CDR2 and HC-CDR3 of the V_{H} of SEQ ID NO: 71, and a V_{L} comprising LC-CDR1, LC-CDR2 and LC-CDR3 of the V_{L} of SEQ ID NO: 89. In some embodiments, the anti-TSLP antibody comprises a V_{H} comprising HC-CDR1, HC-CDR2 and HC-CDR3 of the V_{H} of SEQ ID NO: 72, and a V_{L} comprising LC-CDR1, LC-CDR2 and LC-CDR3 of the V_{L} of SEQ ID NO: 90.

In some embodiments, the anti-TSLP antibody comprises a V_{H} comprising the amino acid sequence of any one of SEQ ID NOs: 65-72, or a variant thereof having at least about 90% (for example at least about any of 91%, 92%, 93%, 94%, 95%, 96%, 97%, 98%, or 99%) sequence identity, and a V_{L} comprising the amino acid sequence of any one of SEQ ID NOs: 84-90, or a variant thereof having at least about 90% (for example at least about any of 91%, 92%, 93%, 94%, 95%, 96%, 97%, 98%, or 99%) sequence identity. In some embodiments, the anti-TSLP antibody comprises a V_{H} comprising the amino acid sequence of any one of SEQ ID NOs: 65-72, and a V_{L} comprising the amino acid sequence of any one of SEQ ID NOs: 84-90.

In some embodiments, the anti-TSLP antibody comprises a V_{H} comprising the amino acid sequence of SEQ ID NO: 65, or a variant thereof having at least about 90% (for example at least about any of 91%, 92%, 93%, 94%, 95%, 96%, 97%, 98%, or 99%) sequence identity, and a V_{L} comprising the amino acid sequence of SEQ ID NO: 84, or a variant thereof having at least about 90% (for example at least about any of 91%, 92%, 93%, 94%, 95%, 96%, 97%, 98%, or 99%) sequence identity. In some embodiments, the anti-TSLP antibody comprises a V_{H} comprising the amino acid sequence of SEQ ID NO: 65 and a V_{L} comprising the amino acid sequence of SEQ ID NO: 84.

In some embodiments, the anti-TSLP antibody comprises a V_{H} comprising the amino acid sequence of SEQ ID NO: 65, or a variant thereof having at least about 90% (for example at least about any of 91%, 92%, 93%, 94%, 95%, 96%, 97%, 98%, or 99%) sequence identity, and a V_{L} comprising the amino acid sequence of SEQ ID NO: 85, or a variant thereof having at least about 90% (for example at least about any of 91%, 92%, 93%, 94%, 95%, 96%, 97%, 98%, or 99%) sequence identity. In some embodiments, the anti-TSLP antibody comprises a V_{H} comprising the amino acid sequence of SEQ ID NO: 65 and a V_{L} comprising the amino acid sequence of SEQ ID NO: 85.

In some embodiments, the anti-TSLP antibody comprises a V_{H} comprising the amino acid sequence of SEQ ID NO: 66, or a variant thereof having at least about 90% (for example at least about any of 91%, 92%, 93%, 94%, 95%, 96%, 97%, 98%, or 99%) sequence identity, and a V_{L} comprising the amino acid sequence of SEQ ID NO: 84, or a variant thereof having at least about 90% (for example at least about any of 91%, 92%, 93%, 94%, 95%, 96%, 97%, 98%, or 99%) sequence identity. In some embodiments, the anti-TSLP antibody comprises a V_{H} comprising the amino acid sequence of SEQ ID NO: 66 and a V_{L} comprising the amino acid sequence of SEQ ID NO: 84.

In some embodiments, the anti-TSLP antibody comprises a V_{H} comprising the amino acid sequence of SEQ ID NO: 66, or a variant thereof having at least about 90% (for example at least about any of 91%, 92%, 93%, 94%, 95%, 96%, 97%, 98%, or 99%) sequence identity, and a V_{L} comprising the amino acid sequence of SEQ ID NO: 85, or a variant thereof having at least about 90% (for example at least about any of 91%, 92%, 93%, 94%, 95%, 96%, 97%, 98%, or 99%) sequence identity. In some embodiments, the anti-TSLP antibody comprises a V_{H} comprising the amino acid sequence of SEQ ID NO: 66 and a V_{L} comprising the amino acid sequence of SEQ ID NO: 85.

In some embodiments, the anti-TSLP antibody comprises a V_{H} comprising the amino acid sequence of SEQ ID NO: 67, or a variant thereof having at least about 90% (for example at least about any of 91%, 92%, 93%, 94%, 95%, 96%, 97%, 98%, or 99%) sequence identity, and a V_{L} comprising the amino acid sequence of SEQ ID NO: 84, or a variant thereof having at least about 90% (for example at least about any of 91%, 92%, 93%, 94%, 95%, 96%, 97%, 98%, or 99%) sequence identity. In some embodiments, the anti-TSLP antibody comprises a V_{H} comprising the amino acid sequence of SEQ ID NO: 67 and a V_{L} comprising the amino acid sequence of SEQ ID NO: 84.

In some embodiments, the anti-TSLP antibody comprises a V_{H} comprising the amino acid sequence of SEQ ID NO: 67, or a variant thereof having at least about 90% (for example at least about any of 91%, 92%, 93%, 94%, 95%, 96%, 97%, 98%, or 99%) sequence identity, and a V_{L} comprising the amino acid sequence of SEQ ID NO: 85, or a variant thereof having at least about 90% (for example at least about any of 91%, 92%, 93%, 94%, 95%, 96%, 97%, 98%, or 99%) sequence identity. In some embodiments, the anti-TSLP antibody comprises a V_{H} comprising the amino acid sequence of SEQ ID NO: 67 and a V_{L} comprising the amino acid sequence of SEQ ID NO: 85.

In some embodiments, the anti-TSLP antibody comprises a V_{H} comprising the amino acid sequence of SEQ ID NO: 68, or a variant thereof having at least about 90% (for example at least about any of 91%, 92%, 93%, 94%, 95%, 96%, 97%, 98%, or 99%) sequence identity, and a V_{L} comprising the amino acid sequence of SEQ ID NO: 84, or a variant thereof having at least about 90% (for example at least about any of 91%, 92%, 93%, 94%, 95%, 96%, 97%, 98%, or 99%) sequence identity. In some embodiments, the anti-TSLP antibody comprises a V_{H} comprising the amino acid sequence of SEQ ID NO: 68 and a V_{L} comprising the amino acid sequence of SEQ ID NO: 84.

In some embodiments, the anti-TSLP antibody comprises a V_{H} comprising the amino acid sequence of SEQ ID NO: 68, or a variant thereof having at least about 90% (for example at least about any of 91%, 92%, 93%, 94%, 95%, 96%, 97%, 98%, or 99%) sequence identity, and a V_{L} comprising the amino acid sequence of SEQ ID NO: 85, or a variant thereof having at least about 90% (for example at least about any of 91%, 92%, 93%, 94%, 95%, 96%, 97%, 98%, or 99%) sequence identity. In some embodiments, the anti-TSLP antibody comprises a V_{H} comprising the amino acid sequence of SEQ ID NO: 68 and a V_{L} comprising the amino acid sequence of SEQ ID NO: 85.

In some embodiments, the anti-TSLP antibody comprises a V_{H} comprising the amino acid sequence of SEQ ID NO: 69, or a variant thereof having at least about 90% (for example at least about any of 91%, 92%, 93%, 94%, 95%, 96%, 97%, 98%, or 99%) sequence identity, and a V_{L} comprising the amino acid sequence of SEQ ID NO: 86, or a variant thereof having at least about 90% (for example at least about any of 91%, 92%, 93%, 94%, 95%, 96%, 97%, 98%, or 99%) sequence identity. In some embodiments, the anti-TSLP antibody comprises a V_{H} comprising the amino acid sequence of SEQ ID NO: 69 and a V_{L} comprising the amino acid sequence of SEQ ID NO: 86.

In some embodiments, the anti-TSLP antibody comprises a V_{H} comprising the amino acid sequence of SEQ ID NO: 70, or a variant thereof having at least about 90% (for example at least about any of 91%, 92%, 93%, 94%, 95%, 96%, 97%, 98%, or 99%) sequence identity, and a V_{L} comprising the amino acid sequence of SEQ ID NO: 87, or a variant thereof having at least about 90% (for example at least about any of 91%, 92%, 93%, 94%, 95%, 96%, 97%, 98%, or 99%) sequence identity. In some embodiments, the anti-TSLP antibody comprises a V_{H} comprising the amino acid sequence of SEQ ID NO: 70 and a V_{L} comprising the amino acid sequence of SEQ ID NO: 87.

In some embodiments, the anti-TSLP antibody comprises a V_{H} comprising the amino acid sequence of SEQ ID NO: 69, or a variant thereof having at least about 90% (for example at least about any of 91%, 92%, 93%, 94%, 95%, 96%, 97%, 98%, or 99%) sequence identity, and a V_{L} comprising the amino acid sequence of SEQ ID NO: 88, or a variant thereof having at least about 90% (for example at least about any of 91%, 92%, 93%, 94%, 95%, 96%, 97%, 98%, or 99%) sequence identity. In some embodiments, the anti-TSLP antibody comprises a V_{H} comprising the amino acid sequence of SEQ ID NO: 69 and a V_{L} comprising the amino acid sequence of SEQ ID NO: 88.

In some embodiments, the anti-TSLP antibody comprises a V_{H} comprising the amino acid sequence of SEQ ID NO: 71, or a variant thereof having at least about 90% (for example at least about any of 91%, 92%, 93%, 94%, 95%, 96%, 97%, 98%, or 99%) sequence identity, and a V_{L} comprising the amino acid sequence of SEQ ID NO: 89, or a variant thereof having at least about 90% (for example at least about any of 91%, 92%, 93%, 94%, 95%, 96%, 97%, 98%, or 99%) sequence identity. In some embodiments, the anti-TSLP antibody comprises a V_{H} comprising the amino acid sequence of SEQ ID NO: 71 and a V_{L} comprising the amino acid sequence of SEQ ID NO: 89.

In some embodiments, the anti-TSLP antibody comprises a V_{H} comprising the amino acid sequence of SEQ ID NO: 72, or a variant thereof having at least about 90% (for example at least about any of 91%, 92%, 93%, 94%, 95%, 96%, 97%, 98%, or 99%) sequence identity, and a V_{L} comprising the amino acid sequence of SEQ ID NO: 90, or a variant thereof having at least about 90% (for example at least about any of 91%, 92%, 93%, 94%, 95%, 96%, 97%, 98%, or 99%) sequence identity. In some embodiments, the anti-TSLP antibody comprises a V_{H} comprising the amino acid sequence of SEQ ID NO: 72 and a V_{L} comprising the amino acid sequence of SEQ ID NO: 90.

In some embodiments, the anti-TSLP antibody comprises a V_{H} comprising an HC-CDR1 comprising SYGIX₁ (SEQ ID NO: 108), wherein X₁ is N or S; an HC-CDR2 comprising VIX₁PLX₂X₃VX₄X₅YAEKFQG (SEQ ID NO: 109), wherein X₁ is V or I, X₂ is V or L, X₃ is G or D, X₄ is T or P, and X₅ is I or N; and an HC-CDR3 comprising GX₁EYFYWYFDL (SEQ ID NO: 110), wherein X₁ is Q or A; and a light chain variable domain (V_{L}) comprising a light chain complementarity determining region (LC-CDR) 1 comprising X₁GX₂X₃SDIGGYX₄RVS (SEQ ID NO: 111), wherein X₁ is S or T, X₂ is S or T, X₃ is S, T, I or N, and X₄ is N or D; a LC-CDR2 comprising X₁X₂X₃KRX₄S (SEQ ID NO: 112), wherein X₁ is D, G or E, X₂ is V, F or I, X₃ is S or N, and X₄ is P or S; and a LC-CDR3 comprising X₁SYAX₂X₃X₄X₅FX₆X₇(SEQ ID NO: 113), wherein X₁ is S or T, X₂ is G or S, X₃ is T or G, X₄ is D or H, X₅ is T or I, X₆ is I, G, V or A, and X₇ is L, I or F.

In some embodiments, the anti-TSLP antibody comprises a V_{H} comprising: an HC-CDR1 comprising the amino acid sequence of any one of SEQ ID NOs: 2-3, or a variant thereof comprising up to about 3 (such as about any of 1, 2, or 3) amino acid substitutions, an HC-CDR2 comprising the amino acid sequence of any one of SEQ ID NOs: 10-12, or a variant thereof comprising up to about 3 (such as about any of 1, 2, or 3) amino acid substitutions, and an HC-CDR3 comprising the amino acid sequence of any one of SEQ ID NOs: 19-20, or a variant thereof comprising up to about 3 (such as about any of 1, 2, or 3) amino acid substitutions.

In some embodiments, the anti-TSLP antibody comprises a V_{H} comprising: an HC-CDR1 comprising the amino acid sequence of any one of SEQ ID NOs: 2-3, an HC-CDR2 comprising the amino acid sequence of any one of SEQ ID NOs: 10-12, and an HC-CDR3 comprising the amino acid sequence of any one of SEQ ID NOs: 19-20.

In some embodiments, the anti-TSLP antibody comprises a V_{L} comprising: an LC-CDR1 comprising the amino acid sequence of any one of SEQ ID NOs: 29-34, or a variant thereof comprising up to about 3 (such as about any of 1, 2, or 3) amino acid substitutions, an LC-CDR2 comprising the amino acid sequence of any one of SEQ ID NOs: 42-47, or a variant thereof comprising up to about 3 (such as about any of 1, 2, or 3) amino acid substitutions, and an LC-CDR3 comprising the amino acid sequence of any one of SEQ ID NOs: 54-60, or a variant thereof comprising up to about 3 (such as about any of 1, 2, or 3) amino acid substitutions.

In some embodiments, the anti-TSLP antibody comprises a V_{L} comprising: an LC-CDR1 comprising the amino acid sequence of any one of SEQ ID NOs: 29-34, an LC-CDR2 comprising the amino acid sequence of any one of SEQ ID NOs: 42-47, and an LC-CDR3 comprising the amino acid sequence of any one of SEQ ID NOs: 54-60.

In some embodiments, the anti-TSLP antibody comprises a V_{H} comprising: an HC-CDR1 comprising the amino acid sequence of any one of SEQ ID NOs: 2-3, or a variant thereof comprising up to about 3 (such as about any of 1, 2, or 3) amino acid substitutions, an HC-CDR2 comprising the amino acid sequence of any one of SEQ ID NOs: 10-12, or a variant thereof comprising up to about 3 (such as about any of 1, 2, or 3) amino acid substitutions, and an HC-CDR3 comprising the amino acid sequence of any one of SEQ ID NOs: 19-20, or a variant thereof comprising up to about 3 (such as about any of 1, 2, or 3) amino acid substitutions; and a V_{L} comprising: an LC-CDR1 comprising the amino acid sequence of any one of SEQ ID NOs: 29-34, or a variant thereof comprising up to about 3 (such as about any of 1, 2, or 3) amino acid substitutions, an LC-CDR2 comprising the amino acid sequence of any one of SEQ ID NOs: 42-47, or a variant thereof comprising up to about 3 (such as about any of 1, 2, or 3) amino acid substitutions, and an LC-CDR3 comprising the amino acid sequence of any one of SEQ ID NOs: 54-60, or a variant thereof comprising up to about 3 (such as about any of 1, 2, or 3) amino acid substitutions.

In some embodiments, the anti-TSLP antibody comprises a V_{H} comprising: an HC-CDR1 comprising the amino acid sequence of any one of SEQ ID NOs: 2-3, an HC-CDR2 comprising the amino acid sequence of any one of SEQ ID NOs: 10-12, and an HC-CDR3 comprising the amino acid sequence of any one of SEQ ID NOs: 19-20; and a V_{L} comprising: an LC-CDR1 comprising the amino acid sequence of any one of SEQ ID NOs: 29-34, an LC-CDR2 comprising the amino acid sequence of any one of SEQ ID NOs: 42-47, and an LC-CDR3 comprising the amino acid sequence of any one of SEQ ID NOs: 54-60.

In some embodiments, the anti-TSLP antibody comprises a V_{H} comprising: an HC-CDR1 comprising the amino acid sequence of SEQ ID NO: 2, an HC-CDR2 comprising the amino acid sequence of SEQ ID NO: 10, and an HC-CDR3 comprising the amino acid sequence of SEQ ID NO: 19, or a variant thereof comprising up to about 5 amino acid substitutions in the HC-CDRs; and a V_{L} comprising a LC-CDR1 comprising the amino acid sequence of SEQ ID NO: 29, a LC-CDR2 comprising the amino acid sequence of SEQ ID NO: 42, and a LC-CDR3 comprising the amino acid sequence of SEQ ID NO: 54, or a variant thereof comprising up to about 5 amino acid substitutions in the LC-CDRs.

In some embodiments, the anti-TSLP antibody comprises a V_{H} comprising: an HC-CDR1 comprising the amino acid sequence of SEQ ID NO: 2, an HC-CDR2 comprising the amino acid sequence of SEQ ID NO: 10, and an HC-CDR3 comprising the amino acid sequence of SEQ ID NO: 19; and a V_{L} comprising an LC-CDR1 comprising the amino acid sequence of SEQ ID NO: 29, an LC-CDR2 comprising the amino acid sequence of SEQ ID NO: 42, and an LC-CDR3 comprising the amino acid sequence of SEQ ID NO: 54.

In some embodiments, the anti-TSLP antibody comprises a V_{H} comprising: an HC-CDR1 comprising the amino acid sequence of SEQ ID NO: 2, an HC-CDR2 comprising the amino acid sequence of SEQ ID NO: 10, and an HC-CDR3 comprising the amino acid sequence of SEQ ID NO: 19, or a variant thereof comprising up to about 5 amino acid substitutions in the HC-CDRs; and a V_{L} comprising a LC-CDR1 comprising the amino acid sequence of SEQ ID NO: 30, a LC-CDR2 comprising the amino acid sequence of SEQ ID NO: 43, and a LC-CDR3 comprising the amino acid sequence of SEQ ID NO: 55, or a variant thereof comprising up to about 5 amino acid substitutions in the LC-CDRs.

In some embodiments, the anti-TSLP antibody comprises a V_{H} comprising: an HC-CDR1 comprising the amino acid sequence of SEQ ID NO: 2, an HC-CDR2 comprising the amino acid sequence of SEQ ID NO: 10, and an HC-CDR3 comprising the amino acid sequence of SEQ ID NO: 19; and a V_{L} comprising an LC-CDR1 comprising the amino acid sequence of SEQ ID NO: 30, an LC-CDR2 comprising the amino acid sequence of SEQ ID NO: 43, and an LC-CDR3 comprising the amino acid sequence of SEQ ID NO: 55.

In some embodiments, the anti-TSLP antibody comprises a V_{H} comprising: an HC-CDR1 comprising the amino acid sequence of SEQ ID NO: 2, an HC-CDR2 comprising the amino acid sequence of SEQ ID NO: 10, and an HC-CDR3 comprising the amino acid sequence of SEQ ID NO: 19, or a variant thereof comprising up to about 5 amino acid substitutions in the HC-CDRs; and a V_{L} comprising a LC-CDR1 comprising the amino acid sequence of SEQ ID NO: 31, a LC-CDR2 comprising the amino acid sequence of SEQ ID NO: 44, and a LC-CDR3 comprising the amino acid sequence of SEQ ID NO: 56, or a variant thereof comprising up to about 5 amino acid substitutions in the LC-CDRs.

In some embodiments, the anti-TSLP antibody comprises a V_{H} comprising an HC-CDR1 comprising the amino acid sequence of SEQ ID NO: 2, an HC-CDR2 comprising the amino acid sequence of SEQ ID NO: 10, and an HC-CDR3 comprising the amino acid sequence of SEQ ID NO: 19; and a V_{L} comprising an LC-CDR1 comprising the amino acid sequence of SEQ ID NO: 31, an LC-CDR2 comprising the amino acid sequence of SEQ ID NO: 44, and an LC-CDR3 comprising the amino acid sequence of SEQ ID NO: 56.

In some embodiments, the anti-TSLP antibody comprises a V_{H} comprising: an HC-CDR1 comprising the amino acid sequence of SEQ ID NO: 2, an HC-CDR2 comprising the amino acid sequence of SEQ ID NO: 10, and an HC-CDR3 comprising the amino acid sequence of SEQ ID NO: 19, or a variant thereof comprising up to about 5 amino acid substitutions in the HC-CDRs; and a V_{L} comprising a LC-CDR1 comprising the amino acid sequence of SEQ ID NO: 32, a LC-CDR2 comprising the amino acid sequence of SEQ ID NO: 45, and a LC-CDR3 comprising the amino acid sequence of SEQ ID NO: 57, or a variant thereof comprising up to about 5 amino acid substitutions in the LC-CDRs.

In some embodiments, the anti-TSLP antibody comprises a V_{H} comprising: an HC-CDR1 comprising the amino acid sequence of SEQ ID NO: 2, an HC-CDR2 comprising the amino acid sequence of SEQ ID NO: 10, and an HC-CDR3 comprising the amino acid sequence of SEQ ID NO: 19; and a V_{L} comprising an LC-CDR1 comprising the amino acid sequence of SEQ ID NO: 32, an LC-CDR2 comprising the amino acid sequence of SEQ ID NO: 45, and an LC-CDR3 comprising the amino acid sequence of SEQ ID NO: 57.

In some embodiments, the anti-TSLP antibody comprises a V_{H} comprising: an HC-CDR1 comprising the amino acid sequence of SEQ ID NO: 3, an HC-CDR2 comprising the amino acid sequence of SEQ ID NO: 11, and an HC-CDR3 comprising the amino acid sequence of SEQ ID NO: 19, or a variant thereof comprising up to about 5 amino acid substitutions in the HC-CDRs; and a V_{L} comprising a LC-CDR1 comprising the amino acid sequence of SEQ ID NO: 33, a LC-CDR2 comprising the amino acid sequence of SEQ ID NO: 45, and a LC-CDR3 comprising the amino acid sequence of SEQ ID NO: 58, or a variant thereof comprising up to about 5 amino acid substitutions in the LC-CDRs.

In some embodiments, the anti-TSLP antibody comprises a V_{H} comprising: an HC-CDR1 comprising the amino acid sequence of SEQ ID NO: 3, an HC-CDR2 comprising the amino acid sequence of SEQ ID NO: 11, and an HC-CDR3 comprising the amino acid sequence of SEQ ID NO: 19; and a V_{L} comprising an LC-CDR1 comprising the amino acid sequence of SEQ ID NO: 33, an LC-CDR2 comprising the amino acid sequence of SEQ ID NO: 45, and an LC-CDR3 comprising the amino acid sequence of SEQ ID NO: 58.

In some embodiments, the anti-TSLP antibody comprises a V_{H} comprising: an HC-CDR1 comprising the amino acid sequence of SEQ ID NO: 3, an HC-CDR2 comprising the amino acid sequence of SEQ ID NO: 10, and an HC-CDR3 comprising the amino acid sequence of SEQ ID NO: 19, or a variant thereof comprising up to about 5 amino acid substitutions in the HC-CDRs; and a V_{L} comprising a LC-CDR1 comprising the amino acid sequence of SEQ ID NO: 31, a LC-CDR2 comprising the amino acid sequence of SEQ ID NO: 46, and a LC-CDR3 comprising the amino acid sequence of SEQ ID NO: 59, or a variant thereof comprising up to about 5 amino acid substitutions in the LC-CDRs.

In some embodiments, the anti-TSLP antibody comprises a V_{H} comprising: an HC-CDR1 comprising the amino acid sequence of SEQ ID NO: 3, an HC-CDR2 comprising the amino acid sequence of SEQ ID NO: 10, and an HC-CDR3 comprising the amino acid sequence of SEQ ID NO: 19; and a V_{L} comprising an LC-CDR1 comprising the amino acid sequence of SEQ ID NO: 31, an LC-CDR2 comprising the amino acid sequence of SEQ ID NO: 46, and an LC-CDR3 comprising the amino acid sequence of SEQ ID NO: 59.

In some embodiments, the anti-TSLP antibody comprises a V_{H} comprising: an HC-CDR1 comprising the amino acid sequence of SEQ ID NO: 3, an HC-CDR2 comprising the amino acid sequence of SEQ ID NO: 12, and an HC-CDR3 comprising the amino acid sequence of SEQ ID NO: 20, or a variant thereof comprising up to about 5 amino acid substitutions in the HC-CDRs; and a V_{L} comprising a LC-CDR1 comprising the amino acid sequence of SEQ ID NO: 34, a LC-CDR2 comprising the amino acid sequence of SEQ ID NO: 47, and a LC-CDR3 comprising the amino acid sequence of SEQ ID NO: 60, or a variant thereof comprising up to about 5 amino acid substitutions in the LC-CDRs.

In some embodiments, the anti-TSLP antibody comprises a V_{H} comprising: an HC-CDR1 comprising the amino acid sequence of SEQ ID NO: 3, an HC-CDR2 comprising the amino acid sequence of SEQ ID NO: 12, and an HC-CDR3 comprising the amino acid sequence of SEQ ID NO: 20; and a V_{L} comprising an LC-CDR1 comprising the amino acid sequence of SEQ ID NO: 34, an LC-CDR2 comprising the amino acid sequence of SEQ ID NO: 47, and an LC-CDR3 comprising the amino acid sequence of SEQ ID NO: 60.

In some embodiments, the anti-TSLP antibody comprises a V_{H} comprising the amino acid sequences of SEQ ID NOs: 2-3, 10-12, 19-20 or a variant thereof comprising up to about 5 amino acid substitutions; and a V_{L} comprising the amino acid sequences of SEQ ID NOs: 29-34, 42-47, 54-60 or a variant thereof comprising up to about 5 amino acid substitutions. In some embodiments, the anti-TSLP antibody comprises a V_{H} comprising the amino acid sequences of SEQ ID NOs: 2-3, 10-12, 19-20; and a V_{L} comprising the amino acid sequences of SEQ ID NOs: 29-34, 42-47, 54-60.

In some embodiments, the anti-TSLP antibody comprises a V_{H} comprising the amino acid sequences of SEQ ID NOs: 2, 10 and 19, or a variant thereof comprising up to about 5 amino acid substitutions; and a V_{L} comprising the amino acid sequences of SEQ ID NOs: 29, 42 and 54, or a variant thereof comprising up to about 5 amino acid substitutions. In some embodiments, the anti-TSLP antibody comprises a V_{H} comprising the amino acid sequences of SEQ ID NOs: 2, 10 and 19; and a V_{L} comprising the amino acid sequences of SEQ ID NOs: 29, 42 and 54.

In some embodiments, the anti-TSLP antibody comprises a V_{H} comprising the amino acid sequences of SEQ ID NOs: 2, 10 and 19, or a variant thereof comprising up to about 5 amino acid substitutions; and a V_{L} comprising the amino acid sequences of SEQ ID NOs: 30, 43 and 55, or a variant thereof comprising up to about 5 amino acid substitutions. In some embodiments, the anti-TSLP antibody comprises a V_{H} comprising the amino acid sequences of SEQ ID NOs: 2, 10 and 19; and a V_{L} comprising the amino acid sequences of SEQ ID NOs: 30, 43 and 55.

In some embodiments, the anti-TSLP antibody comprises a V_{H} comprising the amino acid sequences of SEQ ID NOs: 2, 10 and 19, or a variant thereof comprising up to about 5 amino acid substitutions; and a V_{L} comprising the amino acid sequences of SEQ ID NOs: 31, 44 and 56, or a variant thereof comprising up to about 5 amino acid substitutions. In some embodiments, the anti-TSLP antibody comprises a V_{H} comprising the amino acid sequences of SEQ ID NOs: 2, 10 and 19; and a V_{L} comprising the amino acid sequences of SEQ ID NOs: 31, 44 and 56.

In some embodiments, the anti-TSLP antibody comprises a V_{H} comprising the amino acid sequences of SEQ ID NOs: 2, 10 and 19, or a variant thereof comprising up to about 5 amino acid substitutions; and a V_{L} comprising the amino acid sequences of SEQ ID NOs: 32, 45 and 57, or a variant thereof comprising up to about 5 amino acid substitutions. In some embodiments, the anti-TSLP antibody comprises a V_{H} comprising the amino acid sequences of SEQ ID NOs: 2, 10 and 19; and a V_{L} comprising the amino acid sequences of SEQ ID NOs: 32, 45 and 57.

In some embodiments, the anti-TSLP antibody comprises a V_{H} comprising the amino acid sequences of SEQ ID NOs: 3, 11 and 19, or a variant thereof comprising up to about 5 amino acid substitutions; and a V_{L} comprising the amino acid sequences of SEQ ID NOs: 33, 45 and 58, or a variant thereof comprising up to about 5 amino acid substitutions. In some embodiments, the anti-TSLP antibody comprises a V_{H} comprising the amino acid sequences of SEQ ID NOs: 3, 11 and 19; and a V_{L} comprising the amino acid sequences of SEQ ID NOs: 33, 45 and 58.

In some embodiments, the anti-TSLP antibody comprises a V_{H} comprising the amino acid sequences of SEQ ID NOs: 3, 10 and 19, or a variant thereof comprising up to about 5 amino acid substitutions; and a V_{L} comprising the amino acid sequences of SEQ ID NOs: 31, 46 and 59, or a variant thereof comprising up to about 5 amino acid substitutions. In some embodiments, the anti-TSLP antibody comprises a V_{H} comprising the amino acid sequences of SEQ ID NOs: 3, 10 and 19; and a V_{L} comprising the amino acid sequences of SEQ ID NOs: 31, 46 and 59.

In some embodiments, the anti-TSLP antibody comprises a V_{H} comprising the amino acid sequences of SEQ ID NOs: 3, 12 and 20, or a variant thereof comprising up to about 5 amino acid substitutions; and a V_{L} comprising the amino acid sequences of SEQ ID NOs: 34, 47 and 60, or a variant thereof comprising up to about 5 amino acid substitutions. In some embodiments, the anti-TSLP antibody comprises a V_{H} comprising the amino acid sequences of SEQ ID NOs: 3, 12 and 20; and a V_{L} comprising the amino acid sequences of SEQ ID NOs: 34, 47 and 60.

In some embodiments, the anti-TSLP antibody comprises a V_{H} comprising an HC-CDR1, an HC-CDR2 and an HC-CDR3 of the V_{H} comprising the amino acid sequence of any one of SEQ ID NOs: 73-78; and a V_{L} comprising a LC-CDR1, a LC-CDR2, and a LC-CDR3 of the V_{L} comprising the amino acid sequence of any one of SEQ ID NOs: 91-97.

In some embodiments, the anti-TSLP antibody comprises a V_{H} comprising one, two or three HC-CDRs of SEQ ID NO: 73. In some embodiments, the anti-TSLP antibody comprises a V_{H} comprising one, two or three HC-CDRs of SEQ ID NO: 74. In some embodiments, the anti-TSLP antibody comprises a V_{H} comprising one, two or three HC-CDRs of SEQ ID NO: 75. In some embodiments, the anti-TSLP antibody comprises a V_{H} comprising one, two or three HC-CDRs of SEQ ID NO: 76. In some embodiments, the anti-TSLP antibody comprises a V_{H} comprising one, two or three HC-CDRs of SEQ ID NO: 77. In some embodiments, the anti-TSLP antibody comprises a V_{H} comprising one, two or three HC-CDRs of SEQ ID NO: 78.

In some embodiments, the anti-TSLP antibody comprises a V_{L} comprising one, two or three LC-CDRs of SEQ ID NO: 91. In some embodiments, the anti-TSLP antibody comprises a V_{L} comprising one, two or three LC-CDRs of SEQ ID NO: 92. In some embodiments, the anti-TSLP antibody comprises a V_{L} comprising one, two or three LC-CDRs of SEQ ID NO: 93. In some embodiments, the anti-TSLP antibody comprises a V_{L} comprising one, two or three LC-CDRs of SEQ ID NO: 94. In some embodiments, the anti-TSLP antibody comprises a V_{L} comprising one, two or three LC-CDRs of SEQ ID NO: 95. In some embodiments, the anti-TSLP antibody comprises a V_{L} comprising one, two or three LC-CDRs of SEQ ID NO: 96. In some embodiments, the anti-TSLP antibody comprises a V_{L} comprising one, two or three LC-CDRs of SEQ ID NO: 97.

In some embodiments, the anti-TSLP antibody comprises a V_{H} comprising HC-CDR1, HC-CDR2 and HC-CDR3 of the V_{H} of SEQ ID NO: 73, and a V_{L} comprising LC-CDR1, LC-CDR2 and LC-CDR3 of the V_{L} of SEQ ID NO: 91. In some embodiments, the anti-TSLP antibody comprises a V_{H} comprising HC-CDR1, HC-CDR2 and HC-CDR3 of the V_{H} of SEQ ID NO: 74, and a V_{L} comprising LC-CDR1, LC-CDR2 and LC-CDR3 of the V_{L} of SEQ ID NO: 91. In some embodiments, the anti-TSLP antibody comprises a V_{H} comprising HC-CDR1, HC-CDR2 and HC-CDR3 of the V_{H} of SEQ ID NO: 75, and a V_{L} comprising LC-CDR1, LC-CDR2 and LC-CDR3 of the V_{L} of SEQ ID NO: 91. In some embodiments, the anti-TSLP antibody comprises a V_{H} comprising HC-CDR1, HC-CDR2 and HC-CDR3 of the V_{H} of SEQ ID NO: 73, and a V_{L} comprising LC-CDR1, LC-CDR2 and LC-CDR3 of the V_{L} of SEQ ID NO: 92. In some embodiments, the anti-TSLP antibody comprises a V_{H} comprising HC-CDR1, HC-CDR2 and HC-CDR3 of the V_{H} of SEQ ID NO: 73, and a V_{L} comprising LC-CDR1, LC-CDR2 and LC-CDR3 of the V_{L} of SEQ ID NO: 93. In some embodiments, the anti-TSLP antibody comprises a V_{H} comprising HC-CDR1, HC-CDR2 and HC-CDR3 of the V_{H} of SEQ ID NO: 73, and a V_{L} comprising LC-CDR1, LC-CDR2 and LC-CDR3 of the V_{L} of SEQ ID NO: 94. In some embodiments, the anti-TSLP antibody comprises a V_{H} comprising HC-CDR1, HC-CDR2 and HC-CDR3 of the V_{H} of SEQ ID NO: 76, and a V_{L} comprising LC-CDR1, LC-CDR2 and LC-CDR3 of the V_{L} of SEQ ID NO: 95. In some embodiments, the anti-TSLP antibody comprises a V_{H} comprising HC-CDR1, HC-CDR2 and HC-CDR3 of the V_{H} of SEQ ID NO: 77, and a V_{L} comprising LC-CDR1, LC-CDR2 and LC-CDR3 of the V_{L} of SEQ ID NO: 96. In some embodiments, the anti-TSLP antibody comprises a V_{H} comprising HC-CDR1, HC-CDR2 and HC-CDR3 of the V_{H} of SEQ ID NO: 78, and a V_{L} comprising LC-CDR1, LC-CDR2 and LC-CDR3 of the V_{L} of SEQ ID NO: 97.

In some embodiments, the anti-TSLP antibody comprises a V_{H} comprising the amino acid sequence of any one of SEQ ID NOs: 73-78, or a variant thereof having at least about 90% (for example at least about any of 91%, 92%, 93%, 94%, 95%, 96%, 97%, 98%, or 99%) sequence identity, and a V_{L} comprising the amino acid sequence of any one of SEQ ID NOs: 91-97, or a variant thereof having at least about 90% (for example at least about any of 91%, 92%, 93%, 94%, 95%, 96%, 97%, 98%, or 99%) sequence identity. In some embodiments, the anti-TSLP antibody comprises a V_{H} comprising the amino acid sequence of any one of SEQ ID NOs: 73-78, and a V_{L} comprising the amino acid sequence of any one of SEQ ID NOs: 91-97.

In some embodiments, the anti-TSLP antibody comprises a V_{H} comprising the amino acid sequence of SEQ ID NO: 73, or a variant thereof having at least about 90% (for example at least about any of 91%, 92%, 93%, 94%, 95%, 96%, 97%, 98%, or 99%) sequence identity, and a V_{L} comprising the amino acid sequence of SEQ ID NO: 91, or a variant thereof having at least about 90% (for example at least about any of 91%, 92%, 93%, 94%, 95%, 96%, 97%, 98%, or 99%) sequence identity. In some embodiments, the anti-TSLP antibody comprises a V_{H} comprising the amino acid sequence of SEQ ID NO: 73 and a V_{L} comprising the amino acid sequence of SEQ ID NO: 91.

In some embodiments, the anti-TSLP antibody comprises a V_{H} comprising the amino acid sequence of SEQ ID NO: 74, or a variant thereof having at least about 90% (for example at least about any of 91%, 92%, 93%, 94%, 95%, 96%, 97%, 98%, or 99%) sequence identity, and a V_{L} comprising the amino acid sequence of SEQ ID NO: 91, or a variant thereof having at least about 90% (for example at least about any of 91%, 92%, 93%, 94%, 95%, 96%, 97%, 98%, or 99%) sequence identity. In some embodiments, the anti-TSLP antibody comprises a V_{H} comprising the amino acid sequence of SEQ ID NO: 74 and a V_{L} comprising the amino acid sequence of SEQ ID NO: 91.

In some embodiments, the anti-TSLP antibody comprises a V_{H} comprising the amino acid sequence of SEQ ID NO: 75, or a variant thereof having at least about 90% (for example at least about any of 91%, 92%, 93%, 94%, 95%, 96%, 97%, 98%, or 99%) sequence identity, and a V_{L} comprising the amino acid sequence of SEQ ID NO: 91, or a variant thereof having at least about 90% (for example at least about any of 91%, 92%, 93%, 94%, 95%, 96%, 97%, 98%, or 99%) sequence identity. In some embodiments, the anti-TSLP antibody comprises a V_{H} comprising the amino acid sequence of SEQ ID NO: 75 and a V_{L} comprising the amino acid sequence of SEQ ID NO: 91.

In some embodiments, the anti-TSLP antibody comprises a V_{H} comprising the amino acid sequence of SEQ ID NO: 73, or a variant thereof having at least about 90% (for example at least about any of 91%, 92%, 93%, 94%, 95%, 96%, 97%, 98%, or 99%) sequence identity, and a V_{L} comprising the amino acid sequence of SEQ ID NO: 92, or a variant thereof having at least about 90% (for example at least about any of 91%, 92%, 93%, 94%, 95%, 96%, 97%, 98%, or 99%) sequence identity. In some embodiments, the anti-TSLP antibody comprises a V_{H} comprising the amino acid sequence of SEQ ID NO: 73 and a V_{L} comprising the amino acid sequence of SEQ ID NO: 92.

In some embodiments, the anti-TSLP antibody comprises a V_{H} comprising the amino acid sequence of SEQ ID NO: 73, or a variant thereof having at least about 90% (for example at least about any of 91%, 92%, 93%, 94%, 95%, 96%, 97%, 98%, or 99%) sequence identity, and a V_{L} comprising the amino acid sequence of SEQ ID NO: 93, or a variant thereof having at least about 90% (for example at least about any of 91%, 92%, 93%, 94%, 95%, 96%, 97%, 98%, or 99%) sequence identity. In some embodiments, the anti-TSLP antibody comprises a V_{H} comprising the amino acid sequence of SEQ ID NO: 73 and a V_{L} comprising the amino acid sequence of SEQ ID NO: 93.

In some embodiments, the anti-TSLP antibody comprises a V_{H} comprising the amino acid sequence of SEQ ID NO: 73, or a variant thereof having at least about 90% (for example at least about any of 91%, 92%, 93%, 94%, 95%, 96%, 97%, 98%, or 99%) sequence identity, and a V_{L} comprising the amino acid sequence of SEQ ID NO: 94, or a variant thereof having at least about 90% (for example at least about any of 91%, 92%, 93%, 94%, 95%, 96%, 97%, 98%, or 99%) sequence identity. In some embodiments, the anti-TSLP antibody comprises a V_{H} comprising the amino acid sequence of SEQ ID NO: 73 and a V_{L} comprising the amino acid sequence of SEQ ID NO: 94.

In some embodiments, the anti-TSLP antibody comprises a V_{H} comprising the amino acid sequence of SEQ ID NO: 76, or a variant thereof having at least about 90% (for example at least about any of 91%, 92%, 93%, 94%, 95%, 96%, 97%, 98%, or 99%) sequence identity, and a V_{L} comprising the amino acid sequence of SEQ ID NO: 95, or a variant thereof having at least about 90% (for example at least about any of 91%, 92%, 93%, 94%, 95%, 96%, 97%, 98%, or 99%) sequence identity. In some embodiments, the anti-TSLP antibody comprises a V_{H} comprising the amino acid sequence of SEQ ID NO: 76 and a V_{L} comprising the amino acid sequence of SEQ ID NO: 95.

In some embodiments, the anti-TSLP antibody comprises a V_{H} comprising the amino acid sequence of SEQ ID NO: 77, or a variant thereof having at least about 90% (for example at least about any of 91%, 92%, 93%, 94%, 95%, 96%, 97%, 98%, or 99%) sequence identity, and a V_{L} comprising the amino acid sequence of SEQ ID NO: 96, or a variant thereof having at least about 90% (for example at least about any of 91%, 92%, 93%, 94%, 95%, 96%, 97%, 98%, or 99%) sequence identity. In some embodiments, the anti-TSLP antibody comprises a V_{H} comprising the amino acid sequence of SEQ ID NO: 77 and a V_{L} comprising the amino acid sequence of SEQ ID NO: 96.

In some embodiments, the anti-TSLP antibody comprises a V_{H} comprising the amino acid sequence of SEQ ID NO: 78, or a variant thereof having at least about 90% (for example at least about any of 91%, 92%, 93%, 94%, 95%, 96%, 97%, 98%, or 99%) sequence identity, and a V_{L} comprising the amino acid sequence of SEQ ID NO: 97, or a variant thereof having at least about 90% (for example at least about any of 91%, 92%, 93%, 94%, 95%, 96%, 97%, 98%, or 99%) sequence identity. In some embodiments, the anti-TSLP antibody comprises a V_{H} comprising the amino acid sequence of SEQ ID NO: 78 and a V_{L} comprising the amino acid sequence of SEQ ID NO: 97.

In some embodiments, the anti-TSLP antibody comprises a V_{H} comprising an HC-CDR1 comprising NYX₁MT (SEQ ID NO: 114), wherein X₁ is D or G; an HC-CDR2 comprising SITFASSYIYYADSVKG (SEQ ID NO: 13); and an HC-CDR3 comprising GGGAYX₁GGSLDV (SEQ ID NO: 115), wherein X₁ is H or Y; and a light chain variable domain (V_{L}) comprising a light chain complementarity determining region (LC-CDR) 1 comprising RSSQSLLHX₁X₂X₃YTYLH (SEQ ID NO: 116), wherein X₁ is I or S, X₂ is N or Y, and X₃ is G or E; a LC-CDR2 comprising LVSX₁RAS (SEQ ID NO: 117), wherein X₁ is H, or Y; and a LC-CDR3 comprising EQTLQTPX₁X₂ (SEQ ID NO: 118), wherein X₁ is Y or F, X₂ is S or T.

In some embodiments, the anti-TSLP antibody comprises a V_{H} comprising: an HC-CDR1 comprising the amino acid sequence of any one of SEQ ID NOs: 4-5, or a variant thereof comprising up to about 3 (such as about any of 1, 2, or 3) amino acid substitutions, an HC-CDR2 comprising the amino acid sequence of SEQ ID NO: 13, or a variant thereof comprising up to about 3 (such as about any of 1, 2, or 3) amino acid substitutions, and an HC-CDR3 comprising the amino acid sequence of any one of SEQ ID NOs: 21-22, or a variant thereof comprising up to about 3 (such as about any of 1, 2, or 3) amino acid substitutions.

In some embodiments, the anti-TSLP antibody comprises a V_{H} comprising: an HC-CDR1 comprising the amino acid sequence of any one of SEQ ID NOs: 4-5, an HC-CDR2 comprising the amino acid sequence of SEQ ID NO: 13, and an HC-CDR3 comprising the amino acid sequence of any one of SEQ ID NOs: 21-22.

In some embodiments, the anti-TSLP antibody comprises a V_{L} comprising: an LC-CDR1 comprising the amino acid sequence of any one of SEQ ID NOs: 35-37, or a variant thereof comprising up to about 3 (such as about any of 1, 2, or 3) amino acid substitutions, an LC-CDR2 comprising the amino acid sequence of any one of SEQ ID NOs: 48-49, or a variant thereof comprising up to about 3 (such as about any of 1, 2, or 3) amino acid substitutions, and an LC-CDR3 comprising the amino acid sequence of any one of SEQ ID NOs: 61-63, or a variant thereof comprising up to about 3 (such as about any of 1, 2, or 3) amino acid substitutions.

In some embodiments, the anti-TSLP antibody comprises a V_{L} comprising: an LC-CDR1 comprising the amino acid sequence of any one of SEQ ID NOs: 35-37, an LC-CDR2 comprising the amino acid sequence of any one of SEQ ID NOs: 48-49, and an LC-CDR3 comprising the amino acid sequence of any one of SEQ ID NOs: 61-63.

In some embodiments, the anti-TSLP antibody comprises a V_{H} comprising: an HC-CDR1 comprising the amino acid sequence of any one of SEQ ID NOs: 4-5, or a variant thereof comprising up to about 3 (such as about any of 1, 2, or 3) amino acid substitutions, an HC-CDR2 comprising the amino acid sequence of SEQ ID NO: 13, or a variant thereof comprising up to about 3 (such as about any of 1, 2, or 3) amino acid substitutions, and an HC-CDR3 comprising the amino acid sequence of any one of SEQ ID NOs: 21-22, or a variant thereof comprising up to about 3 (such as about any of 1, 2, or 3) amino acid substitutions; and a V_{L} comprising: an LC-CDR1 comprising the amino acid sequence of any one of SEQ ID NOs: 35-37, or a variant thereof comprising up to about 3 (such as about any of 1, 2, or 3) amino acid substitutions, an LC-CDR2 comprising the amino acid sequence of any one of SEQ ID NOs: 48-49, or a variant thereof comprising up to about 3 (such as about any of 1, 2, or 3) amino acid substitutions, and an LC-CDR3 comprising the amino acid sequence of any one of SEQ ID NOs: 61-63, or a variant thereof comprising up to about 3 (such as about any of 1, 2, or 3) amino acid substitutions.

In some embodiments, the anti-TSLP antibody comprises a V_{H} comprising: an HC-CDR1 comprising the amino acid sequence of any one of SEQ ID NOs: 4-5, an HC-CDR2 comprising the amino acid sequence of SEQ ID NO: 13, and an HC-CDR3 comprising the amino acid sequence of any one of SEQ ID NOs: 21-22; and a V_{L} comprising: an LC-CDR1 comprising the amino acid sequence of any one of SEQ ID NOs: 35-37, an LC-CDR2 comprising the amino acid sequence of any one of SEQ ID NOs: 48-49, and an LC-CDR3 comprising the amino acid sequence of any one of SEQ ID NOs: 61-63.

In some embodiments, the anti-TSLP antibody comprises a V_{H} comprising: an HC-CDR1 comprising the amino acid sequence of SEQ ID NO: 4, an HC-CDR2 comprising the amino acid sequence of SEQ ID NO: 13, and an HC-CDR3 comprising the amino acid sequence of SEQ ID NO: 21, or a variant thereof comprising up to about 5 amino acid substitutions in the HC-CDRs; and a V_{L} comprising a LC-CDR1 comprising the amino acid sequence of SEQ ID NO: 35, a LC-CDR2 comprising the amino acid sequence of SEQ ID NO: 48, and a LC-CDR3 comprising the amino acid sequence of SEQ ID NO: 61, or a variant thereof comprising up to about 5 amino acid substitutions in the LC-CDRs.

In some embodiments, the anti-TSLP antibody comprises a V_{H} comprising: an HC-CDR1 comprising the amino acid sequence of SEQ ID NO: 4, an HC-CDR2 comprising the amino acid sequence of SEQ ID NO: 13, and an HC-CDR3 comprising the amino acid sequence of SEQ ID NO: 21; and a V_{L} comprising an LC-CDR1 comprising the amino acid sequence of SEQ ID NO: 35, an LC-CDR2 comprising the amino acid sequence of SEQ ID NO: 48, and an LC-CDR3 comprising the amino acid sequence of SEQ ID NO: 61.

In some embodiments, the anti-TSLP antibody comprises a V_{H} comprising: an HC-CDR1 comprising the amino acid sequence of SEQ ID NO: 4, an HC-CDR2 comprising the amino acid sequence of SEQ ID NO: 13, and an HC-CDR3 comprising the amino acid sequence of SEQ ID NO: 22, or a variant thereof comprising up to about 5 amino acid substitutions in the HC-CDRs; and a V_{L} comprising a LC-CDR1 comprising the amino acid sequence of SEQ ID NO: 36, a LC-CDR2 comprising the amino acid sequence of SEQ ID NO: 48, and a LC-CDR3 comprising the amino acid sequence of SEQ ID NO: 62, or a variant thereof comprising up to about 5 amino acid substitutions in the LC-CDRs.

In some embodiments, the anti-TSLP antibody comprises a V_{H} comprising: an HC-CDR1 comprising the amino acid sequence of SEQ ID NO: 4, an HC-CDR2 comprising the amino acid sequence of SEQ ID NO: 13, and an HC-CDR3 comprising the amino acid sequence of SEQ ID NO: 22; and a V_{L} comprising an LC-CDR1 comprising the amino acid sequence of SEQ ID NO: 36, an LC-CDR2 comprising the amino acid sequence of SEQ ID NO: 48, and an LC-CDR3 comprising the amino acid sequence of SEQ ID NO: 62.

In some embodiments, the anti-TSLP antibody comprises a V_{H} comprising: an HC-CDR1 comprising the amino acid sequence of SEQ ID NO: 5, an HC-CDR2 comprising the amino acid sequence of SEQ ID NO: 13, and an HC-CDR3 comprising the amino acid sequence of SEQ ID NO: 22, or a variant thereof comprising up to about 5 amino acid substitutions in the HC-CDRs; and a V_{L} comprising a LC-CDR1 comprising the amino acid sequence of SEQ ID NO: 37, a LC-CDR2 comprising the amino acid sequence of SEQ ID NO: 49, and a LC-CDR3 comprising the amino acid sequence of SEQ ID NO: 63, or a variant thereof comprising up to about 5 amino acid substitutions in the LC-CDRs.

In some embodiments, the anti-TSLP antibody comprises a V_{H} comprising an HC-CDR1 comprising the amino acid sequence of SEQ ID NO: 5, an HC-CDR2 comprising the amino acid sequence of SEQ ID NO: 13, and an HC-CDR3 comprising the amino acid sequence of SEQ ID NO: 22; and a V_{L} comprising an LC-CDR1 comprising the amino acid sequence of SEQ ID NO: 37, an LC-CDR2 comprising the amino acid sequence of SEQ ID NO: 49, and an LC-CDR3 comprising the amino acid sequence of SEQ ID NO: 63.

In some embodiments, the anti-TSLP antibody comprises a V_{H} comprising the amino acid sequences of SEQ ID NOs: 4-5, 13, 21-22 or a variant thereof comprising up to about 5 amino acid substitutions; and a V_{L} comprising the amino acid sequences of SEQ ID NOs: 35-37, 48-49, 61-63 or a variant thereof comprising up to about 5 amino acid substitutions. In some embodiments, the anti-TSLP antibody comprises a V_{H} comprising the amino acid sequences of SEQ ID NOs: 4-5, 13, 21-22; and a V_{L} comprising the amino acid sequences of SEQ ID NOs: 35-37, 48-49, 61-63.

In some embodiments, the anti-TSLP antibody comprises a V_{H} comprising the amino acid sequences of SEQ ID NOs: 4, 13 and 21, or a variant thereof comprising up to about 5 amino acid substitutions; and a V_{L} comprising the amino acid sequences of SEQ ID NOs: 35, 48 and 61, or a variant thereof comprising up to about 5 amino acid substitutions. In some embodiments, the anti-TSLP antibody comprises a V_{H} comprising the amino acid sequences of SEQ ID NOs: 4, 13 and 21; and a V_{L} comprising the amino acid sequences of SEQ ID NOs: 35, 48 and 61.

In some embodiments, the anti-TSLP antibody comprises a V_{H} comprising the amino acid sequences of SEQ ID NOs: 4, 13 and 22, or a variant thereof comprising up to about 5 amino acid substitutions; and a V_{L} comprising the amino acid sequences of SEQ ID NOs: 36, 48 and 62, or a variant thereof comprising up to about 5 amino acid substitutions. In some embodiments, the anti-TSLP antibody comprises a V_{H} comprising the amino acid sequences of SEQ ID NOs: 4, 13 and 22; and a V_{L} comprising the amino acid sequences of SEQ ID NOs: 36, 48 and 62.

In some embodiments, the anti-TSLP antibody comprises a V_{H} comprising the amino acid sequences of SEQ ID NOs: 5, 13 and 22, or a variant thereof comprising up to about 5 amino acid substitutions; and a V_{L} comprising the amino acid sequences of SEQ ID NOs: 37, 49 and 63, or a variant thereof comprising up to about 5 amino acid substitutions. In some embodiments, the anti-TSLP antibody comprises a V_{H} comprising the amino acid sequences of SEQ ID NOs: 5, 13 and 22; and a V_{L} comprising the amino acid sequences of SEQ ID NOs: 37, 49 and 63.

In some embodiments, the anti-TSLP antibody comprises a V_{H} comprising an HC-CDR1, an HC-CDR2 and an HC-CDR3 of the V_{H} comprising the amino acid sequence of any one of SEQ ID NOs: 79-81; and a V_{L} comprising a LC-CDR1, a LC-CDR2, and a LC-CDR3 of the V_{L} comprising the amino acid sequence of any one of SEQ ID NOs: 98-100.

In some embodiments, the anti-TSLP antibody comprises a V_{H} comprising one, two or three HC-CDRs of SEQ ID NO: 79. In some embodiments, the anti-TSLP antibody comprises a V_{H} comprising one, two or three HC-CDRs of SEQ ID NO: 80. In some embodiments, the anti-TSLP antibody comprises a V_{H} comprising one, two or three HC-CDRs of SEQ ID NO: 81.

In some embodiments, the anti-TSLP antibody comprises a V_{L} comprising one, two or three LC-CDRs of SEQ ID NO: 98. In some embodiments, the anti-TSLP antibody comprises a V_{L} comprising one, two or three LC-CDRs of SEQ ID NO: 99. In some embodiments, the anti-TSLP antibody comprises a V_{L} comprising one, two or three LC-CDRs of SEQ ID NO: 100.

In some embodiments, the anti-TSLP antibody comprises a V_{H} comprising HC-CDR1, HC-CDR2 and HC-CDR3 of the V_{H} of SEQ ID NO: 79, and a V_{L} comprising LC-CDR1, LC-CDR2 and LC-CDR3 of the V_{L} of SEQ ID NO: 98. In some embodiments, the anti-TSLP antibody comprises a V_{H} comprising HC-CDR1, HC-CDR2 and HC-CDR3 of the V_{H} of SEQ ID NO: 80, and a V_{L} comprising LC-CDR1, LC-CDR2 and LC-CDR3 of the V_{L} of SEQ ID NO: 99. In some embodiments, the anti-TSLP antibody comprises a V_{H} comprising HC-CDR1, HC-CDR2 and HC-CDR3 of the V_{H} of SEQ ID NO: 81, and a V_{L} comprising LC-CDR1, LC-CDR2 and LC-CDR3 of the V_{L} of SEQ ID NO: 100.

In some embodiments, the anti-TSLP antibody comprises a V_{H} comprising the amino acid sequence of any one of SEQ ID NOs: 79-81, or a variant thereof having at least about 90% (for example at least about any of 91%, 92%, 93%, 94%, 95%, 96%, 97%, 98%, or 99%) sequence identity, and a V_{L} comprising the amino acid sequence of any one of SEQ ID NOs: 98-100, or a variant thereof having at least about 90% (for example at least about any of 91%, 92%, 93%, 94%, 95%, 96%, 97%, 98%, or 99%) sequence identity. In some embodiments, the anti-TSLP antibody comprises a V_{H} comprising the amino acid sequence of any one of SEQ ID NOs: 79-81, and a V_{L} comprising the amino acid sequence of any one of SEQ ID NOs: 98-100.

In some embodiments, the anti-TSLP antibody comprises a V_{H} comprising the amino acid sequence of SEQ ID NO: 79, or a variant thereof having at least about 90% (for example at least about any of 91%, 92%, 93%, 94%, 95%, 96%, 97%, 98%, or 99%) sequence identity, and a V_{L} comprising the amino acid sequence of SEQ ID NO: 98, or a variant thereof having at least about 90% (for example at least about any of 91%, 92%, 93%, 94%, 95%, 96%, 97%, 98%, or 99%) sequence identity. In some embodiments, the anti-TSLP antibody comprises a V_{H} comprising the amino acid sequence of SEQ ID NO: 79 and a V_{L} comprising the amino acid sequence of SEQ ID NO: 98.

In some embodiments, the anti-TSLP antibody comprises a V_{H} comprising the amino acid sequence of SEQ ID NO: 80, or a variant thereof having at least about 90% (for example at least about any of 91%, 92%, 93%, 94%, 95%, 96%, 97%, 98%, or 99%) sequence identity, and a V_{L} comprising the amino acid sequence of SEQ ID NO: 99, or a variant thereof having at least about 90% (for example at least about any of 91%, 92%, 93%, 94%, 95%, 96%, 97%, 98%, or 99%) sequence identity. In some embodiments, the anti-TSLP antibody comprises a V_{H} comprising the amino acid sequence of SEQ ID NO: 80 and a V_{L} comprising the amino acid sequence of SEQ ID NO: 99.

In some embodiments, the anti-TSLP antibody comprises a V_{H} comprising the amino acid sequence of SEQ ID NO: 81, or a variant thereof having at least about 90% (for example at least about any of 91%, 92%, 93%, 94%, 95%, 96%, 97%, 98%, or 99%) sequence identity, and a V_{L} comprising the amino acid sequence of SEQ ID NO: 100, or a variant thereof having at least about 90% (for example at least about any of 91%, 92%, 93%, 94%, 95%, 96%, 97%, 98%, or 99%) sequence identity. In some embodiments, the anti-TSLP antibody comprises a V_{H} comprising the amino acid sequence of SEQ ID NO: 81 and a V_{L} comprising the amino acid sequence of SEQ ID NO: 100.

In some embodiments, the anti-TSLP antibody comprises a V_{H} comprising an HC-CDR1 comprising SYAIS (SEQ ID NO: 6); an HC-CDR2 comprising MX₁X₂PLLGVTX₃YAEKFQG (SEQ ID NO: 119), wherein X₁ is L or I, X₂ is V or I, and X₃ is N or D; and an HC-CDR3 comprising GGX₁NYLYWYFDL (SEQ ID NO: 120), wherein X₁ is S or T; and a light chain variable domain (V_{L}) comprising a light chain complementarity determining region (LC-CDR) 1 comprising TGTSSDIGGYNRX₁S (SEQ ID NO: 121), wherein X₁ is V or I; a LC-CDR2 comprising X₁VSKRPS (SEQ ID NO: 122), wherein X₁ is D or E; and a LC-CDR3 comprising SX₁YAGTDTFVL (SEQ ID NO: 123), wherein X₁ is S or A.

In some embodiments, the anti-TSLP antibody comprises a V_{H} comprising: an HC-CDR1 comprising the amino acid sequence of SEQ ID NO: 6, or a variant thereof comprising up to about 3 (such as about any of 1, 2, or 3) amino acid substitutions, an HC-CDR2 comprising the amino acid sequence of any one of SEQ ID NOs: 14-15, or a variant thereof comprising up to about 3 (such as about any of 1, 2, or 3) amino acid substitutions, and an HC-CDR3 comprising the amino acid sequence of any one of SEQ ID NOs: 23-24, or a variant thereof comprising up to about 3 (such as about any of 1, 2, or 3) amino acid substitutions.

In some embodiments, the anti-TSLP antibody comprises a V_{H} comprising: an HC-CDR1 comprising the amino acid sequence of SEQ ID NO: 6, an HC-CDR2 comprising the amino acid sequence of any one of SEQ ID NO: 14-15, and an HC-CDR3 comprising the amino acid sequence of any one of SEQ ID NOs: 23-24.

In some embodiments, the anti-TSLP antibody comprises a V_{L} comprising: an LC-CDR1 comprising the amino acid sequence of any one of SEQ ID NOs: 31, 38, or a variant thereof comprising up to about 3 (such as about any of 1, 2, or 3) amino acid substitutions, an LC-CDR2 comprising the amino acid sequence of any one of SEQ ID NOs: 42, 45, or a variant thereof comprising up to about 3 (such as about any of 1, 2, or 3) amino acid substitutions, and an LC-CDR3 comprising the amino acid sequence of any one of SEQ ID NOs: 60, 64, or a variant thereof comprising up to about 3 (such as about any of 1, 2, or 3) amino acid substitutions.

In some embodiments, the anti-TSLP antibody comprises a V_{L} comprising: an LC-CDR1 comprising the amino acid sequence of any one of SEQ ID NOs: 31, 38, an LC-CDR2 comprising the amino acid sequence of any one of SEQ ID NOs: 42, 45, and an LC-CDR3 comprising the amino acid sequence of any one of SEQ ID NOs: 60, 64.

In some embodiments, the anti-TSLP antibody comprises a V_{H} comprising: an HC-CDR1 comprising the amino acid sequence of SEQ ID NO: 6, or a variant thereof comprising up to about 3 (such as about any of 1, 2, or 3) amino acid substitutions, an HC-CDR2 comprising the amino acid sequence of any one of SEQ ID NOs: 14-15, or a variant thereof comprising up to about 3 (such as about any of 1, 2, or 3) amino acid substitutions, and an HC-CDR3 comprising the amino acid sequence of any one of SEQ ID NOs: 23-24, or a variant thereof comprising up to about 3 (such as about any of 1, 2, or 3) amino acid substitutions; and a V_{L} comprising: an LC-CDR1 comprising the amino acid sequence of any one of SEQ ID NOs: 31, 38, or a variant thereof comprising up to about 3 (such as about any of 1, 2, or 3) amino acid substitutions, an LC-CDR2 comprising the amino acid sequence of any one of SEQ ID NOs: 42, 45, or a variant thereof comprising up to about 3 (such as about any of 1, 2, or 3) amino acid substitutions, and an LC-CDR3 comprising the amino acid sequence of any one of SEQ ID NOs: 60, 64, or a variant thereof comprising up to about 3 (such as about any of 1, 2, or 3) amino acid substitutions.

In some embodiments, the anti-TSLP antibody comprises a V_{H} comprising: an HC-CDR1 comprising the amino acid sequence of SEQ ID NO: 6, an HC-CDR2 comprising the amino acid sequence of any one of SEQ ID NOs: 14-15, and an HC-CDR3 comprising the amino acid sequence of any one of SEQ ID NOs: 23-24; and a V_{L} comprising: an LC-CDR1 comprising the amino acid sequence of any one of SEQ ID NOs: 31, 38, an LC-CDR2 comprising the amino acid sequence of any one of SEQ ID NOs: 42, 45, and an LC-CDR3 comprising the amino acid sequence of any one of SEQ ID NOs: 60, 64.

In some embodiments, the anti-TSLP antibody comprises a V_{H} comprising: an HC-CDR1 comprising the amino acid sequence of SEQ ID NO: 6, an HC-CDR2 comprising the amino acid sequence of SEQ ID NO: 14, and an HC-CDR3 comprising the amino acid sequence of SEQ ID NO: 23, or a variant thereof comprising up to about 5 amino acid substitutions in the HC-CDRs; and a V_{L} comprising a LC-CDR1 comprising the amino acid sequence of SEQ ID NO: 38, a LC-CDR2 comprising the amino acid sequence of SEQ ID NO: 42, and a LC-CDR3 comprising the amino acid sequence of SEQ ID NO: 60, or a variant thereof comprising up to about 5 amino acid substitutions in the LC-CDRs.

In some embodiments, the anti-TSLP antibody comprises a V_{H} comprising: an HC-CDR1 comprising the amino acid sequence of SEQ ID NO: 6, an HC-CDR2 comprising the amino acid sequence of SEQ ID NO: 14, and an HC-CDR3 comprising the amino acid sequence of SEQ ID NO: 23; and a V_{L} comprising an LC-CDR1 comprising the amino acid sequence of SEQ ID NO: 38, an LC-CDR2 comprising the amino acid sequence of SEQ ID NO: 42, and an LC-CDR3 comprising the amino acid sequence of SEQ ID NO: 60.

In some embodiments, the anti-TSLP antibody comprises a V_{H} comprising: an HC-CDR1 comprising the amino acid sequence of SEQ ID NO: 6, an HC-CDR2 comprising the amino acid sequence of SEQ ID NO: 15, and an HC-CDR3 comprising the amino acid sequence of SEQ ID NO: 24, or a variant thereof comprising up to about 5 amino acid substitutions in the HC-CDRs; and a V_{L} comprising a LC-CDR1 comprising the amino acid sequence of SEQ ID NO: 31, a LC-CDR2 comprising the amino acid sequence of SEQ ID NO: 45, and a LC-CDR3 comprising the amino acid sequence of SEQ ID NO: 64, or a variant thereof comprising up to about 5 amino acid substitutions in the LC-CDRs.

In some embodiments, the anti-TSLP antibody comprises a V_{H} comprising: an HC-CDR1 comprising the amino acid sequence of SEQ ID NO: 6, an HC-CDR2 comprising the amino acid sequence of SEQ ID NO: 15, and an HC-CDR3 comprising the amino acid sequence of SEQ ID NO: 24; and a V_{L} comprising an LC-CDR1 comprising the amino acid sequence of SEQ ID NO: 31, an LC-CDR2 comprising the amino acid sequence of SEQ ID NO: 45, and an LC-CDR3 comprising the amino acid sequence of SEQ ID NO: 64.

In some embodiments, the anti-TSLP antibody comprises a V_{H} comprising the amino acid sequences of SEQ ID NOs: 6, 14-15, 23-24 or a variant thereof comprising up to about 5 amino acid substitutions; and a V_{L} comprising the amino acid sequences of SEQ ID NOs: 31, 38, 42, 45, 60, 64 or a variant thereof comprising up to about 5 amino acid substitutions. In some embodiments, the anti-TSLP antibody comprises a V_{H} comprising the amino acid sequences of SEQ ID NOs: 6, 14-15, 23-24; and a V_{L} comprising the amino acid sequences of SEQ ID NOs: 31, 38, 42, 45, 60, 64.

In some embodiments, the anti-TSLP antibody comprises a V_{H} comprising the amino acid sequences of SEQ ID NOs: 6, 14 and 23, or a variant thereof comprising up to about 5 amino acid substitutions; and a V_{L} comprising the amino acid sequences of SEQ ID NOs: 38, 42 and 60, or a variant thereof comprising up to about 5 amino acid substitutions. In some embodiments, the anti-TSLP antibody comprises a V_{H} comprising the amino acid sequences of SEQ ID NOs: 6, 14 and 23; and a V_{L} comprising the amino acid sequences of SEQ ID NOs: 38, 42 and 60.

In some embodiments, the anti-TSLP antibody comprises a V_{H} comprising the amino acid sequences of SEQ ID NOs: 6, 15 and 24, or a variant thereof comprising up to about 5 amino acid substitutions; and a V_{L} comprising the amino acid sequences of SEQ ID NOs: 31, 45 and 64, or a variant thereof comprising up to about 5 amino acid substitutions. In some embodiments, the anti-TSLP antibody comprises a V_{H} comprising the amino acid sequences of SEQ ID NOs: 6, 15 and 24; and a V_{L} comprising the amino acid sequences of SEQ ID NOs: 31, 45 and 64.

In some embodiments, the anti-TSLP antibody comprises a V_{H} comprising an HC-CDR1, an HC-CDR2 and an HC-CDR3 of the V_{H} comprising the amino acid sequence of any one of SEQ ID NOs: 82-83; and a V_{L} comprising a LC-CDR1, a LC-CDR2, and a LC-CDR3 of the V_{L} comprising the amino acid sequence of any one of SEQ ID NOs: 101-102.

In some embodiments, the anti-TSLP antibody comprises a V_{H} comprising one, two or three HC-CDRs of SEQ ID NO: 82. In some embodiments, the anti-TSLP antibody comprises a V_{H} comprising one, two or three HC-CDRs of SEQ ID NO: 83.

In some embodiments, the anti-TSLP antibody comprises a V_{L} comprising one, two or three LC-CDRs of SEQ ID NO: 101. In some embodiments, the anti-TSLP antibody comprises a V_{L} comprising one, two or three LC-CDRs of SEQ ID NO: 102.

In some embodiments, the anti-TSLP antibody comprises a V_{H} comprising HC-CDR1, HC-CDR2 and HC-CDR3 of the V_{H} of SEQ ID NO: 82, and a V_{L} comprising LC-CDR1, LC-CDR2 and LC-CDR3 of the V_{L} of SEQ ID NO: 101. In some embodiments, the anti-TSLP antibody comprises a V_{H} comprising HC-CDR1, HC-CDR2 and HC-CDR3 of the V_{H} of SEQ ID NO: 83, and a V_{L} comprising LC-CDR1, LC-CDR2 and LC-CDR3 of the V_{L} of SEQ ID NO: 102.

In some embodiments, the anti-TSLP antibody comprises a V_{H} comprising the amino acid sequence of any one of SEQ ID NOs: 82-83, or a variant thereof having at least about 90% (for example at least about any of 91%, 92%, 93%, 94%, 95%, 96%, 97%, 98%, or 99%) sequence identity, and a V_{L} comprising the amino acid sequence of any one of SEQ ID NOs: 101-102, or a variant thereof having at least about 90% (for example at least about any of 91%, 92%, 93%, 94%, 95%, 96%, 97%, 98%, or 99%) sequence identity. In some embodiments, the anti-TSLP antibody comprises a V_{H} comprising the amino acid sequence of any one of SEQ ID NOs: 82-83, and a V_{L} comprising the amino acid sequence of any one of SEQ ID NOs: 101-102.

In some embodiments, the anti-TSLP antibody comprises a V_{H} comprising the amino acid sequence of SEQ ID NO: 82, or a variant thereof having at least about 90% (for example at least about any of 91%, 92%, 93%, 94%, 95%, 96%, 97%, 98%, or 99%) sequence identity, and a V_{L} comprising the amino acid sequence of SEQ ID NO: 101, or a variant thereof having at least about 90% (for example at least about any of 91%, 92%, 93%, 94%, 95%, 96%, 97%, 98%, or 99%) sequence identity. In some embodiments, the anti-TSLP antibody comprises a V_{H} comprising the amino acid sequence of SEQ ID NO: 82 and a V_{L} comprising the amino acid sequence of SEQ ID NO: 101.

In some embodiments, the anti-TSLP antibody comprises a V_{H} comprising the amino acid sequence of SEQ ID NO: 83, or a variant thereof having at least about 90% (for example at least about any of 91%, 92%, 93%, 94%, 95%, 96%, 97%, 98%, or 99%) sequence identity, and a V_{L} comprising the amino acid sequence of SEQ ID NO: 102, or a variant thereof having at least about 90% (for example at least about any of 91%, 92%, 93%, 94%, 95%, 96%, 97%, 98%, or 99%) sequence identity. In some embodiments, the anti-TSLP antibody comprises a V_{H} comprising the amino acid sequence of SEQ ID NO: 83 and a V_{L} comprising the amino acid sequence of SEQ ID NO: 102.

In some embodiments, functional epitopes can be mapped by combinatorial alanine scanning. In this process, a combinatorial alanine-scanning strategy can be used to identify amino acids in the TSLP protein that are necessary for interaction with TSLP antibodies. In some embodiments, the epitope is conformational and crystal structure of anti-TSLP antibodies bound to TSLP may be employed to identify the epitopes.

In some embodiments, the amino acid substitutions described above are limited to “exemplary substitutions” shown in Table 4 of this application. In some embodiments, the amino acid substitutions are limited to “preferred substitutions” shown in Table 4 of this application.

In some embodiments, the present application provides antibodies which compete with any one of the TSLP antibodies described herein for binding to TSLP. In some embodiments, the present application provides antibodies which compete with any one of the anti-TSLP antibodies provided herein for binding to an epitope on the TSLP. In some embodiments, an anti-TSLP antibody is provided that binds to the same epitope as an anti-TSLP antibody comprising a V_{H} comprising the amino acid sequence of any one of SEQ ID NOs: 65-83, and a V_{L} comprising the amino acid sequence of any one of SEQ ID NOs: 84-102. In some embodiments, an anti-TSLP antibody is provided that specifically binds to TSLP competitively with an anti-TSLP antibody comprising a V_{H} comprising the amino acid sequence of any one of SEQ ID NOs: 65-83 and a V_{L} comprising the amino acid sequence of any one of SEQ ID NOs: 84-102.

In some embodiments, competition assays may be used to identify a monoclonal antibody that competes with an anti-TSLP antibody described herein for binding to TSLP. Competition assays can be used to determine whether two antibodies bind to the same epitope by recognizing identical or sterically overlapping epitopes or one antibody competitively inhibits binding of another antibody to the antigen. In certain embodiments, such a competing antibody binds to the same epitope that is bound by an antibody described herein. Exemplary competition assays include, but are not limited to, routine assays such as those provided in Harlow and Lane (1988) Antibodies: A Laboratory Manual ch. 14 (Cold Spring Harbor Laboratory, Cold Spring Harbor, N.Y.). Detailed exemplary methods for mapping an epitope to which an antibody binds are provided in Morris (1996) “Epitope Mapping Protocols,” in Methods in Molecular Biology vol. 66 (Humana Press, Totowa, N.J.). In some embodiments, two antibodies are said to bind to the same epitope if each blocks binding of the other by 5000 or more. In some embodiments, the antibody that competes with an anti-TSLP antibody described herein is a chimeric, humanized or human antibody.

Exemplary anti-TSLP antibody sequences are shown in Tables 2 and 3, wherein the CDR numbering is according to the EU index of Kabat. Those skilled in the art will recognize that many algorithms are known for prediction of CDR positions and for delimitation of antibody heavy chain and light chain variable regions. Anti-TSLP antibodies comprising CDRs, V_{H} and/or V_{L} sequences from antibodies described herein, but based on prediction algorithms other than those exemplified in the tables below, are within the scope of this invention.

TABLE 2
Exemplary anti-TSLP antibody CDR sequences.

| Antibody | | | |
|---|---|---|---|
| Name | HC-CDR1 | HC-CDR2 | HC-CDR3 |
| TSLP-01 | SGYGWS | YISYYGSISYNPSLKS | TNLLYFDS |
| | (SEQ ID NO: 1) | (SEQ ID NO: 7) | (SEQ ID NO: 16) |
| TSLP- | SGYGWS | YISYYGSISYNPSLKS | TNLLYFDS |
| 0101 | (SEQ ID NO: 1) | (SEQ ID NO: 7) | (SEQ ID NO: 16) |
| TSLP- | SGYGWS | YISYYGSISYNPSLKS | TNLLYFDS |
| 0102 | (SEQ ID NO: 1) | (SEQ ID NO: 7) | (SEQ ID NO: 16) |
| TSLP- | SGYGWS | YISYYGSISYNPSLKS | TNLLYFDS |
| 0103 | (SEQ ID NO: 1) | (SEQ ID NO: 7) | (SEQ ID NO: 16) |
| TSLP- | SGYGWS | YISYYGSISYNPSLKS | TNLLYFDS |
| 0104 | (SEQ ID NO: 1) | (SEQ ID NO: 7) | (SEQ ID NO: 16) |
| TSLP- | SGYGWS | YISYYGSISYNPSLKS | TNLLYFDS |
| 0105 | (SEQ ID NO: 1) | (SEQ ID NO: 7) | (SEQ ID NO: 16) |
| TSLP- | SGYGWS | YISYYGSISYNPSLKS | TNLLYFDS |
| 0106 | (SEQ ID NO: 1) | (SEQ ID NO: 7) | (SEQ ID NO: 16) |
| TSLP- | SGYGWS | YISYYGSISYNPSLKS | TNLLYFDS |
| 0107 | (SEQ ID NO: 1) | (SEQ ID NO: 7) | (SEQ ID NO: 16) |
| TSLP- | SGYGWS | YISYYGSTSYNPSLKS | TNLLYFDS |
| 0108 | (SEQ ID NO: 1) | (SEQ ID NO: 8) | (SEQ ID NO: 16) |
| TSLP- | SGYGWS | YISYYGSTSYNPSLKS | TNLLYFES |
| 0109 | (SEQ ID NO: 1) | (SEQ ID NO: 8) | (SEQ ID NO: 17) |
| TSLP- | SGYGWS | YISYYGSTSYNPSLKS | TNLLYFDS |
| 0110 | (SEQ ID NO: 1) | (SEQ ID NO: 8) | (SEQ ID NO: 16) |
| TSLP- | SGYGWS | YFSYYGSTSYNPSLKS | TNLLYFDS |
| 0111 | (SEQ ID NO: 1) | (SEQ ID NO: 9) | (SEQ ID NO: 16) |
| TSLP- | SGYGWS | YISYYGSTSYNPSLKS | TNLLYFDY |
| 0112 | (SEQ ID NO: 1) | (SEQ ID NO: 8) | (SEQ ID NO: 18) |
| Composite | SGYGWS | YX₁SYYGSX₂SYNPSLKS | TNLLYFX₁X₂ |
| 1 | (SEQ ID NO: 1) | (SEQ ID NO: 103) | (SEQ ID NO: 104) |
| | | Wherein, X₁ is I or F; | Wherein, X₁ is D or E; |
| | | And X₂ is I or T | And X₂ is S or Y |
| TSLP-02 | SYGIN | VIVPLVGVTIYAEKFQG | GQEYFYWYFDL (SEQ |
| | (SEQ ID NO: 2) | (SEQ ID NO: 10) | ID NO: 19) |
| TSLP- | SYGIN | VIVPLVGVTIYAEKFQG | GQEYFYWYFDL (SEQ |
| 0201 | (SEQ ID NO: 2) | (SEQ ID NO: 10) | ID NO: 19) |
| TSLP- | SYGIN | VIVPLVGVTIYAEKFQG | GQEYFYWYFDL (SEQ |
| 0202 | (SEQ ID NO: 2) | (SEQ ID NO: 10) | ID NO: 19) |
| TSLP- | SYGIN | VIVPLVGVTIYAEKFQG | GQEYFYWYFDL (SEQ |
| 0203 | (SEQ ID NO: 2) | (SEQ ID NO: 10) | ID NO: 19) |
| TSLP- | SYGIN | VIVPLVGVTIYAEKFQG | GQEYFYWYFDL (SEQ |
| 0204 | (SEQ ID NO: 2) | (SEQ ID NO: 10) | ID NO: 19) |
| TSLP- | SYGIN | VIVPLVGVTIYAEKFQG | GQEYFYWYFDL (SEQ |
| 0205 | (SEQ ID NO: 2) | (SEQ ID NO: 10) | ID NO: 19) |
| TSLP- | SYGIS | VIVPLVDVTIYAEKFQG | GQEYFYWYFDL (SEQ |
| 0206 | (SEQ ID NO: 3) | (SEQ ID NO: 11) | ID NO: 19) |
| TSLP- | SYGIS | VIVPLVGVTIYAEKFQG | GQEYFYWYFDL (SEQ |
| 0207 | (SEQ ID NO: 3) | (SEQ ID NO: 10) | ID NO: 19) |
| TSLP- | SYGIS | VIIPLLGVPNYAEKFQG | GAEYFYWYFDL |
| 0208 | (SEQ ID NO: 3) | (SEQ ID NO: 12) | (SEQ ID NO: 20) |
| Composite | SYGIX₁ | VIX₁PLX₂X₃VX₄X₅YAEKFQG | GX₁EYFYWYFDL |
| 2 | (SEQ ID NO: 108) | (SEQ ID NO: 109) | (SEQ ID NO: 110) |
| | X₁ is N or S | Wherein, X₁ is V or I; X₂ is V or | Wherein, X₁ is Q or A |
| | | L; X₃ is G or D; X₄ is T or P; | |
| | | And X₅ is I or N | |
| TSLP-03 | NYDMT | SITFASSYIYYADSVKG | GGGAYHGGSLDV |
| | (SEQ ID NO: 4) | (SEQ ID NO: 13) | (SEQ ID NO: 21) |
| TSLP- | NYDMT | SITFASSYIYYADSVKG | GGGAYYGGSLDV |
| 0301 | (SEQ ID NO: 4) | (SEQ ID NO: 13) | (SEQ ID NO: 22) |
| TSLP- | NYGMT | SITFASSYIYYADSVKG | GGGAYYGGSLDV |
| 0302 | (SEQ ID NO: 5) | (SEQ ID NO: 13) | (SEQ ID NO: 22) |
| Composite | NYX₁MT | SITFASSYIYYADSVKG | GGGAYX₁GGSLDV |
| 3 | (SEQ ID NO: 114) | (SEQ ID NO: 13) | (SEQ ID NO: 115) |
| | Wherein, X₁ is D or G | | Wherein, X₁ is H or Y |
| TSLP-04 | SYAIS | MLVPLLGVTNYAEKFQG | GGSNYLYWYFDL |
| | (SEQ ID NO: 6) | (SEQ ID NO: 14) | (SEQ ID NO: 23) |
| TSLP- | SYAIS | MIIPLLGVTDYAEKFQG | GGTNYLYWYFDL |
| 0401 | (SEQ ID NO: 6) | (SEQ ID NO: 15) | (SEQ ID NO: 24) |
| Composite | SYAIS | MX₁X₂PLLGVTX₃YAEKFQG | GGX₁NYLYWYFDL |
| 4 | (SEQ ID NO: 6) | (SEQ ID NO: 119) | (SEQ ID NO: 120) |
| | | Wherein, X₁ is L or I; X₂ is V | Wherein, X₁ is S or T |
| | | or I; and X₃ is N or D | |
| Antibody | | | |
| Name | LC-CDR1 | LC-CDR2 | LC-CDR3 |
| TSLP-01 | RASQSVSNNLA | DASSRAT | QQYSDWPQYT |
| | (SEQ ID NO: 25) | (SEQ ID NO: 39) | (SEQ ID NO: 50) |
| TSLP- | RASQSVSNNLA | DASSRAT | QQYSDWPQYT |
| 0101 | (SEQ ID NO: 25) | (SEQ ID NO: 39) | (SEQ ID NO: 50) |
| TSLP- | RASQSVSNNLA | DASSRAT | QQYSDWPQYT |
| 0102 | (SEQ ID NO: 25) | (SEQ ID NO: 39) | (SEQ ID NO: 50) |
| TSLP- | RASQSVSNNLA | DASSRAT | QQYSDWPQYT |
| 0103 | (SEQ ID NO: 25) | (SEQ ID NO: 39) | (SEQ ID NO: 50) |
| TSLP- | RASQSVSNNLA | DASSRAT | QQYSDWPQYT |
| 0104 | (SEQ ID NO: 25) | (SEQ ID NO: 39) | (SEQ ID NO: 50) |
| TSLP- | RASQSVSNNLA | DASSRAT | QQYSDWPQYT |
| 0105 | (SEQ ID NO: 25) | (SEQ ID NO: 39) | (SEQ ID NO: 50) |
| TSLP- | RASQSVSNNLA | DASSRAT | QQYSDWPQYT |
| 0106 | (SEQ ID NO: 25) | (SEQ ID NO: 39) | (SEQ ID NO: 50) |
| TSLP- | RASQSVSNNLA | DASSRAT | QQYSDWPQYT |
| 0107 | (SEQ ID NO: 25) | (SEQ ID NO: 39) | (SEQ ID NO: 50) |
| TSLP- | RASQSVSNNLA | DASSRAT | QQYSDWPEYS |
| 0108 | (SEQ ID NO: 25) | (SEQ ID NO: 39) | (SEQ ID NO: 51) |
| TSLP- | RASQGISSYLA | DASNLQS | QQYSNWPQYS |
| 0109 | (SEQ ID NO: 26) | (SEQ ID NO: 40) | (SEQ ID NO: 52) |
| TSLP- | RASQSVSSYLA | DTSSRAT | QQYSNWPQYS |
| 0110 | (SEQ ID NO: 27) | (SEQ ID NO: 41) | (SEQ ID NO: 52) |
| TSLP- | RASQSVSSNLA | DASSRAT | QQYSNWPQYS |
| 0111 | (SEQ ID NO: 28) | (SEQ ID NO: 39) | (SEQ ID NO: 52) |
| TSLP- | RASQSVSSYLA | DASSRAT | QQYSDWPQYS |
| 0112 | (SEQ ID NO: 27) | (SEQ ID NO: 39) | (SEQ ID NO: 53) |
| Composite | RASQX₁X₂SX₃X₄LA | DX₁SX₂X₃X₄X₅ | QQYSX₁WPX₂YX₃ |
| 1 | (SEQ ID NO: 105) | (SEQ ID NO: 106) | (SEQ ID NO: 107) |
| | Wherein, X₁ is G or | Wherein, X₁ is A or T; X₂ is S | Wherein, X₁ is D or N; |
| | S; X₂ is V or I; X₃ | or N; X₃ is R or L; X₄ is A or Q; | X₂ is Q or E; And X₃ is |
| | is N or S; And X₄ is | And X₅ is T or S | T or S |
| | N or Y | | |
| TSLP-02 | SGSSSDIGGYNRVS | DVSKRPS | SSYAGTDTFIL (SEQ ID |
| | (SEQ ID NO: 29) | (SEQ ID NO: 42) | NO: 54) |
| TSLP- | SGSSSDIGGYNRVS | DVSKRPS | SSYAGTDTFIL (SEQ ID |
| 0201 | (SEQ ID NO: 29) | (SEQ ID NO: 42) | NO: 54) |
| TSLP- | SGSSSDIGGYNRVS | DVSKRPS | SSYAGTDTFIL (SEQ ID |
| 0202 | (SEQ ID NO: 29) | (SEQ ID NO: 42) | NO: 54) |
| TSLP- | TGTSSDIGGYDRVS | GVSKRPS | SSYASGHTFGL (SEQ ID |
| 0203 | (SEQ ID NO: 30) | (SEQ ID NO: 43) | NO: 55) |
| TSLP- | TGTSSDIGGYNRVS | EVNKRSS | TSYAGTDTFVL (SEQ ID |
| 0204 | (SEQ ID NO: 31) | (SEQ ID NO: 44) | NO: 56) |
| TSLP- | TGTTSDIGGYNRVS | EVSKRPS | SSYAGTDTFAI (SEQ ID |
| 0205 | (SEQ ID NO: 32) | (SEQ ID NO: 45) | NO: 57) |
| TSLP- | TGTISDIGGYNRVS | EVSKRPS | SSYAGTDTFVF |
| 0206 | (SEQ ID NO: 33) | (SEQ ID NO: 45) | (SEQ ID NO: 58) |
| TSLP- | TGTSSDIGGYNRVS | EFNKRPS | SSYAGTDIFVL |
| 0207 | (SEQ ID NO: 31) | (SEQ ID NO: 46) | (SEQ ID NO: 59) |
| TSLP- | TGSNSDIGGYNRVS | EISKRPS | SSYAGTDTFVL |
| 0208 | (SEQ ID NO: 34) | (SEQ ID NO: 47) | (SEQ ID NO: 60) |
| Composite | X₁GX₂X₃SDIGGYX₄RVS | X₁X₂X₃KRX₄S | X₁SYAX₂X₃X₄X₅FX₆X₇ |
| 2 | (SEQ ID NO: 111) | (SEQ ID NO: 112) | (SEQ ID NO: 113) |
| | Wherein, X₁ is S or T; | Wherein, X₁ is D, G or E; X₂ is | Wherein, X₁ is S or T; |
| | X₂ is S or T; X₃ is S, | V, F or I; X₃ is S or N; And X₄ | X₂ is G or S; X₃ is T or |
| | T, I, or N; And X₄ is | is P or S | G; X₄ is D or H; X₅ is T |
| | N or D | | or I; X₆ is I, G, V or |
| | | | A; And X₇ is L, I or F |
| TSLP-03 | RSSQSLLHINGYTYLH | LVSHRAS | EQTLQTPYS |
| | (SEQ ID NO: 35) | (SEQ ID NO: 48) | (SEQ ID NO: 61) |
| TSLP- | RSSQSLLHSNEYTYLH | LVSHRAS | EQTLQTPFT |
| 0301 | (SEQ ID NO: 36) | (SEQ ID NO: 48) | (SEQ ID NO: 62) |
| TSLP- | RSSQSLLHSYGYTYLH | LVSYRAS | EQTLQTPYT |
| 0302 | (SEQ ID NO: 37) | (SEQ ID NO: 49) | (SEQ ID NO: 63) |
| Composite | RSSQSLLHX₁X₂X₃YTYLH | LVSX₁RAS | EQTLQTPX₁X₂ |
| 3 | (SEQ ID NO: 116) | (SEQ ID NO: 117) | (SEQ ID NO: 118) |
| | Wherein, X₁ is I or S; | Wherein, X₁ is H or Y | Wherein, X₁ is Y or F; |
| | X₂ is N or Y; And X₃ | | And X₂ is S or T |
| | is G or E | | |
| TSLP-04 | TGTSSDIGGYNRIS | DVSKRPS | SSYAGTDTFVL |
| | (SEQ ID NO: 38) | (SEQ ID NO: 42) | (SEQ ID NO: 60) |
| TSLP- | TGTSSDIGGYNRVS (SEQ | EVSKRPS | SAYAGTDTFVL |
| 0401 | ID NO: 31) | (SEQ ID NO: 45) | (SEQ ID NO: 64) |
| Composite | TGTSSDIGGYNRX₁S | X₁VSKRPS | SX₁YAGTDTFVL |
| 4 | (SEQ ID NO: 121) | (SEQ ID NO: 122) | (SEQ ID NO: 123) |
| | Wherein, X₁ is V or I | Wherein, X₁ is D or E | Wherein, X₁ is S or A |

TABLE 3
Exemplary sequences.

| SEQ ID | | |
|---|---|---|
| NO | Description | Sequence |
| 65 | TSLP-01 V_{H} | QVQLQESGPGLVKPSETLSLTCAVSGYSISSGYGWSWIRQPPGKGLEWIGY |
| | TSLP-0101 V_{H} | ISYYGSISYNPSLKSRVTISRDTSKNQFSLKLSSVTAADTAVYYCARTNLLY |
| | | FDSWGQGTLVTVSS |
| 66 | TSLP-0102 V_{H} | QVQLQESGPGLVKPSETLSLTCTVSGYSISSGYGWSWIRQPPGKGLEWIGY |
| | TSLP-0103 V_{H} | ISYYGSISYNPSLKSRVTISRDTSKNQFSLKLSSVTAADTAVYYCARTNLLY |
| | | FDSWGQGTLVTVSS |
| 67 | TSLP-0104 V_{H} | QVQLQESGPGLVKPSETLSLTCAVSGYSISSGYGWSWIRQPPGKGLEWIGY |
| | TSLP-0105 V_{H} | ISYYGSISYNPSLKSRVTISVDTSKNQFSLKLSSVTAADTAVYYCARTNLLY |
| | | FDSWGQGTLVTVSS |
| 68 | TSLP-0106 V_{H} | QVQLQESGPGLVKPSETLSLTCTVSGYSISSGYGWSWIRQPPGKGLEWIGY |
| | TSLP-0107 V_{H} | ISYYGSISYNPSLKSRVTISVDTSKNQFSLKLSSVTAADTAVYYCARTNLLY |
| | | FDSWGQGTLVTVSS |
| 69 | TSLP-0108 V_{H} | QVQLQESGPGLVKPSETLSLTCAVSGYSISSGYGWSWIRQPPGKGLEWIGY |
| | TSLP-0110 V_{H} | ISYYGSTSYNPSLKSRVTISRDTSKNQFSLKLSSVTAADTAVYYCARTNLL |
| | | YFDSWGQGTLVTVSS |
| 70 | TSLP-0109 V_{H} | QVQLQESGPGLVKPSETLSLTCAVSGYSISSGYGWSWIRQPPGKGLEWIGY |
| | | ISYYGSTSYNPSLKSRVTISRDTSKNQFSLKLSSVTAADTAVYYCARTNLL |
| | | YFESWGQGTLVTVSS |
| 71 | TSLP-0111 V_{H} | QVQLQESGPGLVKPSETLSLTCAVSGYSISSGYGWSWIRQPPGKGLEWIGY |
| | | FSYYGSTSYNPSLKSRVTISRDTSKNQFSLKLSSVTAADTAVYYCARTNLL |
| | | YFDSWGQGTLVTVSS |
| 72 | TSLP-0112 V_{H} | QVQLQESGPGLVKPSETLSLTCAVSGYSISSGYGWSWIRQPPGKGLEWIGY |
| | | ISYYGSTSYNPSLKSRVTISRDTSKNQFSLKLSSVTAADTAVYYCARTNLL |
| | | YFDYWGQGTLVTVSS |
| 73 | TSLP-02 V_{H} | QVQLVQSGAEVKKPGSSVKVSCKASGFTFGSYGINWVRQAPGQGLEWLG |
| | TSLP-0203 V_{H} | VIVPLVGVTIYAEKFQGRVTITADTSTSTAYMELSSLRSEDTAVYYCARGQ |
| | TSLP-0204 V_{H} | EYFYWYFDLWGQGTLVTVSS |
| | TSLP-0205 V_{H} | |
| 74 | TSLP-0201 V_{H} | QVQLVQSGAEVKKPGSSVKVSCKASGFTFGSYGINWVRQAPGQGLEWM |
| | | GVIVPLVGVTIYAEKFQGRVTITADTSTSTAYMELSSLRSEDTAVYYCARG |
| | | QEYFYWYFDLWGQGTLVTVSS |
| 75 | TSLP-0202 V_{H} | QVQLVQSGAEVKKPGSSVKVSCKASGFTFGSYGINWVRQAPGQGLEWM |
| | | GVIVPLVGVTIYAEKFQGRVTITADESTSTAYMELSSLRSEDTAVYYCARG |
| | | QEYFYWYFDLWGQGTLVTVSS |
| 76 | TSLP-0206 V_{H} | QVQLVQSGAEVKKPGSSVKVSCKASGFTFGSYGISWVRQAPGQGLEWLG |
| | | VIVPLVDVTIYAEKFQGRVTITADTSTSTAYMELSSLRSEDTAVYYCARGQ |
| | | EYFYWYFDLWGQGTLVTVSS |
| 77 | TSLP-0207 V_{H} | QVQLVQSGAEVKKPGSSVKVSCKASGFTFGSYGISWVRQAPGQGLEWLG |
| | | VIVPLVGVTIYAEKFQGRVTITADTSTSTAYMELSSLRSEDTAVYYCARGQ |
| | | EYFYWYFDLWGQGTLVTVSS |
| 78 | TSLP-0208 V_{H} | QVQLVQSGAEVKKPGSSVKVSCKASGFTFGSYGISWVRQAPGQGLEWLG |
| | | VIIPLLGVPNYAEKFQGRVTITADTSTSTAYMELSSLRSEDTAVYYCARGA |
| | | EYFYWYFDLWGQGTLVTVSS |
| 79 | TSLP-03 V_{H} | EVQLVESGGGLVQPGGSLRLSCAASGFTFSNYDMTWVRQAPGKGLEWVS |
| | | SITFASSYIYYADSVKGRFTISRDNAKNSLSLQMNSLRAEDTAVYYCTRGG |
| | | GAYHGGSLDVWGQGVLVTVSS |
| 80 | TSLP-0301 V_{H} | EVQLVESGGGLVQPGGSLRLSCAASGFTFSNYDMTWVRQAPGKGLEWVS |
| | | SITFASSYIYYADSVKGRFTISRDNAKNSLSLQMNSLRAEDTAVYYCTRGG |
| | | GAYYGGSLDVWGQGVLVTVSS |
| 81 | TSLP-0302 V_{H} | EVQLVESGGGLVQPGGSLRLSCAASGFTFSNYGMTWVRQAPGKGLEWVS |
| | | SITFASSYIYYADSVKGRFTISRDNAKNSLSLQMNSLRAEDTAVYYCTRGG |
| | | GAYYGGSLDVWGQGVLVTVSS |
| 82 | TSLP-04 V_{H} | QEQLVQSGDEVKKPGASVKVSCKSSGFTFGSYAISWVRQAPGQGLEWMG |
| | | MLVPLLGVTNYAEKFQGRVTITADPSTTTTSMELSSLRSEDTAVYYCARG |
| | | GSNYLYWYFDLWGPGTPITVSS |
| 83 | TSLP-0401 V_{H} | QEQLVQSGDEVKKPGASVKVSCKSSGFTFGSYAISWVRQAPGQGLEWMG |
| | | MIIPLLGVTDYAEKFQGRVTITADPSTTTTSMELSSLRSEDTAVYYCARGG |
| | | TNYLYWYFDLWGPGTPITVSS |
| 84 | TSLP-01 V_{L} | EIVMTQSPATLSLSPGERATLSCRASQSVSNNLAWYQQKPGQAPRLLIYDA |
| | TSLP-0102 V_{L} | SSRATGIPARFSGSGSGTDFTLTISSLEPEDFAVYYCQQYSDWPQYTFGQG |
| | TSLP-0104 V_{L} | TKVEIK |
| | TSLP-0106 V_{L} | |
| 85 | TSLP-0101 V_{L} | EIVLTQSPATLSLSPGERATLSCRASQSVSNNLAWYQQKPGQAPRLLIYDA |
| | TSLP-0103 V_{L} | SSRATGIPARFSGSGSGTDFTLTISSLEPEDFAVYYCQQYSDWPQYTFGQG |
| | TSLP-0105 V_{L} | TKVEIK |
| | TSLP-0107 V_{L} | |
| 86 | TSLP-0108 V_{L} | EIVMTQSPATLSLSPGERATLSCRASQSVSNNLAWYQQKPGQAPRLLIYDA |
| | | SSRATGIPARFSGSGSGTDFTLTISSLEPEDFAVYYCQQYSDWPEYSFGQGT |
| | | KVEIK |
| 87 | TSLP-0109 V_{L} | EIVMTQSPATLSLSPGERATLSCRASQGISSYLAWYQQKPGQAPRLLIYDA |
| | | SNLQSGIPARFSGSGSGTDFTLTISSLEPEDFAVYYCQQYSNWPQYSFGQG |
| | | TKVEIK |
| 88 | TSLP-0110 V_{L} | EIVMTQSPATLSLSPGERATLSCRASQSVSSYLAWYQQKPGQAPRLLIYDT |
| | | SSRATGIPARFSGSGSGTDFTLTISSLEPEDFAVYYCQQYSNWPQYSFGQGT |
| | | KVEIK |
| 89 | TSLP-0111 V_{L} | EIVMTQSPATLSLSPGERATLSCRASQSVSSNLAWYQQKPGQAPRLLIYDA |
| | | SSRATGIPARFSGSGSGTDFTLTISSLEPEDFAVYYCQQYSNWPQYSFGQGT |
| | | KVEIK |
| 90 | TSLP-0112 V_{L} | EIVMTQSPATLSLSPGERATLSCRASQSVSSYLAWYQQKPGQAPRLLIYDA |
| | | SSRATGIPARFSGSGSGTDFTLTISSLEPEDFAVYYCQQYSDWPQYSFGQGT |
| | | KVEIK |
| 91 | TSLP-02 V_{L} | QSALTQPPSASGSPGQSVTISCSGSSSDIGGYNRVSWYQQHPGKAPKLMIY |
| | TSLP-0201 V_{L} | DVSKRPSGVPDRFSGSKSGNTASLTVSGLQAEDEADYYCSSYAGTDTFILF |
| | TSLP-0202 V_{L} | GGGTKLTVL |
| 92 | TSLP-0203 V_{L} | QSALTQPPSASGSPGQSVTISCTGTSSDIGGYDRVSWYQQHPGKAPKLMIY |
| | | GVSKRPSGVPDRFSGSKSGNTASLTVSGLQAEDEADYYCSSYASGHTFGL |
| | | FGGGTKLTVL |
| 93 | TSLP-0204 V_{L} | QSALTQPPSASGSPGQSVTISCTGTSSDIGGYNRVSWYQQHPGKAPKLMIY |
| | | EVNKRSSGVPDRFSGSKSGNTASLTVSGLQAEDEADYYCTSYAGTDTFVL |
| | | FGGGTKLTVL |
| 94 | TSLP-0205 V_{L} | QSALTQPPSASGSPGQSVTISCTGTTSDIGGYNRVSWYQQHPGKAPKLMIY |
| | | EVSKRPSGVPDRFSGSKSGNTASLTVSGLQAEDEADYYCSSYAGTDTFAIF |
| | | GGGTKLTVL |
| 95 | TSLP-0206 V_{L} | QSALTQPPSASGSPGQSVTISCTGTISDIGGYNRVSWYQQHPGKAPKLMIY |
| | | EVSKRPSGVPDRFSGSKSGNTASLTVSGLQAEDEADYYCSSYAGTDTFVFF |
| | | GGGTKLTVL |
| 96 | TSLP-0207 V_{L} | QSALTQPPSASGSPGQSVTISCTGTSSDIGGYNRVSWYQQHPGKAPKLMIY |
| | | EFNKRPSGVPDRFSGSKSGNTASLTVSGLQAEDEADYYCSSYAGTDIFVLF |
| | | GGGTKLTVL |
| 97 | TSLP-0208 V_{L} | QSALTQPPSASGSPGQSVTISCTGSNSDIGGYNRVSWYQQHPGKAPKLMIY |
| | | EISKRPSGVPDRFSGSKSGNTASLTVSGLQAEDEADYYCSSYAGTDTFVLF |
| | | GGGTKLTVL |
| 98 | TSLP-03 V_{L} | DIVMTQTPLSLSVTPGEPASISCRSSQSLLHINGYTYLHWYLQKPGQSPQLL |
| | | IYLVSHRASGVPDRFSGSGSDTDFTLKISRVEAEDVGVYYCEQTLQTPYSF |
| | | GGGTKVEIK |
| 99 | TSLP-0301 V_{L} | DIVMTQTPLSLSVTPGEPASISCRSSQSLLHSNEYTYLHWYLQKPGQSPQLL |
| | | IYLVSHRASGVPDRFSGSGSDTDFTLKISRVEAEDVGVYYCEQTLQTPFTF |
| | | GGGTKVEIK |
| 100 | TSLP-0302 V_{L} | DIVMTQTPLSLSVTPGEPASISCRSSQSLLHSYGYTYLHWYLQKPGQSPQL |
| | | LIYLVSYRASGVPDRFSGSGSDTDFTLKISRVEAEDVGVYYCEQTLQTPYT |
| | | FGGGTKVEIK |
| 101 | TSLP-04 V_{L} | QSAPIQPRSVSGSPGQSVTISCTGTSSDIGGYNRISWYQQHPGKAPKLMIYD |
| | | VSKRPSGVSDRFSGSKSGNTASLTISGLQAEDEADYYCSSYAGTDTFVLFG |
| | | GGTQLTIV |
| 102 | TSLP-0401 V_{L} | QSAPIQPRSVSGSPGQSVTISCTGTSSDIGGYNRVSWYQQHPGKAPKLMIY |
| | | EVSKRPSGVSDRFSGSKSGNTASLTISGLQAEDEADYYCSAYAGTDTFVLF |
| | | GGGTQLTIV |
| 124 | IgG1 heavy | ASTKGPSVFPLAPSSKSTSGGTAALGCLVKDYFPEPVTVSWNSGALTSGV |
| | chain constant | HTFPAVLQSSGLYSLSSVVTVPSSSLGTQTYICNVNHKPSNTKVDKKVEPK |
| | region | SCDKTHTCPPCPAPELLGGPSVFLFPPKPKDTLMISRTPEVTCVVVDVSHE |
| | | DPEVKFNWYVDGVEVHNAKTKPREEQYNSTYRVVSVLTVLHQDWLNGK |
| | | EYKCKVSNKALPAPIEKTISKAKGQPREPQVYTLPPSREEMTKNQVSLTCL |
| | | VKGFYPSDIAVEWESNGQPENNYKTTPPVLDSDGSFFLYSKLTVDKSRWQ |
| | | QGNVFSCSVMHEALHNHYTQKSLSLSPGK |
| 125 | IgG4 heavy | ASTKGPSVFPLAPCSRSTSESTAALGCLVKDYFPEPVTVSWNSGALTSGVH |
| | chain constant | TFPAVLQSSGLYSLSSVVTVPSSSLGTKTYTCNVDHKPSNTKVDKRVESKY |
| | region | GPPCPSCPAPEFLGGPSVFLFPPKPKDTLMISRTPEVTCVVVDVSQEDPEVQ |
| | | FNWYVDGVEVHNAKTKPREEQFNSTYRVVSVLTVLHQDWLNGKEYKCK |
| | | VSNKGLPSSIEKTISKAKGQPREPQVYTLPPSQEEMTKNQVSLTCLVKGFY |
| | | PSDIAVEWESNGQPENNYKTTPPVLDSDGSFFLYSRLTVDKSRWQEGNVF |
| | | SCSVMHEALHNHYTQKSLSLSLGK |
| 126 | Light chain | RTVAAPSVFIFPPSDEQLKSGTASVVCLLNNFYPREAKVQWKVDNALQSG |
| | constant | NSQESVTEQDSKDSTYSLSSTLTLSKADYEKHKVYACEVTHQGLSSPVTK |
| | region(kappa) | SFNRGEC |
| 127 | Light chain | GQPKAAPSVTLFPPSSEELQANKATLVCLISDFYPGAVTVAWKADSSPVK |
| | constant | AGVETTTPSKQSNNKYAASSYLSLTPEQWKSHRSYSCQVTHEGSTVEKTV |
| | region(lambda) | APTECS |
| 128 | human TSLP | MFPFALLYVLSVSFRKIFILQLVGLVLTYDFTNCDFEKIKAAYLSTISKDLIT |
| | | YMSGTKSTEFNNTVSCSNRPHCLTEIQSLTFNPTAGCASLAKEMFAMKTK |
| | | AALAIWCPGYSETQINATQAMKKRRKRKVTTNKCLEQVSQLQGLWRRFN |
| | | RPLLKQQ |
| 129 | Mutational | MFPFALLYVLSVSFRKIFILQLVGLVLTYDFTNCDFEKIKAAYLSTISKDLIT |
| | human TSLP | YMSGTKSTEFNNTVSCSNRPHCLTEIQSLTFNPTAGCASLAKEMFAMKTK |
| | | AALAIWCPGYSETQINATQAMKKARKSKVTTNKCLEQVSQLQGLWRRFN |
| | | RPLLKQQ |

### TSLP

TSLP is an epithelial cell-derived cytokine constitutively expressed in skin, gut, lung, and thymus (He R, et al, Ann NY Acad Sci 2010; 1183:13-24). Of which, the highest levels of TSLP are seen in lung-derived and skin-derived epithelial cells (Ziegler S F, Curr Opin Immunol 2010; 22:795-799). TSLP was first identified, as a distant paralog of IL-7, in the supernatant of the mouse thymic stromal cell line, Z210R.1. Subsequent expression cloning revealed that the mouse TSLP (mTSLP) is a member of the hematopoietic cytokine family (Sims J E, et al, J Exp Med 2000; 192:671-680). Sequence prediction displayed a similar four helix structured cytokine with two N-glycosylation sites and six cysteine residues (Kashyap M, et al, J Immunol 2011; 187:1207-1211). Human TSLP (hTSLP) and mTSLP exhibit poor homology with only 43% amino acid identity (Reche P A, et al, J Immunol 2001; 167:336-343; Quentmeier H, et al, Leukemia 2001; 15:1286-1292)

Two isoforms of TSLP, short and long isoforms, have been described in mice, but the functional consequences of this variation are unclear. In humans, the main isoform expressed during steady state conditions is the short form of TSLP, whereas the long form of TSLP is upregulated in inflammatory conditions (Fornasa G, et al, J Allergy Clin Immunol 2015; 136:413-22; Tsilingiri K, et al, Cell Mol Gastroenterol Hepatol 2017; 3:174-82). In addition, there is evidence that in pathological conditions, TSLP can be cleaved by several endogenous proteases (Poposki J A, et al, J Allergy Clin Immunol 2017; 139: 1559-67. e8; Nagarkar D R, et al, J Allergy Clin Immunol 2013; 132:593-600. e12). For example, in celiac disease patients, the protease furin, which is upregulated in patient biopsies, can cleave the long isoform of TSLP, producing 10 kDa and 4 kDa fragments with different activities on human peripheral blood mononuclear cells compared to mature TSLP (Biancheri P, et al, Gut 2016; 65:1670-80).

TSLP induces innate allergic immune responses by targeting mDCs, mast cells, eosinophils, basophils, and NKT cells (Al-Shami A, et al, J Exp Med 2004; 200:159-168; Wu W H, et al, J Allergy Clin Immunol 2010; 126:290-299, 299.e1-e4; Soumelis V, et al, Nat Immunol 2002; 3:673-680). It is evident that TSLP strongly upregulates the expression of MHC class II, CD54, CD80, CD83, CD86, and DC-lamp in mDCs (Liu Y J, J Exp Med 2006; 203:269-273). Of note, unlike CD40L and Toll-like receptor (TLR) ligands, TSLP does not stimulate mDCs to produce either Thl-polarizing cytokines (IL-12 and type 1 interferons) or proinflammatory cytokines (TNF-α, IL-1β and IL-6).

Recent evidence implicates a pivotal role of TSLP in the pathobiology of allergic asthma and atopic dermatitis (AD). It is compelling to highlight TSLP as a potential treatment for allergic disorders. Further dissection of the mechanisms regarding TSLP-mediated immune response toward a Th2 phenotype will shed new light on this quest. Moreover, TSLP strongly creates a Th2-permissive microenvironment attributable to the development of asthma (Willart M A, et al, Allergol Int 2010; 59:95-103).

### TSLP Receptor (TSLPR)

TSLP receptor (TSLPR) complex consists of TSLPR and IL-7 receptor alpha (IL-7R)(Park L S, et al, J Exp Med 2000; 192:659-670). TSLP binds to a heterodimeric receptor consisting of the IL-7 receptor α-chain (IL-7Rα) and the TSLPR chain, which is closely related to the common receptor γ chain (γc), in order to exert its biological activity on a broad range of cell types. TSLPR alone has a low affinity for TSLP but binding of TSLPR to IL-7Ra creates a high affinity binding site for TSLP and triggers signaling (He R, et al, Ann N Y Acad Sci 2010; 1183:13-24). A combination of TSLPR and IL-7Rα chain results in high affinity binding and activation of signal transducer and activator of transcription 3 (STAT3) and STAT5 (Pandey A, et al, Nat Immunol 2000; 1:59-64). Expression of TSLPR transcript can be detected in early B and T cell progenitors, peripheral CD4+ T cells, mast cells, and DCs in both species (He S H, et al, Clin Exp Immunol 2011; 165:29-37).

Neutralization of TSLP binding to TSLPR is therefore a therapeutic approach to treating diseases and conditions mediated through TSLP signaling.

### Full-Length Anti-TSLP Antibody

The anti-TSLP antibody in some embodiments is a full-length anti-TSLP antibody. In some embodiments, the full-length anti-TSLP antibody is an IgA, IgD, IgE, IgG, or IgM. In some embodiments, the full-length anti-TSLP antibody comprises IgG constant domains, such as constant domains of any of IgG1, IgG2, IgG3, and IgG4 including variants thereof. In some embodiments, the full-length anti-TSLP antibody comprises a lambda light chain constant region. In some embodiments, the full-length anti-TSLP antibody comprises a kappa light chain constant region. In some embodiments, the full-length anti-TSLP antibody is a full-length human anti-TSLP antibody. In some embodiments, the full-length anti-TSLP antibody comprises an Fc sequence of a mouse immunoglobulin. In some embodiments, the full-length anti-TSLP antibody comprises an Fc sequence that has been altered or otherwise changed so that it has enhanced antibody dependent cellular cytotoxicity (ADCC) or complement dependent cytotoxicity (CDC) effector function.

Thus, for example, in some embodiments, there is provided a full-length anti-TSLP antibody comprising IgG1 constant domains, wherein the anti-TSLP antibody specifically binds to TSLP. In some embodiments, the IgG1 is human IgG1. In some embodiments, the heavy chain constant region comprises or consists of the amino acid sequence of SEQ ID NO: 124. In some embodiments, the light chain constant region comprises or consists of the amino acid sequence of SEQ ID NO: 126. In some embodiments, the light chain constant region comprises or consists of the amino acid sequence of SEQ ID NO: 127. In some embodiments, the heavy chain constant region comprises or consists of the amino acid sequence of SEQ ID NO: 124 and the light chain constant region comprises or consists of the amino acid sequence of SEQ ID NO: 126. In some embodiments, the heavy chain constant region comprises or consists of the amino acid sequence of SEQ ID NO: 124 and the light chain constant region comprises or consists of the amino acid sequence of SEQ ID NO: 127.

In some embodiments, there is provided a full-length anti-TSLP antibody comprising IgG2 constant domains, wherein the anti-TSLP antibody specifically binds to TSLP. In some embodiments, the IgG2 is human IgG2. In some embodiments, the light chain constant region comprises or consists of the amino acid sequence of SEQ ID NO: 126. In some embodiments, the light chain constant region comprises or consists of the amino acid sequence of SEQ ID NO: 127.

In some embodiments, there is provided a full-length anti-TSLP antibody comprising IgG3 constant domains, wherein the anti-TSLP antibody specifically binds to TSLP. In some embodiments, the IgG3 is human IgG3. In some embodiments, the light chain constant region comprises or consists of the amino acid sequence of SEQ ID NO: 126. In some embodiments, the light chain constant region comprises or consists of the amino acid sequence of SEQ ID NO: 127.

In some embodiments, there is provided a full-length anti-TSLP antibody comprising IgG4 constant domains, wherein the anti-TSLP antibody specifically binds to TSLP. In some embodiments, the IgG4 is human IgG4. In some embodiments, the heavy chain constant region comprises or consists of the amino acid sequence of SEQ ID NO: 125.

In some embodiments, the light chain constant region comprises or consists of the amino acid sequence of SEQ ID NO: 126. In some embodiments, the light chain constant region comprises or consists of the amino acid sequence of SEQ ID NO: 127. In some embodiments, the heavy chain constant region comprises or consists of the amino acid sequence of SEQ ID NO: 125 and the light chain constant region comprises or consists of the amino acid sequence of SEQ ID NO: 126. In some embodiments, the heavy chain constant region comprises or consists of the amino acid sequence of SEQ ID NO: 125 and the light chain constant region comprises or consists of the amino acid sequence of SEQ ID NO: 127.

In some embodiments, there is provided a full-length anti-TSLP antibody comprising IgG1 constant domains, wherein the anti-TSLP antibody comprises a) a heavy chain variable domain comprising an HC-CDR1 comprising the amino acid sequence of any one of SEQ ID NOs: 1-6, or a variant thereof comprising up to about 3 (such as about any of 1, 2, or 3) amino acid substitutions, an HC-CDR2 comprising the amino acid sequence of any one of SEQ ID NOs: 7-15, or a variant thereof comprising up to about 3 (such as about any of 1, 2, or 3) amino acid substitutions, and an HC-CDR3 comprising the amino acid sequence of any one of SEQ ID NOs: 16-24, or a variant thereof comprising up to about 3 (such as about any of 1, 2, or 3) amino acid substitutions; and b) a light chain variable domain comprising an LC-CDR1 comprising the amino acid sequence of any one of SEQ ID NOs: 25-38, or a variant thereof comprising up to about 3 (such as about any of 1, 2, or 3) amino acid substitutions, an LC-CDR2 comprising the amino acid sequence of any one of SEQ ID NOs: 39-49, or a variant thereof comprising up to about 3 (such as about any of 1, 2, or 3) amino acid substitutions, and an LC-CDR3 comprising the amino acid sequence of any one of SEQ ID NOs: 50-64, or a variant thereof comprising up to about 3 (such as about any of 1, 2, or 3) amino acid substitutions. In some embodiments, the IgG1 is human IgG1. In some embodiments, the heavy chain constant region comprises or consists of the amino acid sequence of SEQ ID NO: 124. In some embodiments, the light chain constant region comprises or consists of the amino acid sequence of SEQ ID NO: 126. In some embodiments, the light chain constant region comprises or consists of the amino acid sequence of SEQ ID NO: 127. In some embodiments, the heavy chain constant region comprises or consists of the amino acid sequence of SEQ ID NO: 124 and the light chain constant region comprises or consists of the amino acid sequence of SEQ ID NO: 126. In some embodiments, the heavy chain constant region comprises or consists of the amino acid sequence of SEQ ID NO: 124 and the light chain constant region comprises or consists of the amino acid sequence of SEQ ID NO: 127.

In some embodiments, there is provided a full-length anti-TSLP antibody comprising IgG2 constant domains, wherein the anti-TSLP antibody comprises a) a heavy chain variable domain comprising an HC-CDR1 comprising the amino acid sequence of any one of SEQ ID NOs: 1-6, or a variant thereof comprising up to about 3 (such as about any of 1, 2, or 3) amino acid substitutions, an HC-CDR2 comprising the amino acid sequence of any one of SEQ ID NOs: 7-15, or a variant thereof comprising up to about 3 (such as about any of 1, 2, or 3) amino acid substitutions, and an HC-CDR3 comprising the amino acid sequence of any one of SEQ ID NOs: 16-24, or a variant thereof comprising up to about 3 (such as about any of 1, 2, or 3) amino acid substitutions; and b) a light chain variable domain comprising an LC-CDR1 comprising the amino acid sequence of any one of SEQ ID NOs: 25-38, or a variant thereof comprising up to about 3 (such as about any of 1, 2, or 3) amino acid substitutions, an LC-CDR2 comprising the amino acid sequence of any one of SEQ ID NOs: 39-49, or a variant thereof comprising up to about 3 (such as about any of 1, 2, or 3) amino acid substitutions, and an LC-CDR3 comprising the amino acid sequence of any one of SEQ ID NOs: 50-64, or a variant thereof comprising up to about 3 (such as about any of 1, 2, or 3) amino acid substitutions. In some embodiments, the IgG2 is human IgG2. In some embodiments, the light chain constant region comprises or consists of the amino acid sequence of SEQ ID NO: 126. In some embodiments, the light chain constant region comprises or consists of the amino acid sequence of SEQ ID NO: 127.

In some embodiments, there is provided a full-length anti-TSLP antibody comprising IgG3 constant domains, wherein the anti-TSLP antibody comprises a) a heavy chain variable domain comprising an HC-CDR1 comprising the amino acid sequence of any one of SEQ ID NOs: 1-6, or a variant thereof comprising up to about 3 (such as about any of 1, 2, or 3) amino acid substitutions, an HC-CDR2 comprising the amino acid sequence of any one of SEQ ID NOs: 7-15, or a variant thereof comprising up to about 3 (such as about any of 1, 2, or 3) amino acid substitutions, and an HC-CDR3 comprising the amino acid sequence of any one of SEQ ID NOs: 16-24, or a variant thereof comprising up to about 3 (such as about any of 1, 2, or 3) amino acid substitutions; and b) a light chain variable domain comprising an LC-CDR1 comprising the amino acid sequence of any one of SEQ ID NOs: 25-38, or a variant thereof comprising up to about 3 (such as about any of 1, 2, or 3) amino acid substitutions, an LC-CDR2 comprising the amino acid sequence of any one of SEQ ID NOs: 39-49, or a variant thereof comprising up to about 3 (such as about any of 1, 2, or 3) amino acid substitutions, and an LC-CDR3 comprising the amino acid sequence of any one of SEQ ID NOs: 50-64, or a variant thereof comprising up to about 3 (such as about any of 1, 2, or 3) amino acid substitutions. In some embodiments, the IgG3 is human IgG3. In some embodiments, the light chain constant region comprises or consists of the amino acid sequence of SEQ ID NO: 126. In some embodiments, the light chain constant region comprises or consists of the amino acid sequence of SEQ ID NO: 127.

In some embodiments, there is provided a full-length anti-TSLP antibody comprising IgG4 constant domains, wherein the anti-TSLP antibody comprises a) a heavy chain variable domain comprising an HC-CDR1 comprising the amino acid sequence of any one of SEQ ID NOs: 1-6, or a variant thereof comprising up to about 3 (such as about any of 1, 2, or 3) amino acid substitutions, an HC-CDR2 comprising the amino acid sequence of any one of SEQ ID NOs: 7-15, or a variant thereof comprising up to about 3 (such as about any of 1, 2, or 3) amino acid substitutions, and an HC-CDR3 comprising the amino acid sequence of any one of SEQ ID NOs: 16-24, or a variant thereof comprising up to about 3 (such as about any of 1, 2, or 3) amino acid substitutions; and b) a light chain variable domain comprising an LC-CDR1 comprising the amino acid sequence of any one of SEQ ID NOs: 25-38, or a variant thereof comprising up to about 3 (such as about any of 1, 2, or 3) amino acid substitutions, an LC-CDR2 comprising the amino acid sequence of any one of SEQ ID NOs: 39-49, or a variant thereof comprising up to about 3 (such as about any of 1, 2, or 3) amino acid substitutions, and an LC-CDR3 comprising the amino acid sequence of any one of SEQ ID NOs: 50-64, or a variant thereof comprising up to about 3 (such as about any of 1, 2, or 3) amino acid substitutions. In some embodiments, the IgG4 is human IgG4. In some embodiments, the heavy chain constant region comprises or consists of the amino acid sequence of SEQ ID NO: 125. In some embodiments, the light chain constant region comprises or consists of the amino acid sequence of SEQ ID NO: 126. In some embodiments, the light chain constant region comprises or consists of the amino acid sequence of SEQ ID NO: 127. In some embodiments, the heavy chain constant region comprises or consists of the amino acid sequence of SEQ ID NO: 125 and the light chain constant region comprises or consists of the amino acid sequence of SEQ ID NO: 126. In some embodiments, the heavy chain constant region comprises or consists of the amino acid sequence of SEQ ID NO: 125 and the light chain constant region comprises or consists of the amino acid sequence of SEQ ID NO: 127.

In some embodiments, there is provided a full-length anti-TSLP antibody comprising IgG1 constant domains, wherein the anti-TSLP antibody comprises a) a heavy chain variable domain comprising an HC-CDR1 comprising the amino acid sequence of any one of SEQ ID NOs: 1-6, an HC-CDR2 comprising the amino acid sequence of any one of SEQ ID NOs: 7-15, and an HC-CDR3 comprising the amino acid sequence of any one of SEQ ID NOs: 16-24, or a variant thereof comprising up to about 3 (such as about any of 1, 2, or 3) amino acid substitutions in the HC-CDR sequences; and b) a light chain variable domain comprising an LC-CDR1 comprising the amino acid sequence of any one of SEQ ID NOs: 25-38, an LC-CDR2 comprising the amino acid sequence of any one of SEQ ID NOs: 39-49, and an LC-CDR3 comprising the amino acid sequence of any one of SEQ ID NOs: 50-64, or a variant thereof comprising up to about 3 (such as about any of 1, 2, or 3) amino acid substitutions in the LC-CDR sequences. In some embodiments, the IgG1 is human IgG1. In some embodiments, the anti-TSLP heavy chain constant region comprises or consists of the amino acid sequence of SEQ ID NO: 124. In some embodiments, the anti-TSLP light chain constant region comprises or consists of the amino acid sequence of SEQ ID NO: 126. In some embodiments, the anti-TSLP light chain constant region comprises or consists of the amino acid sequence of SEQ ID NO: 127. In some embodiments, the heavy chain constant region comprises or consists of the amino acid sequence of SEQ ID NO: 124 and the light chain constant region comprises or consists of the amino acid sequence of SEQ ID NO: 126. In some embodiments, the heavy chain constant region comprises or consists of the amino acid sequence of SEQ ID NO: 124 and the light chain constant region comprises or consists of the amino acid sequence of SEQ ID NO: 127.

In some embodiments, there is provided a full-length anti-TSLP antibody comprising IgG4 constant domains, wherein the anti-TSLP antibody comprises a) a heavy chain variable domain comprising an HC-CDR1 comprising the amino acid sequence of any one of SEQ ID NOs: 1-6, an HC-CDR2 comprising the amino acid sequence of any one of SEQ ID NOs: 7-15, and an HC-CDR3 comprising the amino acid sequence of any one of SEQ ID NOs: 16-24, or a variant thereof comprising up to about 3 (such as about any of 1, 2, or 3) amino acid substitutions in the HC-CDR sequences; and b) a light chain variable domain comprising an LC-CDR1 comprising the amino acid sequence of any one of SEQ ID NOs: 25-38, an LC-CDR2 comprising the amino acid sequence of any one of SEQ ID NOs: 39-49, and an LC-CDR3 comprising the amino acid sequence of any one of SEQ ID NOs: 50-64, or a variant thereof comprising up to about 3 (such as about any of 1, 2, or 3) amino acid substitutions in the LC-CDR sequences. In some embodiments, the IgG4 is human IgG4. In some embodiments, the heavy chain constant region comprises or consists of the amino acid sequence of SEQ ID NO: 125. In some embodiments, the light chain constant region comprises or consists of the amino acid sequence of SEQ ID NO: 126. In some embodiments, the light chain constant region comprises or consists of the amino acid sequence of SEQ ID NO: 127. In some embodiments, the heavy chain constant region comprises or consists of the amino acid sequence of SEQ ID NO: 125 and the light chain constant region comprises or consists of the amino acid sequence of SEQ ID NO: 126. In some embodiments, the heavy chain constant region comprises or consists of the amino acid sequence of SEQ ID NO: 125 and the light chain constant region comprises or consists of the amino acid sequence of SEQ ID NO: 127.

In some embodiments, there is provided a full-length anti-TSLP antibody comprising IgG1 constant domains, wherein the anti-TSLP antibody comprises a) a heavy chain variable domain comprising an HC-CDR1 comprising the amino acid sequence of any one of SEQ ID NOs: 1-6, an HC-CDR2 comprising the amino acid sequence of any one of SEQ ID NOs: 7-15, and an HC-CDR3 comprising the amino acid sequence of any one of SEQ ID NOs: 16-24; and b) a light chain variable domain comprising an LC-CDR1 comprising the amino acid sequence of any one of SEQ ID NOs: 25-38, an LC-CDR2 comprising the amino acid sequence of any one of SEQ ID NOs: 39-49, and an LC-CDR3 comprising the amino acid sequence of any one of SEQ ID NOs: 50-64. In some embodiments, the IgG1 is human IgG1. In some embodiments, the heavy chain constant region comprises or consists of the amino acid sequence of SEQ ID NO: 124. In some embodiments, the light chain constant region comprises or consists of the amino acid sequence of SEQ ID NO: 126. In some embodiments, the light chain constant region comprises or consists of the amino acid sequence of SEQ ID NO: 127. In some embodiments, the heavy chain constant region comprises or consists of the amino acid sequence of SEQ ID NO: 124 and the light chain constant region comprises or consists of the amino acid sequence of SEQ ID NO: 126. In some embodiments, the heavy chain constant region comprises or consists of the amino acid sequence of SEQ ID NO: 124 and the light chain constant region comprises or consists of the amino acid sequence of SEQ ID NO: 127.

In some embodiments, there is provided a full-length anti-TSLP antibody comprising IgG4 constant domains, wherein the anti-TSLP antibody comprises a) a heavy chain variable domain comprising an HC-CDR1 comprising the amino acid sequence of any one of SEQ ID NOs: 1-6, an HC-CDR2 comprising the amino acid sequence of any one of SEQ ID NOs: 7-15, and an HC-CDR3 comprising the amino acid sequence of any one of SEQ ID NOs: 16-24; and b) a light chain variable domain comprising an LC-CDR1 comprising the amino acid sequence of any one of SEQ ID NOs: 25-38, an LC-CDR2 comprising the amino acid sequence of any one of SEQ ID NOs: 39-49, and an LC-CDR3 comprising the amino acid sequence of any one of SEQ ID NOs: 50-64. In some embodiments, the IgG4 is human IgG4. In some embodiments, the heavy chain constant region comprises or consists of the amino acid sequence of SEQ ID NO: 125. In some embodiments, the light chain constant region comprises or consists of the amino acid sequence of SEQ ID NO: 126. In some embodiments, the light chain constant region comprises or consists of the amino acid sequence of SEQ ID NO: 127. In some embodiments, the heavy chain constant region comprises or consists of the amino acid sequence of SEQ ID NO: 125 and the light chain constant region comprises or consists of the amino acid sequence of SEQ ID NO: 126. In some embodiments, the heavy chain constant region comprises or consists of the amino acid sequence of SEQ ID NO: 125 and the light chain constant region comprises or consists of the amino acid sequence of SEQ ID NO: 127.

In some embodiments, there is provided a full-length anti-TSLP antibody comprising IgG1 constant domains, wherein the anti-TSLP antibody comprises a) a heavy chain variable domain comprising an HC-CDR1 comprising the amino acid sequence of SEQ ID NO: 1, an HC-CDR2 comprising the amino acid sequence of SEQ ID NO: 7, and an HC-CDR3 comprising the amino acid sequence of SEQ ID NO: 16; and b) a light chain variable domain comprising an LC-CDR1 comprising the amino acid sequence of SEQ ID NO: 25, an LC-CDR2 comprising the amino acid sequence of SEQ ID NO: 39, and an LC-CDR3 comprising the amino acid sequence of SEQ ID NO: 50. In some embodiments, the IgG1 is human IgG1. In some embodiments, the heavy chain constant region comprises or consists of the amino acid sequence of SEQ ID NO: 124. In some embodiments, the light chain constant region comprises or consists of the amino acid sequence of SEQ ID NO: 126. In some embodiments, the light chain constant region comprises or consists of the amino acid sequence of SEQ ID NO: 127. In some embodiments, the heavy chain constant region comprises or consists of the amino acid sequence of SEQ ID NO: 124 and the light chain constant region comprises or consists of the amino acid sequence of SEQ ID NO: 126. In some embodiments, the heavy chain constant region comprises or consists of the amino acid sequence of SEQ ID NO: 124 and the light chain constant region comprises or consists of the amino acid sequence of SEQ ID NO: 127.

In some embodiments, there is provided a full-length anti-TSLP antibody comprising IgG1 constant domains, wherein the anti-TSLP antibody comprises a) a heavy chain variable domain comprising an HC-CDR1 comprising the amino acid sequence of SEQ ID NO: 1, an HC-CDR2 comprising the amino acid sequence of SEQ ID NO: 8, and an HC-CDR3 comprising the amino acid sequence of SEQ ID NO: 16; and b) a light chain variable domain comprising an LC-CDR1 comprising the amino acid sequence of SEQ ID NO: 25, an LC-CDR2 comprising the amino acid sequence of SEQ ID NO: 39, and an LC-CDR3 comprising the amino acid sequence of SEQ ID NO: 51. In some embodiments, the IgG1 is human IgG1. In some embodiments, the heavy chain constant region comprises or consists of the amino acid sequence of SEQ ID NO: 124. In some embodiments, the light chain constant region comprises or consists of the amino acid sequence of SEQ ID NO: 126. In some embodiments, the light chain constant region comprises or consists of the amino acid sequence of SEQ ID NO: 127. In some embodiments, the heavy chain constant region comprises or consists of the amino acid sequence of SEQ ID NO: 124 and the light chain constant region comprises or consists of the amino acid sequence of SEQ ID NO: 126. In some embodiments, the heavy chain constant region comprises or consists of the amino acid sequence of SEQ ID NO: 124 and the light chain constant region comprises or consists of the amino acid sequence of SEQ ID NO: 127.

In some embodiments, there is provided a full-length anti-TSLP antibody comprising IgG1 constant domains, wherein the anti-TSLP antibody comprises a) a heavy chain variable domain comprising an HC-CDR1 comprising the amino acid sequence of SEQ ID NO: 1, an HC-CDR2 comprising the amino acid sequence of SEQ ID NO: 8, and an HC-CDR3 comprising the amino acid sequence of SEQ ID NO: 17; and b) a light chain variable domain comprising an LC-CDR1 comprising the amino acid sequence of SEQ ID NO: 26, an LC-CDR2 comprising the amino acid sequence of SEQ ID NO: 40, and an LC-CDR3 comprising the amino acid sequence of SEQ ID NO: 52. In some embodiments, the IgG1 is human IgG1. In some embodiments, the heavy chain constant region comprises or consists of the amino acid sequence of SEQ ID NO: 124. In some embodiments, the light chain constant region comprises or consists of the amino acid sequence of SEQ ID NO: 126. In some embodiments, the light chain constant region comprises or consists of the amino acid sequence of SEQ ID NO: 127. In some embodiments, the heavy chain constant region comprises or consists of the amino acid sequence of SEQ ID NO: 124 and the light chain constant region comprises or consists of the amino acid sequence of SEQ ID NO: 126. In some embodiments, the heavy chain constant region comprises or consists of the amino acid sequence of SEQ ID NO: 124 and the light chain constant region comprises or consists of the amino acid sequence of SEQ ID NO: 127.

In some embodiments, there is provided a full-length anti-TSLP antibody comprising IgG1 constant domains, wherein the anti-TSLP antibody comprises a) a heavy chain variable domain comprising an HC-CDR1 comprising the amino acid sequence of SEQ ID NO: 1, an HC-CDR2 comprising the amino acid sequence of SEQ ID NO: 8, and an HC-CDR3 comprising the amino acid sequence of SEQ ID NO: 16; and b) a light chain variable domain comprising an LC-CDR1 comprising the amino acid sequence of SEQ ID NO: 27, an LC-CDR2 comprising the amino acid sequence of SEQ ID NO: 41, and an LC-CDR3 comprising the amino acid sequence of SEQ ID NO: 52. In some embodiments, the IgG1 is human IgG1. In some embodiments, the heavy chain constant region comprises or consists of the amino acid sequence of SEQ ID NO: 124. In some embodiments, the light chain constant region comprises or consists of the amino acid sequence of SEQ ID NO: 126. In some embodiments, the light chain constant region comprises or consists of the amino acid sequence of SEQ ID NO: 127. In some embodiments, the heavy chain constant region comprises or consists of the amino acid sequence of SEQ ID NO: 124 and the light chain constant region comprises or consists of the amino acid sequence of SEQ ID NO: 126. In some embodiments, the heavy chain constant region comprises or consists of the amino acid sequence of SEQ ID NO: 124 and the light chain constant region comprises or consists of the amino acid sequence of SEQ ID NO: 127.

In some embodiments, there is provided a full-length anti-TSLP antibody comprising IgG1 constant domains, wherein the anti-TSLP antibody comprises a) a heavy chain variable domain comprising an HC-CDR1 comprising the amino acid sequence of SEQ ID NO: 1, an HC-CDR2 comprising the amino acid sequence of SEQ ID NO: 9, and an HC-CDR3 comprising the amino acid sequence of SEQ ID NO: 16; and b) a light chain variable domain comprising an LC-CDR1 comprising the amino acid sequence of SEQ ID NO: 28, an LC-CDR2 comprising the amino acid sequence of SEQ ID NO: 39, and an LC-CDR3 comprising the amino acid sequence of SEQ ID NO: 52. In some embodiments, the IgG1 is human IgG1. In some embodiments, the heavy chain constant region comprises or consists of the amino acid sequence of SEQ ID NO: 124. In some embodiments, the light chain constant region comprises or consists of the amino acid sequence of SEQ ID NO: 126. In some embodiments, the light chain constant region comprises or consists of the amino acid sequence of SEQ ID NO: 127. In some embodiments, the heavy chain constant region comprises or consists of the amino acid sequence of SEQ ID NO: 124 and the light chain constant region comprises or consists of the amino acid sequence of SEQ ID NO: 126. In some embodiments, the heavy chain constant region comprises or consists of the amino acid sequence of SEQ ID NO: 124 and the light chain constant region comprises or consists of the amino acid sequence of SEQ ID NO: 127.

In some embodiments, there is provided a full-length anti-TSLP antibody comprising IgG1 constant domains, wherein the anti-TSLP antibody comprises a) a heavy chain variable domain comprising an HC-CDR1 comprising the amino acid sequence of SEQ ID NO: 1, an HC-CDR2 comprising the amino acid sequence of SEQ ID NO: 8, and an HC-CDR3 comprising the amino acid sequence of SEQ ID NO: 18; and b) a light chain variable domain comprising an LC-CDR1 comprising the amino acid sequence of SEQ ID NO: 27, an LC-CDR2 comprising the amino acid sequence of SEQ ID NO: 39, and an LC-CDR3 comprising the amino acid sequence of SEQ ID NO: 53. In some embodiments, the IgG1 is human IgG1. In some embodiments, the heavy chain constant region comprises or consists of the amino acid sequence of SEQ ID NO: 124. In some embodiments, the light chain constant region comprises or consists of the amino acid sequence of SEQ ID NO: 126. In some embodiments, the light chain constant region comprises or consists of the amino acid sequence of SEQ ID NO: 127. In some embodiments, the heavy chain constant region comprises or consists of the amino acid sequence of SEQ ID NO: 124 and the light chain constant region comprises or consists of the amino acid sequence of SEQ ID NO: 126. In some embodiments, the heavy chain constant region comprises or consists of the amino acid sequence of SEQ ID NO: 124 and the light chain constant region comprises or consists of the amino acid sequence of SEQ ID NO: 127.

In some embodiments, there is provided a full-length anti-TSLP antibody comprising IgG1 constant domains, wherein the anti-TSLP antibody comprises a) a heavy chain variable domain comprising an HC-CDR1 comprising the amino acid sequence of SEQ ID NO: 2, an HC-CDR2 comprising the amino acid sequence of SEQ ID NO: 10, and an HC-CDR3 comprising the amino acid sequence of SEQ ID NO: 19; and b) a light chain variable domain comprising an LC-CDR1 comprising the amino acid sequence of SEQ ID NO: 29, an LC-CDR2 comprising the amino acid sequence of SEQ ID NO: 42, and an LC-CDR3 comprising the amino acid sequence of SEQ ID NO: 54. In some embodiments, the IgG1 is human IgG1. In some embodiments, the heavy chain constant region comprises or consists of the amino acid sequence of SEQ ID NO: 124. In some embodiments, the light chain constant region comprises or consists of the amino acid sequence of SEQ ID NO: 126. In some embodiments, the light chain constant region comprises or consists of the amino acid sequence of SEQ ID NO: 127. In some embodiments, the heavy chain constant region comprises or consists of the amino acid sequence of SEQ ID NO: 124 and the light chain constant region comprises or consists of the amino acid sequence of SEQ ID NO: 126. In some embodiments, the heavy chain constant region comprises or consists of the amino acid sequence of SEQ ID NO: 124 and the light chain constant region comprises or consists of the amino acid sequence of SEQ ID NO: 127.

In some embodiments, there is provided a full-length anti-TSLP antibody comprising IgG1 constant domains, wherein the anti-TSLP antibody comprises a) a heavy chain variable domain comprising an HC-CDR1 comprising the amino acid sequence of SEQ ID NO: 2, an HC-CDR2 comprising the amino acid sequence of SEQ ID NO: 10, and an HC-CDR3 comprising the amino acid sequence of SEQ ID NO: 19; and b) a light chain variable domain comprising an LC-CDR1 comprising the amino acid sequence of SEQ ID NO: 30, an LC-CDR2 comprising the amino acid sequence of SEQ ID NO: 43, and an LC-CDR3 comprising the amino acid sequence of SEQ ID NO: 55. In some embodiments, the IgG1 is human IgG1. In some embodiments, the heavy chain constant region comprises or consists of the amino acid sequence of SEQ ID NO: 124. In some embodiments, the light chain constant region comprises or consists of the amino acid sequence of SEQ ID NO: 126. In some embodiments, the light chain constant region comprises or consists of the amino acid sequence of SEQ ID NO: 127. In some embodiments, the heavy chain constant region comprises or consists of the amino acid sequence of SEQ ID NO: 124 and the light chain constant region comprises or consists of the amino acid sequence of SEQ ID NO: 126. In some embodiments, the heavy chain constant region comprises or consists of the amino acid sequence of SEQ ID NO: 124 and the light chain constant region comprises or consists of the amino acid sequence of SEQ ID NO: 127.

In some embodiments, there is provided a full-length anti-TSLP antibody comprising IgG1 constant domains, wherein the anti-TSLP antibody comprises a) a heavy chain variable domain comprising an HC-CDR1 comprising the amino acid sequence of SEQ ID NO: 2, an HC-CDR2 comprising the amino acid sequence of SEQ ID NO: 10, and an HC-CDR3 comprising the amino acid sequence of SEQ ID NO: 19; and b) a light chain variable domain comprising an LC-CDR1 comprising the amino acid sequence of SEQ ID NO: 31, an LC-CDR2 comprising the amino acid sequence of SEQ ID NO: 44, and an LC-CDR3 comprising the amino acid sequence of SEQ ID NO: 56. In some embodiments, the IgG1 is human IgG1. In some embodiments, the heavy chain constant region comprises or consists of the amino acid sequence of SEQ ID NO: 124. In some embodiments, the light chain constant region comprises or consists of the amino acid sequence of SEQ ID NO: 126. In some embodiments, the light chain constant region comprises or consists of the amino acid sequence of SEQ ID NO: 127. In some embodiments, the heavy chain constant region comprises or consists of the amino acid sequence of SEQ ID NO: 124 and the light chain constant region comprises or consists of the amino acid sequence of SEQ ID NO: 126. In some embodiments, the heavy chain constant region comprises or consists of the amino acid sequence of SEQ ID NO: 124 and the light chain constant region comprises or consists of the amino acid sequence of SEQ ID NO: 127.

In some embodiments, there is provided a full-length anti-TSLP antibody comprising IgG1 constant domains, wherein the anti-TSLP antibody comprises a) a heavy chain variable domain comprising an HC-CDR1 comprising the amino acid sequence of SEQ ID NO: 2, an HC-CDR2 comprising the amino acid sequence of SEQ ID NO: 10, and an HC-CDR3 comprising the amino acid sequence of SEQ ID NO: 19; and b) a light chain variable domain comprising an LC-CDR1 comprising the amino acid sequence of SEQ ID NO: 32, an LC-CDR2 comprising the amino acid sequence of SEQ ID NO: 45, and an LC-CDR3 comprising the amino acid sequence of SEQ ID NO: 57. In some embodiments, the IgG1 is human IgG1. In some embodiments, the heavy chain constant region comprises or consists of the amino acid sequence of SEQ ID NO: 124. In some embodiments, the light chain constant region comprises or consists of the amino acid sequence of SEQ ID NO: 126. In some embodiments, the light chain constant region comprises or consists of the amino acid sequence of SEQ ID NO: 127. In some embodiments, the heavy chain constant region comprises or consists of the amino acid sequence of SEQ ID NO: 124 and the light chain constant region comprises or consists of the amino acid sequence of SEQ ID NO: 126. In some embodiments, the heavy chain constant region comprises or consists of the amino acid sequence of SEQ ID NO: 124 and the light chain constant region comprises or consists of the amino acid sequence of SEQ ID NO: 127.

In some embodiments, there is provided a full-length anti-TSLP antibody comprising IgG1 constant domains, wherein the anti-TSLP antibody comprises a) a heavy chain variable domain comprising an HC-CDR1 comprising the amino acid sequence of SEQ ID NO: 3, an HC-CDR2 comprising the amino acid sequence of SEQ ID NO: 11, and an HC-CDR3 comprising the amino acid sequence of SEQ ID NO: 19; and b) a light chain variable domain comprising an LC-CDR1 comprising the amino acid sequence of SEQ ID NO: 33, an LC-CDR2 comprising the amino acid sequence of SEQ ID NO: 45, and an LC-CDR3 comprising the amino acid sequence of SEQ ID NO: 58. In some embodiments, the IgG1 is human IgG1. In some embodiments, the heavy chain constant region comprises or consists of the amino acid sequence of SEQ ID NO: 124. In some embodiments, the light chain constant region comprises or consists of the amino acid sequence of SEQ ID NO: 126. In some embodiments, the light chain constant region comprises or consists of the amino acid sequence of SEQ ID NO: 127. In some embodiments, the heavy chain constant region comprises or consists of the amino acid sequence of SEQ ID NO: 124 and the light chain constant region comprises or consists of the amino acid sequence of SEQ ID NO: 126. In some embodiments, the heavy chain constant region comprises or consists of the amino acid sequence of SEQ ID NO: 124 and the light chain constant region comprises or consists of the amino acid sequence of SEQ ID NO: 127.

In some embodiments, there is provided a full-length anti-TSLP antibody comprising IgG1 constant domains, wherein the anti-TSLP antibody comprises a) a heavy chain variable domain comprising an HC-CDR1 comprising the amino acid sequence of SEQ ID NO: 3, an HC-CDR2 comprising the amino acid sequence of SEQ ID NO: 10, and an HC-CDR3 comprising the amino acid sequence of SEQ ID NO: 19; and b) a light chain variable domain comprising an LC-CDR1 comprising the amino acid sequence of SEQ ID NO: 31, an LC-CDR2 comprising the amino acid sequence of SEQ ID NO: 46, and an LC-CDR3 comprising the amino acid sequence of SEQ ID NO: 59. In some embodiments, the IgG1 is human IgG1. In some embodiments, the heavy chain constant region comprises or consists of the amino acid sequence of SEQ ID NO: 124. In some embodiments, the light chain constant region comprises or consists of the amino acid sequence of SEQ ID NO: 126. In some embodiments, the light chain constant region comprises or consists of the amino acid sequence of SEQ ID NO: 127. In some embodiments, the heavy chain constant region comprises or consists of the amino acid sequence of SEQ ID NO: 124 and the light chain constant region comprises or consists of the amino acid sequence of SEQ ID NO: 126. In some embodiments, the heavy chain constant region comprises or consists of the amino acid sequence of SEQ ID NO: 124 and the light chain constant region comprises or consists of the amino acid sequence of SEQ ID NO: 127.

In some embodiments, there is provided a full-length anti-TSLP antibody comprising IgG1 constant domains, wherein the anti-TSLP antibody comprises a) a heavy chain variable domain comprising an HC-CDR1 comprising the amino acid sequence of SEQ ID NO: 3, an HC-CDR2 comprising the amino acid sequence of SEQ ID NO: 12, and an HC-CDR3 comprising the amino acid sequence of SEQ ID NO: 20; and b) a light chain variable domain comprising an LC-CDR1 comprising the amino acid sequence of SEQ ID NO: 34, an LC-CDR2 comprising the amino acid sequence of SEQ ID NO: 47, and an LC-CDR3 comprising the amino acid sequence of SEQ ID NO: 60. In some embodiments, the IgG1 is human IgG1. In some embodiments, the heavy chain constant region comprises or consists of the amino acid sequence of SEQ ID NO: 124. In some embodiments, the light chain constant region comprises or consists of the amino acid sequence of SEQ ID NO: 126. In some embodiments, the light chain constant region comprises or consists of the amino acid sequence of SEQ ID NO: 127. In some embodiments, the heavy chain constant region comprises or consists of the amino acid sequence of SEQ ID NO: 124 and the light chain constant region comprises or consists of the amino acid sequence of SEQ ID NO: 126. In some embodiments, the heavy chain constant region comprises or consists of the amino acid sequence of SEQ ID NO: 124 and the light chain constant region comprises or consists of the amino acid sequence of SEQ ID NO: 127.

In some embodiments, there is provided a full-length anti-TSLP antibody comprising IgG1 constant domains, wherein the anti-TSLP antibody comprises a) a heavy chain variable domain comprising an HC-CDR1 comprising the amino acid sequence of SEQ ID NO: 4, an HC-CDR2 comprising the amino acid sequence of SEQ ID NO: 13, and an HC-CDR3 comprising the amino acid sequence of SEQ ID NO: 21; and b) a light chain variable domain comprising an LC-CDR1 comprising the amino acid sequence of SEQ ID NO: 35, an LC-CDR2 comprising the amino acid sequence of SEQ ID NO: 48, and an LC-CDR3 comprising the amino acid sequence of SEQ ID NO: 61. In some embodiments, the IgG1 is human IgG1. In some embodiments, the heavy chain constant region comprises or consists of the amino acid sequence of SEQ ID NO: 124. In some embodiments, the light chain constant region comprises or consists of the amino acid sequence of SEQ ID NO: 126. In some embodiments, the light chain constant region comprises or consists of the amino acid sequence of SEQ ID NO: 127. In some embodiments, the heavy chain constant region comprises or consists of the amino acid sequence of SEQ ID NO: 124 and the light chain constant region comprises or consists of the amino acid sequence of SEQ ID NO: 126. In some embodiments, the heavy chain constant region comprises or consists of the amino acid sequence of SEQ ID NO: 124 and the light chain constant region comprises or consists of the amino acid sequence of SEQ ID NO: 127.

In some embodiments, there is provided a full-length anti-TSLP antibody comprising IgG1 constant domains, wherein the anti-TSLP antibody comprises a) a heavy chain variable domain comprising an HC-CDR1 comprising the amino acid sequence of SEQ ID NO: 4, an HC-CDR2 comprising the amino acid sequence of SEQ ID NO: 13, and an HC-CDR3 comprising the amino acid sequence of SEQ ID NO: 22; and b) a light chain variable domain comprising an LC-CDR1 comprising the amino acid sequence of SEQ ID NO: 36, an LC-CDR2 comprising the amino acid sequence of SEQ ID NO: 48, and an LC-CDR3 comprising the amino acid sequence of SEQ ID NO: 62. In some embodiments, the IgG1 is human IgG1. In some embodiments, the heavy chain constant region comprises or consists of the amino acid sequence of SEQ ID NO: 124. In some embodiments, the light chain constant region comprises or consists of the amino acid sequence of SEQ ID NO: 126. In some embodiments, the light chain constant region comprises or consists of the amino acid sequence of SEQ ID NO: 127. In some embodiments, the heavy chain constant region comprises or consists of the amino acid sequence of SEQ ID NO: 124 and the light chain constant region comprises or consists of the amino acid sequence of SEQ ID NO: 126. In some embodiments, the heavy chain constant region comprises or consists of the amino acid sequence of SEQ ID NO: 124 and the light chain constant region comprises or consists of the amino acid sequence of SEQ ID NO: 127.

In some embodiments, there is provided a full-length anti-TSLP antibody comprising IgG1 constant domains, wherein the anti-TSLP antibody comprises a) a heavy chain variable domain comprising an HC-CDR1 comprising the amino acid sequence of SEQ ID NO: 5, an HC-CDR2 comprising the amino acid sequence of SEQ ID NO: 13, and an HC-CDR3 comprising the amino acid sequence of SEQ ID NO: 22; and b) a light chain variable domain comprising an LC-CDR1 comprising the amino acid sequence of SEQ ID NO: 37, an LC-CDR2 comprising the amino acid sequence of SEQ ID NO: 49, and an LC-CDR3 comprising the amino acid sequence of SEQ ID NO: 63. In some embodiments, the IgG1 is human IgG1. In some embodiments, the heavy chain constant region comprises or consists of the amino acid sequence of SEQ ID NO: 124. In some embodiments, the light chain constant region comprises or consists of the amino acid sequence of SEQ ID NO: 126. In some embodiments, the light chain constant region comprises or consists of the amino acid sequence of SEQ ID NO: 127. In some embodiments, the heavy chain constant region comprises or consists of the amino acid sequence of SEQ ID NO: 124 and the light chain constant region comprises or consists of the amino acid sequence of SEQ ID NO: 126. In some embodiments, the heavy chain constant region comprises or consists of the amino acid sequence of SEQ ID NO: 124 and the light chain constant region comprises or consists of the amino acid sequence of SEQ ID NO: 127.

In some embodiments, there is provided a full-length anti-TSLP antibody comprising IgG1 constant domains, wherein the anti-TSLP antibody comprises a) a heavy chain variable domain comprising an HC-CDR1 comprising the amino acid sequence of SEQ ID NO: 6, an HC-CDR2 comprising the amino acid sequence of SEQ ID NO: 14, and an HC-CDR3 comprising the amino acid sequence of SEQ ID NO: 23; and b) a light chain variable domain comprising an LC-CDR1 comprising the amino acid sequence of SEQ ID NO: 38, an LC-CDR2 comprising the amino acid sequence of SEQ ID NO: 42, and an LC-CDR3 comprising the amino acid sequence of SEQ ID NO: 60. In some embodiments, the IgG1 is human IgG1. In some embodiments, the heavy chain constant region comprises or consists of the amino acid sequence of SEQ ID NO: 124. In some embodiments, the light chain constant region comprises or consists of the amino acid sequence of SEQ ID NO: 126. In some embodiments, the light chain constant region comprises or consists of the amino acid sequence of SEQ ID NO: 127. In some embodiments, the heavy chain constant region comprises or consists of the amino acid sequence of SEQ ID NO: 124 and the light chain constant region comprises or consists of the amino acid sequence of SEQ ID NO: 126. In some embodiments, the heavy chain constant region comprises or consists of the amino acid sequence of SEQ ID NO: 124 and the light chain constant region comprises or consists of the amino acid sequence of SEQ ID NO: 127.

In some embodiments, there is provided a full-length anti-TSLP antibody comprising IgG1 constant domains, wherein the anti-TSLP antibody comprises a) a heavy chain variable domain comprising an HC-CDR1 comprising the amino acid sequence of SEQ ID NO: 6, an HC-CDR2 comprising the amino acid sequence of SEQ ID NO: 15, and an HC-CDR3 comprising the amino acid sequence of SEQ ID NO: 24; and b) a light chain variable domain comprising an LC-CDR1 comprising the amino acid sequence of SEQ ID NO: 31, an LC-CDR2 comprising the amino acid sequence of SEQ ID NO: 45, and an LC-CDR3 comprising the amino acid sequence of SEQ ID NO: 64. In some embodiments, the IgG1 is human IgG1. In some embodiments, the heavy chain constant region comprises or consists of the amino acid sequence of SEQ ID NO: 124. In some embodiments, the light chain constant region comprises or consists of the amino acid sequence of SEQ ID NO: 126. In some embodiments, the light chain constant region comprises or consists of the amino acid sequence of SEQ ID NO: 127. In some embodiments, the heavy chain constant region comprises or consists of the amino acid sequence of SEQ ID NO: 124 and the light chain constant region comprises or consists of the amino acid sequence of SEQ ID NO: 126. In some embodiments, the heavy chain constant region comprises or consists of the amino acid sequence of SEQ ID NO: 124 and the light chain constant region comprises or consists of the amino acid sequence of SEQ ID NO: 127.

In some embodiments, there is provided a full-length anti-TSLP antibody comprising IgG4 constant domains, wherein the anti-TSLP antibody comprises a) a heavy chain variable domain comprising an HC-CDR1 comprising the amino acid sequence of SEQ ID NO: 1, an HC-CDR2 comprising the amino acid sequence of SEQ ID NO: 7, and an HC-CDR3 comprising the amino acid sequence of SEQ ID NO: 16; and b) a light chain variable domain comprising an LC-CDR1 comprising the amino acid sequence of SEQ ID NO: 25, an LC-CDR2 comprising the amino acid sequence of SEQ ID NO: 39, and an LC-CDR3 comprising the amino acid sequence of SEQ ID NO: 50. In some embodiments, the IgG4 is human IgG4. In some embodiments, the heavy chain constant region comprises or consists of the amino acid sequence of SEQ ID NO: 125. In some embodiments, the light chain constant region comprises or consists of the amino acid sequence of SEQ ID NO: 126. In some embodiments, the light chain constant region comprises or consists of the amino acid sequence of SEQ ID NO: 127. In some embodiments, the heavy chain constant region comprises or consists of the amino acid sequence of SEQ ID NO: 125 and the light chain constant region comprises or consists of the amino acid sequence of SEQ ID NO: 126. In some embodiments, the heavy chain constant region comprises or consists of the amino acid sequence of SEQ ID NO: 125 and the light chain constant region comprises or consists of the amino acid sequence of SEQ ID NO: 127.

In some embodiments, there is provided a full-length anti-TSLP antibody comprising IgG4 constant domains, wherein the anti-TSLP antibody comprises a) a heavy chain variable domain comprising an HC-CDR1 comprising the amino acid sequence of SEQ ID NO: 1, an HC-CDR2 comprising the amino acid sequence of SEQ ID NO: 8, and an HC-CDR3 comprising the amino acid sequence of SEQ ID NO: 16; and b) a light chain variable domain comprising an LC-CDR1 comprising the amino acid sequence of SEQ ID NO: 25, an LC-CDR2 comprising the amino acid sequence of SEQ ID NO: 39, and an LC-CDR3 comprising the amino acid sequence of SEQ ID NO: 51. In some embodiments, the IgG4 is human IgG4. In some embodiments, the heavy chain constant region comprises or consists of the amino acid sequence of SEQ ID NO: 125. In some embodiments, the light chain constant region comprises or consists of the amino acid sequence of SEQ ID NO: 126. In some embodiments, the light chain constant region comprises or consists of the amino acid sequence of SEQ ID NO: 127. In some embodiments, the heavy chain constant region comprises or consists of the amino acid sequence of SEQ ID NO: 125 and the light chain constant region comprises or consists of the amino acid sequence of SEQ ID NO: 126. In some embodiments, the heavy chain constant region comprises or consists of the amino acid sequence of SEQ ID NO: 125 and the light chain constant region comprises or consists of the amino acid sequence of SEQ ID NO: 127.

In some embodiments, there is provided a full-length anti-TSLP antibody comprising IgG4 constant domains, wherein the anti-TSLP antibody comprises a) a heavy chain variable domain comprising an HC-CDR1 comprising the amino acid sequence of SEQ ID NO: 1, an HC-CDR2 comprising the amino acid sequence of SEQ ID NO: 8, and an HC-CDR3 comprising the amino acid sequence of SEQ ID NO: 17; and b) a light chain variable domain comprising an LC-CDR1 comprising the amino acid sequence of SEQ ID NO: 26, an LC-CDR2 comprising the amino acid sequence of SEQ ID NO: 40, and an LC-CDR3 comprising the amino acid sequence of SEQ ID NO: 52. In some embodiments, the IgG4 is human IgG4. In some embodiments, the heavy chain constant region comprises or consists of the amino acid sequence of SEQ ID NO: 125. In some embodiments, the light chain constant region comprises or consists of the amino acid sequence of SEQ ID NO: 126. In some embodiments, the light chain constant region comprises or consists of the amino acid sequence of SEQ ID NO: 127. In some embodiments, the heavy chain constant region comprises or consists of the amino acid sequence of SEQ ID NO: 125 and the light chain constant region comprises or consists of the amino acid sequence of SEQ ID NO: 126. In some embodiments, the heavy chain constant region comprises or consists of the amino acid sequence of SEQ ID NO: 125 and the light chain constant region comprises or consists of the amino acid sequence of SEQ ID NO: 127.

In some embodiments, there is provided a full-length anti-TSLP antibody comprising IgG4 constant domains, wherein the anti-TSLP antibody comprises a) a heavy chain variable domain comprising an HC-CDR1 comprising the amino acid sequence of SEQ ID NO: 1, an HC-CDR2 comprising the amino acid sequence of SEQ ID NO: 8, and an HC-CDR3 comprising the amino acid sequence of SEQ ID NO: 16; and b) a light chain variable domain comprising an LC-CDR1 comprising the amino acid sequence of SEQ ID NO: 27, an LC-CDR2 comprising the amino acid sequence of SEQ ID NO: 41, and an LC-CDR3 comprising the amino acid sequence of SEQ ID NO: 52. In some embodiments, the IgG4 is human IgG4. In some embodiments, the heavy chain constant region comprises or consists of the amino acid sequence of SEQ ID NO: 125. In some embodiments, the light chain constant region comprises or consists of the amino acid sequence of SEQ ID NO: 126. In some embodiments, the light chain constant region comprises or consists of the amino acid sequence of SEQ ID NO: 127. In some embodiments, the heavy chain constant region comprises or consists of the amino acid sequence of SEQ ID NO: 125 and the light chain constant region comprises or consists of the amino acid sequence of SEQ ID NO: 126. In some embodiments, the heavy chain constant region comprises or consists of the amino acid sequence of SEQ ID NO: 125 and the light chain constant region comprises or consists of the amino acid sequence of SEQ ID NO: 127.

In some embodiments, there is provided a full-length anti-TSLP antibody comprising IgG4 constant domains, wherein the anti-TSLP antibody comprises a) a heavy chain variable domain comprising an HC-CDR1 comprising the amino acid sequence of SEQ ID NO: 1, an HC-CDR2 comprising the amino acid sequence of SEQ ID NO: 9, and an HC-CDR3 comprising the amino acid sequence of SEQ ID NO: 16; and b) a light chain variable domain comprising an LC-CDR1 comprising the amino acid sequence of SEQ ID NO: 28, an LC-CDR2 comprising the amino acid sequence of SEQ ID NO: 39, and an LC-CDR3 comprising the amino acid sequence of SEQ ID NO: 52. In some embodiments, the IgG4 is human IgG4. In some embodiments, the heavy chain constant region comprises or consists of the amino acid sequence of SEQ ID NO: 125. In some embodiments, the light chain constant region comprises or consists of the amino acid sequence of SEQ ID NO: 126. In some embodiments, the light chain constant region comprises or consists of the amino acid sequence of SEQ ID NO: 127. In some embodiments, the heavy chain constant region comprises or consists of the amino acid sequence of SEQ ID NO: 125 and the light chain constant region comprises or consists of the amino acid sequence of SEQ ID NO: 126. In some embodiments, the heavy chain constant region comprises or consists of the amino acid sequence of SEQ ID NO: 125 and the light chain constant region comprises or consists of the amino acid sequence of SEQ ID NO: 127.

In some embodiments, there is provided a full-length anti-TSLP antibody comprising IgG4 constant domains, wherein the anti-TSLP antibody comprises a) a heavy chain variable domain comprising an HC-CDR1 comprising the amino acid sequence of SEQ ID NO: 1, an HC-CDR2 comprising the amino acid sequence of SEQ ID NO: 8, and an HC-CDR3 comprising the amino acid sequence of SEQ ID NO: 18; and b) a light chain variable domain comprising an LC-CDR1 comprising the amino acid sequence of SEQ ID NO: 27, an LC-CDR2 comprising the amino acid sequence of SEQ ID NO: 39, and an LC-CDR3 comprising the amino acid sequence of SEQ ID NO: 53. In some embodiments, the IgG4 is human IgG4. In some embodiments, the heavy chain constant region comprises or consists of the amino acid sequence of SEQ ID NO: 125. In some embodiments, the light chain constant region comprises or consists of the amino acid sequence of SEQ ID NO: 126. In some embodiments, the light chain constant region comprises or consists of the amino acid sequence of SEQ ID NO: 127. In some embodiments, the heavy chain constant region comprises or consists of the amino acid sequence of SEQ ID NO: 125 and the light chain constant region comprises or consists of the amino acid sequence of SEQ ID NO: 126. In some embodiments, the heavy chain constant region comprises or consists of the amino acid sequence of SEQ ID NO: 125 and the light chain constant region comprises or consists of the amino acid sequence of SEQ ID NO: 127.

In some embodiments, there is provided a full-length anti-TSLP antibody comprising IgG4 constant domains, wherein the anti-TSLP antibody comprises a) a heavy chain variable domain comprising an HC-CDR1 comprising the amino acid sequence of SEQ ID NO: 2, an HC-CDR2 comprising the amino acid sequence of SEQ ID NO: 10, and an HC-CDR3 comprising the amino acid sequence of SEQ ID NO: 19; and b) a light chain variable domain comprising an LC-CDR1 comprising the amino acid sequence of SEQ ID NO: 29, an LC-CDR2 comprising the amino acid sequence of SEQ ID NO: 42, and an LC-CDR3 comprising the amino acid sequence of SEQ ID NO: 54. In some embodiments, the IgG4 is human IgG4. In some embodiments, the heavy chain constant region comprises or consists of the amino acid sequence of SEQ ID NO: 125. In some embodiments, the light chain constant region comprises or consists of the amino acid sequence of SEQ ID NO: 126. In some embodiments, the light chain constant region comprises or consists of the amino acid sequence of SEQ ID NO: 127. In some embodiments, the heavy chain constant region comprises or consists of the amino acid sequence of SEQ ID NO: 125 and the light chain constant region comprises or consists of the amino acid sequence of SEQ ID NO: 126. In some embodiments, the heavy chain constant region comprises or consists of the amino acid sequence of SEQ ID NO: 125 and the light chain constant region comprises or consists of the amino acid sequence of SEQ ID NO: 127.

In some embodiments, there is provided a full-length anti-TSLP antibody comprising IgG4 constant domains, wherein the anti-TSLP antibody comprises a) a heavy chain variable domain comprising an HC-CDR1 comprising the amino acid sequence of SEQ ID NO: 2, an HC-CDR2 comprising the amino acid sequence of SEQ ID NO: 10, and an HC-CDR3 comprising the amino acid sequence of SEQ ID NO: 19; and b) a light chain variable domain comprising an LC-CDR1 comprising the amino acid sequence of SEQ ID NO: 30, an LC-CDR2 comprising the amino acid sequence of SEQ ID NO: 43, and an LC-CDR3 comprising the amino acid sequence of SEQ ID NO: 55. In some embodiments, the IgG4 is human IgG4. In some embodiments, the heavy chain constant region comprises or consists of the amino acid sequence of SEQ ID NO: 125. In some embodiments, the light chain constant region comprises or consists of the amino acid sequence of SEQ ID NO: 126. In some embodiments, the light chain constant region comprises or consists of the amino acid sequence of SEQ ID NO: 127. In some embodiments, the heavy chain constant region comprises or consists of the amino acid sequence of SEQ ID NO: 125 and the light chain constant region comprises or consists of the amino acid sequence of SEQ ID NO: 126. In some embodiments, the heavy chain constant region comprises or consists of the amino acid sequence of SEQ ID NO: 125 and the light chain constant region comprises or consists of the amino acid sequence of SEQ ID NO: 127.

In some embodiments, there is provided a full-length anti-TSLP antibody comprising IgG4 constant domains, wherein the anti-TSLP antibody comprises a) a heavy chain variable domain comprising an HC-CDR1 comprising the amino acid sequence of SEQ ID NO: 2, an HC-CDR2 comprising the amino acid sequence of SEQ ID NO: 10, and an HC-CDR3 comprising the amino acid sequence of SEQ ID NO: 19; and b) a light chain variable domain comprising an LC-CDR1 comprising the amino acid sequence of SEQ ID NO: 31, an LC-CDR2 comprising the amino acid sequence of SEQ ID NO: 44, and an LC-CDR3 comprising the amino acid sequence of SEQ ID NO: 56. In some embodiments, the IgG4 is human IgG4. In some embodiments, the heavy chain constant region comprises or consists of the amino acid sequence of SEQ ID NO: 125. In some embodiments, the light chain constant region comprises or consists of the amino acid sequence of SEQ ID NO: 126. In some embodiments, the light chain constant region comprises or consists of the amino acid sequence of SEQ ID NO: 127. In some embodiments, the heavy chain constant region comprises or consists of the amino acid sequence of SEQ ID NO: 125 and the light chain constant region comprises or consists of the amino acid sequence of SEQ ID NO: 126. In some embodiments, the heavy chain constant region comprises or consists of the amino acid sequence of SEQ ID NO: 125 and the light chain constant region comprises or consists of the amino acid sequence of SEQ ID NO: 127.

In some embodiments, there is provided a full-length anti-TSLP antibody comprising IgG4 constant domains, wherein the anti-TSLP antibody comprises a) a heavy chain variable domain comprising an HC-CDR1 comprising the amino acid sequence of SEQ ID NO: 2, an HC-CDR2 comprising the amino acid sequence of SEQ ID NO: 10, and an HC-CDR3 comprising the amino acid sequence of SEQ ID NO: 19; and b) a light chain variable domain comprising an LC-CDR1 comprising the amino acid sequence of SEQ ID NO: 32, an LC-CDR2 comprising the amino acid sequence of SEQ ID NO: 45, and an LC-CDR3 comprising the amino acid sequence of SEQ ID NO: 57. In some embodiments, the IgG4 is human IgG4. In some embodiments, the heavy chain constant region comprises or consists of the amino acid sequence of SEQ ID NO: 125. In some embodiments, the light chain constant region comprises or consists of the amino acid sequence of SEQ ID NO: 126. In some embodiments, the light chain constant region comprises or consists of the amino acid sequence of SEQ ID NO: 127. In some embodiments, the heavy chain constant region comprises or consists of the amino acid sequence of SEQ ID NO: 125 and the light chain constant region comprises or consists of the amino acid sequence of SEQ ID NO: 126. In some embodiments, the heavy chain constant region comprises or consists of the amino acid sequence of SEQ ID NO: 125 and the light chain constant region comprises or consists of the amino acid sequence of SEQ ID NO: 127.

In some embodiments, there is provided a full-length anti-TSLP antibody comprising IgG4 constant domains, wherein the anti-TSLP antibody comprises a) a heavy chain variable domain comprising an HC-CDR1 comprising the amino acid sequence of SEQ ID NO: 3, an HC-CDR2 comprising the amino acid sequence of SEQ ID NO: 11, and an HC-CDR3 comprising the amino acid sequence of SEQ ID NO: 19; and b) a light chain variable domain comprising an LC-CDR1 comprising the amino acid sequence of SEQ ID NO: 33, an LC-CDR2 comprising the amino acid sequence of SEQ ID NO: 45, and an LC-CDR3 comprising the amino acid sequence of SEQ ID NO: 58. In some embodiments, the IgG4 is human IgG4. In some embodiments, the heavy chain constant region comprises or consists of the amino acid sequence of SEQ ID NO: 125. In some embodiments, the light chain constant region comprises or consists of the amino acid sequence of SEQ ID NO: 126. In some embodiments, the light chain constant region comprises or consists of the amino acid sequence of SEQ ID NO: 127. In some embodiments, the heavy chain constant region comprises or consists of the amino acid sequence of SEQ ID NO: 125 and the light chain constant region comprises or consists of the amino acid sequence of SEQ ID NO: 126. In some embodiments, the heavy chain constant region comprises or consists of the amino acid sequence of SEQ ID NO: 125 and the light chain constant region comprises or consists of the amino acid sequence of SEQ ID NO: 127.

In some embodiments, there is provided a full-length anti-TSLP antibody comprising IgG4 constant domains, wherein the anti-TSLP antibody comprises a) a heavy chain variable domain comprising an HC-CDR1 comprising the amino acid sequence of SEQ ID NO: 3, an HC-CDR2 comprising the amino acid sequence of SEQ ID NO: 10, and an HC-CDR3 comprising the amino acid sequence of SEQ ID NO: 19; and b) a light chain variable domain comprising an LC-CDR1 comprising the amino acid sequence of SEQ ID NO: 31, an LC-CDR2 comprising the amino acid sequence of SEQ ID NO: 46, and an LC-CDR3 comprising the amino acid sequence of SEQ ID NO: 59. In some embodiments, the IgG4 is human IgG4. In some embodiments, the heavy chain constant region comprises or consists of the amino acid sequence of SEQ ID NO: 125. In some embodiments, the light chain constant region comprises or consists of the amino acid sequence of SEQ ID NO: 126. In some embodiments, the light chain constant region comprises or consists of the amino acid sequence of SEQ ID NO: 127. In some embodiments, the heavy chain constant region comprises or consists of the amino acid sequence of SEQ ID NO: 125 and the light chain constant region comprises or consists of the amino acid sequence of SEQ ID NO: 126. In some embodiments, the heavy chain constant region comprises or consists of the amino acid sequence of SEQ ID NO: 125 and the light chain constant region comprises or consists of the amino acid sequence of SEQ ID NO: 127.

In some embodiments, there is provided a full-length anti-TSLP antibody comprising IgG4 constant domains, wherein the anti-TSLP antibody comprises a) a heavy chain variable domain comprising an HC-CDR1 comprising the amino acid sequence of SEQ ID NO: 3, an HC-CDR2 comprising the amino acid sequence of SEQ ID NO: 12, and an HC-CDR3 comprising the amino acid sequence of SEQ ID NO: 20; and b) a light chain variable domain comprising an LC-CDR1 comprising the amino acid sequence of SEQ ID NO: 34, an LC-CDR2 comprising the amino acid sequence of SEQ ID NO: 47, and an LC-CDR3 comprising the amino acid sequence of SEQ ID NO: 60. In some embodiments, the IgG4 is human IgG4. In some embodiments, the heavy chain constant region comprises or consists of the amino acid sequence of SEQ ID NO: 125. In some embodiments, the light chain constant region comprises or consists of the amino acid sequence of SEQ ID NO: 126. In some embodiments, the light chain constant region comprises or consists of the amino acid sequence of SEQ ID NO: 127. In some embodiments, the heavy chain constant region comprises or consists of the amino acid sequence of SEQ ID NO: 125 and the light chain constant region comprises or consists of the amino acid sequence of SEQ ID NO: 126. In some embodiments, the heavy chain constant region comprises or consists of the amino acid sequence of SEQ ID NO: 125 and the light chain constant region comprises or consists of the amino acid sequence of SEQ ID NO: 127.

In some embodiments, there is provided a full-length anti-TSLP antibody comprising IgG4 constant domains, wherein the anti-TSLP antibody comprises a) a heavy chain variable domain comprising an HC-CDR1 comprising the amino acid sequence of SEQ ID NO: 4, an HC-CDR2 comprising the amino acid sequence of SEQ ID NO: 13, and an HC-CDR3 comprising the amino acid sequence of SEQ ID NO: 21; and b) a light chain variable domain comprising an LC-CDR1 comprising the amino acid sequence of SEQ ID NO: 35, an LC-CDR2 comprising the amino acid sequence of SEQ ID NO: 48, and an LC-CDR3 comprising the amino acid sequence of SEQ ID NO: 61. In some embodiments, the IgG4 is human IgG4. In some embodiments, the heavy chain constant region comprises or consists of the amino acid sequence of SEQ ID NO: 125. In some embodiments, the light chain constant region comprises or consists of the amino acid sequence of SEQ ID NO: 126. In some embodiments, the light chain constant region comprises or consists of the amino acid sequence of SEQ ID NO: 127. In some embodiments, the heavy chain constant region comprises or consists of the amino acid sequence of SEQ ID NO: 125 and the light chain constant region comprises or consists of the amino acid sequence of SEQ ID NO: 126. In some embodiments, the heavy chain constant region comprises or consists of the amino acid sequence of SEQ ID NO: 125 and the light chain constant region comprises or consists of the amino acid sequence of SEQ ID NO: 127.

In some embodiments, there is provided a full-length anti-TSLP antibody comprising IgG4 constant domains, wherein the anti-TSLP antibody comprises a) a heavy chain variable domain comprising an HC-CDR1 comprising the amino acid sequence of SEQ ID NO: 4, an HC-CDR2 comprising the amino acid sequence of SEQ ID NO: 13, and an HC-CDR3 comprising the amino acid sequence of SEQ ID NO: 22; and b) a light chain variable domain comprising an LC-CDR1 comprising the amino acid sequence of SEQ ID NO: 36, an LC-CDR2 comprising the amino acid sequence of SEQ ID NO: 48, and an LC-CDR3 comprising the amino acid sequence of SEQ ID NO: 62. In some embodiments, the IgG4 is human IgG4. In some embodiments, the heavy chain constant region comprises or consists of the amino acid sequence of SEQ ID NO: 125. In some embodiments, the light chain constant region comprises or consists of the amino acid sequence of SEQ ID NO: 126. In some embodiments, the light chain constant region comprises or consists of the amino acid sequence of SEQ ID NO: 127. In some embodiments, the heavy chain constant region comprises or consists of the amino acid sequence of SEQ ID NO: 125 and the light chain constant region comprises or consists of the amino acid sequence of SEQ ID NO: 126. In some embodiments, the heavy chain constant region comprises or consists of the amino acid sequence of SEQ ID NO: 125 and the light chain constant region comprises or consists of the amino acid sequence of SEQ ID NO: 127.

In some embodiments, there is provided a full-length anti-TSLP antibody comprising IgG4 constant domains, wherein the anti-TSLP antibody comprises a) a heavy chain variable domain comprising an HC-CDR1 comprising the amino acid sequence of SEQ ID NO: 5, an HC-CDR2 comprising the amino acid sequence of SEQ ID NO: 13, and an HC-CDR3 comprising the amino acid sequence of SEQ ID NO: 22; and b) a light chain variable domain comprising an LC-CDR1 comprising the amino acid sequence of SEQ ID NO: 37, an LC-CDR2 comprising the amino acid sequence of SEQ ID NO: 49, and an LC-CDR3 comprising the amino acid sequence of SEQ ID NO: 63. In some embodiments, the IgG4 is human IgG4. In some embodiments, the heavy chain constant region comprises or consists of the amino acid sequence of SEQ ID NO: 125. In some embodiments, the light chain constant region comprises or consists of the amino acid sequence of SEQ ID NO: 126. In some embodiments, the light chain constant region comprises or consists of the amino acid sequence of SEQ ID NO: 127. In some embodiments, the heavy chain constant region comprises or consists of the amino acid sequence of SEQ ID NO: 125 and the light chain constant region comprises or consists of the amino acid sequence of SEQ ID NO: 126. In some embodiments, the heavy chain constant region comprises or consists of the amino acid sequence of SEQ ID NO: 125 and the light chain constant region comprises or consists of the amino acid sequence of SEQ ID NO: 127.

In some embodiments, there is provided a full-length anti-TSLP antibody comprising IgG4 constant domains, wherein the anti-TSLP antibody comprises a) a heavy chain variable domain comprising an HC-CDR1 comprising the amino acid sequence of SEQ ID NO: 6, an HC-CDR2 comprising the amino acid sequence of SEQ ID NO: 14, and an HC-CDR3 comprising the amino acid sequence of SEQ ID NO: 23; and b) a light chain variable domain comprising an LC-CDR1 comprising the amino acid sequence of SEQ ID NO: 38, an LC-CDR2 comprising the amino acid sequence of SEQ ID NO: 42, and an LC-CDR3 comprising the amino acid sequence of SEQ ID NO: 60. In some embodiments, the IgG4 is human IgG4. In some embodiments, the heavy chain constant region comprises or consists of the amino acid sequence of SEQ ID NO: 125. In some embodiments, the light chain constant region comprises or consists of the amino acid sequence of SEQ ID NO: 126. In some embodiments, the light chain constant region comprises or consists of the amino acid sequence of SEQ ID NO: 127. In some embodiments, the heavy chain constant region comprises or consists of the amino acid sequence of SEQ ID NO: 125 and the light chain constant region comprises or consists of the amino acid sequence of SEQ ID NO: 126. In some embodiments, the heavy chain constant region comprises or consists of the amino acid sequence of SEQ ID NO: 125 and the light chain constant region comprises or consists of the amino acid sequence of SEQ ID NO: 127.

In some embodiments, there is provided a full-length anti-TSLP antibody comprising IgG4 constant domains, wherein the anti-TSLP antibody comprises a) a heavy chain variable domain comprising an HC-CDR1 comprising the amino acid sequence of SEQ ID NO: 6, an HC-CDR2 comprising the amino acid sequence of SEQ ID NO: 15, and an HC-CDR3 comprising the amino acid sequence of SEQ ID NO: 24; and b) a light chain variable domain comprising an LC-CDR1 comprising the amino acid sequence of SEQ ID NO: 31, an LC-CDR2 comprising the amino acid sequence of SEQ ID NO: 45, and an LC-CDR3 comprising the amino acid sequence of SEQ ID NO: 64. In some embodiments, the IgG4 is human IgG4. In some embodiments, the heavy chain constant region comprises or consists of the amino acid sequence of SEQ ID NO: 125. In some embodiments, the light chain constant region comprises or consists of the amino acid sequence of SEQ ID NO: 126. In some embodiments, the light chain constant region comprises or consists of the amino acid sequence of SEQ ID NO: 127. In some embodiments, the heavy chain constant region comprises or consists of the amino acid sequence of SEQ ID NO: 125 and the light chain constant region comprises or consists of the amino acid sequence of SEQ ID NO: 126. In some embodiments, the heavy chain constant region comprises or consists of the amino acid sequence of SEQ ID NO: 125 and the light chain constant region comprises or consists of the amino acid sequence of SEQ ID NO: 127.

In some embodiments, there is provided a full-length anti-TSLP antibody comprising IgG1 constant domains, wherein the anti-TSLP antibody comprises a heavy chain variable domain comprising the amino acid sequence of any one of SEQ ID NOs: 65-83, or a variant thereof having at least about 90% (for example at least about any of 91%, 92%, 93%, 94%, 95%, 96%, 97%, 98%, or 99%) sequence identity, and a light chain variable domain comprising the amino acid sequence of any one of SEQ ID NOs: 84-102, or a variant thereof having at least about 90% (for example at least about any of 91%, 92%, 93%, 94%, 95%, 96%, 97%, 98%, or 99%) sequence identity. In some embodiments, the IgG1 is human IgG1. In some embodiments, the heavy chain constant region comprises or consists of the amino acid sequence of SEQ ID NO: 124. In some embodiments, the light chain constant region comprises or consists of the amino acid sequence of SEQ ID NO: 126. In some embodiments, the light chain constant region comprises or consists of the amino acid sequence of SEQ ID NO: 127. In some embodiments, the heavy chain constant region comprises or consists of the amino acid sequence of SEQ ID NO: 124 and the light chain constant region comprises or consists of the amino acid sequence of SEQ ID NO: 126. In some embodiments, the heavy chain constant region comprises or consists of the amino acid sequence of SEQ ID NO: 124 and the light chain constant region comprises or consists of the amino acid sequence of SEQ ID NO: 127.

In some embodiments, there is provided a full-length anti-TSLP antibody comprising IgG2 constant domains, wherein the anti-TSLP antibody comprises a heavy chain variable domain comprising the amino acid sequence of any one of SEQ ID NOs: 65-83, or a variant thereof having at least about 90% (for example at least about any of 91%, 92%, 93%, 94%, 95%, 96%, 97%, 98%, or 99%) sequence identity, and a light chain variable domain comprising the amino acid sequence of any one of SEQ ID NOs: 84-102, or a variant thereof having at least about 90% (for example at least about any of 91%, 92%, 93%, 94%, 95%, 96%, 97%, 98%, or 99%) sequence identity. In some embodiments, the IgG2 is human IgG2. In some embodiments, the light chain constant region comprises or consists of the amino acid sequence of SEQ ID NO: 126. In some embodiments, the light chain constant region comprises or consists of the amino acid sequence of SEQ ID NO: 127.

In some embodiments, there is provided a full-length anti-TSLP antibody comprising IgG3 constant domains, wherein the anti-TSLP antibody comprises a heavy chain variable domain comprising the amino acid sequence of any one of SEQ ID NOs: 65-83, or a variant thereof having at least about 90% (for example at least about any of 91%, 92%, 93%, 94%, 95%, 96%, 97%, 98%, or 99%) sequence identity, and a light chain variable domain comprising the amino acid sequence of any one of SEQ ID NOs: 84-102, or a variant thereof having at least about 90% (for example at least about any of 91%, 92%, 93%, 94%, 95%, 96%, 97%, 98%, or 99%) sequence identity. In some embodiments, the IgG3 is human IgG3. In some embodiments, the light chain constant region comprises or consists of the amino acid sequence of SEQ ID NO: 126. In some embodiments, the light chain constant region comprises or consists of the amino acid sequence of SEQ ID NO: 127.

In some embodiments, there is provided a full-length anti-TSLP antibody comprising IgG4 constant domains, wherein the anti-TSLP antibody comprises a heavy chain variable domain comprising the amino acid sequence of any one of SEQ ID NOs: 65-83, or a variant thereof having at least about 90% (for example at least about any of 91%, 92%, 93%, 94%, 95%, 96%, 97%, 98%, or 99%) sequence identity, and a light chain variable domain comprising the amino acid sequence of any one of SEQ ID NOs: 84-102, or a variant thereof having at least about 90% (for example at least about any of 91%, 92%, 93%, 94%, 95%, 96%, 97%, 98%, or 99%) sequence identity. In some embodiments, the IgG4 is human IgG4. In some embodiments, the heavy chain constant region comprises or consists of the amino acid sequence of SEQ ID NO: 125. In some embodiments, the light chain constant region comprises or consists of the amino acid sequence of SEQ ID NO: 126. In some embodiments, the light chain constant region comprises or consists of the amino acid sequence of SEQ ID NO: 127. In some embodiments, the heavy chain constant region comprises or consists of the amino acid sequence of SEQ ID NO: 125 and the light chain constant region comprises or consists of the amino acid sequence of SEQ ID NO: 126. In some embodiments, the heavy chain constant region comprises or consists of the amino acid sequence of SEQ ID NO: 125 and the light chain constant region comprises or consists of the amino acid sequence of SEQ ID NO: 127.

In some embodiments, there is provided a full-length anti-TSLP antibody comprising IgG1 constant domains, wherein the anti-TSLP antibody comprises a heavy chain variable domain comprising the amino acid sequence of any one of SEQ ID NOs: 65-83, and a light chain variable domain comprising the amino acid sequence of any one of SEQ ID NOs: 84-102. In some embodiments, the IgG1 is human IgG1. In some embodiments, the heavy chain constant region comprises or consists of the amino acid sequence of SEQ ID NO: 124. In some embodiments, the light chain constant region comprises or consists of the amino acid sequence of SEQ ID NO: 126. In some embodiments, the light chain constant region comprises or consists of the amino acid sequence of SEQ ID NO: 127. In some embodiments, the heavy chain constant region comprises or consists of the amino acid sequence of SEQ ID NO: 124 and the light chain constant region comprises or consists of the amino acid sequence of SEQ ID NO: 126. In some embodiments, the heavy chain constant region comprises or consists of the amino acid sequence of SEQ ID NO: 124 and the light chain constant region comprises or consists of the amino acid sequence of SEQ ID NO: 127.

In some embodiments, there is provided a full-length anti-TSLP antibody comprising IgG4 constant domains, wherein the anti-TSLP antibody comprises a heavy chain variable domain comprising the amino acid sequence of any one of SEQ ID NOs: 65-83, and a light chain variable domain comprising the amino acid sequence of any one of SEQ ID NOs: 84-102. In some embodiments, the IgG4 is human IgG4. In some embodiments, the heavy chain constant region comprises or consists of the amino acid sequence of SEQ ID NO: 125. In some embodiments, the light chain constant region comprises or consists of the amino acid sequence of SEQ ID NO: 126. In some embodiments, the light chain constant region comprises or consists of the amino acid sequence of SEQ ID NO: 127. In some embodiments, the heavy chain constant region comprises or consists of the amino acid sequence of SEQ ID NO: 125 and the light chain constant region comprises or consists of the amino acid sequence of SEQ ID NO: 126. In some embodiments, the heavy chain constant region comprises or consists of the amino acid sequence of SEQ ID NO: 125 and the light chain constant region comprises or consists of the amino acid sequence of SEQ ID NO: 127.

In some embodiments, there is provided a full-length anti-TSLP antibody comprising IgG1 constant domains, wherein the anti-TSLP antibody comprises a heavy chain variable domain comprising the amino acid sequence of SEQ ID NO: 65 and a light chain variable domain comprising the amino acid sequence of SEQ ID NO: 84. In some embodiments, the IgG1 is human IgG1. In some embodiments, the heavy chain constant region comprises or consists of the amino acid sequence of SEQ ID NO: 124. In some embodiments, the light chain constant region comprises or consists of the amino acid sequence of SEQ ID NO: 126. In some embodiments, the light chain constant region comprises or consists of the amino acid sequence of SEQ ID NO: 127. In some embodiments, the heavy chain constant region comprises or consists of the amino acid sequence of SEQ ID NO: 124 and the light chain constant region comprises or consists of the amino acid sequence of SEQ ID NO: 126. In some embodiments, the heavy chain constant region comprises or consists of the amino acid sequence of SEQ ID NO: 124 and the light chain constant region comprises or consists of the amino acid sequence of SEQ ID NO: 127.

In some embodiments, there is provided a full-length anti-TSLP antibody comprising IgG1 constant domains, wherein the anti-TSLP antibody comprises a heavy chain variable domain comprising the amino acid sequence of SEQ ID NO: 65 and a light chain variable domain comprising the amino acid sequence of SEQ ID NO: 85. In some embodiments, the IgG1 is human IgG1. In some embodiments, the heavy chain constant region comprises or consists of the amino acid sequence of SEQ ID NO: 124. In some embodiments, the light chain constant region comprises or consists of the amino acid sequence of SEQ ID NO: 126. In some embodiments, the light chain constant region comprises or consists of the amino acid sequence of SEQ ID NO: 127. In some embodiments, the heavy chain constant region comprises or consists of the amino acid sequence of SEQ ID NO: 124 and the light chain constant region comprises or consists of the amino acid sequence of SEQ ID NO: 126. In some embodiments, the heavy chain constant region comprises or consists of the amino acid sequence of SEQ ID NO: 124 and the light chain constant region comprises or consists of the amino acid sequence of SEQ ID NO: 127.

In some embodiments, there is provided a full-length anti-TSLP antibody comprising IgG1 constant domains, wherein the anti-TSLP antibody comprises a heavy chain variable domain comprising the amino acid sequence of SEQ ID NO: 66 and a light chain variable domain comprising the amino acid sequence of SEQ ID NO: 84. In some embodiments, the IgG1 is human IgG1. In some embodiments, the heavy chain constant region comprises or consists of the amino acid sequence of SEQ ID NO: 124. In some embodiments, the light chain constant region comprises or consists of the amino acid sequence of SEQ ID NO: 126. In some embodiments, the light chain constant region comprises or consists of the amino acid sequence of SEQ ID NO: 127. In some embodiments, the heavy chain constant region comprises or consists of the amino acid sequence of SEQ ID NO: 124 and the light chain constant region comprises or consists of the amino acid sequence of SEQ ID NO: 126. In some embodiments, the heavy chain constant region comprises or consists of the amino acid sequence of SEQ ID NO: 124 and the light chain constant region comprises or consists of the amino acid sequence of SEQ ID NO: 127.

In some embodiments, there is provided a full-length anti-TSLP antibody comprising IgG1 constant domains, wherein the anti-TSLP antibody comprises a heavy chain variable domain comprising the amino acid sequence of SEQ ID NO: 66 and a light chain variable domain comprising the amino acid sequence of SEQ ID NO: 85. In some embodiments, the IgG1 is human IgG1. In some embodiments, the heavy chain constant region comprises or consists of the amino acid sequence of SEQ ID NO: 124. In some embodiments, the light chain constant region comprises or consists of the amino acid sequence of SEQ ID NO: 126. In some embodiments, the light chain constant region comprises or consists of the amino acid sequence of SEQ ID NO: 127. In some embodiments, the heavy chain constant region comprises or consists of the amino acid sequence of SEQ ID NO: 124 and the light chain constant region comprises or consists of the amino acid sequence of SEQ ID NO: 126. In some embodiments, the heavy chain constant region comprises or consists of the amino acid sequence of SEQ ID NO: 124 and the light chain constant region comprises or consists of the amino acid sequence of SEQ ID NO: 127.

In some embodiments, there is provided a full-length anti-TSLP antibody comprising IgG1 constant domains, wherein the anti-TSLP antibody comprises a heavy chain variable domain comprising the amino acid sequence of SEQ ID NO: 67 and a light chain variable domain comprising the amino acid sequence of SEQ ID NO: 84. In some embodiments, the IgG1 is human IgG1. In some embodiments, the heavy chain constant region comprises or consists of the amino acid sequence of SEQ ID NO: 124. In some embodiments, the light chain constant region comprises or consists of the amino acid sequence of SEQ ID NO: 126. In some embodiments, the light chain constant region comprises or consists of the amino acid sequence of SEQ ID NO: 127. In some embodiments, the heavy chain constant region comprises or consists of the amino acid sequence of SEQ ID NO: 124 and the light chain constant region comprises or consists of the amino acid sequence of SEQ ID NO: 126. In some embodiments, the heavy chain constant region comprises or consists of the amino acid sequence of SEQ ID NO: 124 and the light chain constant region comprises or consists of the amino acid sequence of SEQ ID NO: 127.

In some embodiments, there is provided a full-length anti-TSLP antibody comprising IgG1 constant domains, wherein the anti-TSLP antibody comprises a heavy chain variable domain comprising the amino acid sequence of SEQ ID NO: 67 and a light chain variable domain comprising the amino acid sequence of SEQ ID NO: 85. In some embodiments, the IgG1 is human IgG1. In some embodiments, the heavy chain constant region comprises or consists of the amino acid sequence of SEQ ID NO: 124. In some embodiments, the light chain constant region comprises or consists of the amino acid sequence of SEQ ID NO: 126. In some embodiments, the light chain constant region comprises or consists of the amino acid sequence of SEQ ID NO: 127. In some embodiments, the heavy chain constant region comprises or consists of the amino acid sequence of SEQ ID NO: 124 and the light chain constant region comprises or consists of the amino acid sequence of SEQ ID NO: 126. In some embodiments, the heavy chain constant region comprises or consists of the amino acid sequence of SEQ ID NO: 124 and the light chain constant region comprises or consists of the amino acid sequence of SEQ ID NO: 127.

In some embodiments, there is provided a full-length anti-TSLP antibody comprising IgG1 constant domains, wherein the anti-TSLP antibody comprises a heavy chain variable domain comprising the amino acid sequence of SEQ ID NO: 68 and a light chain variable domain comprising the amino acid sequence of SEQ ID NO: 84. In some embodiments, the IgG1 is human IgG1. In some embodiments, the heavy chain constant region comprises or consists of the amino acid sequence of SEQ ID NO: 124. In some embodiments, the light chain constant region comprises or consists of the amino acid sequence of SEQ ID NO: 126. In some embodiments, the light chain constant region comprises or consists of the amino acid sequence of SEQ ID NO: 127. In some embodiments, the heavy chain constant region comprises or consists of the amino acid sequence of SEQ ID NO: 124 and the light chain constant region comprises or consists of the amino acid sequence of SEQ ID NO: 126. In some embodiments, the heavy chain constant region comprises or consists of the amino acid sequence of SEQ ID NO: 124 and the light chain constant region comprises or consists of the amino acid sequence of SEQ ID NO: 127.

In some embodiments, there is provided a full-length anti-TSLP antibody comprising IgG1 constant domains, wherein the anti-TSLP antibody comprises a heavy chain variable domain comprising the amino acid sequence of SEQ ID NO: 68 and a light chain variable domain comprising the amino acid sequence of SEQ ID NO: 85. In some embodiments, the IgG1 is human IgG1. In some embodiments, the heavy chain constant region comprises or consists of the amino acid sequence of SEQ ID NO: 124. In some embodiments, the light chain constant region comprises or consists of the amino acid sequence of SEQ ID NO: 126. In some embodiments, the light chain constant region comprises or consists of the amino acid sequence of SEQ ID NO: 127. In some embodiments, the heavy chain constant region comprises or consists of the amino acid sequence of SEQ ID NO: 124 and the light chain constant region comprises or consists of the amino acid sequence of SEQ ID NO: 126. In some embodiments, the heavy chain constant region comprises or consists of the amino acid sequence of SEQ ID NO: 124 and the light chain constant region comprises or consists of the amino acid sequence of SEQ ID NO: 127.

In some embodiments, there is provided a full-length anti-TSLP antibody comprising IgG1 constant domains, wherein the anti-TSLP antibody comprises a heavy chain variable domain comprising the amino acid sequence of SEQ ID NO: 69 and a light chain variable domain comprising the amino acid sequence of SEQ ID NO: 86. In some embodiments, the IgG1 is human IgG1. In some embodiments, the heavy chain constant region comprises or consists of the amino acid sequence of SEQ ID NO: 124. In some embodiments, the light chain constant region comprises or consists of the amino acid sequence of SEQ ID NO: 126. In some embodiments, the light chain constant region comprises or consists of the amino acid sequence of SEQ ID NO: 127. In some embodiments, the heavy chain constant region comprises or consists of the amino acid sequence of SEQ ID NO: 124 and the light chain constant region comprises or consists of the amino acid sequence of SEQ ID NO: 126. In some embodiments, the heavy chain constant region comprises or consists of the amino acid sequence of SEQ ID NO: 124 and the light chain constant region comprises or consists of the amino acid sequence of SEQ ID NO: 127.

In some embodiments, there is provided a full-length anti-TSLP antibody comprising IgG1 constant domains, wherein the anti-TSLP antibody comprises a heavy chain variable domain comprising the amino acid sequence of SEQ ID NO: 70 and a light chain variable domain comprising the amino acid sequence of SEQ ID NO: 87. In some embodiments, the IgG1 is human IgG1. In some embodiments, the heavy chain constant region comprises or consists of the amino acid sequence of SEQ ID NO: 124. In some embodiments, the light chain constant region comprises or consists of the amino acid sequence of SEQ ID NO: 126. In some embodiments, the light chain constant region comprises or consists of the amino acid sequence of SEQ ID NO: 127. In some embodiments, the heavy chain constant region comprises or consists of the amino acid sequence of SEQ ID NO: 124 and the light chain constant region comprises or consists of the amino acid sequence of SEQ ID NO: 126. In some embodiments, the heavy chain constant region comprises or consists of the amino acid sequence of SEQ ID NO: 124 and the light chain constant region comprises or consists of the amino acid sequence of SEQ ID NO: 127.

In some embodiments, there is provided a full-length anti-TSLP antibody comprising IgG1 constant domains, wherein the anti-TSLP antibody comprises a heavy chain variable domain comprising the amino acid sequence of SEQ ID NO: 69 and a light chain variable domain comprising the amino acid sequence of SEQ ID NO: 88. In some embodiments, the IgG1 is human IgG1. In some embodiments, the heavy chain constant region comprises or consists of the amino acid sequence of SEQ ID NO: 124. In some embodiments, the light chain constant region comprises or consists of the amino acid sequence of SEQ ID NO: 126. In some embodiments, the light chain constant region comprises or consists of the amino acid sequence of SEQ ID NO: 127. In some embodiments, the heavy chain constant region comprises or consists of the amino acid sequence of SEQ ID NO: 124 and the light chain constant region comprises or consists of the amino acid sequence of SEQ ID NO: 126. In some embodiments, the heavy chain constant region comprises or consists of the amino acid sequence of SEQ ID NO: 124 and the light chain constant region comprises or consists of the amino acid sequence of SEQ ID NO: 127.

In some embodiments, there is provided a full-length anti-TSLP antibody comprising IgG1 constant domains, wherein the anti-TSLP antibody comprises a heavy chain variable domain comprising the amino acid sequence of SEQ ID NO: 71 and a light chain variable domain comprising the amino acid sequence of SEQ ID NO: 89. In some embodiments, the IgG1 is human IgG1. In some embodiments, the heavy chain constant region comprises or consists of the amino acid sequence of SEQ ID NO: 124. In some embodiments, the light chain constant region comprises or consists of the amino acid sequence of SEQ ID NO: 126. In some embodiments, the light chain constant region comprises or consists of the amino acid sequence of SEQ ID NO: 127. In some embodiments, the heavy chain constant region comprises or consists of the amino acid sequence of SEQ ID NO: 124 and the light chain constant region comprises or consists of the amino acid sequence of SEQ ID NO: 126. In some embodiments, the heavy chain constant region comprises or consists of the amino acid sequence of SEQ ID NO: 124 and the light chain constant region comprises or consists of the amino acid sequence of SEQ ID NO: 127.

In some embodiments, there is provided a full-length anti-TSLP antibody comprising IgG1 constant domains, wherein the anti-TSLP antibody comprises a heavy chain variable domain comprising the amino acid sequence of SEQ ID NO: 72 and a light chain variable domain comprising the amino acid sequence of SEQ ID NO: 90. In some embodiments, the IgG1 is human IgG1. In some embodiments, the heavy chain constant region comprises or consists of the amino acid sequence of SEQ ID NO: 124. In some embodiments, the light chain constant region comprises or consists of the amino acid sequence of SEQ ID NO: 126. In some embodiments, the light chain constant region comprises or consists of the amino acid sequence of SEQ ID NO: 127. In some embodiments, the heavy chain constant region comprises or consists of the amino acid sequence of SEQ ID NO: 124 and the light chain constant region comprises or consists of the amino acid sequence of SEQ ID NO: 126. In some embodiments, the heavy chain constant region comprises or consists of the amino acid sequence of SEQ ID NO: 124 and the light chain constant region comprises or consists of the amino acid sequence of SEQ ID NO: 127.

In some embodiments, there is provided a full-length anti-TSLP antibody comprising IgG1 constant domains, wherein the anti-TSLP antibody comprises a heavy chain variable domain comprising the amino acid sequence of SEQ ID NO: 73 and a light chain variable domain comprising the amino acid sequence of SEQ ID NO: 91. In some embodiments, the IgG1 is human IgG1. In some embodiments, the heavy chain constant region comprises or consists of the amino acid sequence of SEQ ID NO: 124. In some embodiments, the light chain constant region comprises or consists of the amino acid sequence of SEQ ID NO: 126. In some embodiments, the light chain constant region comprises or consists of the amino acid sequence of SEQ ID NO: 127. In some embodiments, the heavy chain constant region comprises or consists of the amino acid sequence of SEQ ID NO: 124 and the light chain constant region comprises or consists of the amino acid sequence of SEQ ID NO: 126. In some embodiments, the heavy chain constant region comprises or consists of the amino acid sequence of SEQ ID NO: 124 and the light chain constant region comprises or consists of the amino acid sequence of SEQ ID NO: 127.

In some embodiments, there is provided a full-length anti-TSLP antibody comprising IgG1 constant domains, wherein the anti-TSLP antibody comprises a heavy chain variable domain comprising the amino acid sequence of SEQ ID NO: 74 and a light chain variable domain comprising the amino acid sequence of SEQ ID NO: 91. In some embodiments, the IgG1 is human IgG1. In some embodiments, the heavy chain constant region comprises or consists of the amino acid sequence of SEQ ID NO: 124. In some embodiments, the light chain constant region comprises or consists of the amino acid sequence of SEQ ID NO: 126. In some embodiments, the light chain constant region comprises or consists of the amino acid sequence of SEQ ID NO: 127. In some embodiments, the heavy chain constant region comprises or consists of the amino acid sequence of SEQ ID NO: 124 and the light chain constant region comprises or consists of the amino acid sequence of SEQ ID NO: 126. In some embodiments, the heavy chain constant region comprises or consists of the amino acid sequence of SEQ ID NO: 124 and the light chain constant region comprises or consists of the amino acid sequence of SEQ ID NO: 127.

In some embodiments, there is provided a full-length anti-TSLP antibody comprising IgG1 constant domains, wherein the anti-TSLP antibody comprises a heavy chain variable domain comprising the amino acid sequence of SEQ ID NO: 75 and a light chain variable domain comprising the amino acid sequence of SEQ ID NO: 91. In some embodiments, the IgG1 is human IgG1. In some embodiments, the heavy chain constant region comprises or consists of the amino acid sequence of SEQ ID NO: 124. In some embodiments, the light chain constant region comprises or consists of the amino acid sequence of SEQ ID NO: 126. In some embodiments, the light chain constant region comprises or consists of the amino acid sequence of SEQ ID NO: 127. In some embodiments, the heavy chain constant region comprises or consists of the amino acid sequence of SEQ ID NO: 124 and the light chain constant region comprises or consists of the amino acid sequence of SEQ ID NO: 126. In some embodiments, the heavy chain constant region comprises or consists of the amino acid sequence of SEQ ID NO: 124 and the light chain constant region comprises or consists of the amino acid sequence of SEQ ID NO: 127.

In some embodiments, there is provided a full-length anti-TSLP antibody comprising IgG1 constant domains, wherein the anti-TSLP antibody comprises a heavy chain variable domain comprising the amino acid sequence of SEQ ID NO: 73 and a light chain variable domain comprising the amino acid sequence of SEQ ID NO: 92. In some embodiments, the IgG1 is human IgG1. In some embodiments, the heavy chain constant region comprises or consists of the amino acid sequence of SEQ ID NO: 124. In some embodiments, the light chain constant region comprises or consists of the amino acid sequence of SEQ ID NO: 126. In some embodiments, the light chain constant region comprises or consists of the amino acid sequence of SEQ ID NO: 127. In some embodiments, the heavy chain constant region comprises or consists of the amino acid sequence of SEQ ID NO: 124 and the light chain constant region comprises or consists of the amino acid sequence of SEQ ID NO: 126. In some embodiments, the heavy chain constant region comprises or consists of the amino acid sequence of SEQ ID NO: 124 and the light chain constant region comprises or consists of the amino acid sequence of SEQ ID NO: 127.

In some embodiments, there is provided a full-length anti-TSLP antibody comprising IgG1 constant domains, wherein the anti-TSLP antibody comprises a heavy chain variable domain comprising the amino acid sequence of SEQ ID NO: 73 and a light chain variable domain comprising the amino acid sequence of SEQ ID NO: 93. In some embodiments, the IgG1 is human IgG1. In some embodiments, the heavy chain constant region comprises or consists of the amino acid sequence of SEQ ID NO: 124. In some embodiments, the light chain constant region comprises or consists of the amino acid sequence of SEQ ID NO: 126. In some embodiments, the light chain constant region comprises or consists of the amino acid sequence of SEQ ID NO: 127. In some embodiments, the heavy chain constant region comprises or consists of the amino acid sequence of SEQ ID NO: 124 and the light chain constant region comprises or consists of the amino acid sequence of SEQ ID NO: 126. In some embodiments, the heavy chain constant region comprises or consists of the amino acid sequence of SEQ ID NO: 124 and the light chain constant region comprises or consists of the amino acid sequence of SEQ ID NO: 127.

In some embodiments, there is provided a full-length anti-TSLP antibody comprising IgG1 constant domains, wherein the anti-TSLP antibody comprises a heavy chain variable domain comprising the amino acid sequence of SEQ ID NO: 73 and a light chain variable domain comprising the amino acid sequence of SEQ ID NO: 94. In some embodiments, the IgG1 is human IgG1. In some embodiments, the heavy chain constant region comprises or consists of the amino acid sequence of SEQ ID NO: 124. In some embodiments, the light chain constant region comprises or consists of the amino acid sequence of SEQ ID NO: 126. In some embodiments, the light chain constant region comprises or consists of the amino acid sequence of SEQ ID NO: 127. In some embodiments, the heavy chain constant region comprises or consists of the amino acid sequence of SEQ ID NO: 124 and the light chain constant region comprises or consists of the amino acid sequence of SEQ ID NO: 126. In some embodiments, the heavy chain constant region comprises or consists of the amino acid sequence of SEQ ID NO: 124 and the light chain constant region comprises or consists of the amino acid sequence of SEQ ID NO: 127.

In some embodiments, there is provided a full-length anti-TSLP antibody comprising IgG1 constant domains, wherein the anti-TSLP antibody comprises a heavy chain variable domain comprising the amino acid sequence of SEQ ID NO: 76 and a light chain variable domain comprising the amino acid sequence of SEQ ID NO: 95. In some embodiments, the IgG1 is human IgG1. In some embodiments, the heavy chain constant region comprises or consists of the amino acid sequence of SEQ ID NO: 124. In some embodiments, the light chain constant region comprises or consists of the amino acid sequence of SEQ ID NO: 126. In some embodiments, the light chain constant region comprises or consists of the amino acid sequence of SEQ ID NO: 127. In some embodiments, the heavy chain constant region comprises or consists of the amino acid sequence of SEQ ID NO: 124 and the light chain constant region comprises or consists of the amino acid sequence of SEQ ID NO: 126. In some embodiments, the heavy chain constant region comprises or consists of the amino acid sequence of SEQ ID NO: 124 and the light chain constant region comprises or consists of the amino acid sequence of SEQ ID NO: 127.

In some embodiments, there is provided a full-length anti-TSLP antibody comprising IgG1 constant domains, wherein the anti-TSLP antibody comprises a heavy chain variable domain comprising the amino acid sequence of SEQ ID NO: 77 and a light chain variable domain comprising the amino acid sequence of SEQ ID NO: 96. In some embodiments, the IgG1 is human IgG1. In some embodiments, the heavy chain constant region comprises or consists of the amino acid sequence of SEQ ID NO: 124. In some embodiments, the light chain constant region comprises or consists of the amino acid sequence of SEQ ID NO: 126. In some embodiments, the light chain constant region comprises or consists of the amino acid sequence of SEQ ID NO: 127. In some embodiments, the heavy chain constant region comprises or consists of the amino acid sequence of SEQ ID NO: 124 and the light chain constant region comprises or consists of the amino acid sequence of SEQ ID NO: 126. In some embodiments, the heavy chain constant region comprises or consists of the amino acid sequence of SEQ ID NO: 124 and the light chain constant region comprises or consists of the amino acid sequence of SEQ ID NO: 127.

In some embodiments, there is provided a full-length anti-TSLP antibody comprising IgG1 constant domains, wherein the anti-TSLP antibody comprises a heavy chain variable domain comprising the amino acid sequence of SEQ ID NO: 78 and a light chain variable domain comprising the amino acid sequence of SEQ ID NO: 97. In some embodiments, the IgG1 is human IgG1. In some embodiments, the heavy chain constant region comprises or consists of the amino acid sequence of SEQ ID NO: 124. In some embodiments, the light chain constant region comprises or consists of the amino acid sequence of SEQ ID NO: 126. In some embodiments, the light chain constant region comprises or consists of the amino acid sequence of SEQ ID NO: 127. In some embodiments, the heavy chain constant region comprises or consists of the amino acid sequence of SEQ ID NO: 124 and the light chain constant region comprises or consists of the amino acid sequence of SEQ ID NO: 126. In some embodiments, the heavy chain constant region comprises or consists of the amino acid sequence of SEQ ID NO: 124 and the light chain constant region comprises or consists of the amino acid sequence of SEQ ID NO: 127.

In some embodiments, there is provided a full-length anti-TSLP antibody comprising IgG1 constant domains, wherein the anti-TSLP antibody comprises a heavy chain variable domain comprising the amino acid sequence of SEQ ID NO: 79 and a light chain variable domain comprising the amino acid sequence of SEQ ID NO: 98. In some embodiments, the IgG1 is human IgG1. In some embodiments, the heavy chain constant region comprises or consists of the amino acid sequence of SEQ ID NO: 124. In some embodiments, the light chain constant region comprises or consists of the amino acid sequence of SEQ ID NO: 126. In some embodiments, the light chain constant region comprises or consists of the amino acid sequence of SEQ ID NO: 127. In some embodiments, the heavy chain constant region comprises or consists of the amino acid sequence of SEQ ID NO: 124 and the light chain constant region comprises or consists of the amino acid sequence of SEQ ID NO: 126. In some embodiments, the heavy chain constant region comprises or consists of the amino acid sequence of SEQ ID NO: 124 and the light chain constant region comprises or consists of the amino acid sequence of SEQ ID NO: 127.

In some embodiments, there is provided a full-length anti-TSLP antibody comprising IgG1 constant domains, wherein the anti-TSLP antibody comprises a heavy chain variable domain comprising the amino acid sequence of SEQ ID NO: 80 and a light chain variable domain comprising the amino acid sequence of SEQ ID NO: 99. In some embodiments, the IgG1 is human IgG1. In some embodiments, the heavy chain constant region comprises or consists of the amino acid sequence of SEQ ID NO: 124. In some embodiments, the light chain constant region comprises or consists of the amino acid sequence of SEQ ID NO: 126. In some embodiments, the light chain constant region comprises or consists of the amino acid sequence of SEQ ID NO: 127. In some embodiments, the heavy chain constant region comprises or consists of the amino acid sequence of SEQ ID NO: 124 and the light chain constant region comprises or consists of the amino acid sequence of SEQ ID NO: 126. In some embodiments, the heavy chain constant region comprises or consists of the amino acid sequence of SEQ ID NO: 124 and the light chain constant region comprises or consists of the amino acid sequence of SEQ ID NO: 127.

In some embodiments, there is provided a full-length anti-TSLP antibody comprising IgG1 constant domains, wherein the anti-TSLP antibody comprises a heavy chain variable domain comprising the amino acid sequence of SEQ ID NO: 81 and a light chain variable domain comprising the amino acid sequence of SEQ ID NO: 100. In some embodiments, the IgG1 is human IgG1. In some embodiments, the heavy chain constant region comprises or consists of the amino acid sequence of SEQ ID NO: 124. In some embodiments, the light chain constant region comprises or consists of the amino acid sequence of SEQ ID NO: 126. In some embodiments, the light chain constant region comprises or consists of the amino acid sequence of SEQ ID NO: 127. In some embodiments, the heavy chain constant region comprises or consists of the amino acid sequence of SEQ ID NO: 124 and the light chain constant region comprises or consists of the amino acid sequence of SEQ ID NO: 126. In some embodiments, the heavy chain constant region comprises or consists of the amino acid sequence of SEQ ID NO: 124 and the light chain constant region comprises or consists of the amino acid sequence of SEQ ID NO: 127.

In some embodiments, there is provided a full-length anti-TSLP antibody comprising IgG1 constant domains, wherein the anti-TSLP antibody comprises a heavy chain variable domain comprising the amino acid sequence of SEQ ID NO: 82 and a light chain variable domain comprising the amino acid sequence of SEQ ID NO: 101. In some embodiments, the IgG1 is human IgG1. In some embodiments, the heavy chain constant region comprises or consists of the amino acid sequence of SEQ ID NO: 124. In some embodiments, the light chain constant region comprises or consists of the amino acid sequence of SEQ ID NO: 126. In some embodiments, the light chain constant region comprises or consists of the amino acid sequence of SEQ ID NO: 127. In some embodiments, the heavy chain constant region comprises or consists of the amino acid sequence of SEQ ID NO: 124 and the light chain constant region comprises or consists of the amino acid sequence of SEQ ID NO: 126. In some embodiments, the heavy chain constant region comprises or consists of the amino acid sequence of SEQ ID NO: 124 and the light chain constant region comprises or consists of the amino acid sequence of SEQ ID NO: 127.

In some embodiments, there is provided a full-length anti-TSLP antibody comprising IgG1 constant domains, wherein the anti-TSLP antibody comprises a heavy chain variable domain comprising the amino acid sequence of SEQ ID NO: 83 and a light chain variable domain comprising the amino acid sequence of SEQ ID NO: 102. In some embodiments, the IgG1 is human IgG1. In some embodiments, the heavy chain constant region comprises or consists of the amino acid sequence of SEQ ID NO: 124. In some embodiments, the light chain constant region comprises or consists of the amino acid sequence of SEQ ID NO: 126. In some embodiments, the light chain constant region comprises or consists of the amino acid sequence of SEQ ID NO: 127. In some embodiments, the heavy chain constant region comprises or consists of the amino acid sequence of SEQ ID NO: 124 and the light chain constant region comprises or consists of the amino acid sequence of SEQ ID NO: 126. In some embodiments, the heavy chain constant region comprises or consists of the amino acid sequence of SEQ ID NO: 124 and the light chain constant region comprises or consists of the amino acid sequence of SEQ ID NO: 127.

In some embodiments, there is provided a full-length anti-TSLP antibody comprising IgG4 constant domains, wherein the anti-TSLP antibody comprises a heavy chain variable domain comprising the amino acid sequence of SEQ ID NO: 65 and a light chain variable domain comprising the amino acid sequence of SEQ ID NO: 84. In some embodiments, the IgG4 is human IgG4. In some embodiments, the heavy chain constant region comprises or consists of the amino acid sequence of SEQ ID NO: 125. In some embodiments, the light chain constant region comprises or consists of the amino acid sequence of SEQ ID NO: 126. In some embodiments, the light chain constant region comprises or consists of the amino acid sequence of SEQ ID NO: 127. In some embodiments, the heavy chain constant region comprises or consists of the amino acid sequence of SEQ ID NO: 125 and the light chain constant region comprises or consists of the amino acid sequence of SEQ ID NO: 126. In some embodiments, the heavy chain constant region comprises or consists of the amino acid sequence of SEQ ID NO: 125 and the light chain constant region comprises or consists of the amino acid sequence of SEQ ID NO: 127.

In some embodiments, there is provided a full-length anti-TSLP antibody comprising IgG4 constant domains, wherein the anti-TSLP antibody comprises a heavy chain variable domain comprising the amino acid sequence of SEQ ID NO: 65 and a light chain variable domain comprising the amino acid sequence of SEQ ID NO: 85. In some embodiments, the IgG4 is human IgG4. In some embodiments, the heavy chain constant region comprises or consists of the amino acid sequence of SEQ ID NO: 125. In some embodiments, the light chain constant region comprises or consists of the amino acid sequence of SEQ ID NO: 126. In some embodiments, the light chain constant region comprises or consists of the amino acid sequence of SEQ ID NO: 127. In some embodiments, the heavy chain constant region comprises or consists of the amino acid sequence of SEQ ID NO: 125 and the light chain constant region comprises or consists of the amino acid sequence of SEQ ID NO: 126. In some embodiments, the heavy chain constant region comprises or consists of the amino acid sequence of SEQ ID NO: 125 and the light chain constant region comprises or consists of the amino acid sequence of SEQ ID NO: 127.

In some embodiments, there is provided a full-length anti-TSLP antibody comprising IgG4 constant domains, wherein the anti-TSLP antibody comprises a heavy chain variable domain comprising the amino acid sequence of SEQ ID NO: 66 and a light chain variable domain comprising the amino acid sequence of SEQ ID NO: 84. In some embodiments, the IgG4 is human IgG4. In some embodiments, the heavy chain constant region comprises or consists of the amino acid sequence of SEQ ID NO: 125. In some embodiments, the light chain constant region comprises or consists of the amino acid sequence of SEQ ID NO: 126. In some embodiments, the light chain constant region comprises or consists of the amino acid sequence of SEQ ID NO: 127. In some embodiments, the heavy chain constant region comprises or consists of the amino acid sequence of SEQ ID NO: 125 and the light chain constant region comprises or consists of the amino acid sequence of SEQ ID NO: 126. In some embodiments, the heavy chain constant region comprises or consists of the amino acid sequence of SEQ ID NO: 125 and the light chain constant region comprises or consists of the amino acid sequence of SEQ ID NO: 127.

In some embodiments, there is provided a full-length anti-TSLP antibody comprising IgG4 constant domains, wherein the anti-TSLP antibody comprises a heavy chain variable domain comprising the amino acid sequence of SEQ ID NO: 66 and a light chain variable domain comprising the amino acid sequence of SEQ ID NO: 85. In some embodiments, the IgG4 is human IgG4. In some embodiments, the heavy chain constant region comprises or consists of the amino acid sequence of SEQ ID NO: 125. In some embodiments, the light chain constant region comprises or consists of the amino acid sequence of SEQ ID NO: 126. In some embodiments, the light chain constant region comprises or consists of the amino acid sequence of SEQ ID NO: 127. In some embodiments, the heavy chain constant region comprises or consists of the amino acid sequence of SEQ ID NO: 125 and the light chain constant region comprises or consists of the amino acid sequence of SEQ ID NO: 126. In some embodiments, the heavy chain constant region comprises or consists of the amino acid sequence of SEQ ID NO: 125 and the light chain constant region comprises or consists of the amino acid sequence of SEQ ID NO: 127.

In some embodiments, there is provided a full-length anti-TSLP antibody comprising IgG4 constant domains, wherein the anti-TSLP antibody comprises a heavy chain variable domain comprising the amino acid sequence of SEQ ID NO: 67 and a light chain variable domain comprising the amino acid sequence of SEQ ID NO: 84. In some embodiments, the IgG4 is human IgG4. In some embodiments, the heavy chain constant region comprises or consists of the amino acid sequence of SEQ ID NO: 125. In some embodiments, the light chain constant region comprises or consists of the amino acid sequence of SEQ ID NO: 126. In some embodiments, the light chain constant region comprises or consists of the amino acid sequence of SEQ ID NO: 127. In some embodiments, the heavy chain constant region comprises or consists of the amino acid sequence of SEQ ID NO: 125 and the light chain constant region comprises or consists of the amino acid sequence of SEQ ID NO: 126. In some embodiments, the heavy chain constant region comprises or consists of the amino acid sequence of SEQ ID NO: 125 and the light chain constant region comprises or consists of the amino acid sequence of SEQ ID NO: 127.

In some embodiments, there is provided a full-length anti-TSLP antibody comprising IgG4 constant domains, wherein the anti-TSLP antibody comprises a heavy chain variable domain comprising the amino acid sequence of SEQ ID NO: 67 and a light chain variable domain comprising the amino acid sequence of SEQ ID NO: 85. In some embodiments, the IgG4 is human IgG4. In some embodiments, the heavy chain constant region comprises or consists of the amino acid sequence of SEQ ID NO: 125. In some embodiments, the light chain constant region comprises or consists of the amino acid sequence of SEQ ID NO: 126. In some embodiments, the light chain constant region comprises or consists of the amino acid sequence of SEQ ID NO: 127. In some embodiments, the heavy chain constant region comprises or consists of the amino acid sequence of SEQ ID NO: 125 and the light chain constant region comprises or consists of the amino acid sequence of SEQ ID NO: 126. In some embodiments, the heavy chain constant region comprises or consists of the amino acid sequence of SEQ ID NO: 125 and the light chain constant region comprises or consists of the amino acid sequence of SEQ ID NO: 127.

In some embodiments, there is provided a full-length anti-TSLP antibody comprising IgG4 constant domains, wherein the anti-TSLP antibody comprises a heavy chain variable domain comprising the amino acid sequence of SEQ ID NO: 68 and a light chain variable domain comprising the amino acid sequence of SEQ ID NO: 84. In some embodiments, the IgG4 is human IgG4. In some embodiments, the heavy chain constant region comprises or consists of the amino acid sequence of SEQ ID NO: 125. In some embodiments, the light chain constant region comprises or consists of the amino acid sequence of SEQ ID NO: 126. In some embodiments, the light chain constant region comprises or consists of the amino acid sequence of SEQ ID NO: 127. In some embodiments, the heavy chain constant region comprises or consists of the amino acid sequence of SEQ ID NO: 125 and the light chain constant region comprises or consists of the amino acid sequence of SEQ ID NO: 126. In some embodiments, the heavy chain constant region comprises or consists of the amino acid sequence of SEQ ID NO: 125 and the light chain constant region comprises or consists of the amino acid sequence of SEQ ID NO: 127.

In some embodiments, there is provided a full-length anti-TSLP antibody comprising IgG4 constant domains, wherein the anti-TSLP antibody comprises a heavy chain variable domain comprising the amino acid sequence of SEQ ID NO: 68 and a light chain variable domain comprising the amino acid sequence of SEQ ID NO: 85. In some embodiments, the IgG4 is human IgG4. In some embodiments, the heavy chain constant region comprises or consists of the amino acid sequence of SEQ ID NO: 125. In some embodiments, the light chain constant region comprises or consists of the amino acid sequence of SEQ ID NO: 126. In some embodiments, the light chain constant region comprises or consists of the amino acid sequence of SEQ ID NO: 127. In some embodiments, the heavy chain constant region comprises or consists of the amino acid sequence of SEQ ID NO: 125 and the light chain constant region comprises or consists of the amino acid sequence of SEQ ID NO: 126. In some embodiments, the heavy chain constant region comprises or consists of the amino acid sequence of SEQ ID NO: 125 and the light chain constant region comprises or consists of the amino acid sequence of SEQ ID NO: 127.

In some embodiments, there is provided a full-length anti-TSLP antibody comprising IgG4 constant domains, wherein the anti-TSLP antibody comprises a heavy chain variable domain comprising the amino acid sequence of SEQ ID NO: 69 and a light chain variable domain comprising the amino acid sequence of SEQ ID NO: 86. In some embodiments, the IgG4 is human IgG4. In some embodiments, the heavy chain constant region comprises or consists of the amino acid sequence of SEQ ID NO: 125. In some embodiments, the light chain constant region comprises or consists of the amino acid sequence of SEQ ID NO: 126. In some embodiments, the light chain constant region comprises or consists of the amino acid sequence of SEQ ID NO: 127. In some embodiments, the heavy chain constant region comprises or consists of the amino acid sequence of SEQ ID NO: 125 and the light chain constant region comprises or consists of the amino acid sequence of SEQ ID NO: 126. In some embodiments, the heavy chain constant region comprises or consists of the amino acid sequence of SEQ ID NO: 125 and the light chain constant region comprises or consists of the amino acid sequence of SEQ ID NO: 127.

In some embodiments, there is provided a full-length anti-TSLP antibody comprising IgG4 constant domains, wherein the anti-TSLP antibody comprises a heavy chain variable domain comprising the amino acid sequence of SEQ ID NO: 70 and a light chain variable domain comprising the amino acid sequence of SEQ ID NO: 87. In some embodiments, the IgG4 is human IgG4. In some embodiments, the heavy chain constant region comprises or consists of the amino acid sequence of SEQ ID NO: 125. In some embodiments, the light chain constant region comprises or consists of the amino acid sequence of SEQ ID NO: 126. In some embodiments, the light chain constant region comprises or consists of the amino acid sequence of SEQ ID NO: 127. In some embodiments, the heavy chain constant region comprises or consists of the amino acid sequence of SEQ ID NO: 125 and the light chain constant region comprises or consists of the amino acid sequence of SEQ ID NO: 126. In some embodiments, the heavy chain constant region comprises or consists of the amino acid sequence of SEQ ID NO: 125 and the light chain constant region comprises or consists of the amino acid sequence of SEQ ID NO: 127.

In some embodiments, there is provided a full-length anti-TSLP antibody comprising IgG4 constant domains, wherein the anti-TSLP antibody comprises a heavy chain variable domain comprising the amino acid sequence of SEQ ID NO: 69 and a light chain variable domain comprising the amino acid sequence of SEQ ID NO: 88. In some embodiments, the IgG4 is human IgG4. In some embodiments, the heavy chain constant region comprises or consists of the amino acid sequence of SEQ ID NO: 125. In some embodiments, the light chain constant region comprises or consists of the amino acid sequence of SEQ ID NO: 126. In some embodiments, the light chain constant region comprises or consists of the amino acid sequence of SEQ ID NO: 127. In some embodiments, the heavy chain constant region comprises or consists of the amino acid sequence of SEQ ID NO: 125 and the light chain constant region comprises or consists of the amino acid sequence of SEQ ID NO: 126. In some embodiments, the heavy chain constant region comprises or consists of the amino acid sequence of SEQ ID NO: 125 and the light chain constant region comprises or consists of the amino acid sequence of SEQ ID NO: 127.

In some embodiments, there is provided a full-length anti-TSLP antibody comprising IgG4 constant domains, wherein the anti-TSLP antibody comprises a heavy chain variable domain comprising the amino acid sequence of SEQ ID NO: 71 and a light chain variable domain comprising the amino acid sequence of SEQ ID NO: 89. In some embodiments, the IgG4 is human IgG4. In some embodiments, the heavy chain constant region comprises or consists of the amino acid sequence of SEQ ID NO: 125. In some embodiments, the light chain constant region comprises or consists of the amino acid sequence of SEQ ID NO: 126. In some embodiments, the light chain constant region comprises or consists of the amino acid sequence of SEQ ID NO: 127. In some embodiments, the heavy chain constant region comprises or consists of the amino acid sequence of SEQ ID NO: 125 and the light chain constant region comprises or consists of the amino acid sequence of SEQ ID NO: 126. In some embodiments, the heavy chain constant region comprises or consists of the amino acid sequence of SEQ ID NO: 125 and the light chain constant region comprises or consists of the amino acid sequence of SEQ ID NO: 127.

In some embodiments, there is provided a full-length anti-TSLP antibody comprising IgG4 constant domains, wherein the anti-TSLP antibody comprises a heavy chain variable domain comprising the amino acid sequence of SEQ ID NO: 72 and a light chain variable domain comprising the amino acid sequence of SEQ ID NO: 90. In some embodiments, the IgG4 is human IgG4. In some embodiments, the heavy chain constant region comprises or consists of the amino acid sequence of SEQ ID NO: 125. In some embodiments, the light chain constant region comprises or consists of the amino acid sequence of SEQ ID NO: 126. In some embodiments, the light chain constant region comprises or consists of the amino acid sequence of SEQ ID NO: 127. In some embodiments, the heavy chain constant region comprises or consists of the amino acid sequence of SEQ ID NO: 125 and the light chain constant region comprises or consists of the amino acid sequence of SEQ ID NO: 126. In some embodiments, the heavy chain constant region comprises or consists of the amino acid sequence of SEQ ID NO: 125 and the light chain constant region comprises or consists of the amino acid sequence of SEQ ID NO: 127.

In some embodiments, there is provided a full-length anti-TSLP antibody comprising IgG4 constant domains, wherein the anti-TSLP antibody comprises a heavy chain variable domain comprising the amino acid sequence of SEQ ID NO: 73 and a light chain variable domain comprising the amino acid sequence of SEQ ID NO: 91. In some embodiments, the IgG4 is human IgG4. In some embodiments, the heavy chain constant region comprises or consists of the amino acid sequence of SEQ ID NO: 125. In some embodiments, the light chain constant region comprises or consists of the amino acid sequence of SEQ ID NO: 126. In some embodiments, the light chain constant region comprises or consists of the amino acid sequence of SEQ ID NO: 127. In some embodiments, the heavy chain constant region comprises or consists of the amino acid sequence of SEQ ID NO: 125 and the light chain constant region comprises or consists of the amino acid sequence of SEQ ID NO: 126. In some embodiments, the heavy chain constant region comprises or consists of the amino acid sequence of SEQ ID NO: 125 and the light chain constant region comprises or consists of the amino acid sequence of SEQ ID NO: 127.

In some embodiments, there is provided a full-length anti-TSLP antibody comprising IgG4 constant domains, wherein the anti-TSLP antibody comprises a heavy chain variable domain comprising the amino acid sequence of SEQ ID NO: 74 and a light chain variable domain comprising the amino acid sequence of SEQ ID NO: 91. In some embodiments, the IgG4 is human IgG4. In some embodiments, the heavy chain constant region comprises or consists of the amino acid sequence of SEQ ID NO: 125. In some embodiments, the light chain constant region comprises or consists of the amino acid sequence of SEQ ID NO: 126. In some embodiments, the light chain constant region comprises or consists of the amino acid sequence of SEQ ID NO: 127. In some embodiments, the heavy chain constant region comprises or consists of the amino acid sequence of SEQ ID NO: 125 and the light chain constant region comprises or consists of the amino acid sequence of SEQ ID NO: 126. In some embodiments, the heavy chain constant region comprises or consists of the amino acid sequence of SEQ ID NO: 125 and the light chain constant region comprises or consists of the amino acid sequence of SEQ ID NO: 127.

In some embodiments, there is provided a full-length anti-TSLP antibody comprising IgG4 constant domains, wherein the anti-TSLP antibody comprises a heavy chain variable domain comprising the amino acid sequence of SEQ ID NO: 75 and a light chain variable domain comprising the amino acid sequence of SEQ ID NO: 91. In some embodiments, the IgG4 is human IgG4. In some embodiments, the heavy chain constant region comprises or consists of the amino acid sequence of SEQ ID NO: 125. In some embodiments, the light chain constant region comprises or consists of the amino acid sequence of SEQ ID NO: 126. In some embodiments, the light chain constant region comprises or consists of the amino acid sequence of SEQ ID NO: 127. In some embodiments, the heavy chain constant region comprises or consists of the amino acid sequence of SEQ ID NO: 125 and the light chain constant region comprises or consists of the amino acid sequence of SEQ ID NO: 126. In some embodiments, the heavy chain constant region comprises or consists of the amino acid sequence of SEQ ID NO: 125 and the light chain constant region comprises or consists of the amino acid sequence of SEQ ID NO: 127.

In some embodiments, there is provided a full-length anti-TSLP antibody comprising IgG4 constant domains, wherein the anti-TSLP antibody comprises a heavy chain variable domain comprising the amino acid sequence of SEQ ID NO: 73 and a light chain variable domain comprising the amino acid sequence of SEQ ID NO: 92. In some embodiments, the IgG4 is human IgG4. In some embodiments, the heavy chain constant region comprises or consists of the amino acid sequence of SEQ ID NO: 125. In some embodiments, the light chain constant region comprises or consists of the amino acid sequence of SEQ ID NO: 126. In some embodiments, the light chain constant region comprises or consists of the amino acid sequence of SEQ ID NO: 127. In some embodiments, the heavy chain constant region comprises or consists of the amino acid sequence of SEQ ID NO: 125 and the light chain constant region comprises or consists of the amino acid sequence of SEQ ID NO: 126. In some embodiments, the heavy chain constant region comprises or consists of the amino acid sequence of SEQ ID NO: 125 and the light chain constant region comprises or consists of the amino acid sequence of SEQ ID NO: 127.

In some embodiments, there is provided a full-length anti-TSLP antibody comprising IgG4 constant domains, wherein the anti-TSLP antibody comprises a heavy chain variable domain comprising the amino acid sequence of SEQ ID NO: 73 and a light chain variable domain comprising the amino acid sequence of SEQ ID NO: 93. In some embodiments, the IgG4 is human IgG4. In some embodiments, the heavy chain constant region comprises or consists of the amino acid sequence of SEQ ID NO: 125. In some embodiments, the light chain constant region comprises or consists of the amino acid sequence of SEQ ID NO: 126. In some embodiments, the light chain constant region comprises or consists of the amino acid sequence of SEQ ID NO: 127. In some embodiments, the heavy chain constant region comprises or consists of the amino acid sequence of SEQ ID NO: 125 and the light chain constant region comprises or consists of the amino acid sequence of SEQ ID NO: 126. In some embodiments, the heavy chain constant region comprises or consists of the amino acid sequence of SEQ ID NO: 125 and the light chain constant region comprises or consists of the amino acid sequence of SEQ ID NO: 127.

In some embodiments, there is provided a full-length anti-TSLP antibody comprising IgG4 constant domains, wherein the anti-TSLP antibody comprises a heavy chain variable domain comprising the amino acid sequence of SEQ ID NO: 73 and a light chain variable domain comprising the amino acid sequence of SEQ ID NO: 94. In some embodiments, the IgG4 is human IgG4. In some embodiments, the heavy chain constant region comprises or consists of the amino acid sequence of SEQ ID NO: 125. In some embodiments, the light chain constant region comprises or consists of the amino acid sequence of SEQ ID NO: 126. In some embodiments, the light chain constant region comprises or consists of the amino acid sequence of SEQ ID NO: 127. In some embodiments, the heavy chain constant region comprises or consists of the amino acid sequence of SEQ ID NO: 125 and the light chain constant region comprises or consists of the amino acid sequence of SEQ ID NO: 126. In some embodiments, the heavy chain constant region comprises or consists of the amino acid sequence of SEQ ID NO: 125 and the light chain constant region comprises or consists of the amino acid sequence of SEQ ID NO: 127.

In some embodiments, there is provided a full-length anti-TSLP antibody comprising IgG4 constant domains, wherein the anti-TSLP antibody comprises a heavy chain variable domain comprising the amino acid sequence of SEQ ID NO: 76 and a light chain variable domain comprising the amino acid sequence of SEQ ID NO: 95. In some embodiments, the IgG4 is human IgG4. In some embodiments, the heavy chain constant region comprises or consists of the amino acid sequence of SEQ ID NO: 125. In some embodiments, the light chain constant region comprises or consists of the amino acid sequence of SEQ ID NO: 126. In some embodiments, the light chain constant region comprises or consists of the amino acid sequence of SEQ ID NO: 127. In some embodiments, the heavy chain constant region comprises or consists of the amino acid sequence of SEQ ID NO: 125 and the light chain constant region comprises or consists of the amino acid sequence of SEQ ID NO: 126. In some embodiments, the heavy chain constant region comprises or consists of the amino acid sequence of SEQ ID NO: 125 and the light chain constant region comprises or consists of the amino acid sequence of SEQ ID NO: 127.

In some embodiments, there is provided a full-length anti-TSLP antibody comprising IgG4 constant domains, wherein the anti-TSLP antibody comprises a heavy chain variable domain comprising the amino acid sequence of SEQ ID NO: 77 and a light chain variable domain comprising the amino acid sequence of SEQ ID NO: 96. In some embodiments, the IgG4 is human IgG4. In some embodiments, the heavy chain constant region comprises or consists of the amino acid sequence of SEQ ID NO: 125. In some embodiments, the light chain constant region comprises or consists of the amino acid sequence of SEQ ID NO: 126. In some embodiments, the light chain constant region comprises or consists of the amino acid sequence of SEQ ID NO: 127. In some embodiments, the heavy chain constant region comprises or consists of the amino acid sequence of SEQ ID NO: 125 and the light chain constant region comprises or consists of the amino acid sequence of SEQ ID NO: 126. In some embodiments, the heavy chain constant region comprises or consists of the amino acid sequence of SEQ ID NO: 125 and the light chain constant region comprises or consists of the amino acid sequence of SEQ ID NO: 127.

In some embodiments, there is provided a full-length anti-TSLP antibody comprising IgG4 constant domains, wherein the anti-TSLP antibody comprises a heavy chain variable domain comprising the amino acid sequence of SEQ ID NO: 78 and a light chain variable domain comprising the amino acid sequence of SEQ ID NO: 97. In some embodiments, the IgG4 is human IgG4. In some embodiments, the heavy chain constant region comprises or consists of the amino acid sequence of SEQ ID NO: 125. In some embodiments, the light chain constant region comprises or consists of the amino acid sequence of SEQ ID NO: 126. In some embodiments, the light chain constant region comprises or consists of the amino acid sequence of SEQ ID NO: 127. In some embodiments, the heavy chain constant region comprises or consists of the amino acid sequence of SEQ ID NO: 125 and the light chain constant region comprises or consists of the amino acid sequence of SEQ ID NO: 126. In some embodiments, the heavy chain constant region comprises or consists of the amino acid sequence of SEQ ID NO: 125 and the light chain constant region comprises or consists of the amino acid sequence of SEQ ID NO: 127.

In some embodiments, there is provided a full-length anti-TSLP antibody comprising IgG4 constant domains, wherein the anti-TSLP antibody comprises a heavy chain variable domain comprising the amino acid sequence of SEQ ID NO: 79 and a light chain variable domain comprising the amino acid sequence of SEQ ID NO: 98. In some embodiments, the IgG4 is human IgG4. In some embodiments, the heavy chain constant region comprises or consists of the amino acid sequence of SEQ ID NO: 125. In some embodiments, the light chain constant region comprises or consists of the amino acid sequence of SEQ ID NO: 126. In some embodiments, the light chain constant region comprises or consists of the amino acid sequence of SEQ ID NO: 127. In some embodiments, the heavy chain constant region comprises or consists of the amino acid sequence of SEQ ID NO: 125 and the light chain constant region comprises or consists of the amino acid sequence of SEQ ID NO: 126. In some embodiments, the heavy chain constant region comprises or consists of the amino acid sequence of SEQ ID NO: 125 and the light chain constant region comprises or consists of the amino acid sequence of SEQ ID NO: 127.

In some embodiments, there is provided a full-length anti-TSLP antibody comprising IgG4 constant domains, wherein the anti-TSLP antibody comprises a heavy chain variable domain comprising the amino acid sequence of SEQ ID NO: 80 and a light chain variable domain comprising the amino acid sequence of SEQ ID NO: 99. In some embodiments, the IgG4 is human IgG4. In some embodiments, the heavy chain constant region comprises or consists of the amino acid sequence of SEQ ID NO: 125. In some embodiments, the light chain constant region comprises or consists of the amino acid sequence of SEQ ID NO: 126. In some embodiments, the light chain constant region comprises or consists of the amino acid sequence of SEQ ID NO: 127. In some embodiments, the heavy chain constant region comprises or consists of the amino acid sequence of SEQ ID NO: 125 and the light chain constant region comprises or consists of the amino acid sequence of SEQ ID NO: 126. In some embodiments, the heavy chain constant region comprises or consists of the amino acid sequence of SEQ ID NO: 125 and the light chain constant region comprises or consists of the amino acid sequence of SEQ ID NO: 127.

In some embodiments, there is provided a full-length anti-TSLP antibody comprising IgG4 constant domains, wherein the anti-TSLP antibody comprises a heavy chain variable domain comprising the amino acid sequence of SEQ ID NO: 81 and a light chain variable domain comprising the amino acid sequence of SEQ ID NO: 100. In some embodiments, the IgG4 is human IgG4. In some embodiments, the heavy chain constant region comprises or consists of the amino acid sequence of SEQ ID NO: 125. In some embodiments, the light chain constant region comprises or consists of the amino acid sequence of SEQ ID NO: 126. In some embodiments, the light chain constant region comprises or consists of the amino acid sequence of SEQ ID NO: 127. In some embodiments, the heavy chain constant region comprises or consists of the amino acid sequence of SEQ ID NO: 125 and the light chain constant region comprises or consists of the amino acid sequence of SEQ ID NO: 126. In some embodiments, the heavy chain constant region comprises or consists of the amino acid sequence of SEQ ID NO: 125 and the light chain constant region comprises or consists of the amino acid sequence of SEQ ID NO: 127.

In some embodiments, there is provided a full-length anti-TSLP antibody comprising IgG4 constant domains, wherein the anti-TSLP antibody comprises a heavy chain variable domain comprising the amino acid sequence of SEQ ID NO: 82 and a light chain variable domain comprising the amino acid sequence of SEQ ID NO: 101. In some embodiments, the IgG4 is human IgG4. In some embodiments, the heavy chain constant region comprises or consists of the amino acid sequence of SEQ ID NO: 125. In some embodiments, the light chain constant region comprises or consists of the amino acid sequence of SEQ ID NO: 126. In some embodiments, the light chain constant region comprises or consists of the amino acid sequence of SEQ ID NO: 127. In some embodiments, the heavy chain constant region comprises or consists of the amino acid sequence of SEQ ID NO: 125 and the light chain constant region comprises or consists of the amino acid sequence of SEQ ID NO: 126. In some embodiments, the heavy chain constant region comprises or consists of the amino acid sequence of SEQ ID NO: 125 and the light chain constant region comprises or consists of the amino acid sequence of SEQ ID NO: 127.

In some embodiments, there is provided a full-length anti-TSLP antibody comprising IgG4 constant domains, wherein the anti-TSLP antibody comprises a heavy chain variable domain comprising the amino acid sequence of SEQ ID NO: 83 and a light chain variable domain comprising the amino acid sequence of SEQ ID NO: 102. In some embodiments, the IgG4 is human IgG4. In some embodiments, the heavy chain constant region comprises or consists of the amino acid sequence of SEQ ID NO: 125. In some embodiments, the light chain constant region comprises or consists of the amino acid sequence of SEQ ID NO: 126. In some embodiments, the light chain constant region comprises or consists of the amino acid sequence of SEQ ID NO: 127. In some embodiments, the heavy chain constant region comprises or consists of the amino acid sequence of SEQ ID NO: 125 and the light chain constant region comprises or consists of the amino acid sequence of SEQ ID NO: 126. In some embodiments, the heavy chain constant region comprises or consists of the amino acid sequence of SEQ ID NO: 125 and the light chain constant region comprises or consists of the amino acid sequence of SEQ ID NO: 127.

### Binding Affinity

Binding affinity can be indicated by Kd, Koff, Kon, or Ka. The term “Koff”, as used herein, is intended to refer to the off-rate constant for dissociation of an antibody from the antibody/antigen complex, as determined from a kinetic selection set up. The term “Kon”, as used herein, is intended to refer to the on-rate constant for association of an antibody to the antigen to form the antibody/antigen complex. The term dissociation constant “Kd”, as used herein, refers to the dissociation constant of a particular antibody-antigen interaction, and describes the concentration of antigen required to occupy one half of all of the antibody-binding domains present in a solution of antibody molecules at equilibrium, and is equal to Koff/Kon. The measurement of Kd presupposes that all binding agents are in solution. In the case where the antibody is tethered to a cell wall, e.g., in a yeast expression system, the corresponding equilibrium rate constant is expressed as EC50, which gives a good approximation of Kd. The affinity constant, Ka, is the inverse of the dissociation constant, Kd.

The dissociation constant (Kd) is used as an indicator showing affinity of antibody moieties to antigens. For example, easy analysis is possible by the Scatchard method using antibodies marked with a variety of marker agents, as well as by using Biacore (made by Amersham Biosciences), analysis of biomolecular interactions by surface plasmon resonance, according to the user's manual and attached kit. The Kd value that can be derived using these methods is expressed in units of M (mol). An antibody that specifically binds to a target may have a Kd of, for example, ≤10⁻⁷ M, ≤10⁻⁸ M, ≤10⁻⁹ M, ≤10⁻¹⁰ M, ≤10⁻¹¹ M, ≤10⁻¹² M, or ≤10⁻¹³ M.

Binding specificity of the antibody can be determined experimentally by methods known in the art. Such methods comprise, but are not limited to, Western blots, ELISA-, RIA-, ECL-, IRMA-, EIA-, BIAcore-tests and peptide scans.

In some embodiments, the anti-TSLP antibody specifically binds to a target TSLP with a Kd of about 10⁻⁷ M to about 10⁻¹³ M (such as about 10⁻⁷ M to about 10⁻¹³ M, about 10⁻⁸ M to about 10⁻¹³ M, about 10⁻⁹ M to about 10⁻¹³ M, or about 10⁻¹⁰ M to about 10⁻¹² M). Thus in some embodiments, the Kd of the binding between the anti-TSLP antibody and TSLP, is about 10⁻⁷ M to about 10⁻¹³ M, about 1×10⁻⁷ M to about 5×10⁻¹³ M, about 10⁻⁷ M to about 10⁻¹² M, about 10⁻⁷ M to about 10⁻¹¹ M, about 10⁻⁷ M to about 10⁻¹⁰ M, about 10⁻⁷ M to about 10⁻⁹ M, about 10⁻⁸ M to about 10⁻¹³ M, about 1×10⁻⁸ M to about 5×10⁻¹³ M, about 10⁻⁸ M to about 10⁻¹² M, about 10⁻⁸ M to about 10⁻¹⁰ M, about 10⁻⁸ M to about 10⁻¹⁰ M, about 10⁻⁸ M to about 10⁻⁹ M, about 5×10⁻⁹ M to about 1×10⁻¹³ M, about 5×10⁻⁹ M to about 1×10⁻¹² M, about 5×10⁻⁹ M to about 1×10⁻¹¹ M, about 5×10⁻⁹ M to about 1×10⁻¹⁰ M, about 10⁻⁹ M to about 10⁻¹³ M, about 10⁻⁹ M to about 10⁻¹² M, about 10⁻⁹ M to about 10⁻¹¹ M, about 10⁻⁹ M to about 10⁻¹⁰ M, about 5×10⁻¹⁰ M to about 1×10⁻¹³ M, about 5×10⁻¹⁰ M to about 1×10⁻¹² M, about 5×10⁻¹⁰ M to about 1×10⁻¹¹ M, about 10⁻¹⁰ M to about 10⁻¹³ M, about 1×10⁻¹⁰ M to about 5×10⁻¹³ M, about 1×10⁻¹⁰ M to about 1×10⁻¹² M, about 1×10⁻¹⁰ M to about 5×10⁻¹² M, about 1×10⁻¹⁰ M to about 1×10⁻¹¹ M, about 10⁻¹¹ M to about 10⁻¹³ M, about 1×10⁻¹¹ M to about 5×10⁻¹³ M, about 10⁻¹³ M to about 10⁻¹² M, or about 10⁻¹² M to about 10⁻¹³ M. In some embodiments, the Kd of the binding between the anti-TSLP antibody and a TSLP is about 10⁻⁷ M to about 10⁻¹³ M.

In some embodiments, the Kd of the binding between the anti-TSLP antibody and a non-target is more than the Kd of the binding between the anti-TSLP antibody and the target, and is herein referred to in some embodiments as the binding affinity of the anti-TSLP antibody to the target (e.g., TSLP) is higher than that to a non-target. In some embodiments, the non-target is an antigen that is not TSLP. In some embodiments, the Kd of the binding between the anti-TSLP antibody (against TSLP) and a non-TSLP target can be at least about times, such as about 10-100 times, about 100-1000 times, about 10³-10⁴ times, about 10⁴-10⁵ times, about 10⁵-10⁶ times, about 10⁶-10⁷ times, about 107-10⁸ times, about 10⁸-10⁹ times, about 10⁹-10¹⁰ times, about 10¹⁰-10¹¹ times, or about 10¹¹-10¹² times of the Kd of the binding between the anti-TSLP antibody and a target TSLP.

In some embodiments, the anti-TSLP antibody binds to a non-target with a Kd of about 10⁻¹ M to about 10⁻⁶ M (such as about 10⁻¹ M to about 10⁻⁶ M, about 10⁻¹ M to about 10⁻⁵ M, or about 10⁻² M to about 10⁻⁴ M). In some embodiments, the non-target is an antigen that is not TSLP. Thus in some embodiments, the Kd of the binding between the anti-TSLP antibody and a non-TSLP target is about 10⁻¹ M to about 10⁻⁶ M, about 1×10⁻¹ M to about 5×10⁻⁶ M, about 10⁻¹ M to about 10⁻⁵ M, about 1×10⁻¹ M to about 5×10⁻⁵ M, about 10⁻¹ M to about 10⁻⁴ M, about 1×10⁻¹ M to about 5×10⁻⁴ M, about 10⁻¹ M to about 10⁻³ M, about 1×10⁻¹ M to about 5×10⁻³ M, about 10⁻¹ M to about 10⁻² M, about 10⁻² M to about 10⁻⁶ M, about 1×10⁻² M to about 5×10⁻⁶ M, about 10⁻² M to about 10⁻⁵ M, about 1×10⁻² M to about 5×10⁻⁵ M, about 10⁻² M to about 10⁻⁴ M, about 1×10⁻² M to about 5×10⁻⁴ M, about 10⁻² M to about 10⁻³ M, about 10⁻³ M to about 10⁻⁶ M, about 1×10⁻³ M to about 5×10⁻⁶ M, about 10⁻³ M to about 10⁻⁵ M, about 1×10⁻³ M to about 5×10⁻⁵ M, about 10⁻³ M to about 10⁻⁴ M, about 10⁻⁴ M to about 10⁻⁶ M, about 1×10⁻⁴ M to about 5×10⁻⁶ M, about 10⁻⁴ M to about 10⁻⁵ M, or about 10⁻⁵ M to about 10⁻⁶ M.

In some embodiments, when referring to that the anti-TSLP antibody specifically recognizes a target TSLP at a high binding affinity, and binds to a non-target at a low binding affinity, the anti-TSLP antibody will bind to the target TSLP with a Kd of about 10⁻⁷ M to about 10⁻¹³ M (such as about 10⁻⁷ M to about 10⁻¹³ M, about 10⁻⁸ M to about 10⁻¹³ M, about 10⁻⁹ M to about 10⁻¹³ M, or about 10⁻¹⁰ M to about 10⁻¹² M), and will bind to the non-target with a Kd of about 10⁻¹ M to about 10⁻⁶ M (such as about 10⁻¹ M to about 10⁻⁶ M, about 10⁻¹ M to about 10⁻⁵ M, or about 10⁻² M to about 10⁻⁴ M).

In some embodiments, when referring to that the anti-TSLP antibody specifically recognizes TSLP, the binding affinity of the anti-TSLP antibody is compared to that of a control anti-TSLP antibody (such as AMG157). In some embodiments, the Kd of the binding between the control anti-TSLP antibody and TSLP can be at least about 2 times, such as about 2 times, about 3 times, about 4 times, about 5 times, about 6 times, about 7 times, about 8 times, about 9 times, about 10 times, about 10-100 times, about 100-1000 times, about 10³-10⁴ times of the Kd of the binding between the anti-TSLP antibody described herein and TSLP.

### Nucleic Acids

Nucleic acid molecules encoding the anti-TSLP antibodies are also contemplated. In some embodiments, there is provided a nucleic acid (or a set of nucleic acids) encoding a full-length anti-TSLP antibody, including any of the full-length anti-TSLP antibodies described herein. In some embodiments, the nucleic acid (or a set of nucleic acids) encoding the anti-TSLP antibody described herein may further comprises a nucleic acid sequence encoding a peptide tag (such as protein purification tag, e.g., His-tag, HA tag).

Also contemplated here are isolated host cells comprising an anti-TSLP antibody, an isolated nucleic acid encoding the polypeptide components of the anti-TSLP antibody, or a vector comprising a nucleic acid encoding the polypeptide components of the anti-TSLP antibody described herein.

The present application also includes variants to these nucleic acid sequences. For example, the variants include nucleotide sequences that hybridize to the nucleic acid sequences encoding the anti-TSLP antibodies of the present application under at least moderately stringent hybridization conditions.

The present application also provides vectors in which a nucleic acid of the present application is inserted.

In brief summary, the expression of an anti-TSLP antibody (e.g., full-length anti-TSLP antibody) by a natural or synthetic nucleic acid encoding the anti-TSLP antibody can be achieved by inserting the nucleic acid into an appropriate expression vector, such that the nucleic acid is operably linked to 5′ and 3′ regulatory elements, including for example a promoter (e.g., a lymphocyte-specific promoter) and a 3′ untranslated region (UTR). The vectors can be suitable for replication and integration in eukaryotic host cells. Typical cloning and expression vectors contain transcription and translation terminators, initiation sequences, and promoters useful for regulation of the expression of the desired nucleic acid sequence.

The nucleic acids of the present application may also be used for nucleic acid immunization and gene therapy, using standard gene delivery protocols. Methods for gene delivery are known in the art. See, e.g., U.S. Pat. Nos. 5,399,346, 5,580,859, 5,589,466, incorporated by reference herein in their entireties. In some embodiments, the application provides a gene therapy vector.

The nucleic acid can be cloned into a number of types of vectors. For example, the nucleic acid can be cloned into a vector including, but not limited to a plasmid, a phagemid, a phage derivative, an animal virus, and a cosmid. Vectors of particular interest include expression vectors, replication vectors, probe generation vectors, and sequencing vectors.

Further, the expression vector may be provided to a cell in the form of a viral vector. Viral vector technology is well known in the art and is described, for example, in Green and Sambrook (2013, Molecular Cloning: A Laboratory Manual, Cold Spring Harbor Laboratory, New York), and in other virology and molecular biology manuals. Viruses which are useful as vectors include, but are not limited to, retroviruses, adenoviruses, adeno-associated viruses, herpes viruses, and lentiviruses. In general, a suitable vector contains an origin of replication functional in at least one organism, a promoter sequence, convenient restriction endonuclease sites, and one or more selectable markers (see, e.g., WO 01/96584; WO 01/29058; and U.S. Pat. No. 6,326,193).

A number of viral based systems have been developed for gene transfer into mammalian cells. For example, retroviruses provide a convenient platform for gene delivery systems. A selected gene can be inserted into a vector and packaged in retroviral particles using techniques known in the art. The recombinant virus can then be isolated and delivered to cells of the subject either in vivo or ex vivo. A number of retroviral systems are known in the art. In some embodiments, adenovirus vectors are used. A number of adenovirus vectors are known in the art. In some embodiments, lentivirus vectors are used. Vectors derived from retroviruses such as the lentivirus are suitable tools to achieve long-term gene transfer since they allow long-term, stable integration of a transgene and its propagation in daughter cells. Lentiviral vectors have the added advantage over vectors derived from onco-retroviruses such as murine leukemia viruses in that they can transduce non-proliferating cells, such as hepatocytes. They also have the added advantage of low immunogenicity.

Additional promoter elements, e.g., enhancers, regulate the frequency of transcriptional initiation. Typically, these are located in the region 30-110 bp upstream of the start site, although a number of promoters have recently been shown to contain functional elements downstream of the start site as well. The spacing between promoter elements frequently is flexible, so that promoter function is preserved when elements are inverted or moved relative to one another. In the thymidine kinase (tk) promoter, the spacing between promoter elements can be increased to 50 bp apart before activity begins to decline.

One example of a suitable promoter is the immediate early cytomegalovirus (CMV) promoter sequence. This promoter sequence is a strong constitutive promoter sequence capable of driving high levels of expression of any polynucleotide sequence operatively linked thereto. Another example of a suitable promoter is Elongation Factor-1α (EF-1α). However, other constitutive promoter sequences may also be used, including, but not limited to the simian virus 40 (SV40) early promoter, mouse mammary tumor virus (MMTV), human immunodeficiency virus (HIV) long terminal repeat (LTR) promoter, MoMuLV promoter, an avian leukemia virus promoter, an Epstein-Barr virus immediate early promoter, a Rous sarcoma virus promoter, as well as human gene promoters such as, but not limited to, the actin promoter, the myosin promoter, the hemoglobin promoter, and the creatine kinase promoter. Further, the application should not be limited to the use of constitutive promoters. Inducible promoters are also contemplated as part of the application. The use of an inducible promoter provides a molecular switch capable of turning on expression of the polynucleotide sequence which it is operatively linked when such expression is desired, or turning off the expression when expression is not desired. Examples of inducible promoters include, but are not limited to a metallothionine promoter, a glucocorticoid promoter, a progesterone promoter, and a tetracycline promoter.

In some embodiments, the expression of the anti-TSLP antibody is inducible. In some embodiments, a nucleic acid sequence encoding the anti-TSLP antibody is operably linked to an inducible promoter, including any inducible promoter described herein.

### Inducible Promoters

The use of an inducible promoter provides a molecular switch capable of turning on expression of the polynucleotide sequence which it is operatively linked when such expression is desired, or turning off the expression when expression is not desired. Exemplary inducible promoter systems for use in eukaryotic cells include, but are not limited to, hormone-regulated elements (e.g., see Mader, S. and White, J. H. (1993) Proc. Natl. Acad. Sci. USA 90:5603-5607), synthetic ligand-regulated elements (see, e.g., Spencer, D. M. et al 1993) Science 262: 1019-1024) and ionizing radiation-regulated elements (e.g., see Manome, Y. et al. (1993) Biochemistry 32: 10607-10613; Datta, R. et al. (1992) Proc. Natl. Acad. Sci. USA 89: 1014-10153). Further exemplary inducible promoter systems for use in in vitro or in vivo mammalian systems are reviewed in Gingrich et al. (1998) Annual Rev. Neurosci 21:377-405. In some embodiments, the inducible promoter system for use to express the anti-TSLP antibody is the Tet system. In some embodiments, the inducible promoter system for use to express the anti-TSLP antibody is the lac repressor system from E. coli.

An exemplary inducible promoter system for use in the present application is the Tet system. Such systems are based on the Tet system described by Gossen et al. (1993). In an exemplary embodiment, a polynucleotide of interest is under the control of a promoter that comprises one or more Tet operator (TetO) sites. In the inactive state, Tet repressor (TetR) will bind to the TetO sites and repress transcription from the promoter. In the active state, e.g., in the presence of an inducing agent such as tetracycline (Tc), anhydrotetracycline, doxycycline (Dox), or an active analog thereof, the inducing agent causes release of TetR from TetO, thereby allowing transcription to take place. Doxycycline is a member of the tetracycline family of antibiotics having the chemical name of 1-dimethylamino-2,4a,5,7,12-pentahydroxy-11-methyl-4,6-dioxo-1,4a,11,11a,12,12a-hexahydrotetracene-3-carboxamide.

In one embodiment, a TetR is codon-optimized for expression in mammalian cells, e.g., murine or human cells. Most amino acids are encoded by more than one codon due to the degeneracy of the genetic code, allowing for substantial variations in the nucleotide sequence of a given nucleic acid without any alteration in the amino acid sequence encoded by the nucleic acid. However, many organisms display differences in codon usage, also known as “codon bias” (i.e., bias for use of a particular codon(s) for a given amino acid). Codon bias often correlates with the presence of a predominant species of tRNA for a particular codon, which in turn increases efficiency of mRNA translation. Accordingly, a coding sequence derived from a particular organism (e.g., a prokaryote) may be tailored for improved expression in a different organism (e.g., a eukaryote) through codon optimization.

Other specific variations of the Tet system include the following “Tet-Off” and “Tet-On” systems. In the Tet-Off system, transcription is inactive in the presence of Tc or Dox. In that system, a tetracycline-controlled transactivator protein (tTA), which is composed of TetR fused to the strong transactivating domain of VP16 from Herpes simplex virus, regulates expression of a target nucleic acid that is under transcriptional control of a tetracycline-responsive promoter element (TRE). The TRE is made up of TetO sequence concatamers fused to a promoter (commonly the minimal promoter sequence derived from the human cytomegalovirus (hCMV) immediate-early promoter). In the absence of Tc or Dox, tTA binds to the TRE and activates transcription of the target gene. In the presence of Tc or Dox, tTA cannot bind to the TRE, and expression from the target gene remains inactive.

Conversely, in the Tet-On system, transcription is active in the presence of Tc or Dox. The Tet-On system is based on a reverse tetracycline-controlled transactivator, rtTA. Like tTA, rtTA is a fusion protein comprised of the TetR repressor and the VP16 transactivation domain. However, a four amino acid change in the TetR DNA binding moiety alters rtTA's binding characteristics such that it can only recognize the tetO sequences in the TRE of the target transgene in the presence of Dox. Thus, in the Tet-On system, transcription of the TRE-regulated target gene is stimulated by rtTA only in the presence of Dox.

Another inducible promoter system is the lac repressor system from E. coli (See Brown et al., Cell 49:603-612 (1987)). The lac repressor system functions by regulating transcription of a polynucleotide of interest operably linked to a promoter comprising the lac operator (lacO). The lac repressor (lacR) binds to LacO, thus preventing transcription of the polynucleotide of interest. Expression of the polynucleotide of interest is induced by a suitable inducing agent, e.g., isopropyl-β-D-thiogalactopyranoside (IPTG).

In order to assess the expression of a polypeptide or portions thereof, the expression vector to be introduced into a cell can also contain either a selectable marker gene or a reporter gene or both to facilitate identification and selection of expressing cells from the population of cells sought to be transfected or infected through viral vectors. In other aspects, the selectable marker may be carried on a separate piece of DNA and used in a co-transfection procedure. Both selectable markers and reporter genes may be flanked with appropriate regulatory sequences to enable expression in the host cells. Useful selectable markers include, for example, antibiotic-resistance genes, such as neo and the like.

Reporter genes are used for identifying potentially transfected cells and for evaluating the functionality of regulatory sequences. In general, a reporter gene is a gene that is not present in or expressed by the recipient organism or tissue and that encodes a polypeptide whose expression is manifested by some easily detectable property, e.g., enzymatic activity. Expression of the reporter gene is assayed at a suitable time after the DNA has been introduced into the recipient cells. Suitable reporter genes may include genes encoding luciferase, β-galactosidase, chloramphenicol acetyl transferase, secreted alkaline phosphatase, or the green fluorescent protein gene (e.g., Ui-Tel et al., 2000 FEBS Letters 479: 79-82). Suitable expression systems are well known and may be prepared using known techniques or obtained commercially. In general, the construct with the minimal 5′ flanking region showing the highest level of expression of reporter gene is identified as the promoter. Such promoter regions may be linked to a reporter gene and used to evaluate agents for the ability to modulate promoter-driven transcription.

In some embodiments, there is provided nucleic acid encoding a full-length anti-TSLP antibody according to any of the full-length anti-TSLP antibodies described herein. In some embodiments, the nucleic acid comprises one or more nucleic acid sequences encoding the heavy and light chains of the full-length anti-TSLP antibody. In some embodiments, each of the one or more nucleic acid sequences are contained in separate vectors. In some embodiments, at least some of the nucleic acid sequences are contained in the same vector. In some embodiments, all of the nucleic acid sequences are contained in the same vector. Vectors may be selected, for example, from the group consisting of mammalian expression vectors and viral vectors (such as those derived from retroviruses, adenoviruses, adeno-associated viruses, herpes viruses, and lentiviruses).

Methods of introducing and expressing genes into a cell are known in the art. In the context of an expression vector, the vector can be readily introduced into a host cell, e.g., mammalian, bacterial, yeast, or insect cell by any method in the art. For example, the expression vector can be transferred into a host cell by physical, chemical, or biological means.

Physical methods for introducing a polynucleotide into a host cell include calcium phosphate precipitation, lipofection, particle bombardment, microinjection, electroporation, and the like. Methods for producing cells comprising vectors and/or exogenous nucleic acids are well-known in the art. See, for example, Green and Sambrook (2013, Molecular Cloning: A Laboratory Manual, Cold Spring Harbor Laboratory, New York). In some embodiments, the introduction of a polynucleotide into a host cell is carried out by calcium phosphate transfection.

Biological methods for introducing a polynucleotide of interest into a host cell include the use of DNA and RNA vectors. Viral vectors, and especially retroviral vectors, have become the most widely used method of inserting genes into mammalian, e.g., human cells. Other viral vectors can be derived from lentivirus, poxviruses, herpes simplex virus 1, adenoviruses and adeno-associated viruses, and the like. See, for example, U.S. Pat. Nos. 5,350,674 and 5,585,362.

Chemical means for introducing a polynucleotide into a host cell include colloidal dispersion systems, such as macromolecule complexes, nanocapsules, microspheres, beads, and lipid-based systems including oil-in-water emulsions, micelles, mixed micelles, and liposomes. An exemplary colloidal system for use as a delivery vehicle in vitro and in vivo is a liposome (e.g., an artificial membrane vesicle).

In the case where a non-viral delivery system is utilized, an exemplary delivery vehicle is a liposome. The use of lipid formulations is contemplated for the introduction of the nucleic acids into a host cell (in vitro, ex vivo or in vivo). In another aspect, the nucleic acid may be associated with a lipid. The nucleic acid associated with a lipid may be encapsulated in the aqueous interior of a liposome, interspersed within the lipid bilayer of a liposome, attached to a liposome via a linking molecule that is associated with both the liposome and the oligonucleotide, entrapped in a liposome, complexed with a liposome, dispersed in a solution containing a lipid, mixed with a lipid, combined with a lipid, contained as a suspension in a lipid, contained or complexed with a micelle, or otherwise associated with a lipid. Lipid, lipid/DNA or lipid/expression vector associated compositions are not limited to any particular structure in solution. For example, they may be present in a bilayer structure, as micelles, or with a “collapsed” structure. They may also simply be interspersed in a solution, possibly forming aggregates that are not uniform in size or shape. Lipids are fatty substances which may be naturally occurring or synthetic lipids. For example, lipids include the fatty droplets that naturally occur in the cytoplasm as well as the class of compounds which contain long-chain aliphatic hydrocarbons and their derivatives, such as fatty acids, alcohols, amines, amino alcohols, and aldehydes.

Regardless of the method used to introduce exogenous nucleic acids into a host cell or otherwise expose a cell to the inhibitor of the present application, in order to confirm the presence of the recombinant DNA sequence in the host cell, a variety of assays may be performed. Such assays include, for example, “molecular biological” assays well known to those of skill in the art, such as Southern and Northern blotting, RT-PCR and PCR; “biochemical” assays, such as detecting the presence or absence of a particular peptide, e.g., by immunological means (ELISAs and Western blots) or by assays described herein to identify agents falling within the scope of the application.

### Preparation of Anti-TSLP Antibodies

In some embodiments, the anti-TSLP antibody is a monoclonal antibody or derived from a monoclonal antibody. In some embodiments, the anti-TSLP antibody comprises V_{H} and V_{L} domains, or variants thereof, from the monoclonal antibody. In some embodiments, the anti-TSLP antibody further comprises C_{H}1 and C_{L} domains, or variants thereof, from the monoclonal antibody. Monoclonal antibodies can be prepared, e.g., using known methods in the art, including hybridoma methods, phage display methods, or using recombinant DNA methods. Additionally, exemplary phage display methods are described herein and in the Examples below.

In a hybridoma method, a hamster, mouse, or other appropriate host animal is typically immunized with an immunizing agent to elicit lymphocytes that produce or are capable of producing antibodies that will specifically bind to the immunizing agent. Alternatively, the lymphocytes can be immunized in vitro. The immunizing agent can include a polypeptide or a fusion protein of the protein of interest. Generally, peripheral blood lymphocytes (“PBLs”) are used if cells of human origin are desired, or spleen cells or lymph node cells are used if non-human mammalian sources are desired. The lymphocytes are then fused with an immortalized cell line using a suitable fusing agent, such as polyethylene glycol, to form a hybridoma cell. Immortalized cell lines are usually transformed mammalian cells, particularly myeloma cells of rodent, bovine, and human origin. Usually, rat or mouse myeloma cell lines are employed. The hybridoma cells can be cultured in a suitable culture medium that preferably contains one or more substances that inhibit the growth or survival of the unfused, immortalized cells. For example, if the parental cells lack the enzyme hypoxanthine guanine phosphoribosyl transferase (HGPRT or HPRT), the culture medium for the hybridomas typically will include hypoxanthine, aminopterin, and thymidine (“HAT medium”), which prevents the growth of HGPRT-deficient cells.

In some embodiments, the immortalized cell lines fuse efficiently, support stable high-level expression of antibody by the selected antibody-producing cells, and are sensitive to a medium such as HAT medium. In some embodiments, the immortalized cell lines are murine myeloma lines, which can be obtained, for instance, from the Salk Institute Cell Distribution Center, San Diego, California and the American Type Culture Collection, Manassas, Virginia. Human myeloma and mouse-human heteromyeloma cell lines also have been described for the production of human monoclonal antibodies.

The culture medium in which the hybridoma cells are cultured can then be assayed for the presence of monoclonal antibodies directed against the polypeptide. The binding specificity of monoclonal antibodies produced by the hybridoma cells can be determined by immunoprecipitation or by an in vitro binding assay, such as radioimmunoassay (RIA) or enzyme-linked immunosorbent assay (ELISA). Such techniques and assays are known in the art. The binding affinity of the monoclonal antibody can, for example, be determined by the Scatchard analysis of Munson and Pollard, Anal. Biochem., 107:220 (1980).

After the desired hybridoma cells are identified, the clones can be sub cloned by limiting dilution procedures and grown by standard methods. Goding, supra. Suitable culture media for this purpose include, for example, Dulbecco's Modified Eagle's Medium and RPMI-1640 medium. Alternatively, the hybridoma cells can be grown in vivo as ascites in a mammal.

The monoclonal antibodies secreted by the sub clones can be isolated or purified from the culture medium or ascites fluid by conventional immunoglobulin purification procedures such as, for example, protein A-Sepharose, hydroxylapatite chromatography, gel electrophoresis, dialysis, or affinity chromatography.

In some embodiments, according to any of the anti-TSLP antibodies described herein, the anti-TSLP antibody comprises sequences from a clone selected from an antibody library (such as a phage library presenting scFv or Fab fragments). The clone may be identified by screening combinatorial libraries for antibody fragments with the desired activity or activities. For example, a variety of methods are known in the art for generating phage display libraries and screening such libraries for antibodies possessing the desired binding characteristics. Such methods are reviewed, e.g., in Hoogenboom et al., Methods in Molecular Biology 178:1-37 (O'Brien et al., ed., Human Press, Totowa, N.J., 2001) and further described, e.g., in McCafferty et al., Nature 348:552-554; Clackson et al., Nature 352: 624-628 (1991); Marks et al., J. Mol. Biol. 222: 581-597 (1992); Marks and Bradbury, Methods in Molecular Biology 248:161-175 (Lo, ed., Human Press, Totowa, N.J., 2003); Sidhu et al., J. Mol. Biol. 338(2): 299-310 (2004); Lee et al., J. Mol. Biol. 340(5): 1073-1093 (2004); Fellouse, Proc. Natl. Acad. Sci. USA 101(34): 12467-12472 (2004); and Lee et al., J. Immunol. Methods 284(1-2): 119-132(2004).

In certain phage display methods, repertoires of V_{H} and V_{L} genes are separately cloned by polymerase chain reaction (PCR) and recombined randomly in phage libraries, which can then be screened for antigen-binding phage as described in Winter et al., Ann. Rev. Immunol., 12: 433-455 (1994). Phage typically display antibody fragments, either as scFv fragments or as Fab fragments. Libraries from immunized sources provide high-affinity antibodies to the immunogen without the requirement of constructing hybridomas. Alternatively, the naive repertoire can be cloned (e.g., from human) to provide a single source of antibodies to a wide range of non-self and also self-antigens without any immunization as described by Griffiths et al., EMBO J, 12: 725-734 (1993). Finally, naive libraries can also be made synthetically by cloning unrearranged V-gene segments from stem cells, and using PCR primers containing random sequence to encode the highly variable CDR3 regions and to accomplish rearrangement in vitro, as described by Hoogenboom and Winter, J. Mol. Biol., 227: 381-388 (1992). Patent publications describing human antibody phage libraries include, for example: U.S. Pat. No. 5,750,373, and US Patent Publication Nos. 2005/0079574, 2005/0119455, 2005/0266000, 2007/0117126, 2007/0160598, 2007/0237764, 2007/0292936, and 2009/0002360.

The anti-TSLP antibodies can be prepared using phage display to screen libraries for anti-TSLP antibody moieties specific to the target TSLP. The library can be a human scFv phage display library having a diversity of at least one×10⁹ (such as at least about any of 1×10⁹, 2.5×10⁹, 5×10⁹, 7.5×10⁹, 1×10¹⁰, 2.5×10¹⁰, 5×10¹⁰, 7.5×10¹⁰, or 1×10¹¹) unique human antibody fragments. In some embodiments, the library is a naïve human library constructed from DNA extracted from human PMBCs and spleens from healthy donors, encompassing all human heavy and light chain subfamilies. In some embodiments, the library is a naïve human library constructed from DNA extracted from PBMCs isolated from patients with various diseases, such as patients with autoimmune diseases, cancer patients, and patients with infectious diseases. In some embodiments, the library is a semi-synthetic human library, wherein heavy chain CDR3 is completely randomized, with all amino acids (with the exception of cysteine) equally likely to be present at any given position (see, e.g., Hoet, R. M. et al., Nat. Biotechnol. 23(3):344-348, 2005). In some embodiments, the heavy chain CDR3 of the semi-synthetic human library has a length from about 5 to about 24 (such as about any of 5, 6, 7, 8, 9, 10, 11, 12, 13, 14, 15, 16, 17, 18, 19, 20, 21, 22, 23, or 24) amino acids. In some embodiments, the library is a fully-synthetic phage display library. In some embodiments, the library is a non-human phage display library.

Phage clones that bind to the target TSLP with high affinity can be selected by iterative binding of phage to the target TSLP, which is bound to a solid support (such as, for example, beads for solution panning or mammalian cells for cell panning), followed by removal of non-bound phage and by elution of specifically bound phage. The bound phage clones are then eluted and used to infect an appropriate host cell, such as E. coli XL1-Blue, for expression and purification. The panning can be performed for multiple (such as about any of 2, 3, 4, 5, 6 or more) rounds with solution panning, cell panning, or a combination of both, to enrich for phage clones binding specifically to the target TSLP. Enriched phage clones can be tested for specific binding to the target TSLP by any methods known in the art, including for example ELISA and FACS.

An alternative method for screening antibody libraries is to display the protein on the surface of yeast cells. Wittrup et al. (U.S. Pat. Nos. 6,699,658 and 6,696,25 1) have developed a method for a yeast cell display library. In this yeast display system, a component involves the yeast agglutinin protein (Aga1), which is anchored to the yeast cell wall. Another component involves a second subunit of the agglutinin protein Aga2, which can display on the surface yeast cells through disulfide bonds to Aga1 protein. The protein Aga1 is expressed from a yeast chromosome after the Aga1 gene integration. A library of single chain variable fragments (scFv) is fused genetically to Aga2 sequence in the yeast display plasmid, which, after transformation, is maintained in yeast episomally with a nutritional marker. Both of the Aga1 and Aga2 proteins were expressed under the control of the galactose-inducible promoter.

Human antibody V gene repertoire (V_{H} and V_{K} fragments) are obtained by PCR method using a pool of degenerate primers (Sblattero, D. & Bradbury, A. Immunotechnology 3, 271-278 1998). The PCR templates are from the commercially available RNAs or cDNAs, including PBMC, spleen, lymph nodes, bone marrow and tonsils. Separate V_{H} and V_{K} PCR libraries were combined, then assembled together in the scFv format by overlap extension PCR (Sheets, M. D. et al. Proc. Natl. Acad. Sci. USA 95, 6157-6162 1998). To construct the yeast scFv display library, the resultant scFv PCR products are cloned into the yeast display plasmid in the yeasts by homologous recombination. (Chao, G, et al, Nat Protoc. 2006; 1(2):755-68. Miller K D, et al. Current Protocols in Cytometry 4.7.1-4.7.30, 2008).

The anti-TSLP antibodies can be discovered using mammalian cell display systems in which antibody moieties are displayed on the cell surface and those specific to the target TSLP are isolated by the antigen-guided screening method, as described in U.S. Pat. No. 7,732,195B2. A Chinese hamster ovary (CHO) cell library representing a large set of human IgG antibody genes can be established and used to discover the clones expressing high-affinity antibody genes. Another display system has been developed to enable simultaneous high-level cell surface display and secretion of the same protein through alternate splicing, where the displayed protein phenotype remains linked to genotype, allowing soluble secreted antibody to be simultaneously characterized in biophysical and cell-based functional assays. This approach overcomes many limitations of previous mammalian cell display, enabling direct selection and maturation of antibodies in the form of full-length, glycosylated IgGs (Peter M. Bowers, et al, Methods 2014, 65:44-56). Transient expression systems are suitable for a single round of antigen selection before recovery of the antibody genes and therefore most useful for the selection of antibodies from smaller libraries. Stable episomal vectors offer an attractive alternative. Episomal vectors can be transfected at high efficiency and stably maintained at low copy number, permitting multiple rounds of panning and the resolution of more complex antibody libraries.

The IgG library is based on germline sequence V-gene segments joined to rearranged (D)J regions isolated from a panel of human donors. RNA collected from 2000 human blood samples was reverse-transcribed into cDNA, and the V_{H} and V_{K} fragments were amplified using V_{H}- and V_{K}-specific primers and purified by gel extraction. IgG libraries were generated by sub-cloning the V_{H} and V_{K} fragments into the display vectors containing IgG1 or K constant regions respectively and then electroporating into or transducing 293T cells. To generate the scFv antibody display library, scFvs were generated by linking V_{H} and V_{K}, and then sub-cloned into the display vector, which were then electroporated into or transduce 293T cells. As we known, the IgG library is based on germline sequence V-gene segments joined to rearranged (D)J regions isolated from a panel of donors, the donor can be a mouse, rat, rabbit, or monkey.

Monoclonal antibodies can also be made by recombinant DNA methods, such as those described in U.S. Pat. No. 4,816,567. DNA encoding the monoclonal antibodies of the application can be readily isolated and sequenced using conventional procedures (e.g., by using oligonucleotide probes that are capable of binding specifically to genes encoding the heavy and light chains of murine antibodies). Hybridoma cells as described above or TSLP-specific phage clones of the application can serve as a source of such DNA. Once isolated, the DNA can be placed into expression vectors, which are then transfected into host cells such as simian COS cells, Chinese hamster ovary (CHO) cells, or myeloma cells that do not otherwise produce immunoglobulin protein, to obtain the synthesis of monoclonal antibodies in the recombinant host cells. The DNA also can be modified, for example, by substituting the coding sequence for human heavy- and light-chain constant domains and/or framework regions in place of the homologous non-human sequences (U.S. Pat. No. 4,816,567; Morrison et al., supra) or by covalently joining to the immunoglobulin coding sequence all or part of the coding sequence for a non-immunoglobulin polypeptide. Such a non-immunoglobulin polypeptide can be substituted for the constant domains of an antibody of the application, or can be substituted for the variable domains of one antigen-combining site of an antibody of the application to create a chimeric bivalent antibody.

The antibodies can be monovalent antibodies. Methods for preparing monovalent antibodies are known in the art. For example, one method involves recombinant expression of immunoglobulin light chain and modified heavy chain. The heavy chain is truncated generally at any point in the Fc region so as to prevent heavy-chain crosslinking. Alternatively, the relevant cysteine residues are substituted with another amino acid residue or are deleted so as to prevent crosslinking.

In vitro methods are also suitable for preparing monovalent antibodies. Digestion of antibodies to produce fragments thereof, particularly Fab fragments, can be accomplished using any method known in the art.

Antibody variable domains with the desired binding specificities (antibody-antigen combining sites) can be fused to immunoglobulin constant-domain sequences. The fusion preferably is with an immunoglobulin heavy-chain constant domain, comprising at least part of the hinge, CH2, and CH3 regions. In some embodiments, the first heavy-chain constant region (CH1) containing the site necessary for light-chain binding is present in at least one of the fusions. DNAs encoding the immunoglobulin heavy-chain fusions and, if desired, the immunoglobulin light chain, are inserted into separate expression vectors, and are co-transfected into a suitable host organism.

### Human and Humanized Antibodies

The anti-TSLP antibodies (e.g., full-length anti-TSLP antibodies) can be humanized antibodies or human antibodies. Humanized forms of non-human (e.g., murine) antibody moieties are chimeric immunoglobulins, immunoglobulin chains, or fragments thereof (such as Fv, Fab, Fab′, F(ab′)₂, scFv, or other antigen-binding subsequences of antibodies) that typically contain minimal sequence derived from non-human immunoglobulin. Humanized antibody moieties include human immunoglobulins, immunoglobulin chains, or fragments thereof (recipient antibody) in which residues from a CDR of the recipient are replaced by residues from a CDR of a non-human species (donor antibody) such as mouse, rat, or rabbit having the desired specificity, affinity, and capacity. In some instances, Fv framework residues of the human immunoglobulin are replaced by corresponding non-human residues. Humanized antibody moieties can also comprise residues that are found neither in the recipient antibody nor in the imported CDR or framework sequences. In general, the humanized antibody can comprise substantially all of at least one, and typically two, variable domains, in which all or substantially all of the CDR regions correspond to those of a non-human immunoglobulin, and all or substantially all of the FR regions are those of a human immunoglobulin consensus sequence.

Generally, a humanized antibody has one or more amino acid residues introduced into it from a source that is non-human. These non-human amino acid residues are often referred to as “import” residues, which are typically taken from an “import” variable domain. According to some embodiments, humanization can be essentially performed following the method of Winter and co-workers (Jones et al., Nature, 321: 522-525 (1986); Riechmann et al., Nature, 332: 323-327 (1988); Verhoeyen et al., Science, 239: 1534-1536 (1988)), by substituting rodent CDRs or CDR sequences for the corresponding sequences of a human antibody. Accordingly, such “humanized” antibody moieties are antibody moieties (U.S. Pat. No. 4,816,567), wherein substantially less than an intact human variable domain has been substituted by the corresponding sequence from a non-human species. In practice, humanized antibody moieties are typically human antibody moieties in which some CDR residues and possibly some FR residues are substituted by residues from analogous sites in rodent antibodies.

As an alternative to humanization, human antibody moieties can be generated. For example, it is now possible to produce transgenic animals (e.g., mice) that are capable, upon immunization, of producing a full repertoire of human antibodies in the absence of endogenous immunoglobulin production. For example, it has been described that the homozygous deletion of the antibody heavy-chain joining region (JH) gene in chimeric and germ-line mutant mice results in complete inhibition of endogenous antibody production. Transfer of the human germ-line immunoglobulin gene array into such germ-line mutant mice will result in the production of human antibodies upon antigen challenge. See, e.g., Jakobovits et al., PNAS USA, 90:2551 (1993); Jakobovits et al., Nature, 362:255-258 (1993); Bruggemann et al., Year in Immunol., 7:33 (1993); U.S. Pat. Nos. 5,545,806, 5,569,825, 5,591,669; 5,545,807; and WO 97/17852. Alternatively, human antibodies can be made by introducing human immunoglobulin loci into transgenic animals, e.g., mice in which the endogenous immunoglobulin genes have been partially or completely inactivated. Upon challenge, human antibody production is observed that closely resembles that seen in humans in all respects, including gene rearrangement, assembly, and antibody repertoire. This approach is described, for example, in U.S. Pat. Nos. 5,545,807; 5,545,806; 5,569,825; 5,625,126; 5,633,425; and 5,661,016, and Marks et al., Bio Technology, 10: 779-783 (1992); Lonberg et al., Nature, 368: 856-859 (1994); Morrison, Nature, 368: 812-813 (1994); Fishwild et al., Nature Biotechnology, 14: 845-851 (1996); Neuberger, Nature Biotechnology, 14: 826 (1996); Lonberg and Huszar, Intern. Rev. Immunol., 13: 65-93 (1995).

Human antibodies may also be generated by in vitro activated B cells (see U.S. Pat. Nos. 5,567,610 and 5,229,275) or by using various techniques known in the art, including phage display libraries. Hoogenboom and Winter, J. Mol. Biol., 227:381 (1991); Marks et al., J. Mol. Biol., 222:581 (1991). The techniques of Cole et al. and Boerner et al. are also available for the preparation of human monoclonal antibodies. Cole et al., Monoclonal Antibodies and Cancer Therapy, Alan R. Liss, p. 77 (1985) and Boerner et al., J. Immunol., 147(1): 86-95 (1991).

### Anti-TSLP Antibody Variants

In some embodiments, amino acid sequences of the anti-TSLP antibody variants (e.g., full-length anti-TSLP antibody) provided herein are contemplated. For example, it may be desirable to improve the binding affinity and/or other biological properties of the antibody. Amino acid sequence of an antibody variant may be prepared by introducing appropriate modifications into the nucleotide sequence encoding the antibody, or by peptide synthesis. Such modifications include, for example, deletions from, and/or insertions into and/or substitutions of residues within the amino acid sequences of the antibody. Any combination of deletion, insertion, and substitution can be made to arrive at the final construct, provided that the final construct possesses the desired characteristics, e.g., antigen-binding.

In some embodiments, anti-TSLP antibody variants having one or more amino acid substitutions are provided. Sites of interest for substitutional mutagenesis include the HVRs and FRs. Amino acid substitutions may be introduced into an antibody of interest and the products screened for a desired activity, e.g., improved bioactivity, retained/improved antigen binding, decreased immunogenicity, or improved ADCC or CDC.

Conservative substitutions are shown in Table 4 below.

TABLE 4
CONSERVATIVE SUBSTITUTIONS

| Original | | Preferred |
|---|---|---|
| Residue | Exemplary Substitutions | Substitutions |
| Ala (A) | Val; Leu; Ile | Val |
| Arg (R) | Lys; Gln; Asn | Lys |
| Asn (N) | Gln; His; Asp, Lys; Arg | Gln |
| Asp (D) | Glu; Asn | Glu |
| Cys (C) | Ser; Ala | Ser |
| Gln (Q) | Asn; Glu | Asn |
| Glu (E) | Asp; Gln | Asp |
| Gly (G) | Ala | Ala |
| His (H) | Asn; Gln; Lys; Arg | Arg |
| Ile (I) | Leu; Val; Met; Ala; Phe; Norleucine | Leu |
| Leu (L) | Norleucine; Ile; Val; Met; Ala; Phe | Ile |
| Lys (K) | Arg; Gln; Asn | Arg |
| Met (M) | Leu; Phe; Ile | Leu |
| Phe (F) | Trp; Leu; Val; Ile; Ala; Tyr | Tyr |
| Pro (P) | Ala | Ala |
| Ser (S) | Thr | Thr |
| Thr (T) | Val; Ser | Ser |
| Trp (W) | Tyr; Phe | Tyr |
| Tyr (Y) | Trp; Phe; Thr; Ser | Phe |
| Val (V) | Ile; Leu; Met; Phe; Ala; Norleucine | Leu |

Amino acids may be grouped into different classes according to common side-chain properties:
a. hydrophobic: Norleucine, Met, Ala, Val, Leu, Ile;
b. neutral hydrophilic: Cys, Ser, Thr, Asn, Gln;
c. acidic: Asp, Glu;
d. basic: His, Lys, Arg;
e. residues that influence chain orientation: Gly, Pro;
f. aromatic: Trp, Tyr, Phe.

Non-conservative substitutions will entail exchanging a member of one of these classes for another class.

An exemplary substitutional variant is an affinity matured antibody, which may be conveniently generated, e.g., using phage display-based affinity maturation techniques. Briefly, one or more CDR residues are mutated and the variant antibody moieties displayed on phage and screened for a particular biological activity (e.g., bioactivity based on inhibition of TSLP dependent Stat5 activation assay or binding affinity). Alterations (e.g., substitutions) may be made in HVRs, e.g., to improve bioactivity based on inhibition of TSLP dependent Stat5 activation assay or antibody affinity. Such alterations may be made in HVR “hotspots,” i.e., residues encoded by codons that undergo mutation at high frequency during the somatic maturation process (see, e.g., Chowdhury, Methods Mol. Biol. 207:179-196 (2008)), and/or specificity determining residues (SDRs), with the resulting variant V_{H} and V_{L} being tested for binding affinity. Affinity maturation by constructing and reselecting from secondary libraries has been described, e.g., in Hoogenboom et al. in Methods in Molecular Biology 178:1-37 (O'Brien et al., ed., Human Press, Totowa, NJ, (2001)).

In some embodiments of affinity maturation, diversity is introduced into the variable genes chosen for maturation by any of a variety of methods (e.g., error-prone PCR, chain shuffling, or oligonucleotide-directed mutagenesis). A secondary library is then created. The library is then screened to identify any antibody variants with the desired affinity. Another method to introduce diversity involves HVR-directed approaches, in which several HVR residues (e.g., 4-6 residues at a time) are randomized. HVR residues involved in antigen binding may be specifically identified, e.g., using alanine scanning mutagenesis or modeling. CDR-H3 and CDR-L3 in particular are often targeted.

In some embodiments, substitutions, insertions, or deletions may occur within one or more HVRs so long as such alterations do not substantially reduce the ability of the antibody to bind antigen. For example, conservative alterations (e.g., conservative substitutions as provided herein) that do not substantially reduce binding affinity may be made in HVRs. Such alterations may be outside of HVR “hotspots” or SDRs. In some embodiments of the variant V_{H} and V_{L} sequences provided above, each HVR either is unaltered, or contains no more than one, two or three amino acid substitutions.

A useful method for identification of residues or regions of an antibody that may be targeted for mutagenesis is called “alanine scanning mutagenesis” as described by Cunningham and Wells (1989) Science, 244:1081-1085. In this method, a residue or group of target residues (e.g., charged residues such as Arg, Asp, His, Lys, and Glu) are identified and replaced by a neutral or negatively charged amino acid (e.g., Ala or Glu) to determine whether the interaction of the antibody with antigen is affected. Further substitutions may be introduced at the amino acid locations to demonstrate functional sensitivity to the initial substitutions. Alternatively, or additionally, a crystal structure of an antigen-antibody complex can be determined to identify contact points between the antibody and antigen. Such contact residues and neighboring residues may be targeted or eliminated as candidates for substitution. Variants may be screened to determine whether they contain the desired properties.

Amino acid sequence insertions include amino- and/or carboxyl-terminal fusions ranging in length from one residue to polypeptides containing a hundred or more residues, as well as intrasequence insertions of single or multiple amino acid residues. Examples of terminal insertions include an antibody with an N-terminal methionyl residue. Other insertional variants of the antibody molecule include the fusion to the N- or C-terminus of the antibody to an enzyme (e.g. for ADEPT) or a polypeptide which increases the serum half-life of the antibody.

### Fc Region Variants

In some embodiments, one or more amino acid modifications may be introduced into the Fc region of an antibody (e.g., a full-length anti-TSLP antibody or anti-TSLP Fc fusion protein) provided herein, thereby generating an Fc region variant. In some embodiments, the Fc region variant has enhanced ADCC effector function, often related to binding to Fc receptors (FcRs). In some embodiments, the Fc region variant has decreased ADCC effector function. There are many examples of changes or mutations to Fc sequences that can alter effector function. For example, WO 00/42072 and Shields et al. J Biol. Chem. 9(2): 6591-6604 (2001) describe antibody variants with improved or diminished binding to FcRs. The contents of those publications are specifically incorporated herein by reference.

Antibody-Dependent Cell-Mediated Cytotoxicity (ADCC) is a mechanism of action of therapeutic antibodies against tumor cells. ADCC is a cell-mediated immune defense whereby an effector cell of the immune system actively lyses a target cell (e.g., a cancer cell), whose membrane-surface antigens have been bound by specific antibodies (e.g., an anti-TSLP antibody). The typical ADCC involves activation of NK cells by antibodies. An NK cell expresses CD16 which is an Fc receptor. This receptor recognizes, and binds to, the Fc portion of an antibody bound to the surface of a target cell. The most common Fc receptor on the surface of an NK cell is called CD16 or FcγRIII. Binding of the Fc receptor to the Fc region of an antibody results in NK cell activation, release of cytolytic granules and consequent target cell apoptosis. The contribution of ADCC to tumor cell killing can be measured with a specific test that uses NK-92 cells that have been transfected with a high-affinity FcR. Results are compared to wild-type NK-92 cells that do not express the FcR.

In some embodiments, the application contemplates an anti-TSLP antibody variant (such as a full-length anti-TSLP antibody variant) comprising an Fc region that possesses some but not all effector functions, which makes it a desirable candidate for applications in which the half-life of the anti-TSLP antibody in vivo is important yet certain effector functions (such as CDC and ADCC) are unnecessary or deleterious. In vitro and/or in vivo cytotoxicity assays can be conducted to confirm the reduction/depletion of CDC and/or ADCC activities. For example, Fc receptor (FcR) binding assays can be conducted to ensure that the antibody lacks FcγR binding (hence likely lacking ADCC activity), but retains FcRn binding ability. The primary cells for mediating ADCC, NK cells, express FcγRIII only, whereas monocytes express FcγRI, FcγRII and FcγRIII. FcR expression on hematopoietic cells is summarized in Table 3 on page 464 of Ravetch and Kinet, Annu. Rev. Immunol. 9:457-492 (1991). Non-limiting examples of in vitro assays to assess ADCC activity of a molecule of interest is described in U.S. Pat. No. 5,500,362 (see, e.g. Hellstrom, I. et al. Proc. Nat'l Acad. Sci. USA 83:7059-7063 (1986)) and Hellstrom, I et al., Proc. Nat'l Acad. Sci. USA 82:1499-1502 (1985); U.S. Pat. No. 5,821,337 (see Bruggemann, M. et al., J. Exp. Med. 166:1351-1361 (1987)). Alternatively, non-radioactive assay methods may be employed (see, for example, ACTI™ non-radioactive cytotoxicity assay for flow cytometry (CellTechnology, Inc. Mountain View, Calif; and CYTOTOX 96™ non-radioactive cytotoxicity assay (Promega, Madison, Wis.). Useful effector cells for such assays include peripheral blood mononuclear cells (PBMC) and Natural Killer (NK) cells. Alternatively, or additionally, ADCC activity of the molecule of interest may be assessed in vivo, e.g., in an animal model such as that disclosed in Clynes et al. Proc. Nat'l Acad. Sci. USA 95:652-656 (1998). C1q binding assays may also be carried out to confirm that the antibody is unable to bind C1q and hence lacks CDC activity. See, e.g., C1q and C3c binding ELISA in WO 2006/029879 and WO 2005/100402. To assess complement activation, a CDC assay may be performed (see, for example, Gazzano-Santoro et al., J. Immunol. Methods 202:163 (1996); Cragg, M. S. et al., Blood 101:1045-1052 (2003); and Cragg, M. S. and M. J. Glennie, Blood 103:2738-2743 (2004)). FcRn binding and in vivo clearance/half-life determinations can also be performed using methods known in the art (see, e.g., Petkova, S. B. et al., Int'l. Immunol. 18(12):1759-1769 (2006)).

Antibodies with reduced effector function include those with substitution of one or more of Fc region residues 238, 265, 269, 270, 297, 327 and 329 (U.S. Pat. No. 6,737,056). Such Fc mutants include Fc mutants with substitutions at two or more of amino acid positions 265, 269, 270, 297 and 327, including the so-called “DANA” Fc mutant with substitution of residues 265 and 297 to alanine (U.S. Pat. No. 7,332,581).

Certain antibody variants with improved or diminished binding to FcRs are described. (See, e.g., U.S. Pat. No. 6,737,056; WO 2004/056312, and Shields et al., J. Biol. Chem. 9(2): 6591-6604 (2001).)

In some embodiments, there is provided an anti-TSLP antibody (such as a full-length anti-TSLP antibody) variant comprising a variant Fc region comprising one or more amino acid substitutions which improve ADCC. In some embodiments, the variant Fc region comprises one or more amino acid substitutions which improve ADCC, wherein the substitutions are at positions 298, 333, and/or 334 of the variant Fc region (EU numbering of residues). In some embodiments, the anti-TSLP antibody (e.g., full-length anti-TSLP antibody) variant comprises the following amino acid substitution in its variant Fc region: S298A, E333A, and K334A.

In some embodiments, alterations are made in the Fc region that result in altered (i.e., either improved or diminished) C1q binding and/or Complement Dependent Cytotoxicity (CDC), e.g., as described in U.S. Pat. No. 6,194,551, WO 99/51642, and Idusogie et al., J. Immunol. 164: 4178-4184 (2000).

In some embodiments, there is provided an anti-TSLP antibody (such as a full-length anti-TSLP antibody) variant comprising a variant Fc region comprising one or more amino acid substitutions which increase half-life and/or improve binding to the neonatal Fc receptor (FcRn). Antibodies with increased half-lives and improved binding to FcRn are described in US2005/0014934A1 (Hinton et al.). Those antibodies comprise an Fc region with one or more substitutions therein which improve binding of the Fc region to FcRn. Such Fc variants include those with substitutions at one or more of Fc region residues: 238, 256, 265, 272, 286, 303, 305, 307, 311, 312, 317, 340, 356, 360, 362, 376, 378, 380, 382, 413, 424 or 434, e.g., substitution of Fc region residue 434 (U.S. Pat. No. 7,371,826).

See also Duncan & Winter, Nature 322:738-40 (1988); U.S. Pat. Nos. 5,648,260; 5,624,821; and WO 94/29351 concerning other examples of Fc region variants.

Anti-TSLP antibodies (such as full-length anti-TSLP antibodies) comprising any of the Fc variants described herein, or combinations thereof, are contemplated.

### Glycosylation Variants

In some embodiments, an anti-TSLP antibody (such as a full-length anti-TSLP antibody) provided herein is altered to increase or decrease the extent to which the anti-TSLP antibody is glycosylated. Addition or deletion of glycosylation sites to an anti-TSLP antibody may be conveniently accomplished by altering the amino acid sequence of the anti-TSLP antibody or polypeptide portion thereof such that one or more glycosylation sites is created or removed.

Wherein the anti-TSLP antibody comprises an Fc region, the carbohydrate attached thereto may be altered. Native antibodies produced by mammalian cells typically comprise a branched, biantennary oligosaccharide that is generally attached by an N-linkage to Asn297 of the CH2 domain of the Fc region. See, e.g., Wright et al., TIBTECH 15:26-32 (1997). The oligosaccharide may include various carbohydrates, e.g., mannose, N-acetyl glucosamine (GlcNAc), galactose, and sialic acid, as well as a fucose attached to a GlcNAc in the “stem” of the biantennary oligosaccharide structure. In some embodiments, modifications of the oligosaccharide in an anti-TSLP antibody of the application may be made in order to create anti-TSLP antibody variants with certain improved properties.

The N-glycans attached to the CH2 domain of Fc is heterogeneous. Antibodies or Fc fusion proteins generated in CHO cells are fucosylated by fucosyltransferase activity. See Shoji-Hosaka et al., J. Biochem. 2006, 140:777-83. Normally, a small percentage of naturally occurring afucosylated IgGs may be detected in human serum. N-glycosylation of the Fc is important for binding to FcγR; and afucosylation of the N-glycan increases Fc's binding capacity to FcγRIIIa. Increased FcγRIIIa binding can enhance ADCC, which can be advantageous in certain antibody therapeutic applications in which cytotoxicity is desirable.

In some embodiments, an enhanced effector function can be detrimental when Fc-mediated cytotoxicity is undesirable. In some embodiments, the Fc fragment or CH2 domain is not glycosylated. In some embodiments, the N-glycosylation site in the CH2 domain is mutated to prevent from glycosylation.

In some embodiments, anti-TSLP antibody (such as a full-length anti-TSLP antibody) variants are provided comprising an Fc region wherein a carbohydrate structure attached to the Fc region has reduced fucose or lacks fucose, which may improve ADCC function. Specifically, anti-TSLP antibodies are contemplated herein that have reduced fucose relative to the amount of fucose on the same anti-TSLP antibody produced in a wild-type CHO cell. That is, they are characterized by having a lower amount of fucose than they would otherwise have if produced by native CHO cells (e.g., a CHO cell that produce a native glycosylation pattern, such as, a CHO cell containing a native FUT8 gene). In some embodiments, the anti-TSLP antibody is one wherein less than about 50%, 40%, 30%, 20%, 10%, or 5% of the N-linked glycans thereon comprise fucose. For example, the amount of fucose in such an anti-TSLP antibody may be from 1% to 80%, from 1% to 65%, from 5% to 65% or from 20% to 40%. In some embodiments, the anti-TSLP antibody is one wherein none of the N-linked glycans thereon comprise fucose, i.e., wherein the anti-TSLP antibody is completely without fucose, or has no fucose or is afucosylated. The amount of fucose is determined by calculating the average amount of fucose within the sugar chain at Asn297, relative to the sum of all glycostructures attached to Asn 297 (e. g. complex, hybrid and high mannose structures) as measured by MALDI-TOF mass spectrometry, as described in WO 2008/077546, for example. Asn297 refers to the asparagine residue located at about position 297 in the Fc region (EU numbering of Fc region residues); however, Asn297 may also be located about ±3 amino acids upstream or downstream of position 297, i.e., between positions 294 and 300, due to minor sequence variations in antibodies. Such fucosylation variants may have improved ADCC function. See, e.g., US Patent Publication Nos. US 2003/0157108 (Presta, L.); US 2004/0093621 (Kyowa Hakko Kogyo Co., Ltd). Examples of publications related to “defucosylated” or “fucose-deficient” antibody variants include: US 2003/0157108; WO 2000/61739; WO 2001/29246; US 2003/0115614; US 2002/0164328; US 2004/0093621; US 2004/0132140; US 2004/0110704; US 2004/0110282; US 2004/0109865; WO 2003/085119; WO 2003/084570; WO 2005/035586; WO 2005/035778; WO2005/053742; WO2002/031140; Okazaki et al. J. Mol. Biol. 336:1239-1249 (2004); Yamane-Ohnuki et al. Biotech. Bioeng. 87: 614 (2004). Examples of cell lines capable of producing defucosylated antibodies include Lec13 CHO cells deficient in protein fucosylation (Ripka et al. Arch. Biochem. Biophys. 249:533-545 (1986); US Pat Appl No US 2003/0157108 A1, Presta, L; and WO 2004/056312 A1, Adams et al., especially at Example 11), and knockout cell lines, such asα-1,6-fucosyltransferase gene, FUT8, knockout CHO cells (see, e.g., Yamane-Ohnuki et al. Biotech. Bioeng. 87: 614 (2004); Kanda, Y. et al., Biotechnol. Bioeng., 94(4):680-688 (2006); and WO2003/085107).

Anti-TSLP antibody (such as a full-length anti-TSLP antibody) variants are further provided with bisected oligosaccharides, e.g., in which a biantennary oligosaccharide attached to the Fc region of the anti-TSLP antibody is bisected by GlcNAc. Such anti-TSLP antibody (such as a full-length anti-TSLP antibody) variants may have reduced fucosylation and/or improved ADCC function. Examples of such antibody variants are described, e.g., in WO 2003/011878 (Jean-Mairet et al.); U.S. Pat. No. 6,602,684 (Umana et al.); US 2005/0123546 (Umana et al.), and Ferrara et al., Biotechnology and Bioengineering, 93(5): 851-861 (2006). Anti-TSLP antibody (such as full-length anti-TSLP antibody) variants with at least one galactose residue in the oligosaccharide attached to the Fc region are also provided. Such anti-TSLP antibody variants may have improved CDC function. Such antibody variants are described, e.g., in WO 1997/30087 (Patel et al.); WO 1998/58964 (Raju, S.); and WO 1999/22764 (Raju, S.).

In some embodiments, the anti-TSLP antibody (such as a full-length anti-TSLP antibody) variants comprising an Fc region are capable of binding to an FcγRIII. In some embodiments, the anti-TSLP antibody (such as a full-length anti-TSLP antibody) variants comprising an Fc region have ADCC activity in the presence of human effector cells (e.g., T cell) or have increased ADCC activity in the presence of human effector cells compared to the otherwise same anti-TSLP antibody (such as a full-length anti-TSLP antibody) comprising a human wild-type IgG1Fc region.

### Cysteine Engineered Variants

In some embodiments, it may be desirable to create cysteine engineered anti-TSLP antibodies (such as a full-length anti-TSLP antibody) in which one or more amino acid residues are substituted with cysteine residues. In some embodiments, the substituted residues occur at accessible sites of the anti-TSLP antibody. By substituting those residues with cysteine, reactive thiol groups are thereby positioned at accessible sites of the anti-TSLP antibody and may be used to conjugate the anti-TSLP antibody to other moieties, such as drug moieties or linker-drug moieties, to create an anti-TSLP immunoconjugate, as described further herein. Cysteine engineered anti-TSLP antibodies (e.g., full-length anti-TSLP antibodies) may be generated as described, e.g., in U.S. Pat. No. 7,521,541.

### Derivatives

In some embodiments, an anti-TSLP antibody (such as a full-length anti-TSLP antibody) provided herein may be further modified to contain additional non-proteinaceous moieties that are known in the art and readily available. The moieties suitable for derivatization of the anti-TSLP antibody include but are not limited to water soluble polymers. Non-limiting examples of water soluble polymers include, but are not limited to, polyethylene glycol (PEG), copolymers of ethylene glycol/propylene glycol, carboxymethylcellulose, dextran, polyvinyl alcohol, polyvinyl pyrrolidone, poly-1,3-dioxolane, poly-1,3,6-trioxane, ethylene/maleic anhydride copolymer, polyaminoacids (either homopolymers or random copolymers), and dextran or poly(n-vinyl pyrrolidone)polyethylene glycol, propropylene glycol homopolymers, prolypropylene oxide/ethylene oxide co-polymers, polyoxyethylated polyols (e.g., glycerol), polyvinyl alcohol, and mixtures thereof. Polyethylene glycol propionaldehyde may have advantages in manufacturing due to its stability in water. The polymer may be of any molecular weight, and may be branched or unbranched. The number of polymers attached to the anti-TSLP antibody may vary, and if more than one polymer are attached, they can be the same or different molecules. In general, the number and/or type of polymers used for derivatization can be determined based on considerations including, but not limited to, the particular properties or functions of anti-TSLP antibody to be improved, whether the anti-TSLP antibody derivative will be used in a therapy under defined conditions, etc.

### Pharmaceutical Compositions

Also provided herein are compositions (such as pharmaceutical compositions, also referred to herein as formulations) comprising any of the anti-TSLP antibodies (such as a full-length anti-TSLP antibody), nucleic acids encoding the antibodies, vectors comprising the nucleic acids encoding the antibodies, or host cells comprising the nucleic acids or vectors described herein. In some embodiments, there is provided a pharmaceutical composition comprising any one of the anti-TSLP antibodies described herein and a pharmaceutically acceptable carrier.

Suitable formulations of the anti-TSLP antibodies are obtained by mixing an anti-TSLP antibody having the desired degree of purity with optional pharmaceutically acceptable carriers, excipients or stabilizers (Remington's Pharmaceutical Sciences 16th edition, Osol, A. Ed. (1980)), in the form of lyophilized formulations or aqueous solutions. Acceptable carriers, excipients, or stabilizers are nontoxic to recipients at the dosages and concentrations employed, and include buffers such as phosphate, citrate, and other organic acids; antioxidants including ascorbic acid and methionine; preservatives (such as octadecyldimethylbenzyl ammonium chloride; hexamethonium chloride; benzalkonium chloride, benzethonium chloride; phenol, butyl or benzyl alcohol; alkyl parabens such as methyl or propylparaben; catechol; resorcinol; cyclohexanol; 3-pentanol; and m-cresol); low molecular weight (less than about 10 residues) polypeptides; proteins, such as serum albumin, gelatin, or immunoglobulins; hydrophilic polymers such as olyvinylpyrrolidone; amino acids such as glycine, glutamine, asparagine, histidine, arginine, or lysine; monosaccharides, disaccharides, and other carbohydrates including glucose, mannose, or dextrins; chelating agents such as EDTA; sugars such as sucrose, mannitol, trehalose or sorbitol; salt-forming counter-ions such as sodium; metal complexes (e.g. Zn-protein complexes); and/or non-ionic surfactants such as TWEEN™, PLURONICS™ or polyethylene glycol (PEG). Exemplary formulations are described in WO98/56418, expressly incorporated herein by reference. Lyophilized formulations adapted for subcutaneous administration are described in WO97/04801. Such lyophilized formulations may be reconstituted with a suitable diluent to a high protein concentration and the reconstituted formulation may be administered subcutaneously to the individual to be treated herein. Lipofectins or liposomes can be used to deliver the anti-TSLP antibodies of this application into cells.

The formulation herein may also contain one or more active compounds in addition to the anti-TSLP antibody (such as a full-length anti-TSLP antibody) as necessary for the particular indication being treated, preferably those with complementary activities that do not adversely affect each other. For example, it may be desirable to further provide for example, another TSLP antagonist, inhaled, intranasal or parenteral corticosteroids, a bronchodilator, an anti-leukotriene antagonist, or an anti-histamine agent, or combinations thereof, or an anti-neoplastic agent, a growth inhibitory agent, a cytotoxic agent, or a chemotherapeutic agent in addition to the anti-TSLP antibody. Such molecules are suitably present in combination in amounts that are effective for the purpose intended. The effective amount of such other agents depends on the amount of anti-TSLP antibody present in the formulation, the type of disease or disorder or treatment, and other factors discussed above. These are generally used in the same dosages and with administration routes as described herein or about from 1 to 99% of the heretofore employed dosages.

The anti-TSLP antibodies (e.g., full-length anti-TSLP antibodies) may also be entrapped in microcapsules prepared, for example, by coacervation techniques or by interfacial polymerization, for example, hydroxymethylcellulose or gelatin-microcapsules and poly-(methylmethacylate) microcapsules, respectively, in colloidal drug delivery systems (for example, liposomes, albumin microspheres, microemulsions, nano-particles and nanocapsules) or in macroemulsions. Sustained-release preparations may be prepared.

Sustained-release preparations of the anti-TSLP antibodies (e.g., full-length anti-TSLP antibodies) can be prepared. Suitable examples of sustained-release preparations include semipermeable matrices of solid hydrophobic polymers containing the antibody (or fragment thereof), which matrices are in the form of shaped articles, e.g., films, or microcapsules. Examples of sustained-release matrices include polyesters, hydrogels (for example, poly(2-hydroxyethyl-methacrylate), or poly(vinylalcohol)), polylactides (U.S. Pat. No. 3,773,919), copolymers of L-glutamic acid and ethyl-L-glutamate, non-degradable ethylene-vinyl acetate, degradable lactic acid-glycolic acid copolymers such as the LUPRON DEPOT™ (injectable microspheres composed of lactic acid-glycolic acid copolymer and leuprolide acetate), and poly-D (−)-3-hydroxybutyric acid. While polymers such as ethylene-vinyl acetate and lactic acid-glycolic acid enable release of molecules for over 100 days, certain hydro gels release proteins for shorter time periods. When encapsulated antibody remain in the body for a long time, they can denature or aggregate as a result of exposure to moisture at 37° C., resulting in a loss of biological activity and possible changes in immunogenicity. Rational strategies can be devised for stabilization of anti-TSLP antibodies depending on the mechanism involved. For example, if the aggregation mechanism is discovered to be intermolecular S—S bond formation through thio-disulfide interchange, stabilization can be achieved by modifying sulfhydryl residues, lyophilizing from acidic solutions, controlling moisture content, using appropriate additives, and developing specific polymer matrix compositions.

In some embodiments, the anti-TSLP antibody (such as a full-length anti-TSLP antibody) is formulated in a buffer comprising a citrate, NaCl, acetate, succinate, glycine, polysorbate 80 (Tween 80), or any combination of the foregoing.

The formulations to be used for in vivo administration must be sterile. This is readily accomplished by, e.g., filtration through sterile filtration membranes.

### Methods of Treatment Using Anti-TSLP Antibodies

The anti-TSLP antibodies (e.g., full-length anti-TSLP antibodies) and/or compositions of the application can be administered to individuals (e.g., mammals such as humans) to treat a disease and/or disorder associated with TSLP signaling. These diseases include, but are not limited to, asthma, idiopathic pulmonary fibrosis, atopic dermatitis, allergic conjunctivitis, allergic rhinitis, Netherton syndrome, eosinophilic esophagitis (EoE), food allergy, allergic diarrhoea, eosinophilic gastroenteritis, allergic bronchopulmonary aspergillosis (ABPA), allergic fungal sinusitis, cancer, rheumatoid arthritis, chronic obstructive pulmonary disease (COPD), systemic sclerosis, keloids, ulcerative colitis, chronic rhinosinusitis (CRS), nasal polyposis, chronic eosinophilic pneumonia, eosinophilic bronchitis, coeliac disease, Churg-Strauss syndrome, eosinophilic myalgia syndrome, hypereosinophilic syndrome, eosinophilic granulomatosis with polyangiitis, and inflammatory bowel disease. The present application thus in some embodiments provides a method of treating a disease or condition associated with TSLP signaling (e.g., inflammatory diseases) in an individual comprising administering to the individual an effective amount of a composition (such as a pharmaceutical composition) comprising an anti-TSLP antibody (e.g., a full-length anti-TSLP antibody), such as any one of the anti-TSLP antibodies (e.g., full-length anti-TSLP antibodies) described herein. In some embodiments, the individual is human.

For example, in some embodiments, there is provided a method of treating an individual having a disease or condition associated with TSLP signaling (e.g., inflammatory diseases) comprising administering to the individual an effective amount of a pharmaceutical composition comprising an anti-TSLP antibody (e.g., full-length anti-TSLP antibody) comprising a heavy chain variable domain (V_{H}) comprising an HC-CDR1 comprising SGYGWS (SEQ ID NO: 1); an HC-CDR2 comprising YX₁SYYGSX₂SYNPSLKS (SEQ ID NO: 103), wherein X₁ is I or F, and X₂ is I or T; and an HC-CDR3 comprising TNLLYFX₁X₂ (SEQ ID NO: 104), wherein X₁ is D or E, and X₂ is S or Y; and a light chain variable domain (V_{L}) comprising a light chain complementarity determining region (LC-CDR) 1 comprising RASQX₁X₂SX₃X₄LA (SEQ ID NO: 105), wherein X₁ is G or S, X₂ is V or I, X₃ is N or S, and X₄ is N or Y; a LC-CDR2 comprising DX₁SX₂X₃X₄X₅(SEQ ID NO: 106), wherein X₁ is A or T, X₂ is S or N, X₃ is R or L, X₄ is A or Q, and X₅ is T or S; and a LC-CDR3 comprising QQYSX₁WPX₂YX₃ (SEQ ID NO: 107), wherein X₁ is D or N, X₂ is Q or E, and X₃ is T or S. In some embodiments, the anti-TSLP antibody is a full-length antibody. In some embodiments, the full-length anti-TSLP antibody is an IgG1 or IgG4 antibody. In some embodiments, the disease or condition is selected from the group consisting of asthma, idiopathic pulmonary fibrosis, atopic dermatitis, allergic conjunctivitis, allergic rhinitis, Netherton syndrome, eosinophilic esophagitis (EoE), food allergy, allergic diarrhoea, eosinophilic gastroenteritis, allergic bronchopulmonary aspergillosis (ABPA), allergic fungal sinusitis, cancer, rheumatoid arthritis, chronic obstructive pulmonary disease (COPD), systemic sclerosis, keloids, ulcerative colitis, chronic rhinosinusitis (CRS), nasal polyposis, chronic eosinophilic pneumonia, eosinophilic bronchitis, coeliac disease, Churg-Strauss syndrome, eosinophilic myalgia syndrome, hypereosinophilic syndrome, eosinophilic granulomatosis with polyangiitis, and inflammatory bowel disease. In some embodiments, the individual is human.

In some embodiments, there is provided a method of treating an individual having a disease or condition associated with TSLP signaling (e.g., inflammatory diseases) comprising administering to the individual an effective amount of a pharmaceutical composition comprising an anti-TSLP antibody (e.g., full-length anti-TSLP antibody) comprising: a V_{H} comprising an HC-CDR1 comprising the amino acid sequence of SEQ ID NO: 1, an HC-CDR2 comprising the amino acid sequence of any one of SEQ ID NOs: 7-9, and an HC-CDR3 comprising the amino acid sequence of any one of SEQ ID NOs: 16-18, or a variant thereof comprising up to 5 amino acid substitutions in the HC-CDRs; and a V_{L} comprising an LC-CDR1 comprising the amino acid sequence of any one of SEQ ID NOs: 25-28, an LC-CDR2 comprising the amino acid sequence of any one of SEQ ID NOs: 39-41, and an LC-CDR3 comprising the amino acid sequence of any one of SEQ ID NOs: 50-53, or a variant thereof comprising up to 5 amino acid substitutions in the LC-CDRs.

In some embodiments, there is provided a method of treating an individual having a disease or condition associated with TSLP signaling (e.g., inflammatory diseases) comprising administering to the individual an effective amount of a pharmaceutical composition comprising an anti-TSLP antibody (e.g., full-length anti-TSLP antibody) comprising a V_{H} comprising the amino acid sequence of any one of SEQ ID NOs: 65-72, or a variant thereof having at least about 90% (for example at least about any of 91%, 92%, 93%, 94%, 95%, 96%, 97%, 98%, or 99%) sequence identity to the amino acid sequence of any one of SEQ ID NOs: 65-72, and a V_{L} comprising the amino acid sequence of any one of SEQ ID NOs: 84-90, or a variant thereof having at least about 90% (for example at least about any of 91%, 92%, 93%, 94%, 95%, 96%, 97%, 98%, or 99%) sequence identity to the amino acid sequence of any one of SEQ ID NOs: 84-90.

In some embodiments, the anti-TSLP antibody provided herein is a full-length anti-TSLP antibody comprising IgG1 or IgG4 constant domains. In some embodiments, the IgG1 is human IgG1. In some embodiments, the IgG4 is human IgG4. In some embodiments, the heavy chain constant region comprises or consists of the amino acid sequence of SEQ ID NO: 124. In some embodiments, the heavy chain constant region comprises or consists of the amino acid sequence of SEQ ID NO: 125. In some embodiments, the light chain constant region comprises or consists of the amino acid sequence of SEQ ID NO: 126. In some embodiments, the light chain constant region comprises or consists of the amino acid sequence of SEQ ID NO: 127.

In some embodiments, there is provided a method of treating an individual having a disease or condition associated with TSLP signaling (e.g., inflammatory diseases) comprising administering to the individual an effective amount of a pharmaceutical composition comprising an anti-TSLP antibody (e.g., full-length anti-TSLP antibody) comprising: a V_{H} comprising an HC-CDR1 comprising the amino acid sequence of SEQ ID NO: 1, an HC-CDR2 comprising the amino acid sequence of SEQ ID NO: 7, and an HC-CDR3 comprising the amino acid sequence of SEQ ID NO: 16, or a variant thereof comprising up to 5 amino acid substitutions in the HC-CDRs; and a V_{L} comprising an LC-CDR1 comprising the amino acid sequence of SEQ ID NO: 25, an LC-CDR2 comprising the amino acid sequence of SEQ ID NO: 39, and an LC-CDR3 comprising the amino acid sequence of SEQ ID NO: 50, or a variant thereof comprising up to 5 amino acid substitutions in the LC-CDRs.

In some embodiments, the anti-TSLP antibody provided herein comprises a V_{H} comprising the amino acid sequence of SEQ ID NO: 65 and a V_{L} comprising the amino acid sequence of SEQ ID NO: 84. In some embodiments, the anti-TSLP antibody provided herein is a full-length anti-TSLP antibody comprising IgG1 or IgG4 constant domains. In some embodiments, the IgG1 is human IgG1. In some embodiments, the IgG4 is human IgG4. In some embodiments, the heavy chain constant region comprises or consists of the amino acid sequence of SEQ ID NO: 124. In some embodiments, the heavy chain constant region comprises or consists of the amino acid sequence of SEQ ID NO: 125. In some embodiments, the light chain constant region comprises or consists of the amino acid sequence of SEQ ID NO: 126. In some embodiments, the light chain constant region comprises or consists of the amino acid sequence of SEQ ID NO: 127.

In some embodiments, the anti-TSLP antibody provided herein comprises a V_{H} comprising the amino acid sequence of SEQ ID NO: 65 and a V_{L} comprising the amino acid sequence of SEQ ID NO: 85. In some embodiments, the anti-TSLP antibody provided herein is a full-length anti-TSLP antibody comprising IgG1 or IgG4 constant domains. In some embodiments, the IgG1 is human IgG1. In some embodiments, the IgG4 is human IgG4. In some embodiments, the heavy chain constant region comprises or consists of the amino acid sequence of SEQ ID NO: 124. In some embodiments, the heavy chain constant region comprises or consists of the amino acid sequence of SEQ ID NO: 125. In some embodiments, the light chain constant region comprises or consists of the amino acid sequence of SEQ ID NO: 126. In some embodiments, the light chain constant region comprises or consists of the amino acid sequence of SEQ ID NO: 127.

In some embodiments, the anti-TSLP antibody provided herein comprises a V_{H} comprising the amino acid sequence of SEQ ID NO: 66 and a V_{L} comprising the amino acid sequence of SEQ ID NO: 84. In some embodiments, the anti-TSLP antibody provided herein is a full-length anti-TSLP antibody comprising IgG1 or IgG4 constant domains. In some embodiments, the IgG1 is human IgG1. In some embodiments, the IgG4 is human IgG4. In some embodiments, the heavy chain constant region comprises or consists of the amino acid sequence of SEQ ID NO: 124. In some embodiments, the heavy chain constant region comprises or consists of the amino acid sequence of SEQ ID NO: 125. In some embodiments, the light chain constant region comprises or consists of the amino acid sequence of SEQ ID NO: 126. In some embodiments, the light chain constant region comprises or consists of the amino acid sequence of SEQ ID NO: 127.

In some embodiments, the anti-TSLP antibody provided herein comprises a V_{H} comprising the amino acid sequence of SEQ ID NO: 66 and a V_{L} comprising the amino acid sequence of SEQ ID NO: 85. In some embodiments, the anti-TSLP antibody provided herein is a full-length anti-TSLP antibody comprising IgG1 or IgG4 constant domains. In some embodiments, the IgG1 is human IgG1. In some embodiments, the IgG4 is human IgG4. In some embodiments, the heavy chain constant region comprises or consists of the amino acid sequence of SEQ ID NO: 124. In some embodiments, the heavy chain constant region comprises or consists of the amino acid sequence of SEQ ID NO: 125. In some embodiments, the light chain constant region comprises or consists of the amino acid sequence of SEQ ID NO: 126. In some embodiments, the light chain constant region comprises or consists of the amino acid sequence of SEQ ID NO: 127.

In some embodiments, the anti-TSLP antibody provided herein comprises a V_{H} comprising the amino acid sequence of SEQ ID NO: 67 and a V_{L} comprising the amino acid sequence of SEQ ID NO: 84. In some embodiments, the anti-TSLP antibody provided herein is a full-length anti-TSLP antibody comprising IgG1 or IgG4 constant domains. In some embodiments, the IgG1 is human IgG1. In some embodiments, the IgG4 is human IgG4. In some embodiments, the heavy chain constant region comprises or consists of the amino acid sequence of SEQ ID NO: 124. In some embodiments, the heavy chain constant region comprises or consists of the amino acid sequence of SEQ ID NO: 125. In some embodiments, the light chain constant region comprises or consists of the amino acid sequence of SEQ ID NO: 126. In some embodiments, the light chain constant region comprises or consists of the amino acid sequence of SEQ ID NO: 127.

In some embodiments, the anti-TSLP antibody provided herein comprises a V_{H} comprising the amino acid sequence of SEQ ID NO: 67 and a V_{L} comprising the amino acid sequence of SEQ ID NO: 85. In some embodiments, the anti-TSLP antibody provided herein is a full-length anti-TSLP antibody comprising IgG1 or IgG4 constant domains. In some embodiments, the IgG1 is human IgG1. In some embodiments, the IgG4 is human IgG4. In some embodiments, the heavy chain constant region comprises or consists of the amino acid sequence of SEQ ID NO: 124. In some embodiments, the heavy chain constant region comprises or consists of the amino acid sequence of SEQ ID NO: 125. In some embodiments, the light chain constant region comprises or consists of the amino acid sequence of SEQ ID NO: 126. In some embodiments, the light chain constant region comprises or consists of the amino acid sequence of SEQ ID NO: 127.

In some embodiments, the anti-TSLP antibody provided herein comprises a V_{H} comprising the amino acid sequence of SEQ ID NO: 68 and a V_{L} comprising the amino acid sequence of SEQ ID NO: 84. In some embodiments, the anti-TSLP antibody provided herein is a full-length anti-TSLP antibody comprising IgG1 or IgG4 constant domains. In some embodiments, the IgG1 is human IgG1. In some embodiments, the IgG4 is human IgG4. In some embodiments, the heavy chain constant region comprises or consists of the amino acid sequence of SEQ ID NO: 124. In some embodiments, the heavy chain constant region comprises or consists of the amino acid sequence of SEQ ID NO: 125. In some embodiments, the light chain constant region comprises or consists of the amino acid sequence of SEQ ID NO: 126. In some embodiments, the light chain constant region comprises or consists of the amino acid sequence of SEQ ID NO: 127.

In some embodiments, the anti-TSLP antibody provided herein comprises a V_{H} comprising the amino acid sequence of SEQ ID NO: 68 and a V_{L} comprising the amino acid sequence of SEQ ID NO: 85. In some embodiments, the anti-TSLP antibody provided herein is a full-length anti-TSLP antibody comprising IgG1 or IgG4 constant domains. In some embodiments, the IgG1 is human IgG1. In some embodiments, the IgG4 is human IgG4. In some embodiments, the heavy chain constant region comprises or consists of the amino acid sequence of SEQ ID NO: 124. In some embodiments, the heavy chain constant region comprises or consists of the amino acid sequence of SEQ ID NO: 125. In some embodiments, the light chain constant region comprises or consists of the amino acid sequence of SEQ ID NO: 126. In some embodiments, the light chain constant region comprises or consists of the amino acid sequence of SEQ ID NO: 127.

In some embodiments, there is provided a method of treating an individual having a disease or condition associated with TSLP signaling (e.g., inflammatory diseases) comprising administering to the individual an effective amount of a pharmaceutical composition comprising an anti-TSLP antibody (e.g., full-length anti-TSLP antibody) comprising: a V_{H} comprising an HC-CDR1 comprising the amino acid sequence of SEQ ID NO: 1, an HC-CDR2 comprising the amino acid sequence of SEQ ID NO: 8, and an HC-CDR3 comprising the amino acid sequence of SEQ ID NO: 16, or a variant thereof comprising up to 5 amino acid substitutions in the HC-CDRs; and a V_{L} comprising an LC-CDR1 comprising the amino acid sequence of SEQ ID NO: 25, an LC-CDR2 comprising the amino acid sequence of SEQ ID NO: 39, and an LC-CDR3 comprising the amino acid sequence of SEQ ID NO: 51, or a variant thereof comprising up to 5 amino acid substitutions in the LC-CDRs.

In some embodiments, the anti-TSLP antibody provided herein comprises a V_{H} comprising the amino acid sequence of SEQ ID NO: 69 and a V_{L} comprising the amino acid sequence of SEQ ID NO: 86. In some embodiments, the anti-TSLP antibody provided herein is a full-length anti-TSLP antibody comprising IgG1 or IgG4 constant domains. In some embodiments, the IgG1 is human IgG1. In some embodiments, the IgG4 is human IgG4. In some embodiments, the heavy chain constant region comprises or consists of the amino acid sequence of SEQ ID NO: 124. In some embodiments, the heavy chain constant region comprises or consists of the amino acid sequence of SEQ ID NO: 125. In some embodiments, the light chain constant region comprises or consists of the amino acid sequence of SEQ ID NO: 126. In some embodiments, the light chain constant region comprises or consists of the amino acid sequence of SEQ ID NO: 127.

In some embodiments, there is provided a method of treating an individual having a disease or condition associated with TSLP signaling (e.g., inflammatory diseases) comprising administering to the individual an effective amount of a pharmaceutical composition comprising an anti-TSLP antibody (e.g., full-length anti-TSLP antibody) comprising: a V_{H} comprising an HC-CDR1 comprising the amino acid sequence of SEQ ID NO: 1, an HC-CDR2 comprising the amino acid sequence of SEQ ID NO: 8, and an HC-CDR3 comprising the amino acid sequence of SEQ ID NO: 17, or a variant thereof comprising up to 5 amino acid substitutions in the HC-CDRs; and a V_{L} comprising an LC-CDR1 comprising the amino acid sequence of SEQ ID NO: 26, an LC-CDR2 comprising the amino acid sequence of SEQ ID NO: 40, and an LC-CDR3 comprising the amino acid sequence of SEQ ID NO: 52, or a variant thereof comprising up to 5 amino acid substitutions in the LC-CDRs.

In some embodiments, the anti-TSLP antibody provided herein comprises a V_{H} comprising the amino acid sequence of SEQ ID NO: 70 and a V_{L} comprising the amino acid sequence of SEQ ID NO: 87. In some embodiments, the anti-TSLP antibody provided herein is a full-length anti-TSLP antibody comprising IgG1 or IgG4 constant domains. In some embodiments, the IgG1 is human IgG1. In some embodiments, the IgG4 is human IgG4. In some embodiments, the heavy chain constant region comprises or consists of the amino acid sequence of SEQ ID NO: 124. In some embodiments, the heavy chain constant region comprises or consists of the amino acid sequence of SEQ ID NO: 125. In some embodiments, the light chain constant region comprises or consists of the amino acid sequence of SEQ ID NO: 126. In some embodiments, the light chain constant region comprises or consists of the amino acid sequence of SEQ ID NO: 127.

In some embodiments, there is provided a method of treating an individual having a disease or condition associated with TSLP signaling (e.g., inflammatory diseases) comprising administering to the individual an effective amount of a pharmaceutical composition comprising an anti-TSLP antibody (e.g., full-length anti-TSLP antibody) comprising: a V_{H} comprising an HC-CDR1 comprising the amino acid sequence of SEQ ID NO: 1, an HC-CDR2 comprising the amino acid sequence of SEQ ID NO: 8, and an HC-CDR3 comprising the amino acid sequence of SEQ ID NO: 16, or a variant thereof comprising up to 5 amino acid substitutions in the HC-CDRs; and a V_{L} comprising an LC-CDR1 comprising the amino acid sequence of SEQ ID NO: 27, an LC-CDR2 comprising the amino acid sequence of SEQ ID NO: 41, and an LC-CDR3 comprising the amino acid sequence of SEQ ID NO: 52, or a variant thereof comprising up to 5 amino acid substitutions in the LC-CDRs.

In some embodiments, the anti-TSLP antibody provided herein comprises a V_{H} comprising the amino acid sequence of SEQ ID NO: 69 and a V_{L} comprising the amino acid sequence of SEQ ID NO: 88. In some embodiments, the anti-TSLP antibody provided herein is a full-length anti-TSLP antibody comprising IgG1 or IgG4 constant domains. In some embodiments, the IgG1 is human IgG1. In some embodiments, the IgG4 is human IgG4. In some embodiments, the heavy chain constant region comprises or consists of the amino acid sequence of SEQ ID NO: 124. In some embodiments, the heavy chain constant region comprises or consists of the amino acid sequence of SEQ ID NO: 125. In some embodiments, the light chain constant region comprises or consists of the amino acid sequence of SEQ ID NO: 126. In some embodiments, the light chain constant region comprises or consists of the amino acid sequence of SEQ ID NO: 127.

In some embodiments, there is provided a method of treating an individual having a disease or condition associated with TSLP signaling (e.g., inflammatory diseases) comprising administering to the individual an effective amount of a pharmaceutical composition comprising an anti-TSLP antibody (e.g., full-length anti-TSLP antibody) comprising: a V_{H} comprising an HC-CDR1 comprising the amino acid sequence of SEQ ID NO: 1, an HC-CDR2 comprising the amino acid sequence of SEQ ID NO: 9, and an HC-CDR3 comprising the amino acid sequence of SEQ ID NO: 16, or a variant thereof comprising up to 5 amino acid substitutions in the HC-CDRs; and a V_{L} comprising an LC-CDR1 comprising the amino acid sequence of SEQ ID NO: 28, an LC-CDR2 comprising the amino acid sequence of SEQ ID NO: 39, and an LC-CDR3 comprising the amino acid sequence of SEQ ID NO: 52, or a variant thereof comprising up to 5 amino acid substitutions in the LC-CDRs.

In some embodiments, the anti-TSLP antibody provided herein comprises a V_{H} comprising the amino acid sequence of SEQ ID NO: 71 and a V_{L} comprising the amino acid sequence of SEQ ID NO: 89. In some embodiments, the anti-TSLP antibody provided herein is a full-length anti-TSLP antibody comprising IgG1 or IgG4 constant domains. In some embodiments, the IgG1 is human IgG1. In some embodiments, the IgG4 is human IgG4. In some embodiments, the heavy chain constant region comprises or consists of the amino acid sequence of SEQ ID NO: 124. In some embodiments, the heavy chain constant region comprises or consists of the amino acid sequence of SEQ ID NO: 125. In some embodiments, the light chain constant region comprises or consists of the amino acid sequence of SEQ ID NO: 126. In some embodiments, the light chain constant region comprises or consists of the amino acid sequence of SEQ ID NO: 127.

In some embodiments, there is provided a method of treating an individual having a disease or condition associated with TSLP signaling (e.g., inflammatory diseases) comprising administering to the individual an effective amount of a pharmaceutical composition comprising an anti-TSLP antibody (e.g., full-length anti-TSLP antibody) comprising: a V_{H} comprising an HC-CDR1 comprising the amino acid sequence of SEQ ID NO: 1, an HC-CDR2 comprising the amino acid sequence of SEQ ID NO: 8, and an HC-CDR3 comprising the amino acid sequence of SEQ ID NO: 18, or a variant thereof comprising up to 5 amino acid substitutions in the HC-CDRs; and a V_{L} comprising an LC-CDR1 comprising the amino acid sequence of SEQ ID NO: 27, an LC-CDR2 comprising the amino acid sequence of SEQ ID NO: 39, and an LC-CDR3 comprising the amino acid sequence of SEQ ID NO: 53, or a variant thereof comprising up to 5 amino acid substitutions in the LC-CDRs.

In some embodiments, the anti-TSLP antibody provided herein comprises a V_{H} comprising the amino acid sequence of SEQ ID NO: 72 and a V_{L} comprising the amino acid sequence of SEQ ID NO: 90. In some embodiments, the anti-TSLP antibody provided herein is a full-length anti-TSLP antibody comprising IgG1 or IgG4 constant domains. In some embodiments, the IgG1 is human IgG1. In some embodiments, the IgG4 is human IgG4. In some embodiments, the heavy chain constant region comprises or consists of the amino acid sequence of SEQ ID NO: 124. In some embodiments, the heavy chain constant region comprises or consists of the amino acid sequence of SEQ ID NO: 125. In some embodiments, the light chain constant region comprises or consists of the amino acid sequence of SEQ ID NO: 126. In some embodiments, the light chain constant region comprises or consists of the amino acid sequence of SEQ ID NO: 127.

For example, in some embodiments, there is provided a method of treating an individual having a disease or condition associated with TSLP signaling (e.g., inflammatory diseases) comprising administering to the individual an effective amount of a pharmaceutical composition comprising an anti-TSLP antibody (e.g., full-length anti-TSLP antibody) comprising a heavy chain variable domain (V_{H}) comprising an HC-CDR1 comprising SYGIX₁ (SEQ ID NO: 108), wherein X₁ is N or S; an HC-CDR2 comprising VIX₁PLX₂X₃VX₄X₅YAEKFQG (SEQ ID NO: 109), wherein X₁ is V or I, X₂ is V or L, X₃ is G or D, X₄ is T or P, and X₅ is I or N; and an HC-CDR3 comprising GX₁EYFYWYFDL (SEQ ID NO: 110), wherein X₁ is Q or A; and a light chain variable domain (V_{L}) comprising a light chain complementarity determining region (LC-CDR) 1 comprising X₁GX₂X₃SDIGGYX₄RVS (SEQ ID NO: 111), wherein X₁ is S or T, X₂ is S or T, X₃ is S, T, I or N, and X₄ is N or D; a LC-CDR2 comprising X₁X₂X₃KRX₄S (SEQ ID NO: 112), wherein X₁ is D, G or E, X₂ is V, F or I, X₃ is S or N, and X₄ is P or S; and a LC-CDR3 comprising X₁SYAX₂X₃X₄X₅FX₆X₇(SEQ ID NO: 113), wherein X₁ is S or T, X₂ is G or S, X₃ is T or G, X₄ is D or H, X₅ is T or I, X₆ is I, G, V or A, and X₇ is L, I or F. In some embodiments, the anti-TSLP antibody is a full-length antibody. In some embodiments, the full-length anti-TSLP antibody is an IgG1 or IgG4 antibody. In some embodiments, the disease or condition is selected from the group consisting of asthma, idiopathic pulmonary fibrosis, atopic dermatitis, allergic conjunctivitis, allergic rhinitis, Netherton syndrome, eosinophilic esophagitis (EoE), food allergy, allergic diarrhoea, eosinophilic gastroenteritis, allergic bronchopulmonary aspergillosis (ABPA), allergic fungal sinusitis, cancer, rheumatoid arthritis, chronic obstructive pulmonary disease (COPD), systemic sclerosis, keloids, ulcerative colitis, chronic rhinosinusitis (CRS), nasal polyposis, chronic eosinophilic pneumonia, eosinophilic bronchitis, coeliac disease, Churg-Strauss syndrome, eosinophilic myalgia syndrome, hypereosinophilic syndrome, eosinophilic granulomatosis with polyangiitis, and inflammatory bowel disease. In some embodiments, the individual is human.

In some embodiments, there is provided a method of treating an individual having a disease or condition associated with TSLP signaling (e.g., inflammatory diseases) comprising administering to the individual an effective amount of a pharmaceutical composition comprising an anti-TSLP antibody (e.g., full-length anti-TSLP antibody) comprising: a V_{H} comprising an HC-CDR1 comprising the amino acid sequence of any one of SEQ ID NOs: 2-3, an HC-CDR2 comprising the amino acid sequence of any one of SEQ ID NOs: 10-12, and an HC-CDR3 comprising the amino acid sequence of any one of SEQ ID NOs: 19-20, or a variant thereof comprising up to 5 amino acid substitutions in the HC-CDRs; and a V_{L} comprising an LC-CDR1 comprising the amino acid sequence of any one of SEQ ID NOs: 29-34, an LC-CDR2 comprising the amino acid sequence of any one of SEQ ID NOs: 42-47, and an LC-CDR3 comprising the amino acid sequence of any one of SEQ ID NOs: 54-60, or a variant thereof comprising up to 5 amino acid substitutions in the LC-CDRs.

In some embodiments, there is provided a method of treating an individual having a disease or condition associated with TSLP signaling (e.g., inflammatory diseases) comprising administering to the individual an effective amount of a pharmaceutical composition comprising an anti-TSLP antibody (e.g., full-length anti-TSLP antibody) comprising a V_{H} comprising the amino acid sequence of any one of SEQ ID NOs: 73-78, or a variant thereof having at least about 90% (for example at least about any of 91%, 92%, 93%, 94%, 95%, 96%, 97%, 98%, or 99%) sequence identity to the amino acid sequence of any one of SEQ ID NOs: 73-78, and a V_{L} comprising the amino acid sequence of any one of SEQ ID NOs: 91-97, or a variant thereof having at least about 90% (for example at least about any of 91%, 92%, 93%, 94%, 95%, 96%, 97%, 98%, or 99%) sequence identity to the amino acid sequence of any one of SEQ ID NOs: 91-97.

In some embodiments, the anti-TSLP antibody provided herein is a full-length anti-TSLP antibody comprising IgG1 or IgG4 constant domains. In some embodiments, the IgG1 is human IgG1. In some embodiments, the IgG4 is human IgG4. In some embodiments, the heavy chain constant region comprises or consists of the amino acid sequence of SEQ ID NO: 124. In some embodiments, the heavy chain constant region comprises or consists of the amino acid sequence of SEQ ID NO: 125. In some embodiments, the light chain constant region comprises or consists of the amino acid sequence of SEQ ID NO: 126. In some embodiments, the light chain constant region comprises or consists of the amino acid sequence of SEQ ID NO: 127.

In some embodiments, there is provided a method of treating an individual having a disease or condition associated with TSLP signaling (e.g., inflammatory diseases) comprising administering to the individual an effective amount of a pharmaceutical composition comprising an anti-TSLP antibody (e.g., full-length anti-TSLP antibody) comprising: a V_{H} comprising an HC-CDR1 comprising the amino acid sequence of SEQ ID NO: 2, an HC-CDR2 comprising the amino acid sequence of SEQ ID NO: 10, and an HC-CDR3 comprising the amino acid sequence of SEQ ID NO: 19, or a variant thereof comprising up to 5 amino acid substitutions in the HC-CDRs; and a V_{L} comprising an LC-CDR1 comprising the amino acid sequence of SEQ ID NO: 29, an LC-CDR2 comprising the amino acid sequence of SEQ ID NO: 42, and an LC-CDR3 comprising the amino acid sequence of SEQ ID NO: 54, or a variant thereof comprising up to 5 amino acid substitutions in the LC-CDRs.

In some embodiments, the anti-TSLP antibody provided herein comprises a V_{H} comprising the amino acid sequence of SEQ ID NO: 73 and a V_{L} comprising the amino acid sequence of SEQ ID NO: 91. In some embodiments, the anti-TSLP antibody provided herein is a full-length anti-TSLP antibody comprising IgG1 or IgG4 constant domains. In some embodiments, the IgG1 is human IgG1. In some embodiments, the IgG4 is human IgG4. In some embodiments, the heavy chain constant region comprises or consists of the amino acid sequence of SEQ ID NO: 124. In some embodiments, the heavy chain constant region comprises or consists of the amino acid sequence of SEQ ID NO: 125. In some embodiments, the light chain constant region comprises or consists of the amino acid sequence of SEQ ID NO: 126. In some embodiments, the light chain constant region comprises or consists of the amino acid sequence of SEQ ID NO: 127.

In some embodiments, the anti-TSLP antibody provided herein comprises a V_{H} comprising the amino acid sequence of SEQ ID NO: 74 and a V_{L} comprising the amino acid sequence of SEQ ID NO: 91. In some embodiments, the anti-TSLP antibody provided herein is a full-length anti-TSLP antibody comprising IgG1 or IgG4 constant domains. In some embodiments, the IgG1 is human IgG1. In some embodiments, the IgG4 is human IgG4. In some embodiments, the heavy chain constant region comprises or consists of the amino acid sequence of SEQ ID NO: 124. In some embodiments, the heavy chain constant region comprises or consists of the amino acid sequence of SEQ ID NO: 125. In some embodiments, the light chain constant region comprises or consists of the amino acid sequence of SEQ ID NO: 126. In some embodiments, the light chain constant region comprises or consists of the amino acid sequence of SEQ ID NO: 127.

In some embodiments, the anti-TSLP antibody provided herein comprises a V_{H} comprising the amino acid sequence of SEQ ID NO: 75 and a V_{L} comprising the amino acid sequence of SEQ ID NO: 91. In some embodiments, the anti-TSLP antibody provided herein is a full-length anti-TSLP antibody comprising IgG1 or IgG4 constant domains. In some embodiments, the IgG1 is human IgG1. In some embodiments, the IgG4 is human IgG4. In some embodiments, the heavy chain constant region comprises or consists of the amino acid sequence of SEQ ID NO: 124. In some embodiments, the heavy chain constant region comprises or consists of the amino acid sequence of SEQ ID NO: 125. In some embodiments, the light chain constant region comprises or consists of the amino acid sequence of SEQ ID NO: 126. In some embodiments, the light chain constant region comprises or consists of the amino acid sequence of SEQ ID NO: 127.

In some embodiments, there is provided a method of treating an individual having a disease or condition associated with TSLP signaling (e.g., inflammatory diseases) comprising administering to the individual an effective amount of a pharmaceutical composition comprising an anti-TSLP antibody (e.g., full-length anti-TSLP antibody) comprising: a V_{H} comprising an HC-CDR1 comprising the amino acid sequence of SEQ ID NO: 2, an HC-CDR2 comprising the amino acid sequence of SEQ ID NO: 10, and an HC-CDR3 comprising the amino acid sequence of SEQ ID NO: 19, or a variant thereof comprising up to 5 amino acid substitutions in the HC-CDRs; and a V_{L} comprising an LC-CDR1 comprising the amino acid sequence of SEQ ID NO: 30, an LC-CDR2 comprising the amino acid sequence of SEQ ID NO: 43, and an LC-CDR3 comprising the amino acid sequence of SEQ ID NO: 55, or a variant thereof comprising up to 5 amino acid substitutions in the LC-CDRs.

In some embodiments, the anti-TSLP antibody provided herein comprises a V_{H} comprising the amino acid sequence of SEQ ID NO: 73 and a V_{L} comprising the amino acid sequence of SEQ ID NO: 92. In some embodiments, the anti-TSLP antibody provided herein is a full-length anti-TSLP antibody comprising IgG1 or IgG4 constant domains. In some embodiments, the IgG1 is human IgG1. In some embodiments, the IgG4 is human IgG4. In some embodiments, the heavy chain constant region comprises or consists of the amino acid sequence of SEQ ID NO: 124. In some embodiments, the heavy chain constant region comprises or consists of the amino acid sequence of SEQ ID NO: 125. In some embodiments, the light chain constant region comprises or consists of the amino acid sequence of SEQ ID NO: 126. In some embodiments, the light chain constant region comprises or consists of the amino acid sequence of SEQ ID NO: 127.

In some embodiments, there is provided a method of treating an individual having a disease or condition associated with TSLP signaling (e.g., inflammatory diseases) comprising administering to the individual an effective amount of a pharmaceutical composition comprising an anti-TSLP antibody (e.g., full-length anti-TSLP antibody) comprising: a V_{H} comprising an HC-CDR1 comprising the amino acid sequence of SEQ ID NO: 2, an HC-CDR2 comprising the amino acid sequence of SEQ ID NO: 10, and an HC-CDR3 comprising the amino acid sequence of SEQ ID NO: 19, or a variant thereof comprising up to 5 amino acid substitutions in the HC-CDRs; and a V_{L} comprising an LC-CDR1 comprising the amino acid sequence of SEQ ID NO: 31, an LC-CDR2 comprising the amino acid sequence of SEQ ID NO: 44, and an LC-CDR3 comprising the amino acid sequence of SEQ ID NO: 56, or a variant thereof comprising up to 5 amino acid substitutions in the LC-CDRs.

In some embodiments, the anti-TSLP antibody provided herein comprises a V_{H} comprising the amino acid sequence of SEQ ID NO: 73 and a V_{L} comprising the amino acid sequence of SEQ ID NO: 93. In some embodiments, the anti-TSLP antibody provided herein is a full-length anti-TSLP antibody comprising IgG1 or IgG4 constant domains. In some embodiments, the IgG1 is human IgG1. In some embodiments, the IgG4 is human IgG4. In some embodiments, the heavy chain constant region comprises or consists of the amino acid sequence of SEQ ID NO: 124. In some embodiments, the heavy chain constant region comprises or consists of the amino acid sequence of SEQ ID NO: 125. In some embodiments, the light chain constant region comprises or consists of the amino acid sequence of SEQ ID NO: 126. In some embodiments, the light chain constant region comprises or consists of the amino acid sequence of SEQ ID NO: 127.

In some embodiments, there is provided a method of treating an individual having a disease or condition associated with TSLP signaling (e.g., inflammatory diseases) comprising administering to the individual an effective amount of a pharmaceutical composition comprising an anti-TSLP antibody (e.g., full-length anti-TSLP antibody) comprising: a V_{H} comprising an HC-CDR1 comprising the amino acid sequence of SEQ ID NO: 2, an HC-CDR2 comprising the amino acid sequence of SEQ ID NO: 10, and an HC-CDR3 comprising the amino acid sequence of SEQ ID NO: 19, or a variant thereof comprising up to 5 amino acid substitutions in the HC-CDRs; and a V_{L} comprising an LC-CDR1 comprising the amino acid sequence of SEQ ID NO: 32, an LC-CDR2 comprising the amino acid sequence of SEQ ID NO: 45, and an LC-CDR3 comprising the amino acid sequence of SEQ ID NO: 57, or a variant thereof comprising up to 5 amino acid substitutions in the LC-CDRs.

In some embodiments, the anti-TSLP antibody provided herein comprises a V_{H} comprising the amino acid sequence of SEQ ID NO: 73 and a V_{L} comprising the amino acid sequence of SEQ ID NO: 94. In some embodiments, the anti-TSLP antibody provided herein is a full-length anti-TSLP antibody comprising IgG1 or IgG4 constant domains. In some embodiments, the IgG1 is human IgG1. In some embodiments, the IgG4 is human IgG4. In some embodiments, the heavy chain constant region comprises or consists of the amino acid sequence of SEQ ID NO: 124. In some embodiments, the heavy chain constant region comprises or consists of the amino acid sequence of SEQ ID NO: 125. In some embodiments, the light chain constant region comprises or consists of the amino acid sequence of SEQ ID NO: 126. In some embodiments, the light chain constant region comprises or consists of the amino acid sequence of SEQ ID NO: 127.

In some embodiments, there is provided a method of treating an individual having a disease or condition associated with TSLP signaling (e.g., inflammatory diseases) comprising administering to the individual an effective amount of a pharmaceutical composition comprising an anti-TSLP antibody (e.g., full-length anti-TSLP antibody) comprising: a V_{H} comprising an HC-CDR1 comprising the amino acid sequence of SEQ ID NO: 3, an HC-CDR2 comprising the amino acid sequence of SEQ ID NO: 11, and an HC-CDR3 comprising the amino acid sequence of SEQ ID NO: 19, or a variant thereof comprising up to 5 amino acid substitutions in the HC-CDRs; and a V_{L} comprising an LC-CDR1 comprising the amino acid sequence of SEQ ID NO: 33, an LC-CDR2 comprising the amino acid sequence of SEQ ID NO: 45, and an LC-CDR3 comprising the amino acid sequence of SEQ ID NO: 58, or a variant thereof comprising up to 5 amino acid substitutions in the LC-CDRs.

In some embodiments, the anti-TSLP antibody provided herein comprises a V_{H} comprising the amino acid sequence of SEQ ID NO: 76 and a V_{L} comprising the amino acid sequence of SEQ ID NO: 95. In some embodiments, the anti-TSLP antibody provided herein is a full-length anti-TSLP antibody comprising IgG1 or IgG4 constant domains. In some embodiments, the IgG1 is human IgG1. In some embodiments, the IgG4 is human IgG4. In some embodiments, the heavy chain constant region comprises or consists of the amino acid sequence of SEQ ID NO: 124. In some embodiments, the heavy chain constant region comprises or consists of the amino acid sequence of SEQ ID NO: 125. In some embodiments, the light chain constant region comprises or consists of the amino acid sequence of SEQ ID NO: 126. In some embodiments, the light chain constant region comprises or consists of the amino acid sequence of SEQ ID NO: 127.

In some embodiments, there is provided a method of treating an individual having a disease or condition associated with TSLP signaling (e.g., inflammatory diseases) comprising administering to the individual an effective amount of a pharmaceutical composition comprising an anti-TSLP antibody (e.g., full-length anti-TSLP antibody) comprising: a V_{H} comprising an HC-CDR1 comprising the amino acid sequence of SEQ ID NO: 3, an HC-CDR2 comprising the amino acid sequence of SEQ ID NO: 10, and an HC-CDR3 comprising the amino acid sequence of SEQ ID NO: 19, or a variant thereof comprising up to 5 amino acid substitutions in the HC-CDRs; and a V_{L} comprising an LC-CDR1 comprising the amino acid sequence of SEQ ID NO: 31, an LC-CDR2 comprising the amino acid sequence of SEQ ID NO: 46, and an LC-CDR3 comprising the amino acid sequence of SEQ ID NO: 59, or a variant thereof comprising up to 5 amino acid substitutions in the LC-CDRs.

In some embodiments, the anti-TSLP antibody provided herein comprises a V_{H} comprising the amino acid sequence of SEQ ID NO: 77 and a V_{L} comprising the amino acid sequence of SEQ ID NO: 96. In some embodiments, the anti-TSLP antibody provided herein is a full-length anti-TSLP antibody comprising IgG1 or IgG4 constant domains. In some embodiments, the IgG1 is human IgG1. In some embodiments, the IgG4 is human IgG4. In some embodiments, the heavy chain constant region comprises or consists of the amino acid sequence of SEQ ID NO: 124. In some embodiments, the heavy chain constant region comprises or consists of the amino acid sequence of SEQ ID NO: 125. In some embodiments, the light chain constant region comprises or consists of the amino acid sequence of SEQ ID NO: 126. In some embodiments, the light chain constant region comprises or consists of the amino acid sequence of SEQ ID NO: 127.

In some embodiments, there is provided a method of treating an individual having a disease or condition associated with TSLP signaling (e.g., inflammatory diseases) comprising administering to the individual an effective amount of a pharmaceutical composition comprising an anti-TSLP antibody (e.g., full-length anti-TSLP antibody) comprising: a V_{H} comprising an HC-CDR1 comprising the amino acid sequence of SEQ ID NO: 3, an HC-CDR2 comprising the amino acid sequence of SEQ ID NO: 12, and an HC-CDR3 comprising the amino acid sequence of SEQ ID NO: 20, or a variant thereof comprising up to 5 amino acid substitutions in the HC-CDRs; and a V_{L} comprising an LC-CDR1 comprising the amino acid sequence of SEQ ID NO: 34, an LC-CDR2 comprising the amino acid sequence of SEQ ID NO: 47, and an LC-CDR3 comprising the amino acid sequence of SEQ ID NO: 60, or a variant thereof comprising up to 5 amino acid substitutions in the LC-CDRs.

In some embodiments, the anti-TSLP antibody provided herein comprises a V_{H} comprising the amino acid sequence of SEQ ID NO: 78 and a V_{L} comprising the amino acid sequence of SEQ ID NO: 97. In some embodiments, the anti-TSLP antibody provided herein is a full-length anti-TSLP antibody comprising IgG1 or IgG4 constant domains. In some embodiments, the IgG1 is human IgG1. In some embodiments, the IgG4 is human IgG4. In some embodiments, the heavy chain constant region comprises or consists of the amino acid sequence of SEQ ID NO: 124. In some embodiments, the heavy chain constant region comprises or consists of the amino acid sequence of SEQ ID NO: 125. In some embodiments, the light chain constant region comprises or consists of the amino acid sequence of SEQ ID NO: 126. In some embodiments, the light chain constant region comprises or consists of the amino acid sequence of SEQ ID NO: 127.

For example, in some embodiments, there is provided a method of treating an individual having a disease or condition associated with TSLP signaling (e.g., inflammatory diseases) comprising administering to the individual an effective amount of a pharmaceutical composition comprising an anti-TSLP antibody (e.g., full-length anti-TSLP antibody) comprising a heavy chain variable domain (V_{H}) comprising an HC-CDR1 comprising NYX₁MT (SEQ ID NO: 114), wherein X₁ is D or G; an HC-CDR2 comprising SITFASSYIYYADSVKG (SEQ ID NO: 13); and an HC-CDR3 comprising GGGAYX₁GGSLDV (SEQ ID NO: 115), wherein X₁ is H or Y; and a light chain variable domain (V_{L}) comprising a light chain complementarity determining region (LC-CDR) 1 comprising RSSQSLLHX₁X₂X₃YTYLH (SEQ ID NO: 116), wherein X₁ is I or S, X₂ is N or Y, and X₃ is G or E; a LC-CDR2 comprising LVSX₁RAS (SEQ ID NO: 117), wherein X₁ is H, or Y; and a LC-CDR3 comprising EQTLQTPX₁X₂(SEQ ID NO: 118), wherein X₁ is Y or F, X₂ is S or T. In some embodiments, the anti-TSLP antibody is a full-length antibody. In some embodiments, the full-length anti-TSLP antibody is an IgG1 or IgG4 antibody. In some embodiments, the disease or condition is selected from the group consisting of asthma, idiopathic pulmonary fibrosis, atopic dermatitis, allergic conjunctivitis, allergic rhinitis, Netherton syndrome, eosinophilic esophagitis (EoE), food allergy, allergic diarrhoea, eosinophilic gastroenteritis, allergic bronchopulmonary aspergillosis (ABPA), allergic fungal sinusitis, cancer, rheumatoid arthritis, chronic obstructive pulmonary disease (COPD), systemic sclerosis, keloids, ulcerative colitis, chronic rhinosinusitis (CRS), nasal polyposis, chronic eosinophilic pneumonia, eosinophilic bronchitis, coeliac disease, Churg-Strauss syndrome, eosinophilic myalgia syndrome, hypereosinophilic syndrome, eosinophilic granulomatosis with polyangiitis, and inflammatory bowel disease. In some embodiments, the individual is human.

In some embodiments, there is provided a method of treating an individual having a disease or condition associated with TSLP signaling (e.g., inflammatory diseases) comprising administering to the individual an effective amount of a pharmaceutical composition comprising an anti-TSLP antibody (e.g., full-length anti-TSLP antibody) comprising: a V_{H} comprising an HC-CDR1 comprising the amino acid sequence of any one of SEQ ID NOs: 4-5, an HC-CDR2 comprising the amino acid sequence of SEQ ID NO: 13, and an HC-CDR3 comprising the amino acid sequence of any one of SEQ ID NOs: 21-22, or a variant thereof comprising up to 5 amino acid substitutions in the HC-CDRs; and a V_{L} comprising an LC-CDR1 comprising the amino acid sequence of any one of SEQ ID NOs: 35-37, an LC-CDR2 comprising the amino acid sequence of any one of SEQ ID NOs: 48-49, and an LC-CDR3 comprising the amino acid sequence of any one of SEQ ID NOs: 61-63, or a variant thereof comprising up to 5 amino acid substitutions in the LC-CDRs.

In some embodiments, there is provided a method of treating an individual having a disease or condition associated with TSLP signaling (e.g., inflammatory diseases) comprising administering to the individual an effective amount of a pharmaceutical composition comprising an anti-TSLP antibody (e.g., full-length anti-TSLP antibody) comprising a V_{H} comprising the amino acid sequence of any one of SEQ ID NOs: 79-81, or a variant thereof having at least about 90% (for example at least about any of 91%, 92%, 93%, 94%, 95%, 96%, 97%, 98%, or 99%) sequence identity to the amino acid sequence of any one of SEQ ID NOs: 79-81, and a V_{L} comprising the amino acid sequence of any one of SEQ ID NOs: 98-100, or a variant thereof having at least about 90% (for example at least about any of 91%, 92%, 93%, 94%, 95%, 96%, 97%, 98%, or 99%) sequence identity to the amino acid sequence of any one of SEQ ID NOs: 98-100.

In some embodiments, the anti-TSLP antibody provided herein is a full-length anti-TSLP antibody comprising IgG1 or IgG4 constant domains. In some embodiments, the IgG1 is human IgG1. In some embodiments, the IgG4 is human IgG4. In some embodiments, the heavy chain constant region comprises or consists of the amino acid sequence of SEQ ID NO: 124. In some embodiments, the heavy chain constant region comprises or consists of the amino acid sequence of SEQ ID NO: 125. In some embodiments, the light chain constant region comprises or consists of the amino acid sequence of SEQ ID NO: 126. In some embodiments, the light chain constant region comprises or consists of the amino acid sequence of SEQ ID NO: 127.

In some embodiments, there is provided a method of treating an individual having a disease or condition associated with TSLP signaling (e.g., inflammatory diseases) comprising administering to the individual an effective amount of a pharmaceutical composition comprising an anti-TSLP antibody (e.g., full-length anti-TSLP antibody) comprising: a V_{H} comprising an HC-CDR1 comprising the amino acid sequence of SEQ ID NO: 4, an HC-CDR2 comprising the amino acid sequence of SEQ ID NO: 13, and an HC-CDR3 comprising the amino acid sequence of SEQ ID NO: 21, or a variant thereof comprising up to 5 amino acid substitutions in the HC-CDRs; and a V_{L} comprising an LC-CDR1 comprising the amino acid sequence of SEQ ID NO: 35, an LC-CDR2 comprising the amino acid sequence of SEQ ID NO: 48, and an LC-CDR3 comprising the amino acid sequence of SEQ ID NO: 61, or a variant thereof comprising up to 5 amino acid substitutions in the LC-CDRs.

In some embodiments, the anti-TSLP antibody provided herein comprises a V_{H} comprising the amino acid sequence of SEQ ID NO: 79 and a V_{L} comprising the amino acid sequence of SEQ ID NO: 98. In some embodiments, the anti-TSLP antibody provided herein is a full-length anti-TSLP antibody comprising IgG1 or IgG4 constant domains. In some embodiments, the IgG1 is human IgG1. In some embodiments, the IgG4 is human IgG4. In some embodiments, the heavy chain constant region comprises or consists of the amino acid sequence of SEQ ID NO: 124. In some embodiments, the heavy chain constant region comprises or consists of the amino acid sequence of SEQ ID NO: 125. In some embodiments, the light chain constant region comprises or consists of the amino acid sequence of SEQ ID NO: 126. In some embodiments, the light chain constant region comprises or consists of the amino acid sequence of SEQ ID NO: 127.

In some embodiments, there is provided a method of treating an individual having a disease or condition associated with TSLP signaling (e.g., inflammatory diseases) comprising administering to the individual an effective amount of a pharmaceutical composition comprising an anti-TSLP antibody (e.g., full-length anti-TSLP antibody) comprising: a V_{H} comprising an HC-CDR1 comprising the amino acid sequence of SEQ ID NO: 4, an HC-CDR2 comprising the amino acid sequence of SEQ ID NO: 13, and an HC-CDR3 comprising the amino acid sequence of SEQ ID NO: 22, or a variant thereof comprising up to 5 amino acid substitutions in the HC-CDRs; and a V_{L} comprising an LC-CDR1 comprising the amino acid sequence of SEQ ID NO: 36, an LC-CDR2 comprising the amino acid sequence of SEQ ID NO: 48, and an LC-CDR3 comprising the amino acid sequence of SEQ ID NO: 62, or a variant thereof comprising up to 5 amino acid substitutions in the LC-CDRs.

In some embodiments, the anti-TSLP antibody provided herein comprises a V_{H} comprising the amino acid sequence of SEQ ID NO: 80 and a V_{L} comprising the amino acid sequence of SEQ ID NO: 99. In some embodiments, the anti-TSLP antibody provided herein is a full-length anti-TSLP antibody comprising IgG1 or IgG4 constant domains. In some embodiments, the IgG1 is human IgG1. In some embodiments, the IgG4 is human IgG4. In some embodiments, the heavy chain constant region comprises or consists of the amino acid sequence of SEQ ID NO: 124. In some embodiments, the heavy chain constant region comprises or consists of the amino acid sequence of SEQ ID NO: 125. In some embodiments, the light chain constant region comprises or consists of the amino acid sequence of SEQ ID NO: 126. In some embodiments, the light chain constant region comprises or consists of the amino acid sequence of SEQ ID NO: 127.

In some embodiments, there is provided a method of treating an individual having a disease or condition associated with TSLP signaling (e.g., inflammatory diseases) comprising administering to the individual an effective amount of a pharmaceutical composition comprising an anti-TSLP antibody (e.g., full-length anti-TSLP antibody) comprising: a V_{H} comprising an HC-CDR1 comprising the amino acid sequence of SEQ ID NO: 5, an HC-CDR2 comprising the amino acid sequence of SEQ ID NO: 13, and an HC-CDR3 comprising the amino acid sequence of SEQ ID NO: 22, or a variant thereof comprising up to 5 amino acid substitutions in the HC-CDRs; and a V_{L} comprising an LC-CDR1 comprising the amino acid sequence of SEQ ID NO: 37, an LC-CDR2 comprising the amino acid sequence of SEQ ID NO: 49, and an LC-CDR3 comprising the amino acid sequence of SEQ ID NO: 63, or a variant thereof comprising up to 5 amino acid substitutions in the LC-CDRs.

In some embodiments, the anti-TSLP antibody provided herein comprises a V_{H} comprising the amino acid sequence of SEQ ID NO: 81 and a V_{L} comprising the amino acid sequence of SEQ ID NO: 100. In some embodiments, the anti-TSLP antibody provided herein is a full-length anti-TSLP antibody comprising IgG1 or IgG4 constant domains. In some embodiments, the IgG1 is human IgG1. In some embodiments, the IgG4 is human IgG4. In some embodiments, the heavy chain constant region comprises or consists of the amino acid sequence of SEQ ID NO: 124. In some embodiments, the heavy chain constant region comprises or consists of the amino acid sequence of SEQ ID NO: 125. In some embodiments, the light chain constant region comprises or consists of the amino acid sequence of SEQ ID NO: 126. In some embodiments, the light chain constant region comprises or consists of the amino acid sequence of SEQ ID NO: 127.

For example, in some embodiments, there is provided a method of treating an individual having a disease or condition associated with TSLP signaling (e.g., inflammatory diseases) comprising administering to the individual an effective amount of a pharmaceutical composition comprising an anti-TSLP antibody (e.g., full-length anti-TSLP antibody) comprising a heavy chain variable domain (V_{H}) comprising an HC-CDR1 comprising SYAIS (SEQ ID NO: 6); an HC-CDR2 comprising MX₁X₂PLLGVTX₃YAEKFQG (SEQ ID NO: 119), wherein X₁ is L or I, X₂ is V or I, and X₃ is N or D; and an HC-CDR3 comprising GGX₁NYLYWYFDL (SEQ ID NO: 120), wherein X₁ is S or T; and a light chain variable domain (V_{L}) comprising a light chain complementarity determining region (LC-CDR) 1 comprising TGTSSDIGGYNRX₁S (SEQ ID NO: 121), wherein X₁ is V or I; a LC-CDR2 comprising X₁VSKRPS (SEQ ID NO: 122), wherein X₁ is D or E; and a LC-CDR3 comprising SX₁YAGTDTFVL (SEQ ID NO: 123), wherein X₁ is S or A. In some embodiments, the anti-TSLP antibody is a full-length antibody. In some embodiments, the full-length anti-TSLP antibody is an IgG1 or IgG4 antibody. In some embodiments, the disease or condition is selected from the group consisting of asthma, idiopathic pulmonary fibrosis, atopic dermatitis, allergic conjunctivitis, allergic rhinitis, Netherton syndrome, eosinophilic esophagitis (EoE), food allergy, allergic diarrhoea, eosinophilic gastroenteritis, allergic bronchopulmonary aspergillosis (ABPA), allergic fungal sinusitis, cancer, rheumatoid arthritis, chronic obstructive pulmonary disease (COPD), systemic sclerosis, keloids, ulcerative colitis, chronic rhinosinusitis (CRS), nasal polyposis, chronic eosinophilic pneumonia, eosinophilic bronchitis, coeliac disease, Churg-Strauss syndrome, eosinophilic myalgia syndrome, hypereosinophilic syndrome, eosinophilic granulomatosis with polyangiitis, and inflammatory bowel disease. In some embodiments, the individual is human.

In some embodiments, there is provided a method of treating an individual having a disease or condition associated with TSLP signaling (e.g., inflammatory diseases) comprising administering to the individual an effective amount of a pharmaceutical composition comprising an anti-TSLP antibody (e.g., full-length anti-TSLP antibody) comprising: a V_{H} comprising an HC-CDR1 comprising the amino acid sequence of SEQ ID NO: 6, an HC-CDR2 comprising the amino acid sequence of any one of SEQ ID NOs: 14-15, and an HC-CDR3 comprising the amino acid sequence of any one of SEQ ID NOs: 23-24, or a variant thereof comprising up to 5 amino acid substitutions in the HC-CDRs; and a V_{L} comprising an LC-CDR1 comprising the amino acid sequence of any one of SEQ ID NOs: 31 and 38, an LC-CDR2 comprising the amino acid sequence of any one of SEQ ID NOs: 42 and 45, and an LC-CDR3 comprising the amino acid sequence of any one of SEQ ID NOs: 60 and 64, or a variant thereof comprising up to 5 amino acid substitutions in the LC-CDRs.

In some embodiments, there is provided a method of treating an individual having a disease or condition associated with TSLP signaling (e.g., inflammatory diseases) comprising administering to the individual an effective amount of a pharmaceutical composition comprising an anti-TSLP antibody (e.g., full-length anti-TSLP antibody) comprising a V_{H} comprising the amino acid sequence of any one of SEQ ID NOs: 82-83, or a variant thereof having at least about 90% (for example at least about any of 91%, 92%, 93%, 94%, 95%, 96%, 97%, 98%, or 99%) sequence identity to the amino acid sequence of any one of SEQ ID NOs: 82-83, and a V_{L} comprising the amino acid sequence of any one of SEQ ID NOs: 101-102, or a variant thereof having at least about 90% (for example at least about any of 91%, 92%, 93%, 94%, 95%, 96%, 97%, 98%, or 99%) sequence identity to the amino acid sequence of any one of SEQ ID NOs: 101-102.

In some embodiments, the anti-TSLP antibody provided herein is a full-length anti-TSLP antibody comprising IgG1 or IgG4 constant domains. In some embodiments, the IgG1 is human IgG1. In some embodiments, the IgG4 is human IgG4. In some embodiments, the heavy chain constant region comprises or consists of the amino acid sequence of SEQ ID NO: 124. In some embodiments, the heavy chain constant region comprises or consists of the amino acid sequence of SEQ ID NO: 125. In some embodiments, the light chain constant region comprises or consists of the amino acid sequence of SEQ ID NO: 126. In some embodiments, the light chain constant region comprises or consists of the amino acid sequence of SEQ ID NO: 127.

In some embodiments, there is provided a method of treating an individual having a disease or condition associated with TSLP signaling (e.g., inflammatory diseases) comprising administering to the individual an effective amount of a pharmaceutical composition comprising an anti-TSLP antibody (e.g., full-length anti-TSLP antibody) comprising: a V_{H} comprising an HC-CDR1 comprising the amino acid sequence of SEQ ID NO: 6, an HC-CDR2 comprising the amino acid sequence of SEQ ID NO: 14, and an HC-CDR3 comprising the amino acid sequence of SEQ ID NO: 23, or a variant thereof comprising up to 5 amino acid substitutions in the HC-CDRs; and a V_{L} comprising an LC-CDR1 comprising the amino acid sequence of SEQ ID NO: 38, an LC-CDR2 comprising the amino acid sequence of SEQ ID NO: 42, and an LC-CDR3 comprising the amino acid sequence of SEQ ID NO: 60, or a variant thereof comprising up to 5 amino acid substitutions in the LC-CDRs.

In some embodiments, the anti-TSLP antibody provided herein comprises a V_{H} comprising the amino acid sequence of SEQ ID NO: 82 and a V_{L} comprising the amino acid sequence of SEQ ID NO: 101. In some embodiments, the anti-TSLP antibody provided herein is a full-length anti-TSLP antibody comprising IgG1 or IgG4 constant domains. In some embodiments, the IgG1 is human IgG1. In some embodiments, the IgG4 is human IgG4. In some embodiments, the heavy chain constant region comprises or consists of the amino acid sequence of SEQ ID NO: 124. In some embodiments, the heavy chain constant region comprises or consists of the amino acid sequence of SEQ ID NO: 125. In some embodiments, the light chain constant region comprises or consists of the amino acid sequence of SEQ ID NO: 126. In some embodiments, the light chain constant region comprises or consists of the amino acid sequence of SEQ ID NO: 127.

In some embodiments, there is provided a method of treating an individual having a disease or condition associated with TSLP signaling (e.g., inflammatory diseases) comprising administering to the individual an effective amount of a pharmaceutical composition comprising an anti-TSLP antibody (e.g., full-length anti-TSLP antibody) comprising: a V_{H} comprising an HC-CDR1 comprising the amino acid sequence of SEQ ID NO: 6, an HC-CDR2 comprising the amino acid sequence of SEQ ID NO: 15, and an HC-CDR3 comprising the amino acid sequence of SEQ ID NO: 24, or a variant thereof comprising up to 5 amino acid substitutions in the HC-CDRs; and a V_{L} comprising an LC-CDR1 comprising the amino acid sequence of SEQ ID NO: 31, an LC-CDR2 comprising the amino acid sequence of SEQ ID NO: 45, and an LC-CDR3 comprising the amino acid sequence of SEQ ID NO: 64, or a variant thereof comprising up to 5 amino acid substitutions in the LC-CDRs.

In some embodiments, the anti-TSLP antibody provided herein comprises a V_{H} comprising the amino acid sequence of SEQ ID NO: 83 and a V_{L} comprising the amino acid sequence of SEQ ID NO: 102. In some embodiments, the anti-TSLP antibody provided herein is a full-length anti-TSLP antibody comprising IgG1 or IgG4 constant domains. In some embodiments, the IgG1 is human IgG1. In some embodiments, the IgG4 is human IgG4. In some embodiments, the heavy chain constant region comprises or consists of the amino acid sequence of SEQ ID NO: 124. In some embodiments, the heavy chain constant region comprises or consists of the amino acid sequence of SEQ ID NO: 125. In some embodiments, the light chain constant region comprises or consists of the amino acid sequence of SEQ ID NO: 126. In some embodiments, the light chain constant region comprises or consists of the amino acid sequence of SEQ ID NO: 127.

In some embodiments, the individual is a mammal (e.g., human, non-human primate, rat, mouse, cow, horse, pig, sheep, goat, dog, cat, etc.). In some embodiments, the individual is a human. In some embodiments, the individual is a clinical patient, a clinical trial volunteer, an experimental animal, etc. In some embodiments, the individual is younger than about 60 years old (including for example younger than about any of 50, 40, 30, 25, 20, 15, or 10 years old). In some embodiments, the individual is older than about 60 years old (including for example older than about any of 70, 80, 90, or 100 years old). In some embodiments, the individual is diagnosed with or genetically prone to one or more of the diseases or disorders described herein (such as inflammatory disease). In some embodiments, the individual has one or more risk factors associated with one or more diseases or disorders described herein.

The present application in some embodiments provides a method of delivering an anti-TSLP antibody (such as any one of the anti-TSLP antibodies described herein, e.g., an isolated anti-TSLP antibody) to a cell producing TSLP in an individual, the method comprising administering to the individual a composition comprising the anti-TSLP antibody.

Many diagnostic methods for inflammatory disease or any other disease associated with TSLP signaling and the clinical delineation of those diseases are known in the art. Such methods include, but are not limited to, e.g., immunohistochemistry, PCR, and fluorescent in situ hybridization (FISH).

In some embodiments, the anti-TSLP antibodies (e.g., full-length anti-TSLP antibodies) and/or compositions of the application are administered in combination with a second, third, or fourth agent (including, e.g., inhaled, intranasal or parenteral corticosteroids, a bronchodilator, an anti-leukotriene antagonist, or an anti-histamine agent) to treat diseases or disorders associated with TSLP signaling.

For example, Asthma is a common chronic inflammatory disease of the airways characterized by variable and recurring symptoms, reversible airflow obstruction and bronchospasm. Common symptoms include wheezing, coughing, chest tightness, and shortness of breath. Asthma is thought to be caused by a combination of genetic and environmental factors and is managed largely by the use of bronchodilators and inhaled or oral corticosteroids.

The goals of treatment are long-term control of asthma, with reduction of symptoms, maintenance of a normal activity level, prevention of exacerbations, and prevention of loss of pulmonary function. Treatment is initiated based on severity of symptoms, physical examination findings, and, for some patients, the forced expiratory volume in the first second of expiration (FEV1) or peak expiratory flow rates. Details of asthma severity classification as recommended by the 2007 National Asthma Education and Prevention Program (NAEPP) Expert Panel Report-3 (EPR-3).

In some embodiments, the efficacy of treatment is measured according to the 2007 National Asthma Education and Prevention Program (NAEPP) Expert Panel Report-3 (EPR-3), the symptom frequency, FEV1 or Asthma Control Test (ACT Score, a commonly used validated questionnaire for assessing asthma control) are used to classify asthma control.

### Dosing and Method of Administering the Anti-TSLP Antibodies

The dose of the anti-TSLP antibody (such as isolated anti-TSLP antibody) compositions administered to an individual (such as a human) may vary with the particular composition, the mode of administration, and the type of disease being treated. In some embodiments, the amount of the composition (such as composition comprising isolated anti-TSLP antibody) is effective to result in an objective response (such as a partial response or a complete response) in the treatment of inflammatory disease. In some embodiments, the amount of the anti-TSLP antibody composition is sufficient to result in a complete response in the individual. In some embodiments, the amount of the anti-TSLP antibody composition is sufficient to result in a partial response in the individual. In some embodiments, the amount of the anti-TSLP antibody composition administered (for example when administered alone) is sufficient to produce an overall response rate of more than about any of 20%, 25%, 30%, 35%, 40%, 45%, 50%, 55%, 60%, 64%, 65%, 70%, 75%, 80%, 85%, or 90% among a population of individuals treated with the anti-TSLP antibody composition. Responses of an individual to the treatment of the methods described herein can be determined, for example, based on ACT Score.

In some embodiments, the amount of the composition (such as composition comprising isolated anti-TSLP antibody) is sufficient to control symptoms and reduce the risk of exacerbations of the individual. In some embodiments, the amount of the composition is sufficient to control symptoms and reduce the risk of exacerbations of the individual. In some embodiments, the amount of the composition (for example when administered along) is sufficient to produce clinical benefit of more than about any of 50%, 60%, 70%, or 77% among a population of individuals treated with the anti-TSLP antibody composition.

In some embodiments, the amount of the composition (such as composition comprising isolated anti-TSLP antibody), alone or in combination with a second, third, and/or fourth agent, is an amount sufficient to control symptoms and reduce the risk of exacerbations in the same subject prior to treatment or compared to the corresponding activity in other subjects not receiving the treatment. Standard methods can be used to measure the magnitude of this effect, such as in vitro assays with purified enzyme, cell-based assays, animal models, or human testing.

In some embodiments, the amount of the anti-TSLP antibody (such as a full-length anti-TSLP antibody) in the composition is below the level that induces a toxicological effect (i.e., an effect above a clinically acceptable level of toxicity) or is at a level where a potential side effect can be controlled or tolerated when the composition is administered to the individual.

In some embodiments, the amount of the composition is close to a maximum tolerated dose (MTD) of the composition following the same dosing regimen. In some embodiments, the amount of the composition is more than about any of 80%, 90%, 95%, or 98% of the MTD.

In some embodiments, the amount of an anti-TSLP antibody (such as a full-length anti-TSLP antibody) in the composition is included in a range of about 0.001 μg to about 1000 μg.

In some embodiments of any of the above aspects, the effective amount of anti-TSLP antibody (such as a full-length anti-TSLP antibody) in the composition is in the range of about 0.1 μg/kg to about 100 mg/kg of total body weight.

The anti-TSLP antibody compositions can be administered to an individual (such as human) via various routes, including, for example, intravenous, intra-arterial, intraperitoneal, intrapulmonary, oral, inhalation, intravascular, intramuscular, intra-tracheal, subcutaneous, intraocular, intrathecal, transmucosal, and transdermal. In some embodiments, sustained continuous release formulation of the composition may be used. In some embodiments, the composition is administered inhaled. In some embodiments, the composition is administered intravenously. In some embodiments, the composition is administered intraportally. In some embodiments, the composition is administered intraarterially. In some embodiments, the composition is administered intraperitoneally. In some embodiments, the composition is administered intrahepatically. In some embodiments, the composition is administered by hepatic arterial infusion. In some embodiments, the administration is to an injection site distal to a first disease site.

### Articles of Manufacture and Kits

In some embodiments of the application, there is provided an article of manufacture containing materials useful for the treatment of disease or condition associated with TSLP signaling, (e.g., inflammatory disease) or for delivering an anti-TSLP antibody (such as a full-length anti-TSLP antibody) to a cell producing TSLP of the individual. The article of manufacture can comprise a container and a label or package insert on or associated with the container. Suitable containers include, for example, bottles, vials, syringes, etc. The containers may be formed from a variety of materials such as glass or plastic. Generally, the container holds a composition which is effective for treating a disease or disorder described herein, and may have a sterile access port (for example the container may be an intravenous solution bag or a vial having a stopper pierceable by a hypodermic injection needle). At least one active agent in the composition is an anti-TSLP antibody of the application. The label or package insert indicates that the composition is used for treating the particular condition. The label or package insert will further comprise instructions for administering the anti-TSLP antibody composition to the patient. Articles of manufacture and kits comprising combinatorial therapies described herein are also contemplated.

Package insert refers to instructions customarily included in commercial packages of therapeutic products that contain information about the indications, usage, dosage, administration, contraindications and/or warnings concerning the use of such therapeutic products. In some embodiments, the package insert indicates that the composition is used for treating disease or condition associated with TSLP signaling (such as inflammatory disease). In some embodiments, the package insert indicates that the composition is used for treating disease or condition selected from the group consisting of asthma, idiopathic pulmonary fibrosis, atopic dermatitis, allergic conjunctivitis, allergic rhinitis, Netherton syndrome, eosinophilic esophagitis (EoE), food allergy, allergic diarrhoea, eosinophilic gastroenteritis, allergic bronchopulmonary aspergillosis (ABPA), allergic fungal sinusitis, cancer, rheumatoid arthritis, chronic obstructive pulmonary disease (COPD), systemic sclerosis, keloids, ulcerative colitis, chronic rhinosinusitis (CRS), nasal polyposis, chronic eosinophilic pneumonia, eosinophilic bronchitis, coeliac disease, Churg-Strauss syndrome, eosinophilic myalgia syndrome, hypereosinophilic syndrome, eosinophilic granulomatosis with polyangiitis, and inflammatory bowel disease.

Additionally, the article of manufacture may further comprise a second container comprising a pharmaceutically-acceptable buffer, such as bacteriostatic water for injection (BWFI), phosphate-buffered saline, Ringer's solution and dextrose solution. It may further include other materials desirable from a commercial and user standpoint, including other buffers, diluents, filters, needles, and syringes.

Kits are also provided that are useful for various purposes, e.g., for treatment of disease or condition associated with TSLP signaling (e.g., inflammatory disease), or for delivering an anti-TSLP antibody (such as a full-length anti-TSLP antibody) to a cell producing TSLP of the individual, optionally in combination with the articles of manufacture. Kits of the application include one or more containers comprising anti-TSLP antibody composition (or unit dosage form and/or article of manufacture), and in some embodiments, further comprise another agent (such as the agents described herein) and/or instructions for use in accordance with any of the methods described herein. The kit may further comprise a description of selection of individuals suitable for treatment. Instructions supplied in the kits of the application are typically written instructions on a label or package insert (e.g., a paper sheet included in the kit), but machine-readable instructions (e.g., instructions carried on a magnetic or optical storage disk) are also acceptable.

For example, in some embodiments, the kit comprises a composition comprising an anti-TSLP antibody (such as a full-length anti-TSLP antibody). In some embodiments, the kit comprises a) a composition comprising any one of the anti-TSLP antibodies described herein, and b) an effective amount of at least one other agent, wherein the other agent enhances the effect (e.g., treatment effect, detecting effect) of the anti-TSLP antibody. In some embodiments, the kit comprises a) a composition comprising any one of the anti-TSLP antibodies described herein, and b) instructions for administering the anti-TSLP antibody composition to an individual for treatment of a disease or condition associated with TSLP signaling (e.g., inflammatory disease). In some embodiments, the kit comprises a) a composition comprising any one of the anti-TSLP antibodies described herein, b) an effective amount of at least one other agent, wherein the other agent enhances the effect (e.g., treatment effect, detecting effect) of the anti-TSLP antibody, and c) instructions for administering the anti-TSLP antibody composition and the other agent(s) to an individual for treatment of a disease or condition associated with TSLP signaling (e.g., inflammatory disease. The anti-TSLP antibody and the other agent(s) can be present in separate containers or in a single container. For example, the kit may comprise one distinct composition or two or more compositions wherein one composition comprises an anti-TSLP antibody and another composition comprises another agent.

In some embodiments, the kit comprises a nucleic acid (or a set of nucleic acids) encoding an anti-TSLP antibody (such as a full-length anti-TSLP antibody). In some embodiments, the kit comprises a) a nucleic acid (or a set of nucleic acids) encoding an anti-TSLP antibody, and b) a host cell for expressing the nucleic acid (or a set of nucleic acids). In some embodiments, the kit comprises a) a nucleic acid (or a set of nucleic acids) encoding an anti-TSLP antibody, and b) instructions for i) expressing the anti-TSLP antibody in a host cell, ii) preparing a composition comprising the anti-TSLP antibody, and iii) administering the composition comprising the anti-TSLP antibody to an individual for the treatment of of a disease or condition associated with TSLP signaling (e.g., inflammatory disease. In some embodiments, the kit comprises a) a nucleic acid (or set of nucleic acids) encoding an anti-TSLP antibody, b) a host cell for expressing the nucleic acid (or set of nucleic acids), and c) instructions for i) expressing the anti-TSLP antibody in the host cell, ii) preparing a composition comprising the anti-TSLP antibody, and iii) administering the composition comprising the anti-TSLP antibody to an individual for the treatment of a disease or condition associated with TSLP signaling (e.g., inflammatory disease).

The kits of the application are in suitable packaging. Suitable packaging includes, but is not limited to, vials, bottles, jars, flexible packaging (e.g., sealed Mylar or plastic bags), and the like. Kits may optionally provide additional components such as buffers and interpretative information. The present application thus also provides articles of manufacture, which include vials (such as sealed vials), bottles, jars, flexible packaging, and the like.

The instructions relating to the use of the anti-TSLP antibody compositions generally include information as to dosage, dosing schedule, and route of administration for the intended treatment. The containers may be unit doses, bulk packages (e.g., multi-dose packages) or sub-unit doses. For example, kits may be provided that contain sufficient dosages of an anti-TSLP antibody (such as a full-length anti-TSLP antibody) as disclosed herein to provide effective treatment of an individual for an extended period, such as any of a week, 8 days, 9 days, 10 days, 11 days, 12 days, 13 days, 2 weeks, 3 weeks, 4 weeks, 6 weeks, 8 weeks, 3 months, 4 months, 5 months, 7 months, 8 months, 9 months, or more. Kits may also include multiple unit doses of the anti-TSLP antibody and pharmaceutical compositions and instructions for use and packaged in quantities sufficient for storage and use in pharmacies, for example, hospital pharmacies and compounding pharmacies.

Those skilled in the art will recognize that several embodiments are possible within the scope and spirit of this application. The application will now be described in greater detail by reference to the following non-limiting examples. The following examples further illustrate the application but, of course, should not be construed as in any way limiting its scope.

### EXAMPLES

### Example 1: Generation of Recombinant Human TSLP and Selection of Anti-TSLP scFv Antibodies

### Generation of Recombinant TSLP

The full-length sequence of human TSLP (hereon referred to as hTSLP) was synthesized (GenScript, Nanjing) and expressed both in E. coli and in mammalian cells. The amino acid sequence of human TSLP was found to contain a unique sequence of amino acids containing a furin cleavage site. In order to inactivate the human TSLP furin cleavage site, the human TSLP with mutations R127A and R130S (SEQ ID NO:129) was expressed in 293F cells. His-tag, mouse Fc-tag, human Fc-tag, Avi-tag or other conventionally used tags were used to tag human TSLP. Several forms of recombinant TSLP were produced, including his-tagged TSLP, N- or C-terminal aiv-his tagged TSLP, mFc-tagged TSLP, and hFc-tagged TSLP (referred as TSLP-His, Avi-His-TSLP, TSLP-Avi-His, TSLP-mFc, and TSLP-hFc, respectively). “his or His” stands for His-tag, “mFc” stands for mouse Fc-tag, “hFc” stands for human Fc-tag, and “Avi” stands for Avi tag.

Additionally, recombinant cynomolgus monkey TSLP (according to the GeneBank: Accession number EHH26699.1) was synthesized (GenScript, Nanjing) and expressed in 293F cells with mutations R127A and R130S.

The expression and purification of recombinant TSLP, including human or cynomolgus monkey TSLP-His, Avi-His-TSLP, TSLP-Avi-His, TSLP-mFc, and TSLP-hFc were carried out according to manufacturer's protocol. Briefly, 293F cells were transfected with the expression vectors respectively, and cultured at 37° C., under 5% CO₂ and 120 rpm for 5 days. The culture media was collected and proteins expressing His-tag were purified using Ni Sepharose purification according to manufacturer's protocol. Specifically, the Qiagen Ni-NTA superflow cartridges were used for immobilized metal affinity chromatography (IMAC) analysis. The cartridges were first equilibrated with buffer A1 (50 mM Na₃PO₄, 0.15 M NaCl, pH 7.2) with a flow rate of 150 cm/h. The pH of the supernatant of the culture media was adjusted to 7.2 and flown through the cartridges at room temperature (RT) at 150 cm/h. Next, buffer A1 (6 times the volume of that of the cartridges) was used to equilibrate the cartridges at 150 cm/h. A 50 mM PB solution (0.15 M NaCl and 0.2 M Imidazole, pH 7.2) with a volume that is 10 times that of the cartridges was used to wash the cartridges and the elution was collected.

The proteins expressing Fc-tag were purified using protein A resin based on the protocol provided by manufacturer's guide. Briefly, Protein A column was first equilibrated with a PBS buffer containing 50 mM PBS and 0.15 M NaCl (pH7.2), at a flow rate of 150 cm/h and with a volume that is six times the volume of the column. The supernatant of the culture media (pH was adjusted to 7.2) was passed through the column at 150 cm/h. Upon full equilibration, 50 mM sodium citrate (pH3.5) was added to the column and the elution containing TSLP-Fc was collected.

### Generation of Biotinylated TSLP Antigen

Biotinylation of recombinant TSLP with different tag using the biotin ligase B0101A (GeneCopoeia) was carried out according to the manufacturer's protocol. Briefly, buffer A/B and BirA ligase were added to recombinant TSLP with different tag, followed by 2 hours of incubation at 30° C. The efficiency of biotinylation was measured using ELISA, determined to be at least 70%.

Selection of Anti-TSLP scFv Antibodies

Generation of yeast scFv antibody display library: RNAs collected from 2000 human blood samples were reverse-transcribed into cDNAs, and the V_{H} and V_{K} fragments were amplified using V_{H}- and V_{K}-specific primers. Upon gel extraction and purification, scFvs were generated by linking V_{H} and V_{K}, and were cloned into the yeast display plasmid PYD1, which were then electroporated into yeast to generate the yeast scFv antibody display library.

Selection of anti-TSLP scFv antibodies: scFvs which recognized TSLP were isolated from the yeast display library. Briefly, magnetic-activated cell sorting (MACS) was used to enrich for cells expressing anti-TSLP scFv antibodies. 1000 OD yeast cells were subjected to centrifugation for 5 minutes at 2500 g. Cell pellet was obtained and resuspended in 1 L of SGCAA culture media with OD600=1 as the starting concentration. Expression was induced for 40-48 hours at 20° C. and 250 rpm. After centrifugation and washing with PBSM, the pellet was resuspended in 5-10 times volume of 1 μM Biotinylated human TSLP (in PBSM), and incubated for an hour at 4° C. After centrifugation and washing with PBSM, unbound antigens were washed off with PBSM. Magnetic beads were added and mixed thoroughly before incubation for 30 minutes at 4° C. on a rotator. The supernatant was discarded after centrifugation at 2500 g for 5 minutes, and the pellet was resuspended in PBSM with 5-10 times the volume. 7 mL of cells was added to the column at a time until all cells were passed through the column. Bound cells were collected and upon further culturing and centrifugation were subjected to plasmid isolation.

Generation of phage display library and selection of scFv antibodies: scFv antibody fragments from the selected yeast cells were PCR amplified using scFv-F and scFv-R primers. To generate phage display libraries, the scFv fragments were then cloned into the phage display vector pDAN5 using Sfi. Upon ligation, the vector was used to transduce TG1 phage display electroporation-competent cells to obtain the phage scFv antibody display library. scFv antibodies specific to TSLP were isolated from the phage display library in a series of repeated selection cycles. Briefly, phage scFv library (2×10¹¹ PFU) was added to biotinylated human TSLP, and incubated for 2 hours at 37° C. TSLP with phage bound was captured on streptavidin coated magnetic beads. Unbound phage were washed away. After washing with TBST for 8-15 times (increasing number of washes for every round of selection), phage that specifically bound to TSLP were washed off with Glycine-HCl (pH2.2). These phages were used to transduce TG1 cells in log phase, with the addition of Ampicillin, and cultured for an hour. Upon the addition of helper phage, the cells were cultured on a rocking bed for overnight at 200 rpm at 28° C. Culture media was collected the next day, centrifuged to obtain the supernatant, and was subjected to the next round of selection. A panel of positive scFv antibodies were obtained at the end of the selection process.

ELISA Screening: Monoclonal scFv antibodies were selected and subjected to ligand binding assays. The first assay was designed to identify scFv antibodies that bound human TSLP and/or cynomolgus TSLP. Briefly, a 96-well plate was coated with human TSLP or cynomolgus TSLP in PBS at 0.1 μg/well and left overnight at 4° C. Before loading the scFv antibodies, the plates were washed with TBST, blocked for 1-2 hours at 37° C. using 5% milk and washed again with TBST. Each scFv sample was first diluted to 40 μg/mL, and 150 μL was added to the first row of wells. The 40 μg/mL scFv samples were then serially diluted at a 1:3 ratio and added to the remaining wells. After incubating for an hour at 37° C., followed by washing with TBST for 6 times, 100 μl of the secondary antibody (HRP labeled mouse anti-flag (1:2500)) was added to each well. After incubation for an hour under 37° C., the plate was washed for 3 times using TBST. TMB was then added at 100 μL/well and incubated for 10-20 minutes at 37° C. 2 M H₂SO₄ was used to stop the reaction. The ELISA results (OD405) were then analyzed and the binding curves were generated by PRISM.

### Inhibition of TSLP Dependent Stat5 Activation:

A bioassay was developed to determine the purified anti-TSLP antibodies to neutralize TSLP-mediated cellular function in vitro. The antibodies were assessed to determine the potency at inhibiting TSLP stimulated activation of signal Transduction and Activator of Transcription 5 (Stat5) in Ba/F3 cells. Stat5 is a downstream effector of TSLP signaling. Ba/F3 cells were co-transfected with human TSLP receptor (hTSLPR), human IL-7Ra (hIL7Ra) and a Stat5-Luciferase reporter constructs. Cells were grown in RPMI1640+10% FBS+0.5 ng/mL mouse IL3+1% Penicillin/Streptomycin+600 μg/ml G418+200 μg/mL Zeocin+300 μg/mL Hygromycin, and then a stable cell line was obtained. In brief, Ba/F3 stable cells were seeded in 96-well plates at a density of 3×10⁴ cells/well/100 μL and incubated overnight at 37° C., 5% CO₂. 25 μL of serially diluted anti-TSLP antibodies at a 1:5 ratio, from a starting concentration of 66.67 nM to 8.67×10⁻⁴ nM were added to the wells, along with 25 μL human or cynomolgus TSLP with a final concentration of 500 μg/mL and incubated at 37° C., 5% CO₂ for 4 hours. The extent of reporter gene expression was measured using Promega ONE-Glo Luciferase assay system.

### Example 2: Generation and Characterization of Full-Length Human Anti-TSLP Antibodies

### Generation of Full-Length Anti-TSLP Antibodies

The most potent scFv antibodies were reformatted as human IgG1 or IgG4 antibody molecules with a human IgG1 or IgG4 heavy chain constant domain, and a human kappa or lambda light chain constant domain. V_{L} and V_{H} were amplified from the prokaryotic expression vector and introduced into eukaryotic expression vectors pTT5-L1 (containing kappa constant domain) or pTT5-L2 (containing lambda constant domain) and pTT5-H1 (containing IgG1 heavy chain constant domain), or pTT5-H4 (containing IgG4 heavy chain constant domain). Plasmids expressing the light or heavy chains were extracted and used to co-transfect 293F cells. Then, the cells were cultured at 37° C., 5% CO₂ and 120 rpm for five days, the culture media was purified using Protein A affinity chromatography. Briefly, Protein A column was first equilibrated with a PBS buffer containing 50 mM PBS and 0.15 M NaCl (pH7.2), at a flow rate of 150 cm/h and with a volume that is six times the volume of the column. The supernatant of the culture media (pH was adjusted to 7.2) was passed through the column at 150 cm/h. Upon further equilibration, the column was washed off using 50 mM sodium citrate (pH3.5) and the elution was collected. Out of the full-length antibodies that were generated, four scFv clones TSLP-01, TSLP-02, TSLP-03, and TSLP-04 were selected as the lead parent antibodies for affinity maturation.

Affinity maturation: To increase antibody affinity and biological activity, using the scFv of the parent antibodies, a phage scFv display library containing mutations in the CDR regions was generated, while the framework regions were kept constant. Further, in order to decrease the potential immunogenecity, two framework residues of TSLP-01 V_{H} were replaced by human germline residues individually or combinedly, one framework residue of TSLP-01 V_{L} was replaced by human germline residues according to its most homologous germline. Seven scFvs (TSLP-0101, TSLP-0102, TSLP-0103, TSLP-0104, TSLP-0105, TSLP-0106, and TSLP-0107) were assembled by paring different heavy chain and light chain variable domain. For the scFv of TSLP-02, in order to decrease the potential immunogenecity, two framework residues of TSLP-02 V_{H} were replaced by human germline residues individually or combinedly, according to its most homologous germline. Two scFvs (TSLP-0201, TSLP-0202) were assembled by paring different heavy chain and light chain variable domain. Variants that were able to bind human TSLP with high affinity, and with low dissociation rate were further assessed for biological activity. scFv antibodies that showed improved biological activity as compared to the parent antibodies were used to generate full-length antibodies. The selected lead-optimized antibodies were then subjected to further biochemical and biological analysis. The amino acid sequences of heavy chain CDRs (HCDRs), light chain CDRs (LCDRs) of all the variants, by Kabat were shown in Table 2.

### Affinity of Anti-TSLP Antibodies

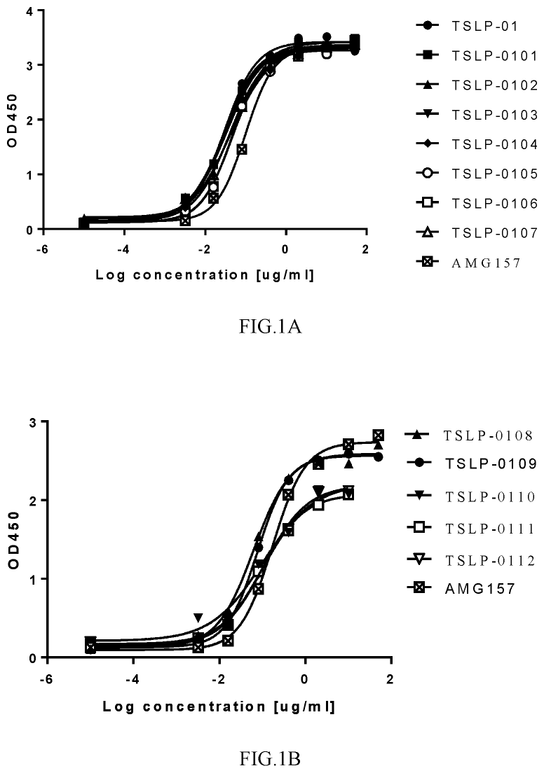

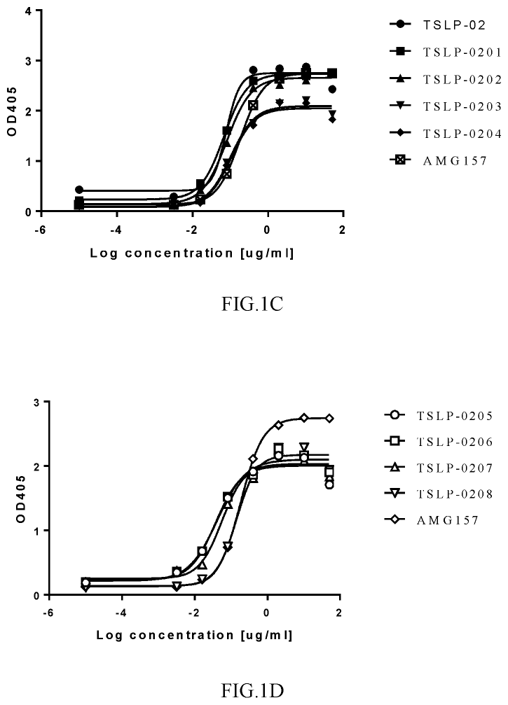

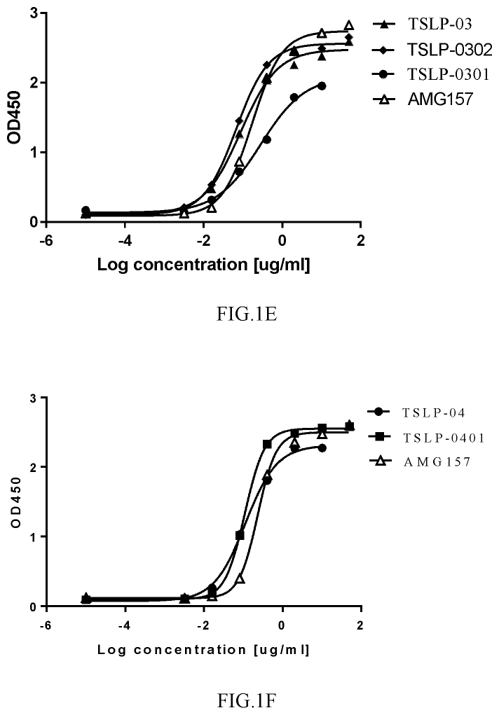

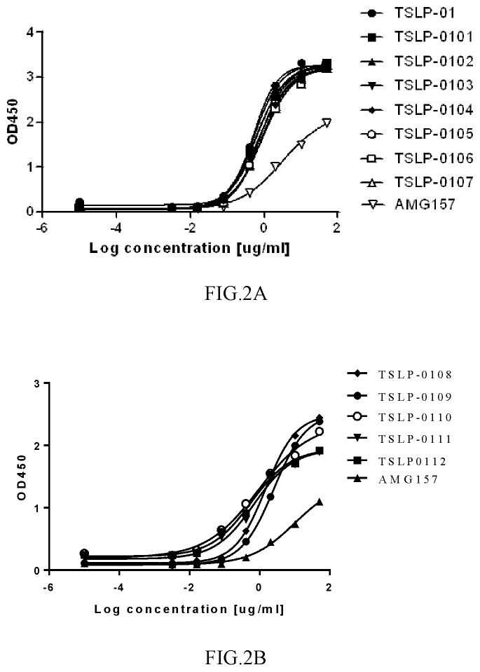

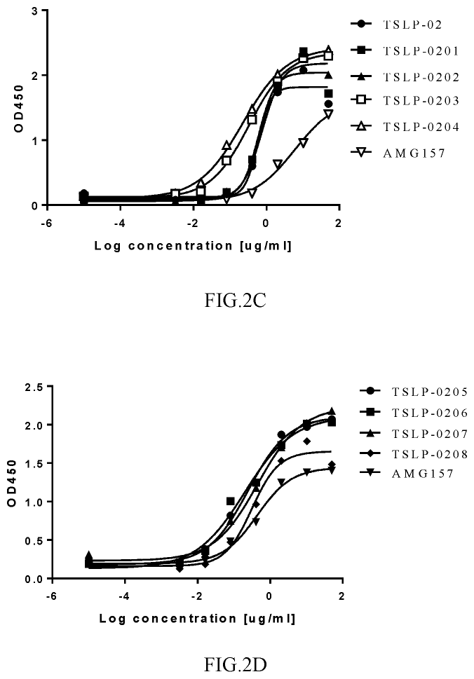

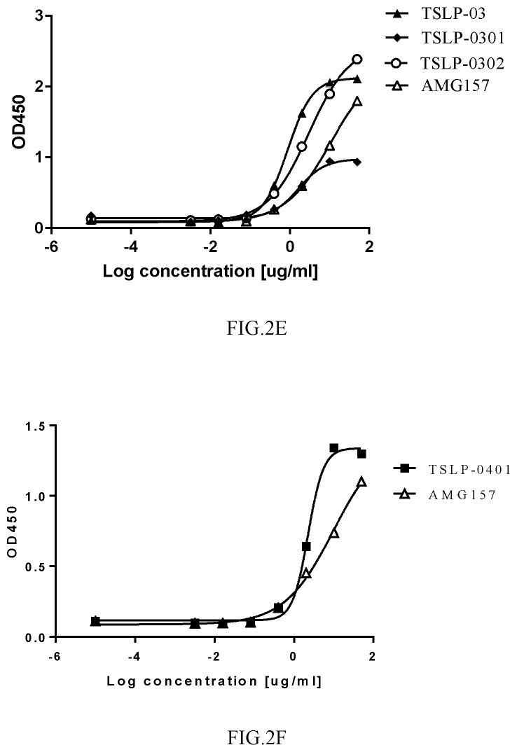

ELISA binding assay: The affinity of the parent antibodies and the lead-optimized antibodies (reformatted as human IgG1) for human or cynomolgus TSLP was evaluated using ELISA as described in example 1. As shown in FIGS. 1A-1F, all the parent antibodies and lead-optimized antibodies exhibited good binding affinity for human TSLP, showed comparable binding affinity as compared to the reference antibody AMG157. Then, the affinity of the parent antibodies and lead-optimized antibodies (reformatted as human IgG1) for cynomolgus monkey TSLP was evaluated using ELISA. As shown in FIG. 2A-2F, the anti-TSLP antibodies were cross-reacted with cynomolgus monkey TSLP, and showed better or comparable binding affinity for cynomolgus monkey TSLP than the reference antibody AMG157.

### Characterization of Binding Affinity (Kd)

The assay of binding affinity of anti-TSLP antibodies (reformatted as human IgG1) was basically performed as described in the literature (Della Ducata D, et al., Solution equilibrium titration for high throughput affinity estimation of unpurified antibodies and antibody fragments, J Biomol Screen. 2015, December; 20(10): 1256-67). AMG157 was used as reference control. Briefly, according to the manufacturer's instructions, the streptavidin MSD plates were blocked 30 mins at RT with PBS with 3% BSA (assay buffer). Serially diluted human TSLP with the concentration from 0 to 50 nM was equilibrated with the tested antibodies with the concentration of 10 pM overnight at RT, wells without antigen were used to determine Bmax values; wells containing only assay buffer were used to determine background. 50 μL of biotinylated human TSLP was coated at 0.2 μg/mL in assay buffer for 30 mins at RT on streptavidin MSD plates. After washing with PBST for 3 times, 50 μL of the equilibrated samples were transferred to those plates and incubated for 30 mins at RT. After washing, 50 μL per well of the SULFO-TAG NHS-Ester-tag labeled goat-anti-human IgG antibodies with a final concentration of 3.2 μg/mL was added to the MSD plate and incubated for 30 mins at RT. After washing the MSD Plate with PBST for 3 times, 150 μL of MSD read buffer was added to each well, electrochemiluminescence signals of the plate was detected using a Sector Imager 6000 (Meso Scale Discovery, Gaithersburg, MD, USA). The data was evaluated with Graphpad Prism software, for Kd determination of IgG, the following fit model for IgG was used (modified according to Piehler et al., 1997).

$$Y = \frac{3 B max}{[ I g G ]} ( [ \frac{I g G}{2} ] - \frac{{( \frac{X ⋆ [ IgG ] + Kd}{2} - \sqrt{\frac{{( X + [ IgG ] * Kd )}^{2}}{4} - X [ IgG ]} )}^{2}}{2 [ IgG ]} )$$

IgG: detected anti-TSLP antibody concentration
X: applied total soluble antigen concentration
Bmax: maximal signal of IgG without antigen
Kd: affinity

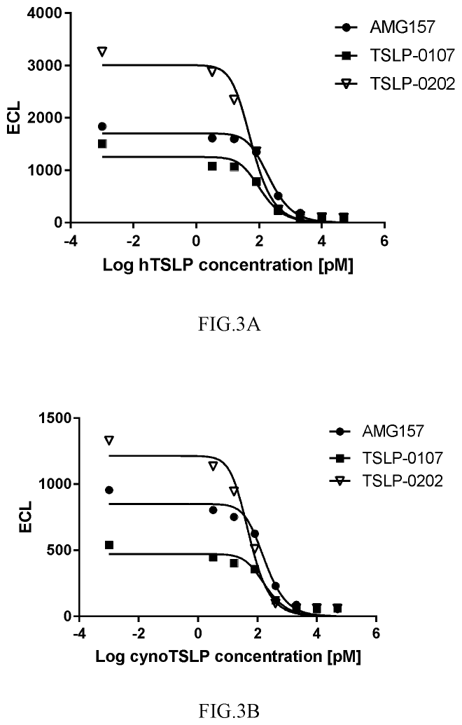

FIG. 3A and Table5 show the affinity Kd of the detected anti-TSLP antibodies TSLP-0107, TSLP-0202 to human TSLP, which have better affinity Kd than the reference antibody AMG157. FIG. 3B and Table6 show the affinity Kd of the detected anti-TSLP antibodies TSLP-0107, TSLP-0202 to cynomolgus monkey TSLP, which have better or comparable affinity Kd as compared to the reference antibody AM157.

TABLE 5

| Antibody | TSLP-0107 | TSLP-0202 | AMG157 |
|---|---|---|---|
| Kd (pM) | 43.97 | 21.1 | 84.85 |

TABLE 6

| Antibody | TSLP-0107 | TSLP-0202 | AMG157 |
|---|---|---|---|
| Kd (pM) | 25.38 | 4.468 | 17.59 |

Anti-TSLP Antibodies Compete with TSLP for Binding to its Receptor

HTRF®-based method was used to determine the ability of the anti-TSLP antibodies to block TSLP binding to its receptor complex, HTRF® is a trademark of Cis-Bio International (Bagnol/Ceze Cedex, France) and HTRF assays are homogeneous time resolved assays that generate a signal by fluorescence resonance energy transfer (FRET) between donor and acceptor molecules. The donor is a Eu³⁺ ion caged in a polycyclic cryptate (Eu-cryptate), while the acceptor is a modified allophycocyanin protein. Laser excitation of the donor at 337 nm results in the transfer of energy to the acceptor at 620 nm when they are in close proximity (≤90 Å) leading to the emission of light at 665 nm over a prolonged period of milliseconds. A 50-μs time delay in recording emissions and analysis of the ratio of the 665/620 nm minimizes interfering fluorescence from media components and unpaired fluorophores. Briefly, HEK293 cells were co-transfected with Flag-human TSLP receptor (Flag-hTSLPR) construct, and human IL-7Ra (hIL7Ra) construct. The transfectant HEK293-Flag-hTSLPR-IL7Ra cells were maintained in log phase, counted and resuspended at a concentration of 3×10⁶/mL, 10 μL of which were seeded in 96 well plate (3×10⁴/well). 5 μL of the serially diluted anti-TSLP antibodies from the concentration of 66.67 nM to 8.53×10⁻³ nM, along with 5 μL of 100 ng/mL TSLP-his were added to each well, the sides of the plate were tapped gently to help with mixing, and incubated for 1 h at RT. Then 5 μL of Eu(Tb) tagged anti-his antibody (Mab anti-His-Eu cryptate, donor) along with 5 μL of the Mab-anti-FlagXL665 (acceptor) were gently mixed again, and incubated for 1 h at RT. Read plate on Spark 10M, with excitation at 337 nm, and dual emission at 665/620 nm. The data was evaluated with Graphpad prism and EXCEL.

As shown in Table7, the anti-TSLP antibodies TSLP-0103, TSLP-0107, TSLP-0108, TSLP-02, TSLP-0201, TSLP-0202, TSLP-0203, TSLP-0204, TSLP-03 and TSLP-0401 (reformatted as human IgG1) were able to block human TSLP from binding to TSLP receptor.

TABLE 7

| Antibody | IC50(ng/mL) |
|---|---|
| TSLP-0103 | 378.8 |
| TSLP-0107 | 115.7 |
| TSLP-0108 | 158.1 |
| TSLP-02 | 228.8 |
| TSLP-0201 | 350.8 |
| TSLP-0202 | 296.9 |
| TSLP-0203 | 383 |
| TSLP-0204 | 427.6 |
| TSLP-03 | 494 |
| TSLP-0401 | 332.5 |

### Specificity of Anti-TSLP Antibodies

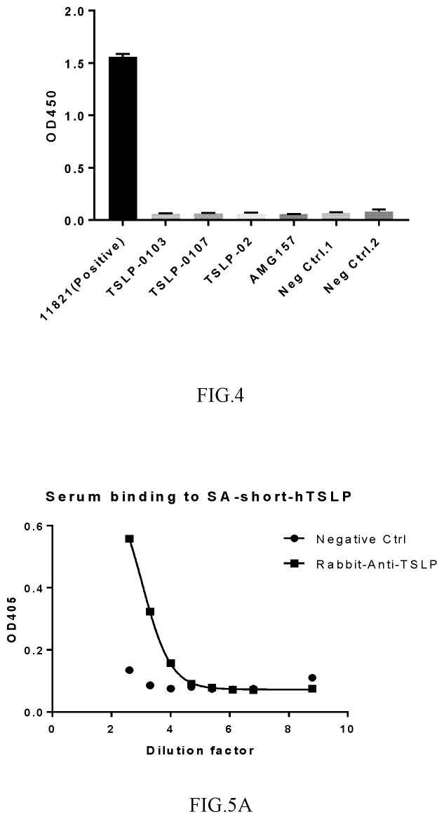

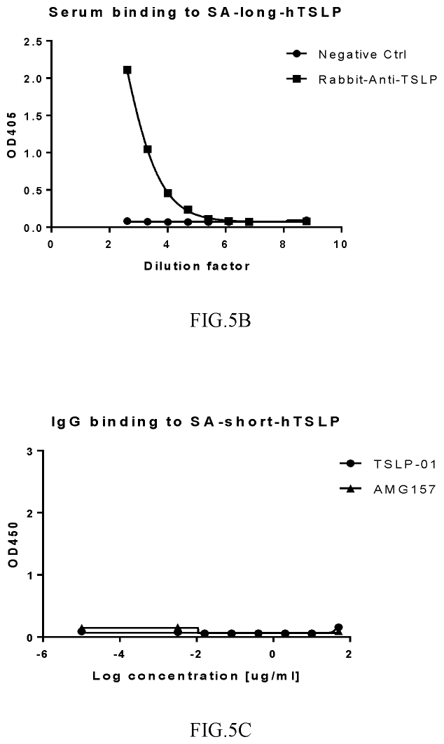

Cross-reactivity to homologousprotein IL-7: Using ELISA, the anti-TSLP antibodies TSLP-0103, TSLP-0107 and TSLP-02 (reformatted as human IgG1) were tested for cross-reactivity with TSLP homologous protein human IL-7. Mouse anti-human IL-7 antibody (Cat #11821, Sino Biogical, China) was used as a positive control, the anti-mouse-FC (Neg Ctrl. 1) and the anti-human-FC (Neg Ctrl. 2) were used as negative controls. As shown in FIG. 4, the anti-TSLP antibodies didn't bind to human IL-7, the same with the reference antibody AMG157, suggesting that the anti-TSLP antibodies didn't cross react with TSLP homologous protein IL-7.

### Anti-TSLP Antibodies Doesn't Bind to the Short Isoform of TSLP

Short isoform of TSLP is a biotinylated peptide comprising residues 69-131 of mature full length human TSLP. Binding of anti-TSLP antibodies to short form TSLP was determined using an ELISA method. A rabbit anti-TSLP polyclonal antibody (Cat #ab47943, Abcam) was used as a positive control for binding to both forms of TSLP. A rabbit serum was used as a negative control. Briefly, a 96-well plate was coated with 100 μl of 1 μg/mL streptavidin overnight. The plates were washed with PBST, then 100 μl of 1 μg/ml biotin labeled short TSLP or long TSLP was added to the plates, incubated at 37° C. for 1 hour. After the plates were washed again with PBST, each anti-TSLP antibody sample was serially diluted at a 1:5 ratio from a concentration of 5×10⁴ ng/ml to 3.2 ng/ml, and added to the wells. After incubating the plates for an hour at 37° C., followed by washing with PBST for 6 times, the secondary antibody anti-human-Fc-HRP or anti-rabbit-Fc-AP was added to each well. After incubation for an hour under 37° C., the plate was washed for 3 times using PBST. TMB or pNPP was then added at 100 μL/well and incubated for 10-20 minutes at room temperature. 2 M H₂SO₄ or 3 M NaOH was used to stop the reaction. The ELISA results were then analyzed and the binding curves were generated by GraphPad Prism 5 software (GraphPad Software).

As shown in FIGS. 5A-5D and Table 8, the ELISA results indicated that, the rabbit anti-TSLP polyclonal antibody bound to both short length isoform TSLP (FIG. 5A) and full length isoform TSLP (FIG. 5B). 50 μg/mL of the tested anti-TSLP antibody TSLP-01 bound to full length isoform TSLP with a stable top level (FIG. 5D), but didn't bind to short isoform TSLP (FIG. 5C). The EC50 of its binding to full length TSLP is at the ng/mL level.

TABLE 8

| Antibody | TSLP-01 | AMG157 |
|---|---|---|
| EC50(μg/mL) | 0.0335 | 0.0283 |

### Example 3: Inhibition of TSLP Induced Stat5 Activation

The optimized full length anti-TSLP antibodies (reformatted as human IgG1) were assessed to determine the potency at inhibiting human TSLP stimulated activation of Signal Transduction and Activator of Transcription 5 in Ba/F3 stable cells as described in Example 1.

Using the cell assay, the IC50 of the anti-TSLP antibodies inhibited human TSLP-induced Stat5 activation was shown in Table 9, the parent anti-TSLP antibody TSLP-01 and the lead-optimized antibodies showed better efficacy in inhibiting human TSLP-induced Stat5 activation than the reference antibody AMG157.

Using the cell assay, the IC50 of the anti-TSLP antibodies inhibited human TSLP-induced Stat5 activation was shown in Table 10, the parent anti-TSLP antibody TSLP-02 and the lead-optimized antibodies showed better efficacy in inhibiting human TSLP-induced Stat5 activation than the reference antibody AMG157.

Using the cell assay, the IC50 of the anti-TSLP antibodies inhibited human TSLP-induced Stat5 activation was shown in Table11, the anti-TSLP antibodies showed improved or comparable efficacy in inhibiting human TSLP-induced Stat5 activation as compared to the reference antibody AMG157.

TABLE 9

| Antibody | IC50 (ng/mL) |
|---|---|
| TSLP-01 | 19.28 |
| TSLP-0101 | 9.204 |
| TSLP-0102 | 11.4 |
| TSLP-0103 | 12.87 |
| TSLP-0104 | 15.41 |
| TSLP-0105 | 18.19 |
| TSLP-0106 | 16.94 |
| TSLP-0107 | 8.844 |
| AMG157 | 26.37 |

TABLE 10

| Antibody | IC50 (ng/mL) |
|---|---|
| TSLP-02 | 6.666 |
| TSLP-0201 | 4.629 |
| TSLP-0202 | 2.57 |
| AMG157 | 38.85 |

TABLE 11

| Antibody | IC50 (ng/mL) |
|---|---|
| TSLP-0108 | 10.79 |
| TSLP-0109 | 22.83 |
| TSLP-0110 | 24.53 |
| TSLP-0111 | 43.81 |
| TSLP-0112 | 30.82 |
| TSLP-0203 | 21.55 |
| TSLP-0204 | 18.9 |
| TSLP-0205 | 42.07 |
| TSLP-0206 | 18.31 |
| TSLP-0207 | 25.01 |
| TSLP-03 | 36.68 |
| TSLP-0301 | 41.69 |
| TSLP-04 | 36.41 |
| TSLP-0401 | 19.54 |
| AMG157 | 42.65 |

Inhibition of E. coli derived TSLP induced STAT5 activation: the antigen presentation mode maybe crucial for the antibody bioactivity, in order to investigate whether the selected anti-TSLP antibodies might target a glyco-epitope, the activity of anti-TSLP antibodies was assessed in inhibiting Stat5 activation induced by the E. coli derived TSLP.

As shown in Table12, the exemplary optimized anti-TSLP antibody TSLP-0107 showed higher efficacy in inhibiting the E. coli derived human TSLP induced Stat5 activation than the reference antibody AMG157.

TABLE 12

| Antibody | TSLP-0107 | AMG157 |
|---|---|---|
| IC50(ng/mL) | 2.594 | 35.81 |

### Example 4: Inhibition of TSLP Induced Ba/F3 Cells Proliferation

TSLP activities include promoting proliferation of BAF cells expressing human TSLPR, as described in PCT patent application publication WO 03/032898. The ability of the anti-TSLP antibodies to inhibit TSLP induced proliferation of Ba/F3 cells that express human TSLP receptor (hTSLPR) and human IL-7Ra (hIL7Ra) was assessed. The transfectant Ba/F3-TSLPR-IL7Ra cells proliferate in response to TSLP and the response can be inhibited by the anti-TSLP antibodies. Briefly, the transfectant Ba/F3-TSLPR-IL7Ra cells were maintained in RPMI1640+10% FBS+0.5 ng/mL mouse IL3+1% Penicillin/Streptomycin+600 μg/mL G418+200 μg/mL Zeocin, collected in logarithmic growth phase, then washed with RPMI1640+10% FBS+1% Penicillin/Streptomycin+600 μg/mL G418+200 μg/mL Zeocin, counted and resuspended at a concentration of 1×10⁵/mL, 100 μL of which were seeded in 96 well plate (1×10⁴/well). 50 μL of the tested anti-TSLP antibodies (reformatted as human IgG1) which were serially diluted at a 1:5 ratio, and 50 μL of human TSLP with a final concentration of 1.6 ng/mL were added to each well, gently mixed together, and incubated at 37° C., 5% CO₂ for 72 hours. Using CellTiter-Glo® Luminescent Cell Viability Assay kit, the cell proliferation was detected according to manufacturer's protocol.

As shown in Table 13, all the anti-TSLP antibodies exhibited good efficacy in inhibiting TSLP induced Ba/F3 cells proliferation. As compared to the reference antibody AMG157, the anti-TSLP antibodies exhibited better or comparable efficacy in inhibiting TSLP induced Ba/F3 cells proliferation.

TABLE 13

| Antibody | IC50 (ng/mL) |
|---|---|
| TSLP-0107 | 13.61 |
| TSLP-0108 | 39.36 |
| TSLP-0109 | 30.37 |
| TSLP-0110 | 129.1 |
| TSLP-0111 | 244.5 |
| TSLP-0112 | 213.9 |
| TSLP-02 | 17.46 |
| TSLP-0201 | 18.54 |
| TSLP-0202 | 13.12 |
| TSLP-0203 | 36.33 |
| TSLP-0204 | 42.23 |
| TSLP-0205 | 284.1 |
| TSLP-0206 | 65.13 |
| TSLP-0207 | 132.6 |
| TSLP-03 | 198.4 |
| TSLP-0301 | 216.9 |
| TSLP-04 | 149.7 |
| TSLP-0401 | 19.54 |
| AMG157 | 152.6 |

### Example 5: Inhibition of TARC Release in Human PBMCs Assay

Thymus and activation-regulated chemokine (TARC) release assay: TSLP is important in promoting release of TARC. To determine if anti-TSLP antibodies were able to neutralize TSLP in the context of a primary cell driven response, human TSLP induced TARC secretion from human PBMCs was tested in the presence or absence of anti-TSLP antibodies. Briefly, PBMCs (peripheral blood mononuclear cells) were isolated from human peripheral blood using Ficoll gradient, washed and the red blood cells were lysed, then the PBMCs were washed again using the RPMI 1640+10% FBS+1% Penicillin/Streptomycin+GlutaMax. Cells were counted and resuspended at a concentration of 1×10⁷/mL, 100 μL of which were seeded in the 96 well plate (1×10⁶ cells/well), and the plate was incubated at 37° C., 5% CO₂ for 1 hour. 50 μL of the tested anti-TSLP antibodies (reformatted as human IgG1) which were serially diluted at a 1:10 ratio, and 50 μL of human TSLP with a final concentration of 500 μg/mL were added to each well, gently mixed together, and incubated at 37° C., 5% CO₂ for 24 hours. TARC ELISA analysis was performed using R&D Quantikine ELISA kit following the manufacturer's protocols (R&D Quantikine).

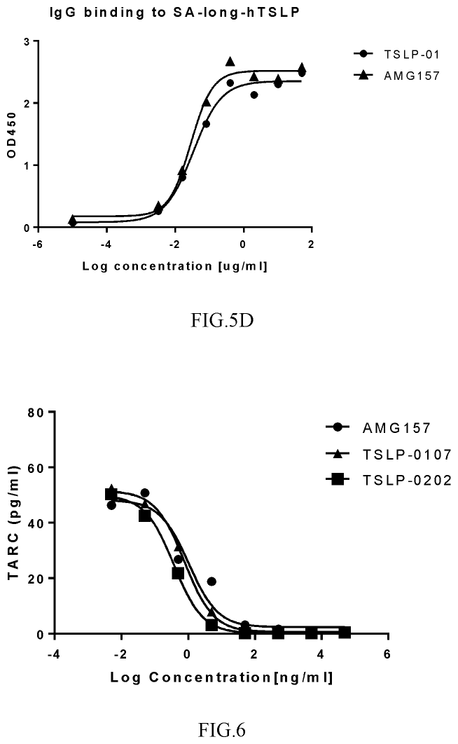

As shown in FIG. 6 and Table14, the exemplary lead-optimized antibodies TSLP-0107 and TSLP-0202 (reformatted as human IgG1) were tested for their ability to inhibit human TSLP (hTSLP) promoting TARC release. All the anti-TSLP antibodies were very potent inhibitor of recombinant human TSLP induced TARC release from human PBMCs, and showed improved ability to inhibit human TSLP promoting TARC release as compared to the reference antibody AMG157.

TABLE 14

| Antibody | TSLP-0107 | TSLP-0202 | AMG157 |
|---|---|---|---|
| IC50(ng/mL) | 0.7582 | 0.3645 | 1.031 |

### Example 6: Pharmacokinetics of Anti-TSLP Antibodies

PK values in rat: 24 healthy adult rats (approximately 0.2 kg by weight) were separated into two groups by weight. Rats in the first group were injected subcutaneously (sc) with 30 mg/kg of TSLP-0107-IgG1, TSLP-0202-IgG1, or AMG157-IgG2 (Amgen), 4 rats for each antibody, while rats in the second group were injected subcutaneously with 10 mg/kg of TSLP-0107-IgG1, TSLP-0202-IgG1, or AMG157-IgG2 (Amgen). Blood was collected first before injection, and subsequently at 0.01 days, 0.083 days, 0.25 days, 1 days, 2 days, 3 days, 5 days, 7 days, 9 days, 12 days, 17 days, 24 days, 28 days and 33 days after injection. After centrifugation, the plasma was used for analyzing antibody concentration using ELISA. Briefly, synthetic TSLP was used to cover the wells of a 96-well plate. On the following day, after washed with PBST, the plate was blocked with 200 μL PBS-milk for an hour, followed by another wash with PBST, then the plasma was added into the plate and incubated for an hour at 37° C. The plate was washed with 0.1% TBST for 6 times before 100 μL of Goat-anti-human Fc antibody-AP (1:10000 in PBS) was added to each well and incubated for an hour. After washing with 0.1% TBST for 6 times, 50 μL of pNPP was added to each well and color was developed for 10⁻²⁰ minutes at 37° C. The results were read by a microplate reader at 405 nm.

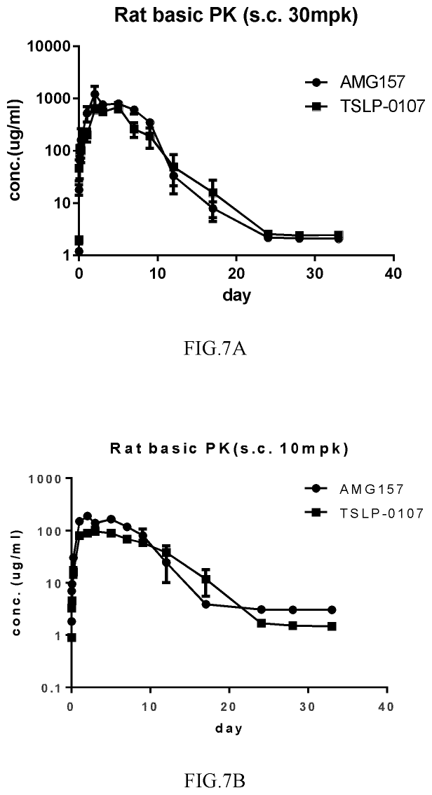

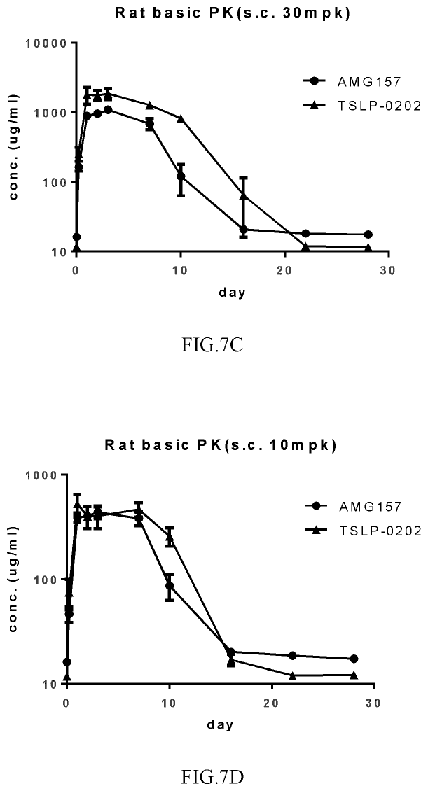

The results were read by a microplate reader at 405 nm, as shown in FIGS. 7A-7D, either in the group of sc 30 mg/kg (FIG. 7A and FIG. 7C) or sc 10 mg/kg (FIG. 7B and FIG. 7D), the half-life of TSLP-0107-IgG1 or TSLP-0202-IgG1 was longer than or comparable with the reference antibody AMG157-IgG2.

### Example 7: Evaluation the Effects of Anti-TSLP Antibodies in a Cynomolgus Monkey Asthma Model

All animal procedures were approved and conducted in compliance with the Institutional Animal Care and Use Committee guidelines. Cynomolgus monkeys (Macaca fascicularis; 2-3 kg) with previously established sensitivity to Ascaris suum antigen were used in the study. First, the animals were subject to antigen challenge on 2 consecutive days (days 1 and 2) and tested for inflammation and lung function before and after antigen challenge. Then the animals were divided into 2 groups based on baseline data, receiving either vehicle or TSLP-mAb (3 mg/kg administered intravenously) weekly for 6 weeks. Allergen challenges were performed at weeks 2 and 6, and inflammation and lung function parameters were assessed. Significance of effects was evaluated by using a nonparametric approach with the Wilcoxon rank sum test.

Lung function measurements: A suum challenge was performed while each animal was anesthetized by using propofol, in which a single dose of A suum antigen was administered through intermittent positive pressure breathing with a ventilator and in-line nebulizer. Acute pulmonary function changes in response to A suum challenge were determined during 15 minutes, including the maximum percentage change from baseline that was calculated by using lung resistance and area under the curve during that time period. For assessment of AHR, increasing doses of histamine (beginning with 0.1 mg/kg) were administered intravenously (0.1 ml/kg) 24 hours after the second A suum challenge. Lung resistance and dynamic compliance values were continuously measured throughout the histamine dose-response period. Preliminary observations indicated that the anti-TSLP antibodies described above exhibited beneficial effects in the animal asthma model.

### Example 8: Evaluation the Effects of Anti-TSLP Antibodies in an HDM Allergic Cynomolgus Monkey Model

The anti-TSLP antibodies were administered to house dust mite (HDM) allergic cynomolgus monkeys to demonstrate the effectiveness of anti-TSLP antibodies to treat allergic lung inflammation. This animal model will make possible the collection of airway tissues, BAL fluid, and associated PBMC's harvested from the control and anti-TSLP antibodies treated animals; and will provide the ability to access efficacy in Early Allergic Reactions (EAR) and Late Allergic Reactions (LAR). Further information regarding non-primate models of chronic allergic asthma is well known in the art. See, e.g., Schelegle et al., Am. J. Pathology 158(1):333-341 (2001); Avdalovic et al., Am. J. Respir. Crit. Care Med. 174:1069-74 (2006) Care and Van Scott et al., J. Appl. Physiol. 99(6):2080-2086 (2005).

Preliminary observations showed that the animals which were administered with anti-TSLP antibodies had reduced bronchoalveolar lavage (BAL) fluid eosinophil counts, reduced airway resistance in response to allergen challenge, and reduced IL4, IL5 IL-13, and IgE levels in BAL fluid compared with values seen in vehicle-treated animals. These results demonstrate the anti-TSLP antibodies exhibited promising efficacy of TSLP blockade in an allergic lung inflammation model.

1. An isolated anti-TSLP antibody, wherein the anti-TSLP antibody comprises:
(a) a heavy chain variable domain (V_{H}) comprising a heavy chain complementarity determining region (HC-CDR) 1 comprising SGYGWS (SEQ ID NO: 1);
an HC-CDR2 comprising YX₁SYYGSX₂SYNPSLKS (SEQ ID NO: 103),
wherein X₁ is I or F, and X₂ is I or T; and
an HC-CDR3 comprising TNLLYFX₁X₂ (SEQ ID NO: 104), wherein X₁ is D or E, and X₂ is S or Y;
and a light chain variable domain (V_{L}) comprising a light chain complementarity determining region (LC-CDR) 1 comprising RASQX₁X₂SX₃X₄LA (SEQ ID NO: 105), wherein X₁ is G or S, X₂ is V or I, X₃ is N or S, and X₄ is N or Y;
a LC-CDR2 comprising DX₁SX₂X₃X₄X₅ (SEQ ID NO: 106), wherein X₁ is A or T, X₂ is S or N, X₃ is R or L, X₄ is A or Q, and X₅ is T or S; and
a LC-CDR3 comprising QQYSX₁WPX₂YX₃ (SEQ ID NO: 107), wherein X₁ is D or N, X₂ is Q or E, and X₃ is T or S; or
(b) a heavy chain variable domain (V_{H}) comprising a heavy chain complementarity determining region (HC-CDR) 1 comprising SYGIX₁ (SEQ ID NO: 108), wherein X₁ is N or S;
an HC-CDR2 comprising VIX₁PLX₂X₃VX₄X₅YAEKFQG (SEQ ID NO: 109),
wherein X₁ is V or I, X₂ is V or L, X₃ is G or D, X₄ is T or P, and X₅ is I or N; and
an HC-CDR3 comprising GX₁EYFYWYFDL (SEQ ID NO: 110), wherein X₁ is Q or A;
and a light chain variable domain (V_{L}) comprising a light chain complementarity determining region (LC-CDR) 1 comprising X₁GX₂X₃SDIGGYX₄RVS (SEQ ID NO: 111), wherein X₁ is S or T, X₂ is S or T, X₃ is S, T, I or N, and X₄ is N or D;
a LC-CDR2 comprising X₁X₂X₃KRX₄S (SEQ ID NO: 112), wherein X₁ is D, G or E, X₂ is V, F or I, X₃ is S or N, and X₄ is P or S; and
a LC-CDR3 comprising X₁SYAX₂X₃X₄X₅FX₆X₇ (SEQ ID NO: 113), wherein X₁ is S or T, X₂ is G or S, X₃ is T or G, X₄ is D or H, X₅ is T or I, X₆ is I, G, V or A, and X₇ is L, I or F; or
(c) a heavy chain variable domain (V_{H}) comprising a heavy chain complementarity determining region (HC-CDR) 1 comprising NYX₁MT (SEQ ID NO: 114), wherein X₁ is D or G;
an HC-CDR2 comprising SITFASSYIYYADSVKG (SEQ ID NO: 13); and
an HC-CDR3 comprising GGGAYX₁GGSLDV (SEQ ID NO: 115), wherein X₁ is H or Y;
and a light chain variable domain (V_{L}) comprising a light chain complementarity determining region (LC-CDR) 1 comprising RSSQSLLHX₁X₂X₃YTYLH (SEQ ID NO: 116), wherein X₁ is I or S, X₂ is N or Y, and X₃ is G or E;
a LC-CDR2 comprising LVSX₁RAS (SEQ ID NO: 117), wherein X₁ is H, or Y; and
a LC-CDR3 comprising EQTLQTPX₁X₂ (SEQ ID NO: 118), wherein X₁ is Y or F, X₂ is S or T; or
(d) a heavy chain variable domain (V_{H}) comprising a heavy chain complementarity determining region (HC-CDR) 1 comprising SYAIS (SEQ ID NO: 6);
an HC-CDR2 comprising MX₁X₂PLLGVTX₃YAEKFQG (SEQ ID NO: 119),
wherein X₁ is L or I, X₂ is V or I, and X₃ is N or D; and
an HC-CDR3 comprising GGX₁NYLYWYFDL (SEQ ID NO: 120), wherein X₁ is S or T;
and a light chain variable domain (V_{L}) comprising a light chain complementarity determining region (LC-CDR) 1 comprising TGTSSDIGGYNRX₁S (SEQ ID NO: 121), wherein X₁ is V or I;
a LC-CDR2 comprising X₁VSKRPS (SEQ ID NO: 122), wherein X₁ is D or E;
and
a LC-CDR3 comprising SX₁YAGTDTFVL (SEQ ID NO: 123), wherein X₁ is S or A.

2. The isolated anti-TSLP antibody of claim 1, comprising:
(a) a V_{H} comprising an HC-CDR1 comprising the amino acid sequence of SEQ ID NO: 1, or a variant thereof comprising up to about 3 amino acid substitutions; an HC-CDR2 comprising the amino acid sequence of any one of SEQ ID NOs: 7-9, or a variant thereof comprising up to about 3 amino acid substitutions; and an HC-CDR3 comprising the amino acid sequence of any one of SEQ ID NOs: 16-18, or a variant thereof comprising up to about 3 amino acid substitutions; and
a V_{L} comprising a LC-CDR1 comprising the amino acid sequence of any one of SEQ ID NOs: 25-28, or a variant thereof comprising up to about 3 amino acid substitutions; a LC-CDR2 comprising the amino acid sequence of any one of SEQ ID NOs: 39-41, or a variant thereof comprising up to about 3 amino acid substitutions; and a LC-CDR3 comprising the amino acid sequence of any one of SEQ ID NOs: 50-53, or a variant thereof comprising up to about 3 amino acid substitutions; or
(b) a V_{H} comprising an HC-CDR1 comprising the amino acid sequence of any one of SEQ ID NOs: 2-3, or a variant thereof comprising up to about 3 amino acid substitutions; an HC-CDR2 comprising the amino acid sequence of any one of SEQ ID NOs: 10-12, or a variant thereof comprising up to about 3 amino acid substitutions; and an HC-CDR3 comprising the amino acid sequence of any one of SEQ ID NOs: 19-20, or a variant thereof comprising up to about 3 amino acid substitutions; and
a V_{L} comprising a LC-CDR1 comprising the amino acid sequence of any one of SEQ ID NOs: 29-34, or a variant thereof comprising up to about 3 amino acid substitutions; a LC-CDR2 comprising the amino acid sequence of any one of SEQ ID NOs: 42-47, or a variant thereof comprising up to about 3 amino acid substitutions; and a LC-CDR3 comprising the amino acid sequence of any one of SEQ ID NOs: 54-60, or a variant thereof comprising up to about 3 amino acid substitutions; or
(c) a V_{H} comprising an HC-CDR1 comprising the amino acid sequence of any one of SEQ ID NOs: 4-5, or a variant thereof comprising up to about 3 amino acid substitutions; an HC-CDR2 comprising the amino acid sequence of SEQ ID NO: 13, or a variant thereof comprising up to about 3 amino acid substitutions; and an HC-CDR3 comprising the amino acid sequence of any one of SEQ ID NOs: 21-22, or a variant thereof comprising up to about 3 amino acid substitutions; and
a V_{L} comprising a LC-CDR1 comprising the amino acid sequence of any one of SEQ ID NOs: 35-37, or a variant thereof comprising up to about 3 amino acid substitutions; a LC-CDR2 comprising the amino acid sequence of any one of SEQ ID NOs: 48-49, or a variant thereof comprising up to about 3 amino acid substitutions; and a LC-CDR3 comprising the amino acid sequence of any one of SEQ ID NOs: 61-63, or a variant thereof comprising up to about 3 amino acid substitutions; or
(d) a V_{H} comprising an HC-CDR1 comprising the amino acid sequence of SEQ ID NO: 6, or a variant thereof comprising up to about 3 amino acid substitutions; an HC-CDR2 comprising the amino acid sequence of any one of SEQ ID NOs: 14-15, or a variant thereof comprising up to about 3 amino acid substitutions; and an HC-CDR3 comprising the amino acid sequence of any one of SEQ ID NOs: 23-24, or a variant thereof comprising up to about 3 amino acid substitutions; and
a V_{L} comprising a LC-CDR1 comprising the amino acid sequence of any one of SEQ ID NOs: 31 and 38, or a variant thereof comprising up to about 3 amino acid substitutions; a LC-CDR2 comprising the amino acid sequence of any one of SEQ ID NOs: 42 and 45, or a variant thereof comprising up to about 3 amino acid substitutions; and a LC-CDR3 comprising the amino acid sequence of any one of SEQ ID NOs: 60 and 64, or a variant thereof comprising up to about 3 amino acid substitutions.

3. The isolated anti-TSLP antibody of claim 2, comprising
(a) a V_{H} comprising an HC-CDR1, an HC-CDR2, and an HC-CDR3 of a V_{H} comprising the amino acid sequence of any one of SEQ ID NOs: 65-72; and a V_{L} comprising a LC-CDR1, a LC-CDR2, and a LC-CDR3 of a V_{L} comprising the amino acid sequence of any one of SEQ ID NOs: 84-90; or
(b) a V_{H} comprising an HC-CDR1, an HC-CDR2, and an HC-CDR3 of a V_{H} comprising the amino acid sequence of any one of SEQ ID NOs: 73-78; and a V_{L} comprising a LC-CDR1, a LC-CDR2, and a LC-CDR3 of a V_{L} comprising the amino acid sequence of any one of SEQ ID NOs: 91-97; or
(c) a V_{H} comprising an HC-CDR1, an HC-CDR2, and an HC-CDR3 of a V_{H} comprising the amino acid sequence of any one of SEQ ID NOs: 79-81; and a V_{L} comprising a LC-CDR1, a LC-CDR2, and a LC-CDR3 of a V_{L} comprising the amino acid sequence of any one of SEQ ID NOs: 98-100; or
(d) comprising a V_{H} comprising an HC-CDR1, an HC-CDR2, and an HC-CDR3 of a V_{H} comprising the amino acid sequence of any one of SEQ ID NOs: 82-83; and a V_{L} comprising a LC-CDR1, a LC-CDR2, and a LC-CDR3 of a V_{L} comprising the amino acid sequence of any one of SEQ ID NOs: 101-102.

4. The isolated anti-TSLP antibody of claim 3, comprising:
(a)
(i) a V_{H} comprising an HC-CDR1, an HC-CDR2, and an HC-CDR3 of a V_{H} comprising the amino acid sequence of SEQ ID NO: 65; and a V_{L} comprising a LC-CDR1, a LC-CDR2, and a LC-CDR3 of a V_{L} comprising the amino acid sequence of SEQ ID NO: 84;
(ii) a V_{H} comprising an HC-CDR1, an HC-CDR2, and an HC-CDR3 of a V_{H} comprising the amino acid sequence of SEQ ID NO: 65; and a V_{L} comprising a LC-CDR1, a LC-CDR2, and a LC-CDR3 of a V_{L} comprising the amino acid sequence of SEQ ID NO: 85;
(iii) a V_{H} comprising an HC-CDR1, an HC-CDR2, and an HC-CDR3 of a V_{H} comprising the amino acid sequence of SEQ ID NO: 66; and a V_{L} comprising a LC-CDR1, a LC-CDR2, and a LC-CDR3 of a V_{L} comprising the amino acid sequence of SEQ ID NO: 84;
(iv) a V_{H} comprising an HC-CDR1, an HC-CDR2, and an HC-CDR3 of a V_{H} comprising the amino acid sequence of SEQ ID NO: 66; and a V_{L} comprising a LC-CDR1, a LC-CDR2, and a LC-CDR3 of a V_{L} comprising the amino acid sequence of SEQ ID NO: 85;
(v) a V_{H} comprising an HC-CDR1, an HC-CDR2, and an HC-CDR3 of a V_{H} comprising the amino acid sequence of SEQ ID NO: 67; and a V_{L} comprising a LC-CDR1, a LC-CDR2, and a LC-CDR3 of a V_{L} comprising the amino acid sequence of SEQ ID NO: 84;
(vi) a V_{H} comprising an HC-CDR1, an HC-CDR2, and an HC-CDR3 of a V_{H} comprising the amino acid sequence of SEQ ID NO: 67; and a V_{L} comprising a LC-CDR1, a LC-CDR2, and a LC-CDR3 of a V_{L} comprising the amino acid sequence of SEQ ID NO: 85;
(vii) a V_{H} comprising an HC-CDR1, an HC-CDR2, and an HC-CDR3 of a V_{H} comprising the amino acid sequence of SEQ ID NO: 68; and a V_{L} comprising a LC-CDR1, a LC-CDR2, and a LC-CDR3 of a V_{L} comprising the amino acid sequence of SEQ ID NO: 84;
(viii) a V_{H} comprising an HC-CDR1, an HC-CDR2, and an HC-CDR3 of a V_{H} comprising the amino acid sequence of SEQ ID NO: 68; and a V_{L} comprising a LC-CDR1, a LC-CDR2, and a LC-CDR3 of a V_{L} comprising the amino acid sequence of SEQ ID NO: 85;
(ix) a V_{H} comprising an HC-CDR1, an HC-CDR2, and an HC-CDR3 of a V_{H} comprising the amino acid sequence of SEQ ID NO: 69; and a V_{L} comprising a LC-CDR1, a LC-CDR2, and a LC-CDR3 of a V_{L} comprising the amino acid sequence of SEQ ID NO: 86;
(x) a V_{H} comprising an HC-CDR1, an HC-CDR2, and an HC-CDR3 of a V_{H} comprising the amino acid sequence of SEQ ID NO: 70; and a V_{L} comprising a LC-CDR1, a LC-CDR2, and a LC-CDR3 of a V_{L} comprising the amino acid sequence of SEQ ID NO: 87;
(xi) a V_{H} comprising an HC-CDR1, an HC-CDR2, and an HC-CDR3 of a V_{H} comprising the amino acid sequence of SEQ ID NO: 69; and a V_{L} comprising a LC-CDR1, a LC-CDR2, and a LC-CDR3 of a V_{L} comprising the amino acid sequence of SEQ ID NO: 88;
(xii) a V_{H} comprising an HC-CDR1, an HC-CDR2, and an HC-CDR3 of a V_{H} comprising the amino acid sequence of SEQ ID NO: 71; and a V_{L} comprising a LC-CDR1, a LC-CDR2, and a LC-CDR3 of a V_{L} comprising the amino acid sequence of SEQ ID NO: 89; or
(xiii) a V_{H} comprising an HC-CDR1, an HC-CDR2, and an HC-CDR3 of a V_{H} comprising the amino acid sequence of SEQ ID NO: 72; and a V_{L} comprising a LC-CDR1, a LC-CDR2, and a LC-CDR3 of a V_{L} comprising the amino acid sequence of SEQ ID NO: 90; or
(b)
(i) a V_{H} comprising an HC-CDR1, an HC-CDR2, and an HC-CDR3 of a V_{H} comprising the amino acid sequence of SEQ ID NO: 73; and a V_{L} comprising a LC-CDR1, a LC-CDR2, and a LC-CDR3 of a V_{L} comprising the amino acid sequence of SEQ ID NO: 91;
(ii) a V_{H} comprising an HC-CDR1, an HC-CDR2, and an HC-CDR3 of a V_{H} comprising the amino acid sequence of SEQ ID NO: 74; and a V_{L} comprising a LC-CDR1, a LC-CDR2, and a LC-CDR3 of a V_{L} comprising the amino acid sequence of SEQ ID NO: 91;
(iii) a V_{H} comprising an HC-CDR1, an HC-CDR2, and an HC-CDR3 of a V_{H} comprising the amino acid sequence of SEQ ID NO: 75; and a V_{L} comprising a LC-CDR1, a LC-CDR2, and a LC-CDR3 of a V_{L} comprising the amino acid sequence of SEQ ID NO: 91;
(iv) a V_{H} comprising an HC-CDR1, an HC-CDR2, and an HC-CDR3 of a V_{H} comprising the amino acid sequence of SEQ ID NO: 73; and a V_{L} comprising a LC-CDR1, a LC-CDR2, and a LC-CDR3 of a V_{L} comprising the amino acid sequence of SEQ ID NO: 92;
(v) a V_{H} comprising an HC-CDR1, an HC-CDR2, and an HC-CDR3 of a V_{H} comprising the amino acid sequence of SEQ ID NO: 73; and a V_{L} comprising a LC-CDR1, a LC-CDR2, and a LC-CDR3 of a V_{L} comprising the amino acid sequence of SEQ ID NO: 93;
(vi) a V_{H} comprising an HC-CDR1, an HC-CDR2, and an HC-CDR3 of a V_{H} comprising the amino acid sequence of SEQ ID NO: 73; and a V_{L} comprising a LC-CDR1, a LC-CDR2, and a LC-CDR3 of a V_{L} comprising the amino acid sequence of SEQ ID NO: 94;
(vii) a V_{H} comprising an HC-CDR1, an HC-CDR2, and an HC-CDR3 of a V_{H} comprising the amino acid sequence of SEQ ID NO: 76; and a V_{L} comprising a LC-CDR1, a LC-CDR2, and a LC-CDR3 of a V_{L} comprising the amino acid sequence of SEQ ID NO: 95;
(viii) a V_{H} comprising an HC-CDR1, an HC-CDR2, and an HC-CDR3 of a V_{H} comprising the amino acid sequence of SEQ ID NO: 77; and a V_{L} comprising a LC-CDR1, a LC-CDR2, and a LC-CDR3 of a V_{L} comprising the amino acid sequence of SEQ ID NO: 96; or
(ix) a V_{H} comprising an HC-CDR1, an HC-CDR2, and an HC-CDR3 of a V_{H} comprising the amino acid sequence of SEQ ID NO: 78; and a V_{L} comprising a LC-CDR1, a LC-CDR2, and a LC-CDR3 of a V_{L} comprising the amino acid sequence of SEQ ID NO: 97; or
(c)
(i) a V_{H} comprising an HC-CDR1, an HC-CDR2, and an HC-CDR3 of a V_{H} comprising the amino acid sequence of SEQ ID NO: 79; and a V_{L} comprising a LC-CDR1, a LC-CDR2, and a LC-CDR3 of a V_{L} comprising the amino acid sequence of SEQ ID NO: 98;
(ii) a V_{H} comprising an HC-CDR1, an HC-CDR2, and an HC-CDR3 of a V_{H} comprising the amino acid sequence of SEQ ID NO: 80; and a V_{L} comprising a LC-CDR1, a LC-CDR2, and a LC-CDR3 of a V_{L} comprising the amino acid sequence of SEQ ID NO: 99; or
(iii) a V_{H} comprising an HC-CDR1, an HC-CDR2, and an HC-CDR3 of a V_{H} comprising the amino acid sequence of SEQ ID NO: 81; and a V_{L} comprising a LC-CDR1, a LC-CDR2, and a LC-CDR3 of a V_{L} comprising the amino acid sequence of SEQ ID NO: 100; or
(d)
(i) a V_{H} comprising an HC-CDR1, an HC-CDR2, and an HC-CDR3 of a V_{H} comprising the amino acid sequence of SEQ ID NO: 82; and a V_{L} comprising a LC-CDR1, a LC-CDR2, and a LC-CDR3 of a V_{L} comprising the amino acid sequence of SEQ ID NO: 101; or
(ii) a V_{H} comprising an HC-CDR1, an HC-CDR2, and an HC-CDR3 of a V_{H} comprising the amino acid sequence of SEQ ID NO: 83; and a V_{L} comprising a LC-CDR1, a LC-CDR2, and a LC-CDR3 of a V_{L} comprising the amino acid sequence of SEQ ID NO: 102.

5. The isolated anti-TSLP antibody of claim 1, comprising:
(a)
(i) a V_{H} comprising an HC-CDR1 comprising the amino acid sequence of SEQ ID NO: 1, an HC-CDR2 comprising the amino acid sequence of SEQ ID NO: 7, and an HC-CDR3 comprising the amino acid sequence of SEQ ID NO: 16, or a variant thereof comprising up to about 5 amino acid substitutions in the HC-CDRs; and a V_{L} comprising a LC-CDR1 comprising the amino acid sequence of SEQ ID NO: 25, a LC-CDR2 comprising the amino acid sequence of SEQ ID NO: 39, and a LC-CDR3 comprising the amino acid sequence of SEQ ID NO: 50, or a variant thereof comprising up to about 5 amino acid substitutions in the LC-CDRs;
(ii) a V_{H} comprising an HC-CDR1 comprising the amino acid sequence of SEQ ID NO: 1, an HC-CDR2 comprising the amino acid sequence of SEQ ID NO: 8, and an HC-CDR3 comprising the amino acid sequence of SEQ ID NO: 16, or a variant thereof comprising up to about 5 amino acid substitutions in the HC-CDRs; and a V_{L} comprising a LC-CDR1 comprising the amino acid sequence of SEQ ID NO: 25, a LC-CDR2 comprising the amino acid sequence of SEQ ID NO: 39, and a LC-CDR3 comprising the amino acid sequence of SEQ ID NO: 51, or a variant thereof comprising up to about 5 amino acid substitutions in the LC-CDRs;
(iii) a V_{H} comprising an HC-CDR1 comprising the amino acid sequence of SEQ ID NO: 1, an HC-CDR2 comprising the amino acid sequence of SEQ ID NO: 8, and an HC-CDR3 comprising the amino acid sequence of SEQ ID NO: 17, or a variant thereof comprising up to about 5 amino acid substitutions in the HC-CDRs; and a V_{L} comprising a LC-CDR1 comprising the amino acid sequence of SEQ ID NO: 26, a LC-CDR2 comprising the amino acid sequence of SEQ ID NO: 40, and a LC-CDR3 comprising the amino acid sequence of SEQ ID NO: 52, or a variant thereof comprising up to about 5 amino acid substitutions in the LC-CDRs;
(iv) a V_{H} comprising an HC-CDR1 comprising the amino acid sequence of SEQ ID NO: 1, an HC-CDR2 comprising the amino acid sequence of SEQ ID NO: 8, and an HC-CDR3 comprising the amino acid sequence of SEQ ID NO: 16, or a variant thereof comprising up to about 5 amino acid substitutions in the HC-CDRs; and a V_{L} comprising a LC-CDR1 comprising the amino acid sequence of SEQ ID NO: 27, a LC-CDR2 comprising the amino acid sequence of SEQ ID NO: 41, and a LC-CDR3 comprising the amino acid sequence of SEQ ID NO: 52, or a variant thereof comprising up to about 5 amino acid substitutions in the LC-CDRs;
(v) a V_{H} comprising an HC-CDR1 comprising the amino acid sequence of SEQ ID NO: 1, an HC-CDR2 comprising the amino acid sequence of SEQ ID NO: 9, and an HC-CDR3 comprising the amino acid sequence of SEQ ID NO: 16, or a variant thereof comprising up to about 5 amino acid substitutions in the HC-CDRs; and a V_{L} comprising a LC-CDR1 comprising the amino acid sequence of SEQ ID NO: 28, a LC-CDR2 comprising the amino acid sequence of SEQ ID NO: 39, and a LC-CDR3 comprising the amino acid sequence of SEQ ID NO: 52, or a variant thereof comprising up to about 5 amino acid substitutions in the LC-CDRs; or
(vi) a V_{H} comprising an HC-CDR1 comprising the amino acid sequence of SEQ ID NO: 1, an HC-CDR2 comprising the amino acid sequence of SEQ ID NO: 8, and an HC-CDR3 comprising the amino acid sequence of SEQ ID NO: 18, or a variant thereof comprising up to about 5 amino acid substitutions in the HC-CDRs; and a V_{L} comprising a LC-CDR1 comprising the amino acid sequence of SEQ ID NO: 27, a LC-CDR2 comprising the amino acid sequence of SEQ ID NO: 39, and a LC-CDR3 comprising the amino acid sequence of SEQ ID NO: 53, or a variant thereof comprising up to about 5 amino acid substitutions in the LC-CDRs; or
(b)
(i) a V_{H} comprising an HC-CDR1 comprising the amino acid sequence of SEQ ID NO: 2, an HC-CDR2 comprising the amino acid sequence of SEQ ID NO: 10, and an HC-CDR3 comprising the amino acid sequence of SEQ ID NO: 19, or a variant thereof comprising up to about 5 amino acid substitutions in the HC-CDRs; and a V_{L} comprising a LC-CDR1 comprising the amino acid sequence of SEQ ID NO: 29, a LC-CDR2 comprising the amino acid sequence of SEQ ID NO: 42, and a LC-CDR3 comprising the amino acid sequence of SEQ ID NO: 54, or a variant thereof comprising up to about 5 amino acid substitutions in the LC-CDRs;
(ii) a V_{H} comprising an HC-CDR1 comprising the amino acid sequence of SEQ ID NO: 2, an HC-CDR2 comprising the amino acid sequence of SEQ ID NO: 10, and an HC-CDR3 comprising the amino acid sequence of SEQ ID NO: 19, or a variant thereof comprising up to about 5 amino acid substitutions in the HC-CDRs; and a V_{L} comprising a LC-CDR1 comprising the amino acid sequence of SEQ ID NO: 30, a LC-CDR2 comprising the amino acid sequence of SEQ ID NO: 43, and a LC-CDR3 comprising the amino acid sequence of SEQ ID NO: 55, or a variant thereof comprising up to about 5 amino acid substitutions in the LC-CDRs;
(iii) a V_{H} comprising an HC-CDR1 comprising the amino acid sequence of SEQ ID NO: 2, an HC-CDR2 comprising the amino acid sequence of SEQ ID NO: 10, and an HC-CDR3 comprising the amino acid sequence of SEQ ID NO: 19, or a variant thereof comprising up to about 5 amino acid substitutions in the HC-CDRs; and a V_{L} comprising a LC-CDR1 comprising the amino acid sequence of SEQ ID NO: 31, a LC-CDR2 comprising the amino acid sequence of SEQ ID NO: 44, and a LC-CDR3 comprising the amino acid sequence of SEQ ID NO: 56, or a variant thereof comprising up to about 5 amino acid substitutions in the LC-CDRs;
(iv) a V_{H} comprising an HC-CDR1 comprising the amino acid sequence of SEQ ID NO: 2, an HC-CDR2 comprising the amino acid sequence of SEQ ID NO: 10, and an HC-CDR3 comprising the amino acid sequence of SEQ ID NO: 19, or a variant thereof comprising up to about 5 amino acid substitutions in the HC-CDRs; and a V_{L} comprising a LC-CDR1 comprising the amino acid sequence of SEQ ID NO: 32, a LC-CDR2 comprising the amino acid sequence of SEQ ID NO: 45, and a LC-CDR3 comprising the amino acid sequence of SEQ ID NO: 57, or a variant thereof comprising up to about 5 amino acid substitutions in the LC-CDRs;
(v) a V_{H} comprising an HC-CDR1 comprising the amino acid sequence of SEQ ID NO: 3, an HC-CDR2 comprising the amino acid sequence of SEQ ID NO: 11, and an HC-CDR3 comprising the amino acid sequence of SEQ ID NO: 19, or a variant thereof comprising up to about 5 amino acid substitutions in the HC-CDRs; and a V_{L} comprising a LC-CDR1 comprising the amino acid sequence of SEQ ID NO: 33, a LC-CDR2 comprising the amino acid sequence of SEQ ID NO: 45, and a LC-CDR3 comprising the amino acid sequence of SEQ ID NO: 58, or a variant thereof comprising up to about 5 amino acid substitutions in the LC-CDRs;
(vi) a V_{H} comprising an HC-CDR1 comprising the amino acid sequence of SEQ ID NO: 3, an HC-CDR2 comprising the amino acid sequence of SEQ ID NO: 10, and an HC-CDR3 comprising the amino acid sequence of SEQ ID NO: 19, or a variant thereof comprising up to about 5 amino acid substitutions in the HC-CDRs; and a V_{L} comprising a LC-CDR1 comprising the amino acid sequence of SEQ ID NO: 31, a LC-CDR2 comprising the amino acid sequence of SEQ ID NO: 46, and a LC-CDR3 comprising the amino acid sequence of SEQ ID NO: 59, or a variant thereof comprising up to about 5 amino acid substitutions in the LC-CDRs; or
(vii) a V_{H} comprising an HC-CDR1 comprising the amino acid sequence of SEQ ID NO: 3, an HC-CDR2 comprising the amino acid sequence of SEQ ID NO: 12, and an HC-CDR3 comprising the amino acid sequence of SEQ ID NO: 20, or a variant thereof comprising up to about 5 amino acid substitutions in the HC-CDRs; and a V_{L} comprising a LC-CDR1 comprising the amino acid sequence of SEQ ID NO: 34, a LC-CDR2 comprising the amino acid sequence of SEQ ID NO: 47, and a LC-CDR3 comprising the amino acid sequence of SEQ ID NO: 60, or a variant thereof comprising up to about 5 amino acid substitutions in the LC-CDRs; or
(c)
(i) a V_{H} comprising an HC-CDR1 comprising the amino acid sequence of SEQ ID NO: 4, an HC-CDR2 comprising the amino acid sequence of SEQ ID NO: 13, and an HC-CDR3 comprising the amino acid sequence of SEQ ID NO: 21, or a variant thereof comprising up to about 5 amino acid substitutions in the HC-CDRs; and a V_{L} comprising a LC-CDR1 comprising the amino acid sequence of SEQ ID NO: 35, a LC-CDR2 comprising the amino acid sequence of SEQ ID NO: 48, and a LC-CDR3 comprising the amino acid sequence of SEQ ID NO: 61, or a variant thereof comprising up to about 5 amino acid substitutions in the LC-CDRs;
(ii) a V_{H} comprising an HC-CDR1 comprising the amino acid sequence of SEQ ID NO: 4, an HC-CDR2 comprising the amino acid sequence of SEQ ID NO: 13, and an HC-CDR3 comprising the amino acid sequence of SEQ ID NO: 22, or a variant thereof comprising up to about 5 amino acid substitutions in the HC-CDRs; and a V_{L} comprising a LC-CDR1 comprising the amino acid sequence of SEQ ID NO: 36, a LC-CDR2 comprising the amino acid sequence of SEQ ID NO: 48, and a LC-CDR3 comprising the amino acid sequence of SEQ ID NO: 62, or a variant thereof comprising up to about 5 amino acid substitutions in the LC-CDRs; or
(iii) a V_{H} comprising an HC-CDR1 comprising the amino acid sequence of SEQ ID NO: 5, an HC-CDR2 comprising the amino acid sequence of SEQ ID NO: 13, and an HC-CDR3 comprising the amino acid sequence of SEQ ID NO: 22, or a variant thereof comprising up to about 5 amino acid substitutions in the HC-CDRs; and a V_{L} comprising a LC-CDR1 comprising the amino acid sequence of SEQ ID NO: 37, a LC-CDR2 comprising the amino acid sequence of SEQ ID NO: 49, and a LC-CDR3 comprising the amino acid sequence of SEQ ID NO: 63, or a variant thereof comprising up to about 5 amino acid substitutions in the LC-CDRs; or
(d)
(i) a V_{H} comprising an HC-CDR1 comprising the amino acid sequence of SEQ ID NO: 6, an HC-CDR2 comprising the amino acid sequence of SEQ ID NO: 14, and an HC-CDR3 comprising the amino acid sequence of SEQ ID NO: 23, or a variant thereof comprising up to about 5 amino acid substitutions in the HC-CDRs; and a V_{L} comprising a LC-CDR1 comprising the amino acid sequence of SEQ ID NO: 38, a LC-CDR2 comprising the amino acid sequence of SEQ ID NO: 42, and a LC-CDR3 comprising the amino acid sequence of SEQ ID NO: 60, or a variant thereof comprising up to about 5 amino acid substitutions in the LC-CDRs; or
(ii) a V_{H} comprising an HC-CDR1 comprising the amino acid sequence of SEQ ID NO: 6, an HC-CDR2 comprising the amino acid sequence of SEQ ID NO: 15, and an HC-CDR3 comprising the amino acid sequence of SEQ ID NO: 24, or a variant thereof comprising up to about 5 amino acid substitutions in the HC-CDRs; and a V_{L} comprising a LC-CDR1 comprising the amino acid sequence of SEQ ID NO: 31, a LC-CDR2 comprising the amino acid sequence of SEQ ID NO: 45, and a LC-CDR3 comprising the amino acid sequence of SEQ ID NO: 64, or a variant thereof comprising up to about 5 amino acid substitutions in the LC-CDRs.

6. The isolated anti-TSLP antibody of claim 1, comprising:
(a) a V_{H} comprising the amino acid sequence of any one of SEQ ID NOs: 65-72, or a variant thereof having at least about 90% sequence identity to the amino acid sequence of any one of SEQ ID NOs: 65-72; and
a V_{L} comprising the amino acid sequence of any one of SEQ ID NOs: 84-90, or a variant thereof having at least about 90% sequence identity to the amino acid sequence of any one of SEQ ID NOs: 84-90; or
(b) a V_{H} comprising the amino acid sequence of any one of SEQ ID NOs: 73-78, or a variant thereof having at least about 90% sequence identity to the amino acid sequence of any one of SEQ ID NOs: 73-78; and
a V_{L} comprising the amino acid sequence of any one of SEQ ID NOs: 91-97, or a variant thereof having at least about 90% sequence identity to the amino acid sequence of any one of SEQ ID NOs: 91-97; or
(c) a V_{H} comprising the amino acid sequence of any one of SEQ ID NOs: 79-81, or a variant thereof having at least about 90% sequence identity to the amino acid sequence of any one of SEQ ID NOs: 79-81; and
a V_{L} comprising the amino acid sequence of any one of SEQ ID NOs: 98-100, or a variant thereof having at least about 90% sequence identity to the amino acid sequence of any one of SEQ ID NOs: 98-100; or
(d) a V_{H} comprising the amino acid sequence of any one of SEQ ID NOs: 82-83, or a variant thereof having at least about 90% sequence identity to the amino acid sequence of any one of SEQ ID NOs: 82-83; and
a V_{L} comprising the amino acid sequence of any one of SEQ ID NOs: 101-102, or a variant thereof having at least about 90% sequence identity to the amino acid sequence of any one of SEQ ID NOs: 101-102.

7. The isolated anti-TSLP antibody of claim 6, comprising:
(a)
(i) a V_{H} comprising the amino acid sequence of SEQ ID NO: 65, or a variant thereof having at least about 90% sequence identity to the amino acid sequence of SEQ ID NO: 65; and a V_{L} comprising the amino acid sequence of SEQ ID NO: 84, or a variant thereof having at least about 90% sequence identity to the amino acid sequence of SEQ ID NO: 84;
(ii) a V_{H} comprising the amino acid sequence of SEQ ID NO: 65, or a variant thereof having at least about 90% sequence identity to the amino acid sequence of SEQ ID NO: 65; and a V_{L} comprising the amino acid sequence of SEQ ID NO: 85, or a variant thereof having at least about 90% sequence identity to the amino acid sequence of SEQ ID NO: 85;
(iii) a V_{H} comprising the amino acid sequence of SEQ ID NO: 66, or a variant thereof having at least about 90% sequence identity to the amino acid sequence of SEQ ID NO: 66; and a V_{L} comprising the amino acid sequence of SEQ ID NO: 84, or a variant thereof having at least about 90% sequence identity to the amino acid sequence of SEQ ID NO: 84;
(iv) a V_{H} comprising the amino acid sequence of SEQ ID NO: 66, or a variant thereof having at least about 90% sequence identity to the amino acid sequence of SEQ ID NO: 66; and a V_{L} comprising the amino acid sequence of SEQ ID NO: 85, or a variant thereof having at least about 90% sequence identity to the amino acid sequence of SEQ ID NO: 85;
(v) a V_{H} comprising the amino acid sequence of SEQ ID NO: 67, or a variant thereof having at least about 90% sequence identity to the amino acid sequence of SEQ ID NO: 67; and a V_{L} comprising the amino acid sequence of SEQ ID NO: 84, or a variant thereof having at least about 90% sequence identity to the amino acid sequence of SEQ ID NO: 84;
(vi) a V_{H} comprising the amino acid sequence of SEQ ID NO: 67, or a variant thereof having at least about 90% sequence identity to the amino acid sequence of SEQ ID NO: 67; and a V_{L} comprising the amino acid sequence of SEQ ID NO: 85, or a variant thereof having at least about 90% sequence identity to the amino acid sequence of SEQ ID NO: 85;
(vii) a V_{H} comprising the amino acid sequence of SEQ ID NO: 68, or a variant thereof having at least about 90% sequence identity to the amino acid sequence of SEQ ID NO: 68; and a V_{L} comprising the amino acid sequence of SEQ ID NO: 84, or a variant thereof having at least about 90% sequence identity to the amino acid sequence of SEQ ID NO: 84;
(viii) a V_{H} comprising the amino acid sequence of SEQ ID NO: 68, or a variant thereof having at least about 90% sequence identity to the amino acid sequence of SEQ ID NO: 68; and a V_{L} comprising the amino acid sequence of SEQ ID NO: 85, or a variant thereof having at least about 90% sequence identity to the amino acid sequence of SEQ ID NO: 85;
(ix) a V_{H} comprising the amino acid sequence of SEQ ID NO: 69, or a variant thereof having at least about 90% sequence identity to the amino acid sequence of SEQ ID NO: 69; and a V_{L} comprising the amino acid sequence of SEQ ID NO: 86, or a variant thereof having at least about 90% sequence identity to the amino acid sequence of SEQ ID NO: 86;
(x) a V_{H} comprising the amino acid sequence of SEQ ID NO: 70, or a variant thereof having at least about 90% sequence identity to the amino acid sequence of SEQ ID NO: 70; and a V_{L} comprising the amino acid sequence of SEQ ID NO: 87, or a variant thereof having at least about 90% sequence identity to the amino acid sequence of SEQ ID NO: 87;
(xi) a V_{H} comprising the amino acid sequence of SEQ ID NO: 69, or a variant thereof having at least about 90% sequence identity to the amino acid sequence of SEQ ID NO: 69; and a V_{L} comprising the amino acid sequence of SEQ ID NO: 88, or a variant thereof having at least about 90% sequence identity to the amino acid sequence of SEQ ID NO: 88;
(xii) a V_{H} comprising the amino acid sequence of SEQ ID NO: 71, or a variant thereof having at least about 90% sequence identity to the amino acid sequence of SEQ ID NO: 71; and a V_{L} comprising the amino acid sequence of SEQ ID NO: 89, or a variant thereof having at least about 90% sequence identity to the amino acid sequence of SEQ ID NO: 89; or
(xiii) a V_{H} comprising the amino acid sequence of SEQ ID NO: 72, or a variant thereof having at least about 90% sequence identity to the amino acid sequence of SEQ ID NO: 72; and a V_{L} comprising the amino acid sequence of SEQ ID NO: 90, or a variant thereof having at least about 90% sequence identity to the amino acid sequence of SEQ ID NO: 90; or
(b)
(i) a V_{H} comprising the amino acid sequence of SEQ ID NO: 73, or a variant thereof having at least about 90% sequence identity to the amino acid sequence of SEQ ID NO: 73; and a V_{L} comprising the amino acid sequence of SEQ ID NO: 91, or a variant thereof having at least about 90% sequence identity to the amino acid sequence of SEQ ID NO: 91;
(ii) a V_{H} comprising the amino acid sequence of SEQ ID NO: 74, or a variant thereof having at least about 90% sequence identity to the amino acid sequence of SEQ ID NO: 74; and a V_{L} comprising the amino acid sequence of SEQ ID NO: 91, or a variant thereof having at least about 90% sequence identity to the amino acid sequence of SEQ ID NO: 91;
(iii) a V_{H} comprising the amino acid sequence of SEQ ID NO: 75, or a variant thereof having at least about 90% sequence identity to the amino acid sequence of SEQ ID NO: 75; and a V_{L} comprising the amino acid sequence of SEQ ID NO: 91, or a variant thereof having at least about 90% sequence identity to the amino acid sequence of SEQ ID NO: 91;
(iv) a V_{H} comprising the amino acid sequence of SEQ ID NO: 73, or a variant thereof having at least about 90% sequence identity to the amino acid sequence of SEQ ID NO: 73; and a V_{L} comprising the amino acid sequence of SEQ ID NO: 92, or a variant thereof having at least about 90% sequence identity to the amino acid sequence of SEQ ID NO: 92;
(v) a V_{H} comprising the amino acid sequence of SEQ ID NO: 73, or a variant thereof having at least about 90% sequence identity to the amino acid sequence of SEQ ID NO: 73; and a V_{L} comprising the amino acid sequence of SEQ ID NO: 93, or a variant thereof having at least about 90% sequence identity to the amino acid sequence of SEQ ID NO: 93;
(vi) a V_{H} comprising the amino acid sequence of SEQ ID NO: 73, or a variant thereof having at least about 90% sequence identity to the amino acid sequence of SEQ ID NO: 73; and a V_{L} comprising the amino acid sequence of SEQ ID NO: 94, or a variant thereof having at least about 90% sequence identity to the amino acid sequence of SEQ ID NO: 94;
(vii) a V_{H} comprising the amino acid sequence of SEQ ID NO: 76, or a variant thereof having at least about 90% sequence identity to the amino acid sequence of SEQ ID NO: 76; and a V_{L} comprising the amino acid sequence of SEQ ID NO: 95, or a variant thereof having at least about 90% sequence identity to the amino acid sequence of SEQ ID NO: 95;
(viii) a V_{H} comprising the amino acid sequence of SEQ ID NO: 77, or a variant thereof having at least about 90% sequence identity to the amino acid sequence of SEQ ID NO: 77; and a V_{L} comprising the amino acid sequence of SEQ ID NO: 96, or a variant thereof having at least about 90% sequence identity to the amino acid sequence of SEQ ID NO: 96; or
(ix) a V_{H} comprising the amino acid sequence of SEQ ID NO: 78, or a variant thereof having at least about 90% sequence identity to the amino acid sequence of SEQ ID NO: 78; and a V_{L} comprising the amino acid sequence of SEQ ID NO: 97, or a variant thereof having at least about 90% sequence identity to the amino acid sequence of SEQ ID NO: 97; or
(c)
(i) a V_{H} comprising the amino acid sequence of SEQ ID NO: 79, or a variant thereof having at least about 90% sequence identity to the amino acid sequence of SEQ ID NO: 79; and a V_{L} comprising the amino acid sequence of SEQ ID NO: 98, or a variant thereof having at least about 90% sequence identity to the amino acid sequence of SEQ ID NO: 98;
(ii) a V_{H} comprising the amino acid sequence of SEQ ID NO: 80, or a variant thereof having at least about 90% sequence identity to the amino acid sequence of SEQ ID NO: 80; and a V_{L} comprising the amino acid sequence of SEQ ID NO: 99, or a variant thereof having at least about 90% sequence identity to the amino acid sequence of SEQ ID NO: 99; or
(iii) a V_{H} comprising the amino acid sequence of SEQ ID NO: 81, or a variant thereof having at least about 90% sequence identity to the amino acid sequence of SEQ ID NO: 81; and a V_{L} comprising the amino acid sequence of SEQ ID NO: 100, or a variant thereof having at least about 90% sequence identity to the amino acid sequence of SEQ ID NO: 100; or
(d)
(i) a V_{H} comprising the amino acid sequence of SEQ ID NO: 82, or a variant thereof having at least about 90% sequence identity to the amino acid sequence of SEQ ID NO: 82; and a V_{L} comprising the amino acid sequence of SEQ ID NO: 101, or a variant thereof having at least about 90% sequence identity to the amino acid sequence of SEQ ID NO: 101; or
(ii) a V_{H} comprising the amino acid sequence of SEQ ID NO: 83, or a variant thereof having at least about 90% sequence identity to the amino acid sequence of SEQ ID NO: 83; and a V_{L} comprising the amino acid sequence of SEQ ID NO: 102, or a variant thereof having at least about 90% sequence identity to the amino acid sequence of SEQ ID NO: 102.

8-28. (canceled)

29. An isolated anti-TSLP antibody that specifically binds to TSLP competitively with the isolated anti-TSLP antibody of claim 1, or specifically binds to the same epitope as the isolated anti-TSLP antibody of claim 1.

30. The isolated anti-TSLP antibody of claim 1, wherein the anti-TSLP antibody binds to the human TSLP with a Kd from about 0.1 pM to about 1 nM.

31. The isolated anti-TSLP antibody according to claim 1, wherein the anti-TSLP antibody comprises an Fc fragment,
optionally, the anti-TSLP antibody is a full-length IgA, IgD, IgE, IgG or IgM antibody,
optionally, the anti-TSLP antibody is a full-length IgG1, IgG2, IgG3 or IgG4 antibody.

32-33. (canceled)

34. The isolated anti-TSLP antibody of claim 1, wherein the anti-TSLP antibody is chimeric, human, or humanized.

35. The isolated anti-TSLP antibody according to claim 1, wherein the anti-TSLP antibody is an antigen binding fragment selected from the group consisting of a Fab, a Fab′, a F(ab)′₂, a Fab′-SH, a single-chain Fv (scFv), an Fv fragment, a dAb, a Fd, a nanobody, a diabody, and a linear antibody.

36. An isolated nucleic acid molecule that encodes the anti-TSLP antibody according to claim 1.

37. A vector comprising the isolated nucleic acid molecule of claim 36.

38. An isolated host cell comprising the nucleic acid of claim 36.

39. A method of producing an anti-TSLP antibody, comprising:
a) culturing the isolated host cell of claim 38 under conditions effective to express the anti-TSLP antibody; and
b) obtaining the expressed anti-TSLP antibody from the host cell.

40. A pharmaceutical composition comprising the isolated anti-TSLP antibody according to claim 1, and a pharmaceutically acceptable carrier.

41. A method of treating a disease or condition in an individual in need thereof, comprising administering to the individual an effective amount of the pharmaceutical composition of claim 40.

42. The method of claim 41, wherein the disease or condition is associated with TSLP signaling, comprising inflammatory or autoimmune disease or condition.

43. The method of claim 42, wherein the disease or condition is selected from the group consisting of asthma, idiopathic pulmonary fibrosis, atopic dermatitis, allergic conjunctivitis, allergic rhinitis, Netherton syndrome, eosinophilic esophagitis (EoE), food allergy, allergic diarrhoea, eosinophilic gastroenteritis, allergic bronchopulmonary aspergillosis (ABPA), allergic fungal sinusitis, cancer, rheumatoid arthritis, chronic obstructive pulmonary disease (COPD), systemic sclerosis, keloids, ulcerative colitis, chronic rhinosinusitis (CRS), nasal polyposis, chronic eosinophilic pneumonia, eosinophilic bronchitis, coeliac disease, Churg-Strauss syndrome, eosinophilic myalgia syndrome, hypereosinophilic syndrome, eosinophilic granulomatosis with polyangiitis, and inflammatory bowel disease.


SEQUENCE LISTING (129 sequences)

SEQ ID NO: 1 - protein, 6 aa, Artificial Sequence
  Synthetic Construct
SGYGWS

SEQ ID NO: 2 - protein, 5 aa, Artificial Sequence
  Synthetic Construct
SYGIN

SEQ ID NO: 3 - protein, 5 aa, Artificial Sequence
  Synthetic Construct
SYGIS

SEQ ID NO: 4 - protein, 5 aa, Artificial Sequence
  Synthetic Construct
NYDMT

SEQ ID NO: 5 - protein, 5 aa, Artificial Sequence
  Synthetic Construct
NYGMT

SEQ ID NO: 6 - protein, 5 aa, Artificial Sequence
  Synthetic Construct
SYAIS

SEQ ID NO: 7 - protein, 16 aa, Artificial Sequence
  Synthetic Construct
YISYYGSISYNPSLKS

SEQ ID NO: 8 - protein, 16 aa, Artificial Sequence
  Synthetic Construct
YISYYGSTSYNPSLKS

SEQ ID NO: 9 - protein, 16 aa, Artificial Sequence
  Synthetic Construct
YFSYYGSTSYNPSLKS

SEQ ID NO: 10 - protein, 17 aa, Artificial Sequence
  Synthetic Construct
VIVPLVGVTIYAEKFQG

SEQ ID NO: 11 - protein, 17 aa, Artificial Sequence
  Synthetic Construct
VIVPLVDVTIYAEKFQG

SEQ ID NO: 12 - protein, 17 aa, Artificial Sequence
  Synthetic Construct
VIIPLLGVPNYAEKFQG

SEQ ID NO: 13 - protein, 17 aa, Artificial Sequence
  Synthetic Construct
SITFASSYIYYADSVKG

SEQ ID NO: 14 - protein, 17 aa, Artificial Sequence
  Synthetic Construct
MLVPLLGVTNYAEKFQG

SEQ ID NO: 15 - protein, 17 aa, Artificial Sequence
  Synthetic Construct
MIIPLLGVTDYAEKFQG

SEQ ID NO: 16 - protein, 8 aa, Artificial Sequence
  Synthetic Construct
TNLLYFDS

SEQ ID NO: 17 - protein, 8 aa, Artificial Sequence
  Synthetic Construct
TNLLYFES

SEQ ID NO: 18 - protein, 8 aa, Artificial Sequence
  Synthetic Construct
TNLLYFDY

SEQ ID NO: 19 - protein, 11 aa, Artificial Sequence
  Synthetic Construct
GQEYFYWYFDL

SEQ ID NO: 20 - protein, 11 aa, Artificial Sequence
  Synthetic Construct
GAEYFYWYFDL

SEQ ID NO: 21 - protein, 12 aa, Artificial Sequence
  Synthetic Construct
GGGAYHGGSLDV

SEQ ID NO: 22 - protein, 12 aa, Artificial Sequence
  Synthetic Construct
GGGAYYGGSLDV

SEQ ID NO: 23 - protein, 12 aa, Artificial Sequence
  Synthetic Construct
GGSNYLYWYFDL

SEQ ID NO: 24 - protein, 12 aa, Artificial Sequence
  Synthetic Construct
GGTNYLYWYFDL

SEQ ID NO: 25 - protein, 11 aa, Artificial Sequence
  Synthetic Construct
RASQSVSNNLA

SEQ ID NO: 26 - protein, 11 aa, Artificial Sequence
  Synthetic Construct
RASQGISSYLA

SEQ ID NO: 27 - protein, 11 aa, Artificial Sequence
  Synthetic Construct
RASQSVSSYLA

SEQ ID NO: 28 - protein, 11 aa, Artificial Sequence
  Synthetic Construct
RASQSVSSNLA

SEQ ID NO: 29 - protein, 14 aa, Artificial Sequence
  Synthetic Construct
SGSSSDIGGYNRVS

SEQ ID NO: 30 - protein, 14 aa, Artificial Sequence
  Synthetic Construct
TGTSSDIGGYDRVS

SEQ ID NO: 31 - protein, 14 aa, Artificial Sequence
  Synthetic Construct
TGTSSDIGGYNRVS

SEQ ID NO: 32 - protein, 14 aa, Artificial Sequence
  Synthetic Construct
TGTTSDIGGYNRVS

SEQ ID NO: 33 - protein, 14 aa, Artificial Sequence
  Synthetic Construct
TGTISDIGGYNRVS

SEQ ID NO: 34 - protein, 14 aa, Artificial Sequence
  Synthetic Construct
TGSNSDIGGYNRVS

SEQ ID NO: 35 - protein, 16 aa, Artificial Sequence
  Synthetic Construct
RSSQSLLHINGYTYLH

SEQ ID NO: 36 - protein, 16 aa, Artificial Sequence
  Synthetic Construct
RSSQSLLHSNEYTYLH

SEQ ID NO: 37 - protein, 16 aa, Artificial Sequence
  Synthetic Construct
RSSQSLLHSYGYTYLH

SEQ ID NO: 38 - protein, 14 aa, Artificial Sequence
  Synthetic Construct
TGTSSDIGGYNRIS

SEQ ID NO: 39 - protein, 7 aa, Artificial Sequence
  Synthetic Construct
DASSRAT

SEQ ID NO: 40 - protein, 7 aa, Artificial Sequence
  Synthetic Construct
DASNLQS

SEQ ID NO: 41 - protein, 7 aa, Artificial Sequence
  Synthetic Construct
DTSSRAT

SEQ ID NO: 42 - protein, 7 aa, Artificial Sequence
  Synthetic Construct
DVSKRPS

SEQ ID NO: 43 - protein, 7 aa, Artificial Sequence
  Synthetic Construct
GVSKRPS

SEQ ID NO: 44 - protein, 7 aa, Artificial Sequence
  Synthetic Construct
EVNKRSS

SEQ ID NO: 45 - protein, 7 aa, Artificial Sequence
  Synthetic Construct
EVSKRPS

SEQ ID NO: 46 - protein, 7 aa, Artificial Sequence
  Synthetic Construct
EFNKRPS

SEQ ID NO: 47 - protein, 7 aa, Artificial Sequence
  Synthetic Construct
EISKRPS

SEQ ID NO: 48 - protein, 7 aa, Artificial Sequence
  Synthetic Construct
LVSHRAS

SEQ ID NO: 49 - protein, 7 aa, Artificial Sequence
  Synthetic Construct
LVSYRAS

SEQ ID NO: 50 - protein, 10 aa, Artificial Sequence
  Synthetic Construct
QQYSDWPQYT

SEQ ID NO: 51 - protein, 10 aa, Artificial Sequence
  Synthetic Construct
QQYSDWPEYS

SEQ ID NO: 52 - protein, 10 aa, Artificial Sequence
  Synthetic Construct
QQYSNWPQYS

SEQ ID NO: 53 - protein, 10 aa, Artificial Sequence
  Synthetic Construct
QQYSDWPQYS

SEQ ID NO: 54 - protein, 11 aa, Artificial Sequence
  Synthetic Construct
SSYAGTDTFIL

SEQ ID NO: 55 - protein, 11 aa, Artificial Sequence
  Synthetic Construct
SSYASGHTFGL

SEQ ID NO: 56 - protein, 11 aa, Artificial Sequence
  Synthetic Construct
TSYAGTDTFVL

SEQ ID NO: 57 - protein, 11 aa, Artificial Sequence
  Synthetic Construct
SSYAGTDTFAI

SEQ ID NO: 58 - protein, 11 aa, Artificial Sequence
  Synthetic Construct
SSYAGTDTFVF

SEQ ID NO: 59 - protein, 11 aa, Artificial Sequence
  Synthetic Construct
SSYAGTDIFVL

SEQ ID NO: 60 - protein, 11 aa, Artificial Sequence
  Synthetic Construct
SSYAGTDTFVL

SEQ ID NO: 61 - protein, 9 aa, Artificial Sequence
  Synthetic Construct
EQTLQTPYS

SEQ ID NO: 62 - protein, 9 aa, Artificial Sequence
  Synthetic Construct
EQTLQTPFT

SEQ ID NO: 63 - protein, 9 aa, Artificial Sequence
  Synthetic Construct
EQTLQTPYT

SEQ ID NO: 64 - protein, 11 aa, Artificial Sequence
  Synthetic Construct
SAYAGTDTFVL

SEQ ID NO: 65 - protein, 117 aa, Artificial Sequence
  Synthetic Construct
QVQLQESGPGLVKPSETLSLTCAVSGYSISSGYGWSWIRQPPGKGLEWIGYISYYGSISYNPSLKSRVTISRDTSKNQFSLKLSSVTAADTAVYYCARTNLLYFDSWGQGTLVTVSS

SEQ ID NO: 66 - protein, 117 aa, Artificial Sequence
  Synthetic Construct
QVQLQESGPGLVKPSETLSLTCTVSGYSISSGYGWSWIRQPPGKGLEWIGYISYYGSISYNPSLKSRVTISRDTSKNQFSLKLSSVTAADTAVYYCARTNLLYFDSWGQGTLVTVSS

SEQ ID NO: 67 - protein, 117 aa, Artificial Sequence
  Synthetic Construct
QVQLQESGPGLVKPSETLSLTCAVSGYSISSGYGWSWIRQPPGKGLEWIGYISYYGSISYNPSLKSRVTISVDTSKNQFSLKLSSVTAADTAVYYCARTNLLYFDSWGQGTLVTVSS

SEQ ID NO: 68 - protein, 117 aa, Artificial Sequence
  Synthetic Construct
QVQLQESGPGLVKPSETLSLTCTVSGYSISSGYGWSWIRQPPGKGLEWIGYISYYGSISYNPSLKSRVTISVDTSKNQFSLKLSSVTAADTAVYYCARTNLLYFDSWGQGTLVTVSS

SEQ ID NO: 69 - protein, 117 aa, Artificial Sequence
  Synthetic Construct
QVQLQESGPGLVKPSETLSLTCAVSGYSISSGYGWSWIRQPPGKGLEWIGYISYYGSTSYNPSLKSRVTISRDTSKNQFSLKLSSVTAADTAVYYCARTNLLYFDSWGQGTLVTVSS

SEQ ID NO: 70 - protein, 117 aa, Artificial Sequence
  Synthetic Construct
QVQLQESGPGLVKPSETLSLTCAVSGYSISSGYGWSWIRQPPGKGLEWIGYISYYGSTSYNPSLKSRVTISRDTSKNQFSLKLSSVTAADTAVYYCARTNLLYFESWGQGTLVTVSS

SEQ ID NO: 71 - protein, 117 aa, Artificial Sequence
  Synthetic Construct
QVQLQESGPGLVKPSETLSLTCAVSGYSISSGYGWSWIRQPPGKGLEWIGYFSYYGSTSYNPSLKSRVTISRDTSKNQFSLKLSSVTAADTAVYYCARTNLLYFDSWGQGTLVTVSS

SEQ ID NO: 72 - protein, 117 aa, Artificial Sequence
  Synthetic Construct
QVQLQESGPGLVKPSETLSLTCAVSGYSISSGYGWSWIRQPPGKGLEWIGYISYYGSTSYNPSLKSRVTISRDTSKNQFSLKLSSVTAADTAVYYCARTNLLYFDYWGQGTLVTVSS

SEQ ID NO: 73 - protein, 120 aa, Artificial Sequence
  Synthetic Construct
QVQLVQSGAEVKKPGSSVKVSCKASGFTFGSYGINWVRQAPGQGLEWLGVIVPLVGVTIYAEKFQGRVTITADTSTSTAYMELSSLRSEDTAVYYCARGQEYFYWYFDLWGQGTLVTVSS

SEQ ID NO: 74 - protein, 120 aa, Artificial Sequence
  Synthetic Construct
QVQLVQSGAEVKKPGSSVKVSCKASGFTFGSYGINWVRQAPGQGLEWMGVIVPLVGVTIYAEKFQGRVTITADTSTSTAYMELSSLRSEDTAVYYCARGQEYFYWYFDLWGQGTLVTVSS

SEQ ID NO: 75 - protein, 120 aa, Artificial Sequence
  Synthetic Construct
QVQLVQSGAEVKKPGSSVKVSCKASGFTFGSYGINWVRQAPGQGLEWMGVIVPLVGVTIYAEKFQGRVTITADESTSTAYMELSSLRSEDTAVYYCARGQEYFYWYFDLWGQGTLVTVSS

SEQ ID NO: 76 - protein, 120 aa, Artificial Sequence
  Synthetic Construct
QVQLVQSGAEVKKPGSSVKVSCKASGFTFGSYGISWVRQAPGQGLEWLGVIVPLVDVTIYAEKFQGRVTITADTSTSTAYMELSSLRSEDTAVYYCARGQEYFYWYFDLWGQGTLVTVSS

SEQ ID NO: 77 - protein, 120 aa, Artificial Sequence
  Synthetic Construct
QVQLVQSGAEVKKPGSSVKVSCKASGFTFGSYGISWVRQAPGQGLEWLGVIVPLVGVTIYAEKFQGRVTITADTSTSTAYMELSSLRSEDTAVYYCARGQEYFYWYFDLWGQGTLVTVSS

SEQ ID NO: 78 - protein, 120 aa, Artificial Sequence
  Synthetic Construct
QVQLVQSGAEVKKPGSSVKVSCKASGFTFGSYGISWVRQAPGQGLEWLGVIIPLLGVPNYAEKFQGRVTITADTSTSTAYMELSSLRSEDTAVYYCARGAEYFYWYFDLWGQGTLVTVSS

SEQ ID NO: 79 - protein, 121 aa, Artificial Sequence
  Synthetic Construct
EVQLVESGGGLVQPGGSLRLSCAASGFTFSNYDMTWVRQAPGKGLEWVSSITFASSYIYYADSVKGRFTISRDNAKNSLSLQMNSLRAEDTAVYYCTRGGGAYHGGSLDVWGQGVLVTVSS

SEQ ID NO: 80 - protein, 121 aa, Artificial Sequence
  Synthetic Construct
EVQLVESGGGLVQPGGSLRLSCAASGFTFSNYDMTWVRQAPGKGLEWVSSITFASSYIYYADSVKGRFTISRDNAKNSLSLQMNSLRAEDTAVYYCTRGGGAYYGGSLDVWGQGVLVTVSS

SEQ ID NO: 81 - protein, 121 aa, Artificial Sequence
  Synthetic Construct
EVQLVESGGGLVQPGGSLRLSCAASGFTFSNYGMTWVRQAPGKGLEWVSSITFASSYIYYADSVKGRFTISRDNAKNSLSLQMNSLRAEDTAVYYCTRGGGAYYGGSLDVWGQGVLVTVSS

SEQ ID NO: 82 - protein, 121 aa, Artificial Sequence
  Synthetic Construct
QEQLVQSGDEVKKPGASVKVSCKSSGFTFGSYAISWVRQAPGQGLEWMGMLVPLLGVTNYAEKFQGRVTITADPSTTTTSMELSSLRSEDTAVYYCARGGSNYLYWYFDLWGPGTPITVSS

SEQ ID NO: 83 - protein, 121 aa, Artificial Sequence
  Synthetic Construct
QEQLVQSGDEVKKPGASVKVSCKSSGFTFGSYAISWVRQAPGQGLEWMGMIIPLLGVTDYAEKFQGRVTITADPSTTTTSMELSSLRSEDTAVYYCARGGTNYLYWYFDLWGPGTPITVSS

SEQ ID NO: 84 - protein, 108 aa, Artificial Sequence
  Synthetic Construct
EIVMTQSPATLSLSPGERATLSCRASQSVSNNLAWYQQKPGQAPRLLIYDASSRATGIPARFSGSGSGTDFTLTISSLEPEDFAVYYCQQYSDWPQYTFGQGTKVEIK

SEQ ID NO: 85 - protein, 108 aa, Artificial Sequence
  Synthetic Construct
EIVLTQSPATLSLSPGERATLSCRASQSVSNNLAWYQQKPGQAPRLLIYDASSRATGIPARFSGSGSGTDFTLTISSLEPEDFAVYYCQQYSDWPQYTFGQGTKVEIK

SEQ ID NO: 86 - protein, 108 aa, Artificial Sequence
  Synthetic Construct
EIVMTQSPATLSLSPGERATLSCRASQSVSNNLAWYQQKPGQAPRLLIYDASSRATGIPARFSGSGSGTDFTLTISSLEPEDFAVYYCQQYSDWPEYSFGQGTKVEIK

SEQ ID NO: 87 - protein, 108 aa, Artificial Sequence
  Synthetic Construct
EIVMTQSPATLSLSPGERATLSCRASQGISSYLAWYQQKPGQAPRLLIYDASNLQSGIPARFSGSGSGTDFTLTISSLEPEDFAVYYCQQYSNWPQYSFGQGTKVEIK

SEQ ID NO: 88 - protein, 108 aa, Artificial Sequence
  Synthetic Construct
EIVMTQSPATLSLSPGERATLSCRASQSVSSYLAWYQQKPGQAPRLLIYDTSSRATGIPARFSGSGSGTDFTLTISSLEPEDFAVYYCQQYSNWPQYSFGQGTKVEIK

SEQ ID NO: 89 - protein, 108 aa, Artificial Sequence
  Synthetic Construct
EIVMTQSPATLSLSPGERATLSCRASQSVSSNLAWYQQKPGQAPRLLIYDASSRATGIPARFSGSGSGTDFTLTISSLEPEDFAVYYCQQYSNWPQYSFGQGTKVEIK

SEQ ID NO: 90 - protein, 108 aa, Artificial Sequence
  Synthetic Construct
EIVMTQSPATLSLSPGERATLSCRASQSVSSYLAWYQQKPGQAPRLLIYDASSRATGIPARFSGSGSGTDFTLTISSLEPEDFAVYYCQQYSDWPQYSFGQGTKVEIK

SEQ ID NO: 91 - protein, 111 aa, Artificial Sequence
  Synthetic Construct
QSALTQPPSASGSPGQSVTISCSGSSSDIGGYNRVSWYQQHPGKAPKLMIYDVSKRPSGVPDRFSGSKSGNTASLTVSGLQAEDEADYYCSSYAGTDTFILFGGGTKLTVL

SEQ ID NO: 92 - protein, 111 aa, Artificial Sequence
  Synthetic Construct
QSALTQPPSASGSPGQSVTISCTGTSSDIGGYDRVSWYQQHPGKAPKLMIYGVSKRPSGVPDRFSGSKSGNTASLTVSGLQAEDEADYYCSSYASGHTFGLFGGGTKLTVL

SEQ ID NO: 93 - protein, 111 aa, Artificial Sequence
  Synthetic Construct
QSALTQPPSASGSPGQSVTISCTGTSSDIGGYNRVSWYQQHPGKAPKLMIYEVNKRSSGVPDRFSGSKSGNTASLTVSGLQAEDEADYYCTSYAGTDTFVLFGGGTKLTVL

SEQ ID NO: 94 - protein, 111 aa, Artificial Sequence
  Synthetic Construct
QSALTQPPSASGSPGQSVTISCTGTTSDIGGYNRVSWYQQHPGKAPKLMIYEVSKRPSGVPDRFSGSKSGNTASLTVSGLQAEDEADYYCSSYAGTDTFAIFGGGTKLTVL

SEQ ID NO: 95 - protein, 111 aa, Artificial Sequence
  Synthetic Construct
QSALTQPPSASGSPGQSVTISCTGTISDIGGYNRVSWYQQHPGKAPKLMIYEVSKRPSGVPDRFSGSKSGNTASLTVSGLQAEDEADYYCSSYAGTDTFVFFGGGTKLTVL

SEQ ID NO: 96 - protein, 111 aa, Artificial Sequence
  Synthetic Construct
QSALTQPPSASGSPGQSVTISCTGTSSDIGGYNRVSWYQQHPGKAPKLMIYEFNKRPSGVPDRFSGSKSGNTASLTVSGLQAEDEADYYCSSYAGTDIFVLFGGGTKLTVL

SEQ ID NO: 97 - protein, 111 aa, Artificial Sequence
  Synthetic Construct
QSALTQPPSASGSPGQSVTISCTGSNSDIGGYNRVSWYQQHPGKAPKLMIYEISKRPSGVPDRFSGSKSGNTASLTVSGLQAEDEADYYCSSYAGTDTFVLFGGGTKLTVL

SEQ ID NO: 98 - protein, 112 aa, Artificial Sequence
  Synthetic Construct
DIVMTQTPLSLSVTPGEPASISCRSSQSLLHINGYTYLHWYLQKPGQSPQLLIYLVSHRASGVPDRFSGSGSDTDFTLKISRVEAEDVGVYYCEQTLQTPYSFGGGTKVEIK

SEQ ID NO: 99 - protein, 112 aa, Artificial Sequence
  Synthetic Construct
DIVMTQTPLSLSVTPGEPASISCRSSQSLLHSNEYTYLHWYLQKPGQSPQLLIYLVSHRASGVPDRFSGSGSDTDFTLKISRVEAEDVGVYYCEQTLQTPFTFGGGTKVEIK

SEQ ID NO: 100 - protein, 112 aa, Artificial Sequence
  Synthetic Construct
DIVMTQTPLSLSVTPGEPASISCRSSQSLLHSYGYTYLHWYLQKPGQSPQLLIYLVSYRASGVPDRFSGSGSDTDFTLKISRVEAEDVGVYYCEQTLQTPYTFGGGTKVEIK

SEQ ID NO: 101 - protein, 111 aa, Artificial Sequence
  Synthetic Construct
QSAPIQPRSVSGSPGQSVTISCTGTSSDIGGYNRISWYQQHPGKAPKLMIYDVSKRPSGVSDRFSGSKSGNTASLTISGLQAEDEADYYCSSYAGTDTFVLFGGGTQLTIV

SEQ ID NO: 102 - protein, 111 aa, Artificial Sequence
  Synthetic Construct
QSAPIQPRSVSGSPGQSVTISCTGTSSDIGGYNRVSWYQQHPGKAPKLMIYEVSKRPSGVSDRFSGSKSGNTASLTISGLQAEDEADYYCSAYAGTDTFVLFGGGTQLTIV

SEQ ID NO: 103 - protein, 16 aa, Artificial Sequence
  Synthetic Construct
  VARIANT 2: Xaa = Ile or Phe
  VARIANT 8: Xaa = Ile or Thr
YXSYYGSXSYNPSLKS

SEQ ID NO: 104 - protein, 8 aa, Artificial Sequence
  Synthetic Construct
  VARIANT 7: Xaa = Asp or Glu
  VARIANT 8: Xaa = Ser or Tyr
TNLLYFXX

SEQ ID NO: 105 - protein, 11 aa, Artificial Sequence
  Synthetic Construct
  VARIANT 5: Xaa = Gly or Ser
  VARIANT 6: Xaa = Val or Ile
  VARIANT 8: Xaa = Asn or Ser
  VARIANT 9: Xaa = Asn or Tyr
RASQXXSXXLA

SEQ ID NO: 106 - protein, 7 aa, Artificial Sequence
  Synthetic Construct
  VARIANT 2: Xaa = Ala or Thr
  VARIANT 4: Xaa = Ser or Asn
  VARIANT 5: Xaa = Arg or Leu
  VARIANT 6: Xaa = Ala or Gln
  VARIANT 7: Xaa = Thr or Ser
DXSXXXX

SEQ ID NO: 107 - protein, 10 aa, Artificial Sequence
  Synthetic Construct
  VARIANT 5: Xaa = Asp or Asn
  VARIANT 8: Xaa = Gln or Glu
  VARIANT 10: Xaa = Thr or Ser
QQYSXWPXYX

SEQ ID NO: 108 - protein, 5 aa, Artificial Sequence
  Synthetic Construct
  VARIANT 5: Xaa = Asn or Ser
SYGIX

SEQ ID NO: 109 - protein, 17 aa, Artificial Sequence
  Synthetic Construct
  VARIANT 3: Xaa = Val or Ile
  VARIANT 6: Xaa = Val or Leu
  VARIANT 7: Xaa = Gly or Asp
  VARIANT 9: Xaa = Thr or Pro
  VARIANT 10: Xaa = Ile or Asn
VIXPLXXVXXYAEKFQG

SEQ ID NO: 110 - protein, 11 aa, Artificial Sequence
  Synthetic Construct
  VARIANT 2: Xaa = Gln or Ala
GXEYFYWYFDL

SEQ ID NO: 111 - protein, 14 aa, Artificial Sequence
  Synthetic Construct
  VARIANT 1: Xaa = Ser or Thr
  VARIANT 3: Xaa = Ser or Thr
  VARIANT 4: Xaa = Ser, Thr, Ile, or Asn
  VARIANT 11: Xaa = Asn or Asp
XGXXSDIGGYXRVS

SEQ ID NO: 112 - protein, 7 aa, Artificial Sequence
  Synthetic Construct
  VARIANT 1: Xaa = Asp, Gly, or Glu
  VARIANT 2: Xaa = Val, Phe, or Ile
  VARIANT 3: Xaa = Ser or Asn
  VARIANT 6: Xaa = Pro or Ser
XXXKRXS

SEQ ID NO: 113 - protein, 11 aa, Artificial Sequence
  Synthetic Construct
  VARIANT 1: Xaa = Ser or Thr
  VARIANT 5: Xaa = Gly or Ser
  VARIANT 6: Xaa = Thr or Gly
  VARIANT 7: Xaa = Asp or His
  VARIANT 8: Xaa = Thr or Ile
  VARIANT 10: Xaa = Ile, Gly, Val, or Ala
  VARIANT 11: Xaa = Leu, Ile, or Phe
XSYAXXXXFXX

SEQ ID NO: 114 - protein, 5 aa, Artificial Sequence
  Synthetic Construct
  VARIANT 3: Xaa = Asp or Gly
NYXMT

SEQ ID NO: 115 - protein, 12 aa, Artificial Sequence
  Synthetic Construct
  VARIANT 6: Xaa = His or Tyr
GGGAYXGGSLDV

SEQ ID NO: 116 - protein, 16 aa, Artificial Sequence
  Synthetic Construct
  VARIANT 9: Xaa = Ile or Ser
  VARIANT 10: Xaa = Asn or Tyr
  VARIANT 11: Xaa = Gly or Glu
RSSQSLLHXXXYTYLH

SEQ ID NO: 117 - protein, 7 aa, Artificial Sequence
  Synthetic Construct
  VARIANT 4: Xaa = His or Tyr
LVSXRAS

SEQ ID NO: 118 - protein, 9 aa, Artificial Sequence
  Synthetic Construct
  VARIANT 8: Xaa = Tyr or Phe
  VARIANT 9: Xaa = Ser or Thr
EQTLQTPXX

SEQ ID NO: 119 - protein, 17 aa, Artificial Sequence
  Synthetic Construct
  VARIANT 2: Xaa = Leu or Ile
  VARIANT 3: Xaa = Val or Ile
  VARIANT 10: Xaa = Asn or Asp
MXXPLLGVTXYAEKFQG

SEQ ID NO: 120 - protein, 12 aa, Artificial Sequence
  Synthetic Construct
  VARIANT 3: Xaa = Ser or Thr
GGXNYLYWYFDL

SEQ ID NO: 121 - protein, 14 aa, Artificial Sequence
  Synthetic Construct
  VARIANT 13: Xaa = Val or Ile
TGTSSDIGGYNRXS

SEQ ID NO: 122 - protein, 7 aa, Artificial Sequence
  Synthetic Construct
  VARIANT 1: Xaa = Asp or Glu
XVSKRPS

SEQ ID NO: 123 - protein, 11 aa, Artificial Sequence
  Synthetic Construct
  VARIANT 2: Xaa = Ser or Ala
SXYAGTDTFVL

SEQ ID NO: 124 - protein, 330 aa, Artificial Sequence
  Synthetic Construct
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 125 - protein, 327 aa, Artificial Sequence
  Synthetic Construct
ASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPSCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 126 - protein, 107 aa, Artificial Sequence [sequence omitted]

SEQ ID NO: 127 - protein, 106 aa, Artificial Sequence
  Synthetic Construct
GQPKAAPSVTLFPPSSEELQANKATLVCLISDFYPGAVTVAWKADSSPVKAGVETTTPSKQSNNKYAASSYLSLTPEQWKSHRSYSCQVTHEGSTVEKTVAPTECS

SEQ ID NO: 128 - protein, 159 aa, Artificial Sequence
  Synthetic Construct
MFPFALLYVLSVSFRKIFILQLVGLVLTYDFTNCDFEKIKAAYLSTISKDLITYMSGTKSTEFNNTVSCSNRPHCLTEIQSLTFNPTAGCASLAKEMFAMKTKAALAIWCPGYSETQINATQAMKKRRKRKVTTNKCLEQVSQLQGLWRRFNRPLLKQQ

SEQ ID NO: 129 - protein, 159 aa, Artificial Sequence
  Synthetic Construct
MFPFALLYVLSVSFRKIFILQLVGLVLTYDFTNCDFEKIKAAYLSTISKDLITYMSGTKSTEFNNTVSCSNRPHCLTEIQSLTFNPTAGCASLAKEMFAMKTKAALAIWCPGYSETQINATQAMKKARKSKVTTNKCLEQVSQLQGLWRRFNRPLLKQQ